SEQUENCE LISTING (283 sequences)

SEQ ID NO: 1 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 2 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 3 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 4 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 227 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 223 aa, Homo sapiens
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 13 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 14 - protein, 112 aa, Homo sapiens
FTVRETASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 15 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 17 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 18 - protein, 266 aa, Homo sapiens
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 19 - protein, 266 aa, Homo sapiens [sequence omitted]

SEQ ID NO: 20 - protein, 266 aa, Homo sapiens [sequence omitted]

SEQ ID NO: 21 - protein, 266 aa, Homo sapiens
MVCLRLPGGSCMAVLTVTLMVLSSPLALAGDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKMLSGVGGFVLGLLFLGAGLFIYFRNQKGHSGLQPRGFLS

SEQ ID NO: 22 - protein, 266 aa, Homo sapiens [sequence omitted]

SEQ ID NO: 23 - protein, 266 aa, Homo sapiens [sequence omitted]

SEQ ID NO: 24 - protein, 266 aa, Homo sapiens [sequence omitted]

SEQ ID NO: 25 - protein, 266 aa, Homo sapiens [sequence omitted]

SEQ ID NO: 26 - protein, 266 aa, Homo sapiens
MVCLKFPGGSCMAALTVTLMVLSSPLALAGDTRPRFLEQVKHECHFFNGTERVRFLDRYFYHQEEYVRFDSDVGEYRAVTELGRPDAEYWNSQKDLLEQRRAEVDTYCRHNYGVVESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKMLSGVGGFVLGLLFLGAGLFIYFRNQKGHSGLQPTGFLS

SEQ ID NO: 27 - protein, 266 aa, Homo sapiens [sequence omitted]

SEQ ID NO: 28 - protein, 266 aa, Homo sapiens [sequence omitted]

SEQ ID NO: 29 - protein, 266 aa, Homo sapiens [sequence omitted]

SEQ ID NO: 30 - protein, 266 aa, Homo sapiens [sequence omitted]

SEQ ID NO: 31 - protein, 266 aa, Homo sapiens
MVCLKLPGGSCMAALTVTLMVLSSPLALAGDTQPRFLWQGKYKCHFFNGTERVQFLERLFYNQEEFVRFDSDVGEYRAVTELGRPVAESWNSQKDILEDRRGQVDTVCRHNYGVGESFTVQRRVHPEVTVYPAKTQPLQHHNLLVCSVSGFYPGSIEVRWFRNGQEEKAGVVSTGLIQNGDWTFQTLVMLETVPRSGEVYTCQVEHPSVMSPLTVEWRARSESAQSKMLSGVGGFVLGLLFLGAGLFIYFRNQKGHSGLQPTGFLS

SEQ ID NO: 32 - protein, 266 aa, Homo sapiens [sequence omitted]

SEQ ID NO: 33 - protein, 266 aa, Homo sapiens [sequence omitted]

SEQ ID NO: 34 - protein, 266 aa, Homo sapiens [sequence omitted]

SEQ ID NO: 35 - protein, 266 aa, Homo sapiens [sequence omitted]

SEQ ID NO: 36 - protein, 266 aa, Homo sapiens [sequence omitted]

SEQ ID NO: 37 - protein, 266 aa, Homo sapiens [sequence omitted]

SEQ ID NO: 38 - protein, 266 aa, Homo sapiens
MVCLRLPGGSCMAVLTVTLMVLSSPLALAGDTRPRFLEYSTSECHFFNGTERVRFLDRYFYNQEEYVRFDSDVGEFRAVTELGRPDEEYWNSQKDFLEDERAAVDTYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKMLSGVGGFVLGLLFLGAGLFIYFRNQKGHSGLQPRGFLS

SEQ ID NO: 39 - protein, 266 aa, Homo sapiens [sequence omitted]

SEQ ID NO: 40 - protein, 266 aa, Homo sapiens [sequence omitted]

SEQ ID NO: 41 - protein, 266 aa, Homo sapiens [sequence omitted]

SEQ ID NO: 42 - protein, 266 aa, Homo sapiens [sequence omitted]

SEQ ID NO: 43 - protein, 266 aa, Homo sapiens [sequence omitted]

SEQ ID NO: 44 - protein, 266 aa, Homo sapiens [sequence omitted]

SEQ ID NO: 45 - protein, 266 aa, Homo sapiens [sequence omitted]

SEQ ID NO: 46 - protein, 266 aa, Homo sapiens [sequence omitted]

SEQ ID NO: 47 - protein, 266 aa, Homo sapiens
MVCLKLPGGSCMTALTVTLMVLSSPLALSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 48 - protein, 266 aa, Homo sapiens [sequence omitted]

SEQ ID NO: 49 - protein, 266 aa, Homo sapiens [sequence omitted]

SEQ ID NO: 50 - protein, 266 aa, Homo sapiens [sequence omitted]

SEQ ID NO: 51 - protein, 266 aa, Homo sapiens; identical to SEQ ID NO: 47

SEQ ID NO: 52 - protein, 266 aa, Homo sapiens; identical to SEQ ID NO: 47

SEQ ID NO: 53 - protein, 266 aa, Homo sapiens [sequence omitted]

SEQ ID NO: 54 - protein, 266 aa, Homo sapiens [sequence omitted]

SEQ ID NO: 55 - protein, 266 aa, Homo sapiens
MVCLKLPGGSSLAALTVTLMVLSSRLAFAGDTRPRFLELRKSECHFFNGTERVRYLDRYFHNQEEFLRFDSDVGEYRAVTELGRPVAESWNSQKDLLEQKRGRVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSKMLSGVGGFVLGLLFLGAGLFIYFRNQKGHSGLQPTGFLS

SEQ ID NO: 56 - protein, 266 aa, Homo sapiens [sequence omitted]

SEQ ID NO: 57 - protein, 266 aa, Homo sapiens
MVCLKLPGGSSLAALTVTLMVLSSRLAFAGDTRPRFLELLKSECHFFNGTERVRFLERHFHNQEEYARFDSDVGEYRAVRELGRPDAEYWNSQKDLLEQKRGQVDNYCRHNYGVVESFTVQRRVHPQVTVYPAKTQPLQHHNLLVCSVSGFYPGSIEVRWFRNGQEEKAGVVSTGLIQNGDWTFQTLVMLETFPRSGEVYTCQVEHPSVTSPLTVEWSARSESAQSKMLSGVGGFVLGLLFLGAGLFIYFRNQKGHSGLQPTGFLS

SEQ ID NO: 58 - protein, 266 aa, Homo sapiens [sequence omitted]

SEQ ID NO: 59 - protein, 266 aa, Homo sapiens
MVCLKLPGGSCMAALTVTLTVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNSQEEKAGVVSTGLIQNGDWTFQTLVMLETVPRSGEVYTCQVEHPSMMSPLTVQWSARSESAQSKMLSGVGGFVLGLLFLGTGLFIYFRNQKGHSGLQPTGLLS

SEQ ID NO: 60 - protein, 266 aa, Homo sapiens
MVCLKLPGGSCMAALTVTLTVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSKMLSGVGGFVLGLLFLGTGLFIYFRNQKGHSGLQPTGLLS

SEQ ID NO: 61 - protein, 266 aa, Homo sapiens
MVCLKLPGGSYMAKLTVTLMVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRVEPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQSKMLSGVGGFVLGLLFLGAGLFIYFKNQKGHSGLHPTGLVS

SEQ ID NO: 62 - protein, 261 aa, Homo sapiens
MGHEQNQGAALLQMLPLLWLLPHSWAVPEAPTPMWPDDLQNHTFLHTVYCQDGSPSVGLSEAYDEDQLFFFDFSQNTRVPRLPEFADWAQEQGDAPAILFDKEFCEWMIQQIGPKLDGKIPVSRGFPIAEVFTLKPLEFGKPNTLVCFVSNLFPPMLTVNWQHHSVPVEGFGPTFVSAVDGLSFQAFSYLNFTPEPSDIFSCIVTHEIDRYTAIAYWVPRNALPSDLLENVLCGVAFGLGVLGIIVGIVLIIYFRKPCSGD

SEQ ID NO: 63 - protein, 263 aa, Homo sapiens
MITFLPLLLGLSLGCTGAGGFVAHVESTCLLDDAGTPKDFTYCISFNKDLLTCWDPEENKMAPCEFGVLNSLANVLSQHLNQKDTLMQRLRNGLQNCATHTQPFWGSLTNRTRPPSVQVAKTTPFNTREPVMLACYVWGFYPAEVTITWRKNGKLVMPHSSAHKTAQPNGDWTYQTLSHLALTPSYGDTYTCVVEHTGAPEPILRDWTPGLSPMQTLKVSVSAVTLGLGLIIFSLGVISWRRAGHSSYTPLPGSNYSEGWHIS

SEQ ID NO: 64 - protein, 250 aa, Homo sapiens
MALRAGLVLGFHTLMTLLSPQEAGATKADHMGSYGPAFYQSYGASGQFTHEFDEEQLFSVDLKKSEAVWRLPEFGDFARFDPQGGLAGIAAIKAHLDILVERSNRSRAINVPPRVTVLPKSRVELGQPNILICIVDNIFPPVINITWLRNGQTVTEGVAQTSFYSQPDHLFRKFHYLPFVPSAEDVYDCQVEHWGLDAPLLRHWELQVPIPPPDAMETLVCALGLAIGLVGFLVGTVLIIMGTYVSSVPR

SEQ ID NO: 65 - protein, 273 aa, Homo sapiens
MGSGWVPWVVALLVNLTRLDSSMTQGTDSPEDFVIQAKADCYFTNGTEKVQFVVRFIFNLEEYVRFDSDVGMFVALTKLGQPDAEQWNSRLDLLERSRQAVDGVCRHNYRLGAPFTVGRKVQPEVTVYPERTPLLHQHNLLHCSVTGFYPGDIKIKWFLNGQEERAGVMSTGPIRNGDWTFQTVVMLEMTPELGHVYTCLVDHSSLLSPVSVEWRAQSEYSWRKMLSGIAAFLLGLIFLLVGIVIQLRAQKGYVRTQMSGNEVSRAVLLPQSC

SEQ ID NO: 66 - protein, 260 aa, Homo sapiens
MRPEDRMFHIRAVILRALSLAFLLSLRGAGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 67 - protein, 260 aa, Homo sapiens [sequence omitted]

SEQ ID NO: 68 - protein, 258 aa, Homo sapiens [sequence omitted]

SEQ ID NO: 69 - protein, 258 aa, Homo sapiens
MMVLQVSAAPRTVALTALLMVLLTSVVQGRATPENYLFQGRQECYAFNGTQRFLERYIYNREEFVRFDSDVGEFRAVTELGRPDEEYWNSQKDILEEERAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQSDSARSKTLTGAGGFVLGLIICGVGIFMHRRSKKVQRGSA

SEQ ID NO: 70 - protein, 258 aa, Homo sapiens
MMVLQVSAAPRTVALTALLMVLLTSVVQGRATPENYVYQLRQECYAFNGTQRFLERYIYNREEFVRFDSDVGEFRAVTELGRPDEDYWNSQKDLLEEKRAVPDRVCRHNYELDEAVTLQRRVQPKVNVSPSKKGPLQHHNLLVCHVTDFYPGSIQVRWFLNGQEETAGVVSTNLIRNGDWTFQILVMLEMTPQQGDVYICQVEHTSLDSPVTVEWKAQSDSARSKTLTGAGGFVLGLIICGVGIFMHRRSKKVQRGSA

SEQ ID NO: 71 - protein, 258 aa, Homo sapiens
MMVLQVSAAPRTVALTALLMVLLTSVVQGRATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQSDSARSKTLTGAGGFVLGLIICGVGIFMHRRSKKVQRGSA

SEQ ID NO: 72 - protein, 258 aa, Homo sapiens [sequence omitted]

SEQ ID NO: 73 - protein, 258 aa, Homo sapiens [sequence omitted]

SEQ ID NO: 74 - protein, 258 aa, Homo sapiens [sequence omitted]

SEQ ID NO: 75 - protein, 258 aa, Homo sapiens [sequence omitted]

SEQ ID NO: 76 - protein, 258 aa, Homo sapiens [sequence omitted]

SEQ ID NO: 77 - protein, 258 aa, Homo sapiens [sequence omitted]

SEQ ID NO: 78 - protein, 258 aa, Homo sapiens [sequence omitted]

SEQ ID NO: 79 - protein, 258 aa, Homo sapiens [sequence omitted]

SEQ ID NO: 80 - protein, 255 aa, Homo sapiens [sequence omitted]

SEQ ID NO: 81 - protein, 255 aa, Homo sapiens
MILNKALLLGALALTTVMSPCGGEDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTETVVCALGLSVGLMGIVVGTVFIIQGLRSVGASRHQGPL

SEQ ID NO: 82 - protein, 255 aa, Homo sapiens [sequence omitted]

SEQ ID NO: 83 - protein, 255 aa, Homo sapiens [sequence omitted]

SEQ ID NO: 84 - protein, 254 aa, Homo sapiens
MILNKALMLGALALTTVMSPCGGEDIVADHVASYGVNLYQSYGPSGQFTHEFDGDEEFYVDLERKETVWKLPLFHRLRFDPQFALTNIAVLKHNLNILIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPAPMSELTETVVCALGLSVGLVGIVVGTVLIIRGLRSVGASRHQGPL

SEQ ID NO: 85 - protein, 254 aa, Homo sapiens
MILNKALMLGALALTTVMSPCGGEDIVADHVASYGVNLYQSYGPSGQYSHEFDGDEEFYVDLERKETVWQLPLFRRFRRFDPQFALTNIAVLKHNLNIVIKRSNSTAATNEVPEVTVFSKSPVTLGQPNTLICLVDNIFPPVVNITWLSNGHSVTEGVSTSFLSKSDHSFFKISYLTFLPSADEIYDCKVEHWGLDEPLLKHWEPEIPTPMSELTETVVCALGLSVGLVGIVVGTVLIIRGLRSVGASRHQGPL

SEQ ID NO: 86 - protein, 255 aa, Homo sapiens [sequence omitted]

SEQ ID NO: 87 - protein, 254 aa, Homo sapiens
MILNKALLLGALALTTVMSPCGGEDIVADHVASYGVNLYQSYGPSGQYTHEFDGDEQFYVDLGRKETVWCLPVLRQFRFDPQFALTNIAVTKHNLNILIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPAPMSELTETVVCALGLSVGLVGIVVGTVFIIRGLRSVGASRHQGPL

SEQ ID NO: 88 - protein, 254 aa, Homo sapiens
MILNKALMLGALALTTVMSPCGGEDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 89 - protein, 254 aa, Homo sapiens
MILNKALMLGTLALTTVMSPCGGEDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 90 - protein, 254 aa, Homo sapiens [sequence omitted]

SEQ ID NO: 91 - protein, 255 aa, Homo sapiens
MILNKALLLGALALTAVMSPCGGEDIVADHVASYGVNFYQSHGPSGQYTHEFDGDEEFYVDLETKETVWQLPMFSKFISFDPQSALRNMAVGKHTLEFMMRQSNSTAATNEVPEVTVFSKFPVTLGQPNTLICLVDNIFPPVVNITWLSNGHSVTEGVSETSFLSKSDHSFFKISYLTFLPSADEIYDCKVEHWGLDEPLLKHWEPEIPAPMSELTETLVCALGLSVGLMGIVVGTVFIIQGLRSVGASRHQGLL

SEQ ID NO: 92 - protein, 261 aa, Homo sapiens
MSWKKALRIPGGLRAATVTLMLSMLSTPVAEGRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKMLSGIGGFVLGLIFLGLGLIIHHRSQKGLLH

SEQ ID NO: 93 - protein, 261 aa, Homo sapiens
MSWKKALRIPGGLRAATVTLMLSMLSTPVAEGRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNGQEETAGVVSTPLIRNGDWTFQILVMLEMTPQRGDVYTCHVEHPSLQSPITVEWRAQSESAQSKMLSGIGGFVLGLIFLGLGLIIHHRSQKGLLH

SEQ ID NO: 94 - protein, 261 aa, Homo sapiens
MSWKKALRIPGGLRAATVTLMLAMLSTPVAEGRDSPEDFVYQFKAMCYFTNGTERVRYVTRYIYNREEYARFDSDVEVYRAVTPLGPPDAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQHGDVYTCHVEHPSLQNPITVEWRAQSESAQSKMLSGIGGFVLGLIFLGLGLIIHHRSQKGLLH

SEQ ID NO: 95 - protein, 261 aa, Homo sapiens [sequence omitted]

SEQ ID NO: 96 - protein, 261 aa, Homo sapiens [sequence omitted]

SEQ ID NO: 97 - protein, 261 aa, Homo sapiens [sequence omitted]

SEQ ID NO: 98 - protein, 261 aa, Homo sapiens [sequence omitted]

SEQ ID NO: 99 - protein, 261 aa, Homo sapiens [sequence omitted]

SEQ ID NO: 100 - protein, 261 aa, Homo sapiens [sequence omitted]

SEQ ID NO: 101 - protein, 269 aa, Homo sapiens [sequence omitted]

SEQ ID NO: 102 - protein, 269 aa, Homo sapiens [sequence omitted]

SEQ ID NO: 103 - protein, 269 aa, Homo sapiens
MSWKKALRIPGDLRVATVTLMLAMLSSLLAEGRDSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSKMLSGVGGFVLGLIFLGLGLIIRQRSQKGPQGPPPAGLLH

SEQ ID NO: 104 - protein, 227 aa, Homo sapiens
MALQIPGGFWAAAVTVMLVMLSTPVAEARDFPKDFLVQFKGMCYFTNGTERVRGVARYIYNREEYGRFDSDVGEFQAVTELGRSIEDWNNYKDFLEQERAAVDKVCRHNYEAELRTTLQRQVEPTVTISPSRTEALNHHNLLVCSVTDFYPAQIKVRWFRNDQEETAGVVSTSLIRNGDWTFQILVMLEITPQRGDIYTCQVEHPSLQSPITVEWRPRGPPPAGLLH

SEQ ID NO: 105 - protein, 264 aa, Homo sapiens
MALQIPGGFWAAAVTVMLVMLSTPVAEARDFPKDFLVQFKGMCYFTNGTERVRGVARYIYNREEYGRFDSDVGEFQAVTELGRSIEDWNNYKDFLEQERAAVDKVCRHNYEAELRTTLQRQVEPTVTISPSRTEALNHHNLLVCSVTDFYPAQIKVRWFRNDQEETAGVVSTSLIRNGDWTFQILVMLEITPQRGDIYTCQVEHPSLQSPITVEWRAQSESAQSKMLSGIGGFVLGLIFLGLGLIIRHRGQKGPRGPPPAGLLH

SEQ ID NO: 106 - protein, 33 aa, Homo sapiens
RMKQIEDKIEEILSKIYHIENEIARIKKLIGER

SEQ ID NO: 107 - protein, 32 aa, Homo sapiens
LSSIEKKQEEQTSWLIWISNELTLIRNELAQS

SEQ ID NO: 108 - protein, 31 aa, Homo sapiens
LSSIEKKLEEITSQLIQISNELTLIRNELAQ

SEQ ID NO: 109 - protein, 31 aa, Homo sapiens
LSSIEKKLEEITSQLIQIRNELTLIRNELAQ

SEQ ID NO: 110 - protein, 31 aa, Homo sapiens
LSSIEKKLEEITSQLQQIRNELTLIRNELAQ

SEQ ID NO: 111 - protein, 31 aa, Homo sapiens
LSSLEKKLEELTSQLIQLRNELTLLRNELAQ

SEQ ID NO: 112 - protein, 31 aa, Homo sapiens
ISSLEKKIEELTSQIQQLRNEITLLRNEIAQ

SEQ ID NO: 113 - protein, 47 aa, Homo sapiens
LEIEAAFLERENTALETRVAELRQRVQRLRNRVSQYRTRYGPLGGGK

SEQ ID NO: 114 - protein, 31 aa, Homo sapiens
LKSVENRLAVVENQLKTVIEELKTVKDLLSN

SEQ ID NO: 115 - protein, 31 aa, Homo sapiens
LARIEEKLKTIKAQLSEIASTLNMIREQLAQ

SEQ ID NO: 116 - protein, 31 aa, Homo sapiens
VSRLEEKVKTLKSQVTELASTVSLLREQVAQ

SEQ ID NO: 117 - protein, 30 aa, Homo sapiens
IQSEKKIEDISSLIGQIQSEITLIRNEIAQ

SEQ ID NO: 118 - protein, 31 aa, Homo sapiens
LMSLEKKLEELTQTLMQLQNELSMLKNELAQ

SEQ ID NO: 119 - protein, 18 aa, Homo sapiens
VDLEGSTSNGRQCAGIRL

SEQ ID NO: 120 - protein, 27 aa, Homo sapiens
EDDVTTTEELAPALVPPPKGTCAGWMA

SEQ ID NO: 121 - protein, 27 aa, Homo sapiens
GHDQETTTQGPGVLLPLPKGACTGQMA

SEQ ID NO: 122 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 123 - protein, 64 aa, Homo sapiens
VTAFSNMDDMLQKAHLVIEGTFIYLRDSTEFFIRVRDGWKKLQLGELIPIPADSPPPPALSSNP

SEQ ID NO: 124 - protein, 79 aa, Homo sapiens
VIIQAEFYLNPDQSGEFMFDFDGDEIFHVDMAKKETVWRLEEFGRFASFEAQGALANIAVDKANLEIMTKRSNYTPITN

SEQ ID NO: 125 - protein, 178 aa, Homo sapiens
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 126 - protein, 94 aa, Homo sapiens
VPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 127 - protein, 98 aa, Homo sapiens
VPEAPTPMWPDDLQNHTFLHTVYCQDGSPSVGLSEAYDEDQLFFFDFSQNTRVPRLPEFADWAQEQGDAPAILFDKEFCEWMIQQIGPKLDGKIPVSR

SEQ ID NO: 128 - protein, 93 aa, Homo sapiens
GFPIAEVFTLKPLEFGKPNTLVCFVSNLFPPMLTVNWQHHSVPVEGFGPTFVSAVDGLSFQAFSYLNFTPEPSDIFSCIVTHEIDRYTAIAYW

SEQ ID NO: 129 - protein, 85 aa, Homo sapiens
TKADHMGSYGPAFYQSYGASGQFTHEFDEEQLFSVDLKKSEAVWRLPEFGDFARFDPQGGLAGIAAIKAHLDILVERSNRSRAIN

SEQ ID NO: 130 - protein, 94 aa, Homo sapiens
VPPRVTVLPKSRVELGQPNILICIVDNIFPPVINITWLRNGQTVTEGVAQTSFYSQPDHLFRKFHYLPFVPSAEDVYDCQVEHWGLDAPLLRHW

SEQ ID NO: 131 - protein, 85 aa, Homo sapiens
AIKADHVSTYAAFVQTHRPTGEFMFEFDEDEMFYVDLDKKETVWHLEEFGQAFSFEAQGGLANIAILNNNLNTLIQRSNHTQATN

SEQ ID NO: 132 - protein, 94 aa, Homo sapiens
DPPEVTVFPKEPVELGQPNTLICHIDKFFPPVLNVTWLCNGELVTEGVAESLFLPRTDYSFHKFHYLTFVPSAEDFYDCRVEHWGLDQPLLKHW

SEQ ID NO: 133 - protein, 181 aa, Homo sapiens
EDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHW

SEQ ID NO: 134 - protein, 87 aa, Homo sapiens
EDIVADHVASCGVNLYQFYGPSGQYTHEFDGDEQFYVDLERKETAWRWPEFSKFGGFDPQGALRNMAVAKHNLNIMIKRYNSTAATN

SEQ ID NO: 135 - protein, 94 aa, Homo sapiens
EVPEVTVFSKSPVTLGQPNTLICLVDNIFPPVVNITWLSNGQSVTEGVSETSFLSKSDHSFFKISYLTFLPSADEIYDCKVEHWGLDQPLLKHW

SEQ ID NO: 136 - protein, 181 aa, Homo sapiens
EDIVADHVASYGVNFYQSHGPSGQYTHEFDGDEEFYVDLETKETVWQLPMFSKFISFDPQSALRNMAVGKHTLEFMMRQSNSTAATNEVPEVTVFSKFPVTLGQPNTLICLVDNIFPPVVNITWLSNGHSVTEGVSETSFLSKSDHSFFKISYLTFLPSADEIYDCKVEHWGLDEPLLKHW

SEQ ID NO: 137 - protein, 87 aa, Homo sapiens
EDIVADHVASYGVNFYQSHGPSGQYTHEFDGDEEFYVDLETKETVWQLPMFSKFISFDPQSALRNMAVGKHTLEFMMRQSNSTAATN

SEQ ID NO: 138 - protein, 94 aa, Homo sapiens
EVPEVTVFSKFPVTLGQPNTLICLVDNIFPPVVNITWLSNGHSVTEGVSETSFLSKSDHSFFKISYLTFLPSADEIYDCKVEHWGLDEPLLKHW

SEQ ID NO: 139 - protein, 199 aa, Homo sapiens
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 140 - protein, 94 aa, Homo sapiens
DTRPRFLEQVKHECHFFNGTERVRFLDRYFYHQEEYVRFDSDVGEYRAVTELGRPDAEYWNSQKDLLEQKRAAVDTYCRHNYGVGESFTVQRRV

SEQ ID NO: 141 - protein, 95 aa, Homo sapiens
GDTRCRFLEQVKHECHFFNGTERVRFLDRYFYHQEEYVRFDSDVGEYRAVTELGRPDAEYWNSQKDLLEQKRAAVDTYCRHNYGVGESFTVQRRV

SEQ ID NO: 142 - protein, 103 aa, Homo sapiens
YPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 143 - protein, 104 aa, Homo sapiens
YPEVTVYPAKTQPLQHHNLLVCSVNGFYPASIEVRWFRNGQEEKTGVVSTGLIQNGDCTFQTLVMLETVPRSGEVYTCQVEHPSLTSPLTVEWRARSESAQSKM

SEQ ID NO: 144 - protein, 197 aa, Homo sapiens
DTRPRFLELRKSECHFFNGTERVRYLDRYFHNQEEFLRFDSDVGEYRAVTELGRPVAESWNSQKDLLEQKRGRVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSK

SEQ ID NO: 145 - protein, 29 aa, Homo sapiens
MVCLKLPGGSSLAALTVTLMVLSSRLAFA

SEQ ID NO: 146 - protein, 94 aa, Homo sapiens
DTRPRFLELRKSECHFFNGTERVRYLDRYFHNQEEFLRFDSDVGEYRAVTELGRPVAESWNSQKDLLEQKRGRVDNYCRHNYGVGESFTVQRRV

SEQ ID NO: 147 - protein, 103 aa, Homo sapiens
HPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSK

SEQ ID NO: 148 - protein, 19 aa, Homo sapiens
MVCLKLPGGSCMAALTVTL

SEQ ID NO: 149 - protein, 105 aa, Homo sapiens
TVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRV

SEQ ID NO: 150 - protein, 103 aa, Homo sapiens
QPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSK

SEQ ID NO: 151 - protein, 105 aa, Homo sapiens
MVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRV

SEQ ID NO: 152 - protein, 102 aa, Homo sapiens
EPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQS

SEQ ID NO: 153 - protein, 94 aa, Homo sapiens
GGFVAHVESTCLLDDAGTPKDFTYCISFNKDLLTCWDPEENKMAPCEFGVLNSLANVLSQHLNQKDTLMQRLRNGLQNCATHTQPFWGSLTNRT

SEQ ID NO: 154 - protein, 95 aa, Homo sapiens
RPPSVQVAKTTPFNTREPVMLACYVWGFYPAEVTITWRKNGKLVMPHSSAHKTAQPNGDWTYQTLSHLALTPSYGDTYTCVVEHTGAPEPILRDW

SEQ ID NO: 155 - protein, 94 aa, Homo sapiens
TDSPEDFVIQAKADCYFTNGTEKVQFVVRFIFNLEEYVRFDSDVGMFVALTKLGQPDAEQWNSRLDLLERSRQAVDGVCRHNYRLGAPFTVGRK

SEQ ID NO: 156 - protein, 94 aa, Homo sapiens
VQPEVTVYPERTPLLHQHNLLHCSVTGFYPGDIKIKWFLNGQEERAGVMSTGPIRNGDWTFQTVVMLEMTPELGHVYTCLVDHSSLLSPVSVEW

SEQ ID NO: 157 - protein, 186 aa, Homo sapiens
RATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEW

SEQ ID NO: 158 - protein, 92 aa, Homo sapiens
RATPENYLFQGRQECYAFNGTQRFLERYIYNREEFARFDSDVGEFRAVTELGRPAAEYWNSQKDILEEKRAVPDRMCRHNYELGGPMTLQRR

SEQ ID NO: 159 - protein, 94 aa, Homo sapiens
VQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEW

SEQ ID NO: 160 - protein, 94 aa, Homo sapiens
RDSPEDFVFQFKGMCYFTNGTERVRLVTRYIYNREEYARFDSDVGVYRAVTPQGRPDAEYWNSQKEVLEGTRAELDTVCRHNYEVAFRGILQRR

SEQ ID NO: 161 - protein, 94 aa, Homo sapiens
VEPTVTISPSRTEALNHHNLLVCSVTDFYPGQIKVRWFRNDQEETAGVVSTPLIRNGDWTFQILVMLEMTPQRGDVYTCHVEHPSLQSPITVEW

SEQ ID NO: 162 - protein, 182 aa, Homo sapiens
DFLVQFKGMCYFTNGTERVRGVARYIYNREEYGRFDSDVGEFQAVTELGRSIEDWNNYKDFLEQERAAVDKVCRHNYEAELRTTLQRQVEPTVTISPSRTEALNHHNLLVCSVTDFYPAQIKVRWFRNDQEETAGVVSTSLIRNGDWTFQILVMLEITPQRGDIYTCQVEHPSLQSPITVEW

SEQ ID NO: 163 - protein, 93 aa, Homo sapiens
DFLVQFKGMCYFTNGTERVRGVARYIYNREEYGRFDSDVGEFQAVTELGRSIEDWNNYKDFLEQERAAVDKVCRHNYEAELRTTLQRQVEPTV

SEQ ID NO: 164 - protein, 89 aa, Homo sapiens
TISPSRTEALNHHNLLVCSVTDFYPAQIKVRWFRNDQEETAGVVSTSLIRNGDWTFQILVMLEITPQRGDIYTCQVEHPSLQSPITVEW

SEQ ID NO: 165 - protein, 20 aa, Artificial Sequence
  Peptide suitable for use as a linker.
GCGGSGGGGSGGGGSGGGGS

SEQ ID NO: 166 - protein, 133 aa, Homo sapiens [sequence omitted]

SEQ ID NO: 167 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 168 - protein, 524 aa, Homo sapiens
VNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 169 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 170 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 171 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 172 - protein, 150 aa, Homo sapiens
IGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYK

SEQ ID NO: 173 - protein, 179 aa, Homo sapiens
QLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 174 - protein, 245 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 175 - protein, 219 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 176 - protein, 227 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERLNVSIKI

SEQ ID NO: 177 - protein, 124 aa, Homo sapiens
AFTVTVPKDLYVVEYGSNMTIECKFPVEKQLDLAALIVYWEMEDKNIIQFVHGEEDLKTQHSSYRQRARLLKDQLSLGNAALQITDVKLQDAGVYRCMISYGGADYKRITVKVNAPYAAALHEH

SEQ ID NO: 178 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 179 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 180 - protein, 112 aa, Homo sapiens
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 181 - protein, 112 aa, Homo sapiens [sequence omitted]

SEQ ID NO: 182 - protein, 208 aa, Homo sapiens [sequence omitted]

SEQ ID NO: 183 - protein, 104 aa, Homo sapiens
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSV

SEQ ID NO: 184 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 185 - protein, 123 aa, Homo sapiens
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 186 - protein, 101 aa, Homo sapiens
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 187 - protein, 224 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 188 - protein, 110 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVL

SEQ ID NO: 189 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 190 - protein, 174 aa, Homo sapiens
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 191 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 192 - protein, 167 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 193 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 194 - protein, 5 aa, Artificial Sequence
  Peptide suitable for use as a linker.
GSGGS

SEQ ID NO: 195 - protein, 4 aa, Artificial Sequence
  Peptide suitable for use as a linker.
GGGS

SEQ ID NO: 196 - protein, 4 aa, Artificial Sequence
  Peptide suitable for use as a linker.
GGSG

SEQ ID NO: 197 - protein, 5 aa, Artificial Sequence
  Peptide suitable for use as a linker.
GGSGG

SEQ ID NO: 198 - protein, 5 aa, Artificial Sequence
  Peptide suitable for use as a linker.
GSGSG

SEQ ID NO: 199 - protein, 5 aa, Artificial Sequence
  Peptide suitable for use as a linker.
GSGGG

SEQ ID NO: 200 - protein, 5 aa, Artificial Sequence
  Peptide suitable for use as a linker.
GGGSG

SEQ ID NO: 201 - protein, 5 aa, Artificial Sequence
  Peptide suitable for use as a linker.
GSSSG

SEQ ID NO: 202 - protein, 5 aa, Artificial Sequence
  Peptide suitable for use as a linker.
GSSSS

SEQ ID NO: 203 - protein, 5 aa, Artificial Sequence
  Peptide suitable for use as a linker.
GGGGS

SEQ ID NO: 204 - protein, 5 aa, Artificial Sequence
  Peptide suitable for use as a linker.
AAAGG

SEQ ID NO: 205 - protein, 5 aa, Artificial Sequence
  Peptide suitable for use as a linker. The EAAAK may be repeated 2 to 20 times.
EAAAK

SEQ ID NO: 206 - protein, 7 aa, Artificial Sequence
  Peptide suitable for use as a linker. The EAAAK subset may be repeated 2 to 20 times.
AEAAAKA

SEQ ID NO: 207 - protein, 16 aa, Artificial Sequence
  Peptide suitable for use as a linker. The EAAAK subset may be repeated independently 2 to 20 times.
AEAAAKALEAEAAAKA

SEQ ID NO: 208 - protein, 5 aa, Artificial Sequence
  Peptide suitable for use as a linker
EAAAK

SEQ ID NO: 209 - protein, 10 aa, Artificial Sequence
  Peptide suitable for use as a linker.
EAAAKEAAAK

SEQ ID NO: 210 - protein, 15 aa, Artificial Sequence
  Peptide suitable for use as a linker.
EAAAKEAAAKEAAAK

SEQ ID NO: 211 - protein, 46 aa, Artificial Sequence
  Peptide suitable for use as a linker.
AEAAAKEAAAKEAAAKEAAAKALEAEAAAKEAAAKEAAAKEAAAKA

SEQ ID NO: 212 - protein, 12 aa, Artificial Sequence
  Peptide suitable for use as a linker.
AEAAAKEAAAKA

SEQ ID NO: 213 - protein, 5 aa, Artificial Sequence
  Peptide suitable for use as a linker.
PAPAP

SEQ ID NO: 214 - protein, 8 aa, Artificial Sequence
  Peptide suitable for use as a linker.
APAPAPAP

SEQ ID NO: 215 - protein, 12 aa, Artificial Sequence
  Peptide suitable for use as a linker.
APAPAPAPAPAP

SEQ ID NO: 216 - protein, 16 aa, Artificial Sequence
  Peptide suitable for use as a linker.
APAPAPAPAPAPAPAP

SEQ ID NO: 217 - protein, 20 aa, Artificial Sequence
  Peptide suitable for use as a linker.
APAPAPAPAPAPAPAPAPAP

SEQ ID NO: 218 - protein, 4 aa, Artificial Sequence
  Peptide suitable for use as a linker.
KPKP

SEQ ID NO: 219 - protein, 8 aa, Artificial Sequence
  Peptide suitable for use as a linker.
KPKPKPKP

SEQ ID NO: 220 - protein, 12 aa, Artificial Sequence
  Peptide suitable for use as a linker.
KPKPKPKPKPKP

SEQ ID NO: 221 - protein, 16 aa, Artificial Sequence
  Peptide suitable for use as a linker.
KPKPKPKPKPKPKPKP

SEQ ID NO: 222 - protein, 20 aa, Artificial Sequence
  Peptide suitable for use as a linker.
KPKPKPKPKPKPKPKPKPKP

SEQ ID NO: 223 - protein, 4 aa, Artificial Sequence
  Peptide suitable for use as a linker.
EPEP

SEQ ID NO: 224 - protein, 8 aa, Artificial Sequence
  Peptide suitable for use as a linker.
EPEPEPEP

SEQ ID NO: 225 - protein, 12 aa, Artificial Sequence
  Peptide suitable for use as a linker.
EPEPEPEPEPEP

SEQ ID NO: 226 - protein, 16 aa, Artificial Sequence
  Peptide suitable for use as a linker.
EPEPEPEPEPEPEPEP

SEQ ID NO: 227 - protein, 20 aa, Artificial Sequence
  Peptide suitable for use as a linker.
EPEPEPEPEPEPEPEPEPEP

SEQ ID NO: 228 - protein, 15 aa, Artificial Sequence
  Linker
GCGASGGGGSGGGGS

SEQ ID NO: 229 - protein, 15 aa, Artificial Sequence
  Peptide suitable for use as a linker.
GCGGSGGGGSGGGGS

SEQ ID NO: 230 - protein, 9 aa, Human papillomavirus type 1a
YMLDLQPET

SEQ ID NO: 231 - protein, 10 aa, Human papillomavirus type 1a
YMLDLQPETT

SEQ ID NO: 232 - protein, 9 aa, Human papillomavirus type 1a
LLMGTLGIV

SEQ ID NO: 233 - protein, 8 aa, Human papillomavirus type 1a
TLGIVCPI

SEQ ID NO: 234 - protein, 15 aa, Homo sapiens
ENPVVHFFKNIVTPR

SEQ ID NO: 235 - protein, 15 aa, Homo sapiens
GKTKEGVLYVGSKTK

SEQ ID NO: 236 - protein, 15 aa, Homo sapiens
KTKEGVLYVGSKTKE

SEQ ID NO: 237 - protein, 15 aa, Homo sapiens
MPVDPDNEAYEMPSE

SEQ ID NO: 238 - protein, 15 aa, Homo sapiens
DNEAYEMPSEEGYQD

SEQ ID NO: 239 - protein, 14 aa, Homo sapiens
EMPSEEGYQDYEPE

SEQ ID NO: 240 - protein, 12 aa, Homo sapiens
SEEGYQDYEPEA

SEQ ID NO: 241 - protein, 8 aa, Arachis hypogaea
PGQFEDFF

SEQ ID NO: 242 - protein, 8 aa, Arachis hypogaea
YLQGFSRN

SEQ ID NO: 243 - protein, 10 aa, Arachis hypogaea
FNAEFNEIRR

SEQ ID NO: 244 - protein, 8 aa, Arachis hypogaea
QEERGQRR

SEQ ID NO: 245 - protein, 10 aa, Arachis hypogaea
DITNPINLRE

SEQ ID NO: 246 - protein, 10 aa, Arachis hypogaea
NNFGKLFEVK

SEQ ID NO: 247 - protein, 6 aa, Arachis hypogaea
GNLELV

SEQ ID NO: 248 - protein, 10 aa, Arachis hypogaea
RRYTARLKEG

SEQ ID NO: 249 - protein, 10 aa, Arachis hypogaea
ELHLLGFGIN

SEQ ID NO: 250 - protein, 10 aa, Arachis hypogaea
HRIFLAGDKD

SEQ ID NO: 251 - protein, 10 aa, Arachis hypogaea
IDQIEKQAKD

SEQ ID NO: 252 - protein, 10 aa, Arachis hypogaea
KDLAFPGSGE

SEQ ID NO: 253 - protein, 10 aa, Arachis hypogaea
KESHFVSARP

SEQ ID NO: 254 - protein, 25 aa, Arachis hypogaea
NEGVIVKVSKEHVEELTKHAKSVSK

SEQ ID NO: 255 - protein, 10 aa, Arachis hypogaea
HASARQQWEL

SEQ ID NO: 256 - protein, 10 aa, Arachis hypogaea
QWELQGDRRC

SEQ ID NO: 257 - protein, 10 aa, Arachis hypogaea
DRRCQSQLER

SEQ ID NO: 258 - protein, 10 aa, Arachis hypogaea
LRPCEQHLMQ

SEQ ID NO: 259 - protein, 10 aa, Arachis hypogaea
KIQRDEDSYE

SEQ ID NO: 260 - protein, 10 aa, Arachis hypogaea
YERDPYSPSQ

SEQ ID NO: 261 - protein, 10 aa, Arachis hypogaea
SQDPYSPSPY

SEQ ID NO: 262 - protein, 10 aa, Arachis hypogaea
DRLQGRQQEQ

SEQ ID NO: 263 - protein, 10 aa, Arachis hypogaea
KRELRNLPQQ

SEQ ID NO: 264 - protein, 10 aa, Arachis hypogaea
QRCDLDVESG

SEQ ID NO: 265 - protein, 15 aa, Arachis hypogaea
IETWNPNNQEFECAG

SEQ ID NO: 266 - protein, 15 aa, Arachis hypogaea
GNIFSGFTPEFLAQA

SEQ ID NO: 267 - protein, 15 aa, Arachis hypogaea
VTVRGGLRILSPDRK

SEQ ID NO: 268 - protein, 15 aa, Arachis hypogaea
DEDEYEYDEEDRRRG

SEQ ID NO: 269 - protein, 9 aa, Artificial Sequence
  Epitope tag hemagglutinin
YPYDVPDYA

SEQ ID NO: 270 - protein, 8 aa, Artificial Sequence
  Epitope tag FLAG
DYKDDDDK

SEQ ID NO: 271 - protein, 10 aa, Artificial Sequence
  Epitope tag  c-myc
EQKLISEEDL

SEQ ID NO: 272 - protein, 5 aa, Artificial Sequence
  Affinity domain
HHHHH

SEQ ID NO: 273 - protein, 6 aa, Artificial Sequence
  Affinity domain
HHHHHH

SEQ ID NO: 274 - protein, 5 aa, Artificial Sequence
  Affinity domain
RYIRS

SEQ ID NO: 275 - protein, 4 aa, Artificial Sequence
  Affinity domain
FHHT

SEQ ID NO: 276 - protein, 17 aa, Artificial Sequence
  Affinity domain
WEAAAREACCRECCARA

SEQ ID NO: 277 - protein, 8 aa, Artificial Sequence
  StrepTag
WSHPQFEK

SEQ ID NO: 278 - protein, 5 aa, Artificial Sequence
  Motif in Class II HLA molecule
QKRAA

SEQ ID NO: 279 - protein, 5 aa, Artificial Sequence
  Motif in Class II HLA molecule
QQRAA

SEQ ID NO: 280 - protein, 5 aa, Artificial Sequence
  Motif in Class II HLA molecule
KKRAA

SEQ ID NO: 281 - protein, 5 aa, Artificial Sequence
  Motif in Class II HLA molecule
RRRAA

SEQ ID NO: 282 - protein, 5 aa, Artificial Sequence
  Motif in Class II HLA molecule
QRRAA

SEQ ID NO: 283 - protein, 5 aa, Artificial Sequence
  Motif in Class II HLA molecule
RKRAA

(19) United States
(12) Patent Application Publication
(10) Pub. No.: US 2023/0218731 A1
(43) Pub. Date: Jul. 13, 2023

(54) Antigen Presenting Polypeptide Complexes and Methods of Use Thereof

(71) Applicant: Cue Biopharma, Inc., Boston, MA (US)

(72) Inventors: Ronald D. SEIDEL, III, Natick, MA (US); Rodolfo J. CHAPARRO, Cambridge, MA (US); John F. ROSS, Arlington, MA (US); Chee Meng LOW, Boston, MA (US)

(21) Appl. No.: 17/927,501

(22) PCT Filed: May 26, 2021
(86) PCT No.: PCT/US2021/034373

§ 371 Date: Nov. 23, 2022

Related U.S. Application Data
(60) Provisional application No. 63/030,274, filed on May 26, 2020.

(51) Int. Cl. A61K 39/00; C07K 14/74; C07K 14/47

(52) U.S. Cl. CPC A61K 39/0011; C07K 14/70539; C07K 14/4748; A61K 2039/605; C07K 2319/30; A61K 2039/64

(57) ABSTRACT

The present disclosure provides Multimeric Antigen-Presenting Polypeptides (MAPPs) for the presentation of epitopes in the context of a class II MHC receptor. The present disclosure provides nucleic acids comprising nucleotide sequences encoding those MAPPs, as well as cells genetically modified with the nucleic acids encoding the MAPPs. MAPPs are useful for selectively modulating activity of T cells having T cell receptors that recognize the antigens. Thus, the present disclosure provides compositions and methods for modulating the activity of T cells, as well as compositions and methods for treating persons who have diseases and/or disorders including cancers, autoimmune diseases and/or allergies.

This application claims the benefit of U.S. Provisional Patent Appln. No. 63/030,274 filed May 26, 2020.

This application contains a sequence listing submitted electronically via EFS-web, which serves as both the paper copy and the computer readable form (CRF) and consists of a file entitled “123640-8014.W000_seqlist.txt”, which was created on May 26, 2021, which is 357,002 bytes in size, and which is herein incorporated by reference in its entirety.

### I. INTRODUCTION

An adaptive immune response involves the engagement of the T cell receptor (TCR), present on the surface of a T cell, with a small antigenic molecule (e.g., a peptide antigen) non-covalently presented on the surface of an antigen presenting cell (APC) by a major histocompatibility complex (MHC; also referred to in humans as a human leukocyte antigen (“HLA”) complex). This engagement represents the immune system's targeting mechanism and is a requisite molecular interaction for T cell modulation (activation or inhibition) and effector function. In addition to epitope-specific cell targeting, T cells may be targeted by immunomodulatory proteins found in, for example, APCs, that affect various functions of the target cells (e.g., activation or inhibition of various T cell functions) through of their costimulatory proteins found, for example, on the APC with counterpart costimulatory proteins (e.g., receptors) on the T cells. Both signals—epitope/TCR binding and engagement of APC costimulatory proteins with T cell costimulatory proteins—are required to drive T cell specificity and activation or inhibition. The TCR is specific for a given epitope; however, costimulatory proteins are not epitope specific, and instead are generally expressed on all T cells or on subsets of cells.

APCs generally serve to capture and break the proteins from foreign organisms, or abnormal proteins (e.g., from genetic mutation in cancer cells), into smaller fragments suitable as signals for scrutiny by the larger immune system, including T cells. In particular, APCs break down proteins into small peptide fragments, which are then paired with proteins of the major histocompatibility complex (“MHC”) and displayed on the cell surface. Cell surface display of an MHC together with a peptide fragment, also known as a T cell epitope, provides the underlying scaffold surveilled by T cells, allowing for specific recognition. The peptide fragments can be pathogen-derived (infectious agent-derived), tumor-derived, or derived from natural host proteins (self-proteins). Moreover, APCs can recognize other foreign components, such as bacterial toxins, viral proteins, viral DNA, viral RNA, etc., whose presence denotes an escalated threat level. The APCs relay this information to T cells through additional costimulatory signals in order to generate a more effective response.

T cells recognize peptide-major histocompatibility complex (“pMHC”) complexes through a specialized cell surface receptor, the T cell receptor (“TCR”). The TCR is unique to each T cell; as a consequence, each T cell is highly specific for a particular pMHC target. In order to adequately address the universe of potential threats, a very large number (10,000,000) of distinct T cells with distinct TCRs exist in the human body. Further, any given T cell, specific for a particular T cell peptide, is initially a very small fraction of the total T cell population. Although normally dormant and in limited numbers, T cells bearing specific TCRs can be readily activated and amplified by APCs to generate highly potent T cell responses that involve many millions of T cells. Such activated T cell responses are capable of attacking and clearing viral infections, bacterial infections, and other cellular threats including tumors. Conversely, the broad, non-specific activation of overly active T cell responses against self-antigens or shared antigens can give rise to T cells that inappropriately attack and destroy healthy tissues or cells.

MHC proteins are referred to as human leukocyte antigens (HLA) in humans. HLA proteins are divided into two major classes, class I and class II proteins, which are encoded by separate loci. Unless expressly stated otherwise, for the purpose of this disclosure, references to MHC or HLA proteins are directed to class II MHC or HLA proteins. HLA class II proteins each comprise alpha and beta polypeptide chains encoded by separate loci. HLA class II gene loci include HLA-DM (HLA-DMA and HLA-DMB that encode HLA-DM α chain and HLA-DM β chain, respectively), HLA-DO (HLA-DOA and HLA-DOB that encode HLA-DO α chain and HLA-DO β chain, respectively), HLA-DP (HLA-DPA and HLA-DPB that encode HLA-DP α chain and HLA-DP β chain, respectively), HLA-DQ (HLA-DQA and HLA-DQB that encode HLA-DQ α chain and HLA-DQ β chain, respectively), and HLA-DR (HLA-DRA and HLA-DRB that encode HLA-DR α chain and HLA-DR β chain, respectively).

Although the immune system is designed to avoid the development of immune responses to proteins and other potentially antigenic materials of the body, in some instances the immune system develops T cells with specificity for an epitope of an autoantigen (self-antigen) leading to autoimmune diseases. The immune system may also fail to respond to certain self and non-self antigens allowing cancer cells to grow unchecked by the immune system.

### II. SUMMARY

The present disclosure provides multimeric antigen-presenting polypeptide complexes (“MAPP” singular and “MAPPs” plural) that are at least heterodimeric and include at least one framework polypeptide and at least one dimerization polypeptide. Framework polypeptides comprise one or more polypeptide dimerization sequence that permits specific binding with other polypeptides (dimerization polypeptides) having a counterpart dimerization sequence thereby forming at least a heterodimer (See FIG. 1A). Framework polypeptides also comprise a multimerization sequence(s) that permits two or more framework polypeptides to associate, thereby forming a higher order structure (e.g., a duplex of the two or more heterodimers, a “duplex MAPP” see, e.g., FIGS. 1A and 1B). Neither the dimerization sequence nor the multimerization sequence of the framework polypeptide (or the counterpart dimerization sequence) comprises an MHC class II (e.g., HLA) α chain or β chain polypeptide sequence; and as such, interaction brought about by those sequences are not consider dimerization or multimerization of framework and/or dimerization peptides. Accordingly, the framework polypeptides provide a structure upon which other polypeptides (e.g., immunomodulatory and/or MHC polypeptides) can be organized by interactions at the dimerization sequences, and which can interact with other framework polypeptides by way of multimerization sequences.

The framework and dimerization peptide containing MAPPs, duplex MAPPs, and MAPPs of higher order (e.g., triplex MAPPs) described herein provide a means by which epitope-presenting peptides (“peptide epitopes” or simply “epitopes”) may be presented in the context of an MHC (e.g., HLA) to a target T cell displaying a TCR specific for the epitope, while at the same time permitting for the flexible presentation of one or more immunomodulatory polypeptides (“MODs”). The MAPPs, duplex MAPPs, and higher order MAPPs thereby permit delivery of one or more MODs in an epitope selective (e.g., dependent/specific) manner that permits (i) formation of an active immune synapse with a target T cell selective for the epitope, and (ii) modulation (e.g., control/regulation) of the target T cell's response to the epitope.

The presentation by a MAPP of a peptide epitope to a target T cell is accomplished via a moiety that comprises MHC Class II polypeptides and the peptide epitope. Such moieties may be either (i) a single polypeptide chain, or (ii) a complex comprising two or more polypeptide chains.

Where the peptide epitope, MHC Class II polypeptides, and optionally one or more MODs are provided in a single polypeptide chain, it is termed a “presenting sequence” See, e.g., FIG. 25. The presenting sequences may be integrated into a MAPP as part of a framework polypeptide or a dimerization polypeptide. A MAPP may have presenting sequences as part of either or both of framework or dimerization polypeptide. Compare, for example, FIG. 19 structures A-D and FIG. 20 structures A-D.

As an alternative to utilizing a single polypeptide to present an epitope, the MHC components (e.g., α1, α2, β1 and β2 domain sequences) and the epitope may be divided among two separate polypeptide sequences, which together are denoted herein as a “presenting complex.” See, e.g., FIGS. 27 to 30. A presenting complex is integrated into a MAPP by having a presenting complex first amino acid sequence (“presenting complex 1st sequence”) as part of a framework or dimerization polypeptide. The remaining MHC sequence(s) are part of a polypeptide termed the presenting complex second amino acid sequence (“presenting complex 2nd sequence”). The peptide epitope and any independently selected MODs that are present may be part of the polypeptide comprising either the presenting complex 1st sequence or the presenting complex 2nd sequence. The presenting complex 1st sequence and presenting complex 2nd sequence generally associate through non-covalent interactions between the α chain and β chain polypeptide sequence, and may be stabilized by disulfide bonds between either the MHC sequences or peptide/polypeptide linkers attached to the N- or C-t terminus of the MHC sequences. The presenting complex 1^{st} sequence and presenting complex 2^{nd} sequence may also associate through dimerization or interspecific dimerization sequences if present in those polypeptides.

Although an individual MAPP may not comprise a presenting sequence or presenting complex, for the purpose of this disclosure the MAPPs are, unless stated otherwise, understood to comprise at least one presenting sequence or presenting complex.

MAPPs that comprise a presenting sequence typically contain one or two presenting sequences. Duplex MAPPS thus typically comprise two, three or four presenting sequences, but also may comprise one presenting sequence (e.g., if one of the MAPPS does not comprise a presenting sequence). MAPPs and duplex MAPPs may comprise more presenting sequences depending on, for example, the number of dimerization sequences in the framework polypeptide. The presenting sequences may be integrated into a MAPP as part of a framework polypeptide, a dimerization polypeptide, or both. Compare, for example, FIG. 19 structures A-D and FIG. 20 structures A-D.

Likewise, MAPPs with presenting complexes typically contain one or two presenting complexes, and accordingly, duplex MAPPs with presenting complexes typically comprise two, three or four presenting complexes, but also may comprise one presenting complex (e.g., if one of the MAPPs does not comprise a presenting complex). As discussed above, MAPPs and duplex MAPPs may comprise more presenting complexes depending on, for example, the number of dimerization sequences in the framework polypeptide.

MAPPs, and accordingly their higher order complexes (duplexes, triplexes etc.), comprising MHC Class II polypeptide sequences and a peptide epitope for presentation to a TCR, may present peptides to T cells (e.g., CD4+ T cells) that have a TCR specific for the epitope. Once engaged with the TCR of a T cell, the effect of a MAPP on the T cell depends on which MODs, if any, are present as part of the MAPP.

MOD-containing MAPPs can function as a means of selectively delivering the MODs to T cells specific for the MAPP associated epitope, thereby resulting in MOD-driven responses to those MAPPs (e.g., the reduction in number and/or suppression of CD4+ effector T cells reactive with MAPP-associated epitopes). Depending on the chosen MOD, the incorporation of one or more MODs with increased affinity for their cognate receptor on T cells (“co-MOD”) may reduce the specificity of MAPPs and duplex MAPPs for epitope specific T cells where MOD-co-MOD binding interactions significantly compete with MHC/epitope binding to target cell TCR. Conversely, and again depending on the chosen MOD, the inclusion of MODs with reduced affinity for their co-MOD(s), and the affinity of the epitope for a TCR, may provide for enhanced selectivity of MAPPs and duplex MAPPs, while retaining the desired activity of the MODs. Where a MOD already possesses a relatively low affinity for its cognate receptor, mutations that reduce the affinity may be unnecessary and/or undesirable.

The ability of MAPPs (e.g., duplex MAPPs) to modulate T cells provides methods of modulating T cell activity in vitro and in vivo, and accordingly the use of MAPPs as therapeutics useful in methods of treating a variety of diseases and conditions including cancers, autoimmune diseases, and allergies.

The present disclosure provides nucleic acids comprising nucleotide sequences and vectors encoding individual MAPP polypeptides and MAPPs (e.g., all polypeptides of a MAPP), as well as cells genetically modified with the nucleic acids and vectors for producing individual MAPP polypeptides and/or MAPP proteins (e.g., duplex MAPPs). The present disclosure also provides methods of producing MAPPs, duplex MAPPs, and higher order MAPPs utilizing such cells.

### III. BRIEF DESCRIPTION OF THE DRAWINGS

FIG. 1A is provided to illustrate of the terminology used to describe MAPPs and duplex MAPPs with presenting sequences. The peptides are oriented from N-terminus (left) to C-terminus (right). The figure shows first and second framework polypeptides, which in this case are different and, in this instance, have specific multimerization sequences comprising a knob and counterpart hole. Such “knob-in-hole” configurations may include knob-in-hole configurations without a stabilizing disulfide bond (herein “KiH”) or with a stabilizing disulfide bond (herein “KiHs-s”). Also shown are first and second dimerization polypeptides having an N-terminal epitope and counterpart dimerization sequences. The dashed circles indicate five potential locations for the addition of polypeptide sequences, including MODs (discussed below). The figure depicts the formation of a first and second heterodimer MAPPs, each comprising a framework polypeptide and dimerization polypeptide. The heterodimers may interact through the multimerization sequence to form a multimer (a duplex MAPP as shown). The use of knob-in-hole sequences permit the assembly of an asymmetric interspecific duplex MAPP where, for example, different MOD sequences are provided at positions 1 and 1′ and/or positions 3 and 3′. While interactions between polypeptide chains through peptide interaction sequences may initially be non-covalent in nature, interchain disulfide bond formation reactions may occur thereby providing covalently linked polypeptides at, for example, either dimerization sequences or multimerization sequences. Throughout the figures, lines connecting various elements of MAPP polypeptides are optional amino acids serving as linkers (e.g., peptide linkers).

FIG. 1B parallels FIGS. 1A and 1s provided to illustrate of the terminology used to describe MAPPs and duplex MAPPs with presenting complexes. The word “sequence” may be abbreviated by “seq.”.

FIGS. 2A-2H provide amino acid sequences of immunoglobulin polypeptides including their heavy chain constant regions (“Ig Fc” or “Fc”, e.g., the CH2-CH3 domain of IgG1) (SEQ ID NOs:1-13).

FIG. 2I provides the sequence of an Ig CH1 domain (SEQ ID NO:14).

FIG. 2J provides the sequence of a human Ig-J chain (SEQ ID NO:122).

FIG. 3A provides the sequence of an Ig κ chain (kappa chain) constant region (SEQ ID NO:15).

FIG. 3B provides the sequence of an Ig λ chain (lambda chain) constant region (SEQ ID NO:16).

FIG. 4 provides an amino acid sequence of a HLA Class II DRA (sometimes referred to as DRA1) a chains (SEQ ID NO:17).

FIG. 5 provides amino acid sequences of HLA Class II DRB1 β chains (SEQ ID NOs:18-54).

FIG. 6 provides amino acid sequences of HLA Class II DRB3 β chains (SEQ ID NOs:55-58).

FIG. 7 provides an amino acid sequence of a HLA Class II DRB4 β chain (SEQ ID NOs:59-60).

FIG. 8 provides an amino acid sequence of a HLA Class II DRB5 β chain (SEQ ID NO:61).

FIG. 9 provides an amino acid sequence of a HLA Class II DMA α chain (SEQ ID NO:62).

FIG. 10 provides an amino acid sequence of a HLA Class II DMB β chain (SEQ ID NO:63).

FIG. 11 provides an amino acid sequence of a HLA Class II DOA α chain (SEQ ID NO:64).

FIG. 12 provides an amino acid sequence of a HLA Class II DOB β chain (SEQ ID NO:65).

FIG. 13 provides amino acid sequences of HLA Class II DPA1 α chains (SEQ ID NOs:66-67).

FIG. 14 provides amino acid sequences of HLA Class II DPB1 β chains (SEQ ID NOs:68-79).

FIG. 15 provides amino acid sequences of HLA Class II DQA1 α chains (SEQ ID NOs:80-90).

FIG. 16 provides an amino acid sequence of a HLA Class II DQA2 α chain (SEQ ID NO:91).

FIG. 17 provides amino acid sequences of HLA Class II DQB1 β chains (SEQ ID NOs:92-103).

FIGS. 18A-18B provide amino acid sequences of HLA Class II DQB2 β chains (SEQ ID NO:104-105).

FIG. 19 provides a series of duplex MAPP structures based on framework polypeptides having both (i) a multimerization sequence, and (ii) first and second dimerization sequences that may be the same or different. The structure is shown generically in A with locations 1-5 and 1 ‘-5’ indicating locations for additional peptide sequences (e.g., MOD polypeptide sequences). The MHC/epitope moiety is illustrated generically, and can be either a presenting sequence (see, e.g., FIGS. 25-26), or a presenting complex (see FIGS. 27-32). Locations 4 and 4′ are shown at the N-terminus of a presenting sequence or the N-terminus of a presenting complex polypeptide, and locations 5 and 5′ are shown at the C-termini of those polypeptides. Locations 1 and 1′ are shown at the N-terminus of the framework peptide and locations 3 and 3′ at the C-terminus of the framework polypeptide. In A and C, the framework polypeptides are multimerized to form a duplex of heterodimers via non-covalent binding between the multimerization sequences. In B and D, the framework polypeptides are multimerized to form a duplex of heterodimers using an immunoglobulin Fc region knob-in-hole motif, although other methods of non-covalently or covalently bonding the multimerization sequences may be used. In C the duplexes contain heterodimers in which two different asymmetric interspecific dimerization sequences bind together the framework peptides and their associated dimerization peptides. In D the framework peptides are joined together by a knob-in-hole Fc motif and the dimerization peptide and framework peptide are joined together by different dimerization sequences to form a duplex of heterodimers.

FIG. 20 provides in A to D a series of MAPP structures as in FIG. 19, with the addition of presenting sequences or presenting complexes at the N-terminus of the framework peptides. Positions 4 and 4′ may still serve as locations for peptide addition (e.g., MOD polypeptide addition).

FIG. 21 provides in A to D a series of MAPP structures as in FIG. 19, where the dimerization sequences are Ig CH1 sequences (CH1) that pair with Ig light chain sequences (CL). The framework peptides are multimerized (dimers in this instance) through the interaction of Ig Fc (e.g., CH2 and CH3) regions, with the structures in B and D having knob-in-hole motifs to permit heteroduplexes to be formed. The peptides are also joined by disulfide bonds (e.g., those that form between Ig Fc region peptides).

FIG. 22 provides a series of MAPP structures as in FIG. 21, with the addition of presenting sequences or presenting complexes at the N-terminus of the framework peptides. Positions 4 and 4′ may still serve as locations for peptide addition (e.g., MOD polypeptide addition).

FIG. 23 provides in A to J a series of MAPP structures as in FIG. 11. In each instance, a presentation sequence lacking a MOD sequence is present on the dimerization peptide (marked as a single chain MHC and epitope). Locations 2, 2′, 4, 4′, 5 and 5′ are unfiled and not shown. Locations 1 and 1′ are substituted with one or more MODs, e.g., for illustration purposes wild type and/or variants of IL-2, PD-L1, and CD80, although other MODs may be used, e.g., wild type and/or variant TGF-β or 4-1BBL. Positions 3 and 3′ are shown for orientation in A to G. In H to J the 3 and 3′ locations are either unfiled, e.g., for illustration purposes a wild type and/or variant TGF-β or 4-1BBL MODs may be located there, although other MODs may be used, e.g., wild type and/or variants of IL-2, PD-L1, and CD80.

FIG. 24 shows four MAPP heterodimer constructs as structures A-D that can form duplex MAPPs. In the polypeptide sequences of structures A to D the “MOD” can be, for example, PDL1.

FIG. 25 shows in A to C three different MHC Class II presenting sequences (from the epitope at the N-terminus to C-terminus. The sequences optionally comprise one or more independently selected MODs (including two or more MODs in tandem) at the indicated locations.

FIG. 26 shows in A to H different embodiments of MHC Class II presenting sequences (from left to right N-terminus to C-terminus).

FIGS. 27 to 32 show a series of MHC Class II presenting complexes from left to right N- to C-terminus. The sequence bearing the symbol “-//-” is the presenting complex 1^{st} sequence. The other sequence is its associated presenting complex 2^{nd} sequence. The symbol “-//-” denotes the point of attachment of the complex to the remainder of the dimerization or framework peptide. In FIG. 30A-L, the presenting complex 1^{st} sequence and its associated presenting complex 2^{nd} sequence include dimerization sequences to unite the peptides (shown as an Ig Fc region associated with the Ig light chain constant region Cκ (kappa chain), although other sequences could be utilized). In FIGS. 31A-F and 32A-F, the presenting complex 1^{st} sequence and its associated presenting complex 2^{nd} sequence include dimerization sequences to unite the peptides (shown as a leucine zipper pair, although other sequences could be utilized).

FIG. 33 provides a table showing associations of HLA class II alleles and haplotypes with risk of an autoimmune disease. The table also provides epitopes of autoantigens (self-epitopes) associated with a number of the diseases listed.

### IV. DETAILED DESCRIPTION

### A. Definitions

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” and “protein” are used interchangeably herein, and refer to a polymeric form of amino acids, which unless stated otherwise are the naturally occurring proteinogenic L-amino acids that are incorporated biosynthetically into proteins during translation in a mammalian cell. Furthermore, as used herein, a “polypeptide” and “protein” include modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References to a specific residue or residue number in a known polypeptide, e.g., position 72 or 75 of human DRA MHC class II polypeptide, are understood to refer to the amino acid at that position in the wild-type polypeptide (i.e. I72 or K75). To the extent that the sequence of the wild-type polypeptide is altered, either by addition or deletion of one or more amino acids, the specific residue or residue number will refer to the same specific amino acid in the altered polypeptide (e.g., in the addition of one amino acid at the N-terminus of a peptide reference as position I72, will be understood to indicate the amino acid, Ile, that is now position 73). Substitution of an amino acid at a specific position is denoted by an abbreviation comprising, in order, the original amino acid, the position number, and the substituted amino acid, e.g., substituting the Ile at position 72 with a cysteine is denoted as I72C.

A nucleic acid or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including blast.ncbi.nlm.nih.gov/Blast.cgi for BLAST+2.10.0, ebi.ac.uk/Tools/msa/tcoffee/, ebi.ac.uk/Tools/msa/muscle/, and mafft.cbrc.jp/alignment/software/. See, e.g., Altschul et al. (1990), J. Mol. Biol. 215:403-10. Unless otherwise indicated, the percent sequence identities described herein are those determined using the BLAST program. In the event of a conflict between the results produced by different release versions of BLAST, BLAST+2.10.0 released Dec. 23, 2019, is employed as the basis for determining sequence identity.

As used herein amino acid (“aa” singular or “aas” plural) means the naturally occurring proteogenic amino acids incorporated into polypeptides and proteins in mammalian cell translation. Unless stated otherwise: L (Leu, leucine), A (Ala, alanine), G (Gly, glycine), S (Ser, serine), V (Val, valine), F (Phe, phenylalanine), Y (Tyr, tyrosine), H (His, histidine), R (Arg, arginine), N (Asn, asparagine), E (Glu, glutamic acid), D (Asp, asparagine), C (Cys, cysteine), Q (Gln, glutamine), I (Ile, isoleucine), M (Met, methionine), P (Pro, proline), T (Thr, threonine), K (Lys, lysine), and W (Trp, tryptophan). Amino acid also includes the amino acids hydroxyproline and selenocysteine, which appear in some proteins found in mammalian cells, however, unless their presence is expressly indicated they are not understood to be included.

As used herein the term “in vivo” refers to any process or procedure occurring inside of the body, e.g., of a patient.

As used herein, “in vitro” refers to any process or procedure occurring outside of the body.

The term “conservative amino acid substitution” refers to the interchangeability in proteins of aa residues having similar side chains. For example, a group of aas having aliphatic side chains consists of glycine, alanine, valine, leucine, and isoleucine; a group of aas having aliphatic-hydroxyl side chains consists of serine and threonine; a group of aas having amide containing side chains consists of asparagine and glutamine; a group of aas having aromatic side chains consists of phenylalanine, tyrosine, and tryptophan; a group of aas having basic side chains consists of lysine, arginine, and histidine; a group of aas having acidic side chains consists of glutamate and aspartate; and a group of aas having sulfur containing side chains consists of cysteine and methionine. Exemplary conservative aa substitution groups are: valine-leucine-isoleucine, phenylalanine-tyrosine, lysine-arginine, alanine-valine-glycine, and asparagine-glutamine.

The term “binding” refers to a direct association between molecules and/or atoms, due to, for example, covalent, electrostatic, hydrophobic, and ionic and/or hydrogen-bond interactions, including interactions such as salt bridges and water bridges. Non-covalent interactions/binding refers to a direct association between two molecules, due to, for example, electrostatic, hydrophobic, ionic, and/or hydrogen-bond interactions, including interactions such as salt bridges and water bridges. Non-covalent binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Covalent bonding,” or “covalent binding” as used herein, refers to the formation of one or more covalent chemical bonds between two different molecules. The term “binding,” as used with reference to the interaction between a MAPP and a T cell receptor (TCR) on a T cell, refers to a non-covalent interaction between the MAPP and TCR.

“Affinity” as used herein generally refers to the strength of non-covalent binding, increased binding affinity being correlated with a lower K_{D}. As used herein, the term “affinity” may be described by the dissociation constant (K_{D}) for the reversible binding of two agents (e.g., an antibody and an antigen. Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 40-fold greater, at least 60-fold greater, at least 80-fold greater, at least 100-fold greater, or at least 1,000-fold greater, or more, than the affinity of an antibody or receptor for an unrelated aa sequence (e.g., ligand). Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution.

“T cell” includes all types of immune cells expressing CD3, including T-helper cells (CD4⁺ cells), cytotoxic T cells (CD8⁺ cells), T-regulatory cells (Treg), and NK-T cells.

The term “immunomodulatory polypeptide” (also referred to as a “costimulatory polypeptide” or, as noted above, “MOD”), as used herein includes a wild-type or variant of a polypeptide or portion thereof that can specifically bind a cognate co-immunomodulatory polypeptide (“co-MOD”) present on a T cell, and provide a modulatory signal to the T cell when the TCR of the T cell is engaged with an MHC-epitope moiety that is specific for the TCR. Unless stated otherwise the term “MOD” includes wild-type and/or variant MODs, and statements including reference to both wild-type and variant MODs are made to emphasize that one, the other, or both are being referenced. The signal provided by the MOD engaging its co-MOD mediates (e.g., directs) a T cell response. Such responses include, but are not limited to, proliferation, activation, differentiation, suppression/inhibition of proliferation, activation and/or differentiation, and the like. A MOD can include, but is not limited to, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, lymphotoxin beta receptor, 3/TR6, ILT3, ILT4, HVEM, an agonist or antibody that binds Toll ligand receptor and a ligand that specifically binds with B7-H3. A MOD also encompasses, inter alia, an antibody or antibody fragment that specifically binds with and activates a cognate co-stimulatory molecule (co-MOD) present on a T cell, such as, but not limited to antibodies against the receptors for any of IL-2, CD27, CD28, 4-1BB, OX40, CD30, CD40, PD-1, ICOS, lymphocyte function-associated antigen-1 (LFA-1), CD2, LIGHT, NKG2C, B7-DC, B7-H2, B7-H3, and CD83.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

The terms “treatment,” “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient” are used interchangeably herein and refer to any mammalian subject for whom diagnosis, treatment, or therapy is desired. Mammals include humans and non-human primates, and in addition include rodents (e.g., rats; mice), lagomorphs (e.g., rabbits), ungulates (e.g., cows, sheep, pigs, horses, goats, and the like), felines, canines, etc.

Unless indicated otherwise, the term “substantially” is intended to encompass both “wholly” and “largely but not wholly”. For example, an Ig Fc that “substantially does not induce cell lysis” means an Ig Fc that induces no cell lysis at all or that largely but not wholly induces no cell lysis.

As used herein, the term “about” used in connection with an amount indicates that the amount can vary by 10%. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The terms “purifying”, “isolating”, and the like, refer to the removal of a desired substance, e.g., a MAPP, from a solution containing undesired substances, e.g., contaminates, or the removal of undesired substances from a solution containing a desired substance, leaving behind essentially only the desired substance. In some instances, a purified substance may be essentially free of other substances, e.g., contaminates. Purifying, as used herein, may refer to a range of different resultant purities, e.g., wherein the purified substance makes up more than 80% of all the substance in the solution, including more than 85%, more than 90%, more than 91%, more than 92%, more than 93%, more than 94%, more than 95%, more than 96%, more than 97%, more than 98%, more than 99%, more than 99.5%, more than 99.9%, and the like. As will be understood by those of skill in the art, generally, components of the solution itself, e.g., water or buffer, or salts are not considered when determining the purity of a substance.

Where a range of values is provided, it is understood that each intervening value between the upper and lower limit of that range to a tenth of the lower limit of the range is encompassed within the disclosure along with any other stated or intervening value in the range. The upper and lower limits of these smaller ranges may independently be included in smaller ranges, that are also encompassed within the disclosure subject to any specifically excluded limit in the stated range. Where the stated range a value (e.g., an upper or lower limit), ranges excluding those values are also included.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a Treg” includes a plurality of such Tregs and reference to “the MHC Class II alpha chain” includes reference to one or more MHC Class II alpha chains and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### B. Description

1. MAPP Structure and the Role of Framework and Dimerization Peptides

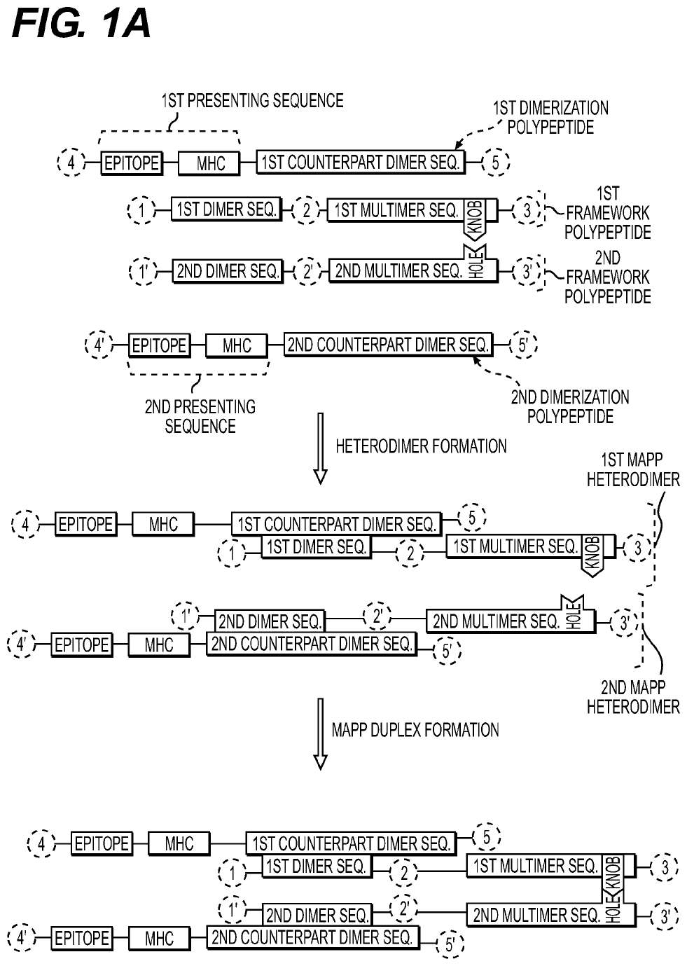

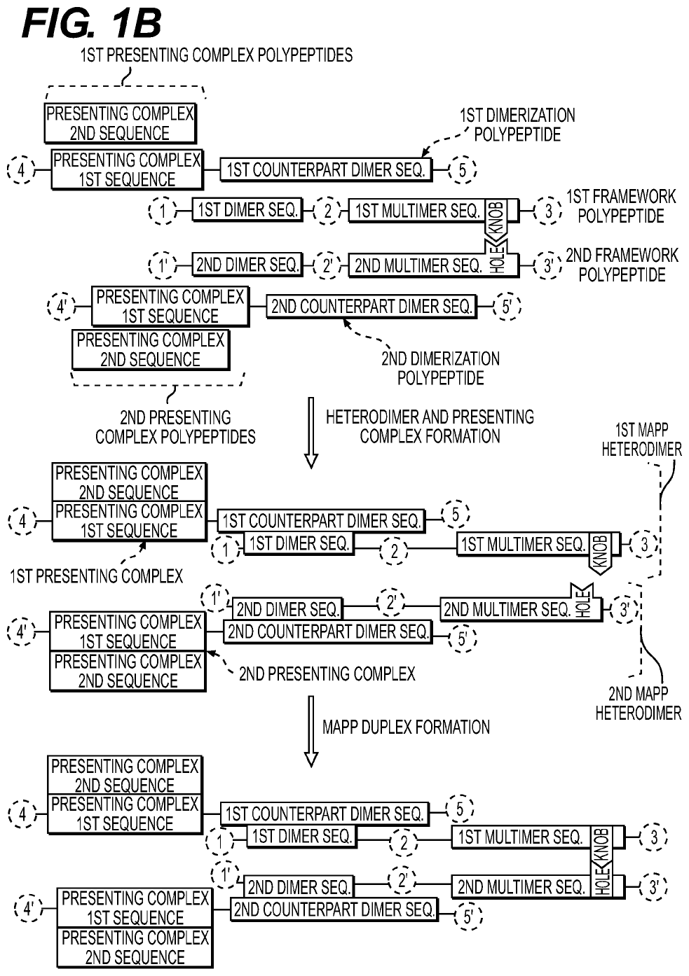

The present disclosure provides MAPPs for, among other things, use in the treatment of disease and disorders including cancers, autoimmune diseases, and allergies. As discussed above, the MAPPs include at least one framework polypeptide and at least one dimerization polypeptide. Framework polypeptides comprise one or more polypeptide dimerization sequence that permits specific binding with other polypeptides (dimerization polypeptides) having a counterpart dimerization sequence thereby forming at least a heterodimer (See FIGS. 1A and 1B). Framework polypeptides also comprise a multimerization sequence(s) that permits two or more framework polypeptides to associate, thereby forming a higher order structure (e.g., a duplex of the two or more heterodimers, a “duplex MAPP” see, e.g., FIGS. 1A and 1B). Neither the dimerization sequence nor the multimerization sequence of the framework polypeptide (or the counterpart dimerization sequence) comprises an MHC class II (e.g., HLA) α chain or β chain polypeptide sequence; and as such, interaction brought about by those sequences are not consider dimerization or multimerization of framework and/or dimerization peptides. Accordingly, the framework polypeptides provide a structure upon which other polypeptides can be organized by interactions at the dimerization sequences, and which can interact with other framework polypeptides by way of multimerization sequences. The terms “MAPP” and “MAPPs” as used herein will be understood to refer in different contexts to the heterodimer comprising a framework and dimerization peptide structure as well as higher order complexes of those MAPP heterodimers, such as duplexes (duplex MAPPs). It will be clear to the skilled artisan when specific reference to only higher order structures are intended (e.g., by reference to duplex MAPPs etc.).

As discussed above, the framework and dimerization peptide containing MAPPs, duplex MAPPs, and MAPPs of higher order (e.g., triplex MAPPs) described herein provide a means by which peptide epitopes may be delivered in the context of MHC (e.g., HLA) polypeptides to a target T cell displaying a TCR specific for the epitope, while at the same time permitting for the flexible presentation of one or more MODs. The MAPPs, duplex MAPPs, and higher order MAPPs thereby permit deliver of one or more MODs in an epitope selective (e.g., dependent/specific) manner that permits formation of an active immune synapse with a target T cell selective for the epitope, and control/regulation of the target T cell's response to the epitope. Accordingly, where MAPPs comprise stimulatory or activating MODs (e.g., IL-2, CD80, CD86, and/or 4-1BBL) that increase T cell proliferation and/or effector functions in an epitope selective manner. In contrast, where MAPPs comprise suppressive/inhibitory MODs (e.g., FasL and/or PD-L1) they decrease T cell activation, proliferation, differentiation, and/or effector functions in an epitope selective manner.

The framework/dimerization polypeptide architecture of MAPPs and their higher order structures may also be understood to provide flexibility in locating MODs and epitope presenting complexes or epitope presenting sequences. Duplex MAPP and higher order MAPP architecture can be particularly useful when both the MOD and the epitope presenting complexes (or epitope presenting sequences) are positioned so as to provide the desired biological activity as well as other desired properties of the MAPP, e.g., thermal stability and manufacturability. In some cases, acceptable combinations of properties may be obtained when the MOD and presenting complex or presenting sequence are positioned at the N-terminus of a polypeptide, e.g., each may be located at the N-terminus of different framework and/or dimerization polypeptide sequences. In some cases, acceptable combinations of properties may be obtained when the MOD and presenting complex or presenting sequence are positioned at the C-terminus of a polypeptide, e.g., each may be located at the C-terminus of different framework and/or dimerization polypeptide sequences. In some cases, acceptable combinations of properties may be obtained when the MOD and presenting complex or presenting sequence are positioned at the N-terminus and C-terminus of a polypeptide, respectively, e.g., the MOD may be located at the N-terminus and the presenting complex or presenting sequence may be located at the C-terminus of different framework and/or dimerization polypeptide sequences. In some cases, acceptable combinations of properties may be obtained when the MOD and presenting complex or presenting sequence are positioned at the C-terminus and N-terminus of a polypeptide, respectively, e.g., the MOD may be located at the C-terminus and the presenting complex or presenting sequence may be located at the N-terminus of different framework and/or dimerization polypeptide sequences.

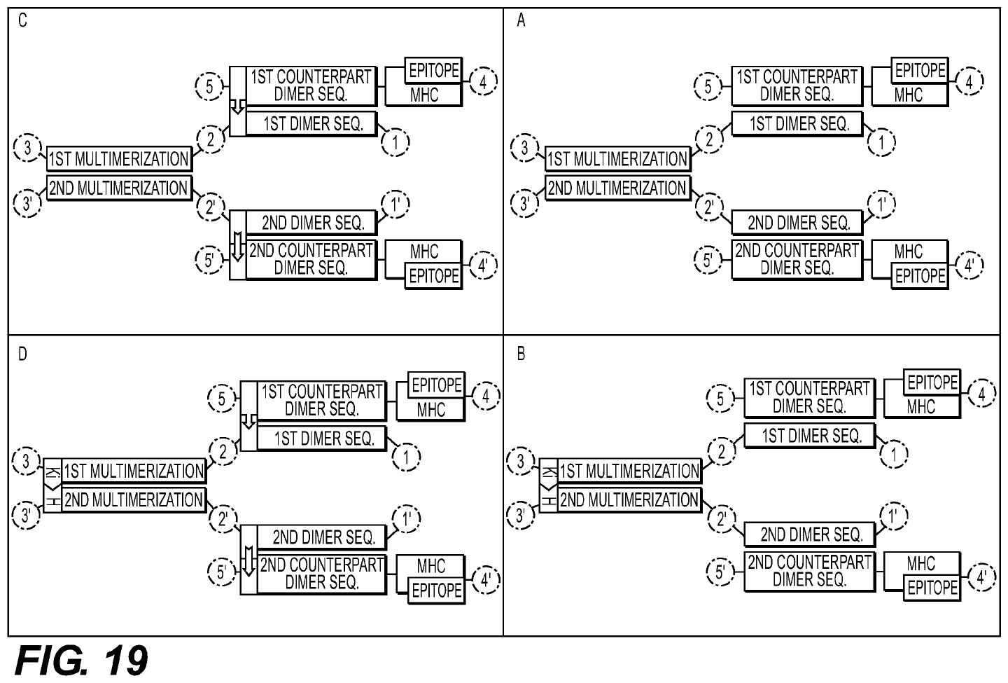

The structure of MAPPs, and particularly higher order MAPPs such as duplexes, may be specified by the use of pairs of polypeptides having different sequences that specifically pair with each other. Multimerization of framework polypeptides results from interactions between multimerization sequences, and dimerization (the interaction of a framework and dimerization polypeptide) results from the interaction of a dimerization sequence on the framework polypeptide and a counterpart dimerization on a dimerization polypeptide. For example, in a duplex MAPP the multimerization sequences may be Ig Fc heavy chain (e.g., CH2-CH3) sequences, and the dimerization sequence and counterpart dimerization sequences may be the same (e.g., all leucine zipper sequences). An additional degree of control may be obtained by utilizing non-identical peptide sequences that specifically/selectively pair with each other that are referred to herein generally as “interspecific sequences,” in the case of dimerization sequences “interspecific dimerization sequences,” or in the case of multimerization sequences “interspecific multimerization sequences,” and which give rise to asymmetric interspecific pairs of sequences. The structure of MAPPs thus permits diverse and effective placement of each polypeptide into the MAPP architecture MAPP (see, e.g., FIGS. 19-23). Interspecific sequences include Ig heavy chain Fc (e.g., CH2-CH3) region modified with, for example, knob-in-hole variations; and Fos peptide sequences paired with Jun peptide sequences. Accordingly, MAPP architectures include, but are not limited to, MAPPs where each, or some, of the dimerization sequences are different (permit different peptide pairings). For example, in duplex MAPPs where each of the multimerization and dimerization sequence are different and provides separate peptide pairings.

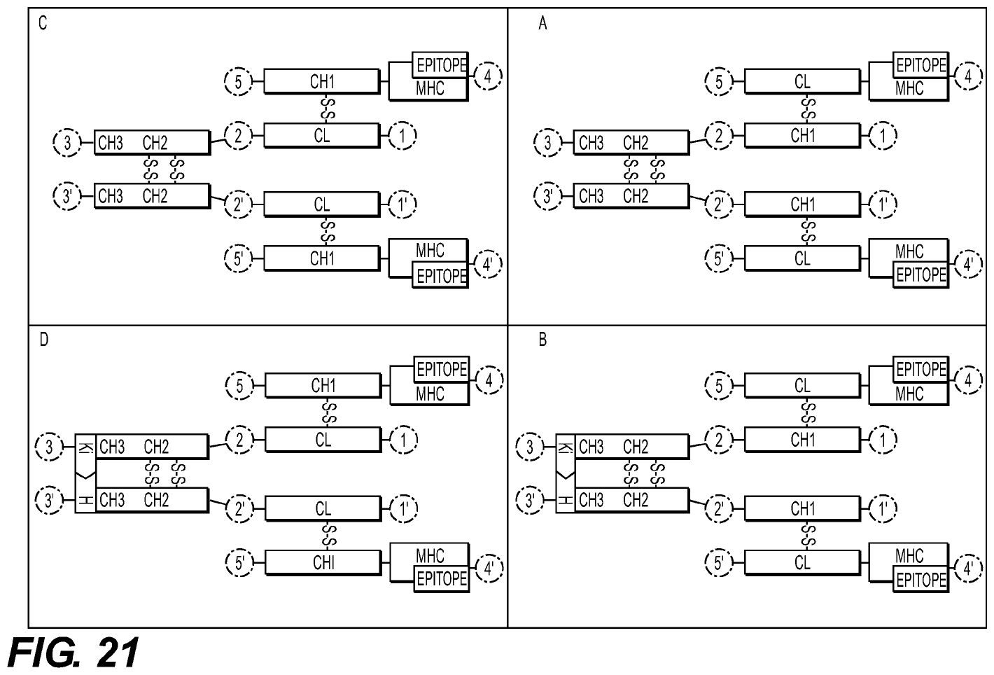

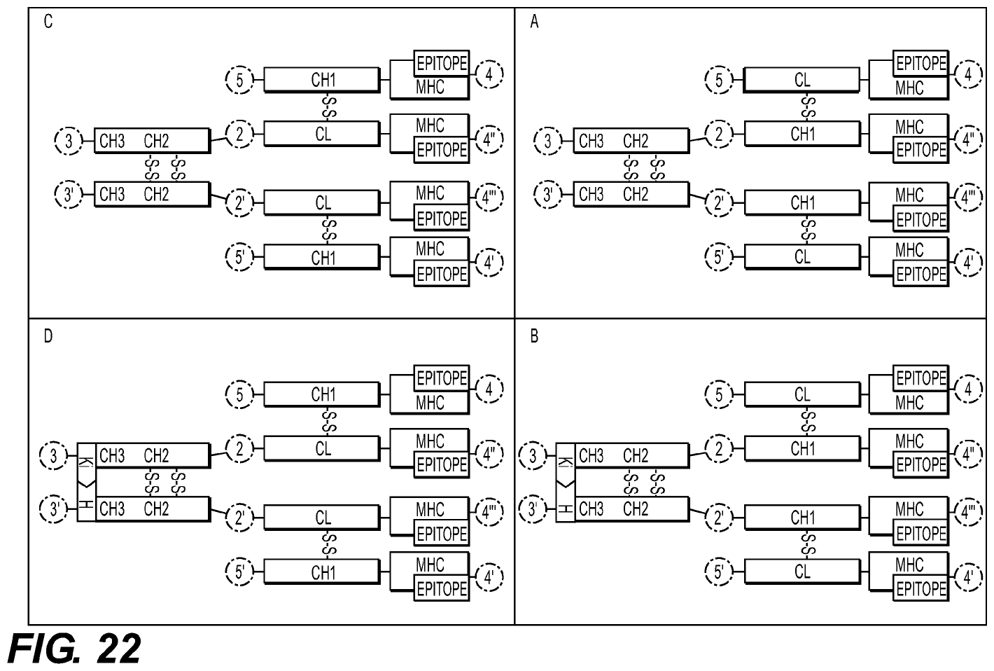

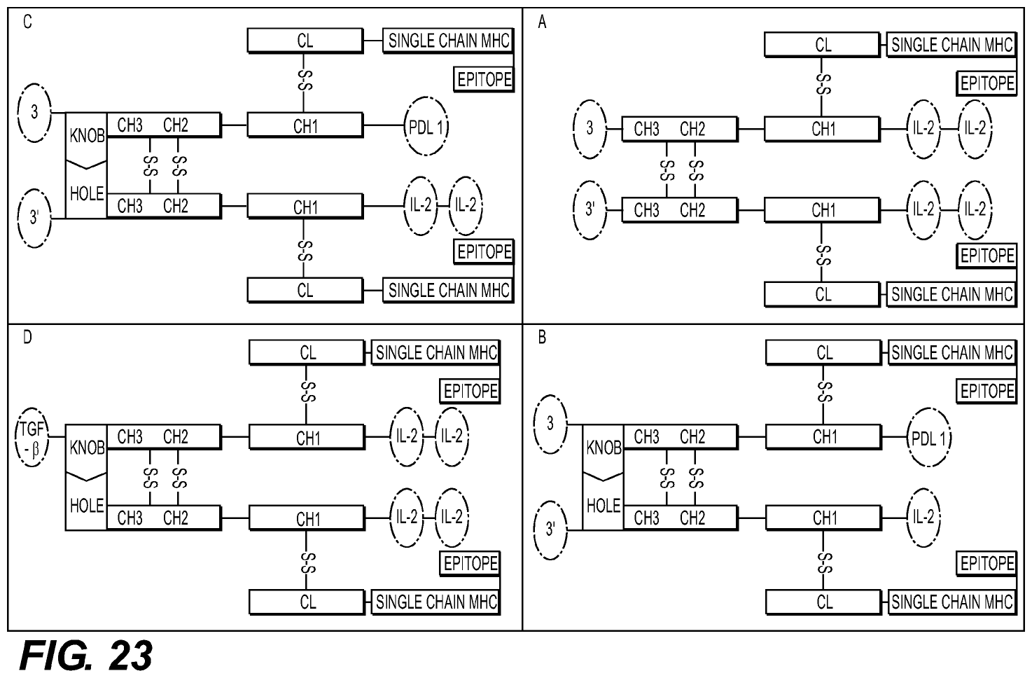

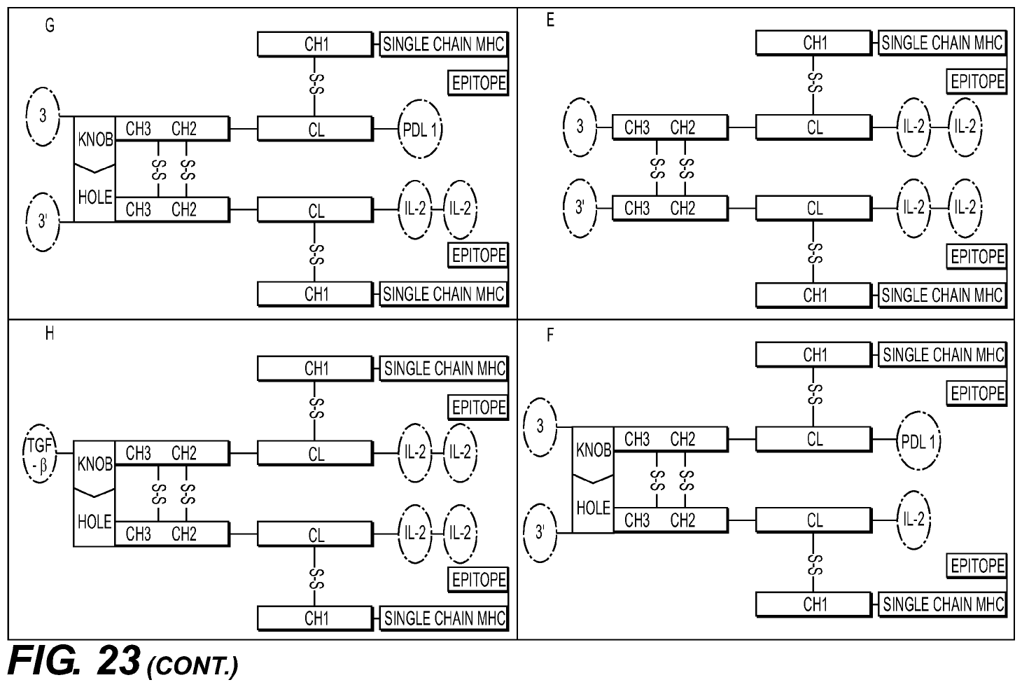

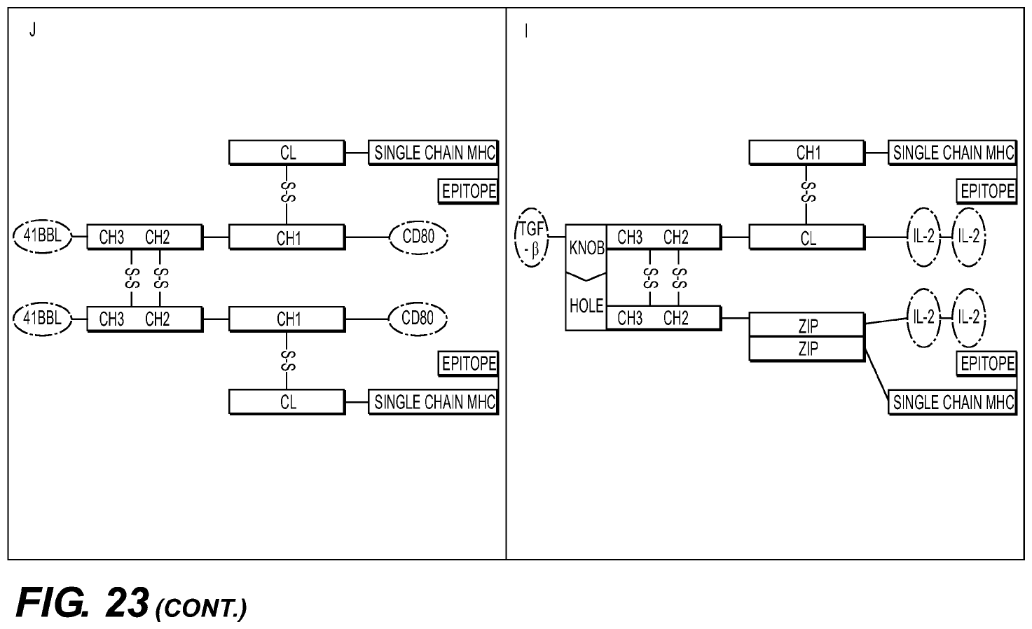

In an embodiment, the framework peptide multimerization sequence is an Fc heavy chain region (optionally a knob-in hole Fc sequence pair) and the dimerization sequences are the same (e.g., Ig CH1 sequences paired with light chain λ or κ constant region sequences) (see, for example, FIGS. 21 and 22, structures A to D). In another embodiment, the framework peptide multimerization site is an Fc heavy chain region (optionally a knob-in hole Fc sequence pair) and the dimerization sequences are selected to be different (e.g., a dimerization sequence pair comprising an Ig CH1 paired with light chain λ or κ sequence and a dimerization sequence comprising a leucine zipper pair, see for example, FIG. 23, structures E to H). For example, in a duplex MAPP the multimerization sequences may be a knob-in-hole Ig sequence, one dimerization sequence and its counterpart dimerization sequence may be leucine zipper sequences, and second dimerization sequence and its counterpart dimerization sequence may be an Ig CH1 and Ig CL λ domain pair.

MAPPs and accordingly their higher order complexes (duplexes, triplexes etc.) comprise MHC Class II polypeptide sequences that bind an epitope for presentation to a TCR, and accordingly may present peptides to T cells (e.g., CD4⁺ T cells). The effect of MAPPs on T cells with TCRs specific to the epitope depends on which, if any, MODs are present in the MAPP. As noted above, MAPPs, duplex MAPPS and higher order MAPPs comprising MOD(s) permit MOD delivery to T cells in an epitope selective manner and the MODs principally dictate the effect of MAPP-T cell engagement in light of the specific cell type stimulated and the environment. While not wishing to be bound by any particular theory, the effect of MAPP (e.g., duplex MAPP) presentation of MOD(s) and epitope to a T cells in some cases may be enhanced relative to the situation encountered in antigen presenting cells (APC) where epitope can diffuse away from the MHC (e.g., HLA) complex and any MODs the APC is presenting. This may not occur with a MAPP, however, where the epitope and MOD(s) are part of the MAPP polypeptide(s) and cannot diffuse away even if the epitope's affinity for the MHC complex would normally permit it to leave the comparable cell complex. The inability of epitope to diffuse away from MHC and MOD components of a MAPP, duplex MAPP, or higher order MAPP may be further limited where the polypeptide(s) of the MAPP (e.g., framework, dimerization sequence, and if present, the presenting complex 2^{nd} sequence) are covalently attached to each other (e.g., by disulfide bonds). Consequently, MAPPs and their higher order structures may be able to prolong delivery of MOD(s) to T cells in an epitope selective manner relative to systems where epitopes can diffuse away from the presenting MHC.

Incorporation of one or more MODs with affinity for their cognate receptor on T cells (“co-MOD”) can reduce the specificity of MAPPs (e.g., duplex MAPPs) for epitope selective/specific T cells. The reduction in epitope selectivity/specificity of the MAPPs becomes more pronounced where MOD/co-MOD binding interactions increase in strength (binding energy) and significantly compete with MHC/epitope binding to target cell TCR. The inclusion of variant MODs with reduced affinity for their co-MOD(s) thus may provide a lower contribution of MOD binding energy, thereby permitting MHC-epitope interactions in which the TCR dominates the binding and provides epitope selective interactions with T cells while retaining the activity of the MODs. Variant MODs with one or more substitutions (or deletions or insertions) that reduced the affinity of the MOD for their co-MOD may be incorporated into MAPPs and their higher order complexes alone or in combination with wild-type MODs polypeptide sequences. Wild-type and variant MODs are described further below.

The ability of MAPPs to modulate T cells in an epitope selective/specific manner thus provides methods of modulating activity of a T cell in vitro and in vivo, and accordingly, methods of treating disease such as cancers, infections, and disorders related to immune dysregulation/disfunction, including allergies and autoimmune diseases.

The present disclosure provides nucleic acids comprising nucleotide sequences encoding MAPP polypeptides, cells genetically modified with the nucleic acids and capable of producing the MAPP, and methods of producing MAPPs and their higher order complexes utilizing such cells.

Each presenting sequence or presenting complex present in a MAPP comprises MHC class II alpha and beta chain polypeptide sequences (e.g., human MHC class II sequences) sufficient to bind a peptide epitope and present it to a TCR. MHC Class II peptides, may include sequence variations that are designed to stabilize the MHC, stabilize the MHC peptide epitope complex, and/or stabilize the MAPP. Sequence variations may also serve to enhance cellular expression of MAPPs prepared in cell-based systems as well as the stability (e.g., thermal stability) of MAPPs and their higher order complexes such as duplex MAPPs. Some MHC class II sequences suitable for use in MAPPs are described below.

As indicated in the description of the drawings, MAPPs may comprise one or more independently selected peptide sequences or (one or more “linker” or “linkers”) between any two or more components of the MAPP, which in the figures may be shown as a line between peptide and/or polypeptide elements of the MAPPs. The same sequences used as linkers may also be located at the N- and/or C-termini of the MAPP peptides to prevent, for example, proteolytic degradation. Linker sequences include but are not limited to polypeptides comprising: glycine; glycine and serine; glycine and alanine; alanine and serine; and glycine, alanine and serine; any one which may comprise a cysteine for formation of an intra or interpolypeptide disulfide bond. Various linkers are described in more detail below.

2. Exemplary MAPP Architectures

MAPPs of the present disclosure comprise (i) framework polypeptides with a multimerization sequence and at least one dimerization sequence, and (ii) dimerization polypeptides with a counterpart dimerization sequence that binds with the framework polypeptide's dimerization sequence. As discussed above, MAPPs typically will further comprise either one or more epitope presenting sequences or one or more epitope presenting complexes. Exemplary structures for such MAPPs appear in FIG. 1A, 1B, and FIGS. 19-23. The structures depicted in FIG. 23 represents MAPPs with multimerizing framework polypeptides and epitope presenting sequences (the “Single Chain MHC” with the “Epitope”). In FIGS. 19-22, the structures represent MAPPs with multimerizing framework polypeptides where the epitope MHC combination represents either epitope presenting sequences or epitope presenting complexes.

Interactions of MHC (e.g., HLA) sequences are not considered herein to result in multimerization and/or dimerization. In an embodiment, neither the dimerization sequence nor the multimerization sequence of the framework polypeptide, nor the counterpart dimerization sequence of the dimerization polypeptide comprises a Class II MHC polypeptide sequence having at least 90% (e.g., 95% or 98%) sequence identity to at least 15 (e.g., at least 20, 30, 40, 50, 60 or 70) contiguous as of a MHC Class II polypeptide (e.g., a polypeptide in any of FIGS. 4 to 18B). In embodiments, MAPPs comprise at least one, or at least two, dimerization peptides that comprise an epitope presenting sequence. See, e.g., FIG. 1A.

One group of MAPPs, those having epitope presenting sequences, comprise: a multimerizing framework polypeptide having, from N-terminus to C-terminus, a dimerization sequence and multimerization sequence; and a dimerization polypeptide comprising a counterpart dimerization sequence complementary to the dimerization sequence of the framework polypeptide and dimerizing therewith through covalent and/or non-covalent interactions to form a heterodimer; wherein at least one (e.g., one, or both) of a dimerization polypeptide and the framework polypeptide comprise a presenting sequence located on the N-terminal side of their dimerization or counterpart dimerization sequences. In such a MAPP the presenting sequence may comprise a peptide epitope and one or more MHC polypeptide sequences, with the peptide epitope sequence located: (i) at or within 10 aa, 15 aa, 20 aa, or 25 aa of the N-terminus of the presenting sequence, or (ii) in a polypeptide located at the N-terminus of the presenting sequence comprising, from N-terminus to C-terminus, a MOD, one or more optional linkers, and the peptide epitope; optionally at least one (e.g., one, two or each) of the framework polypeptide, dimerization peptide, and presenting sequence comprises one or more independently selected MODs located at their N-terminus and/or C-terminus (or on the N-terminal or C-terminal side of the dimerization or counterpart dimerization sequences); wherein the MHC polypeptide sequences are MHC class II polypeptide sequences and they comprise MHC class II α1, α2, β1, and β2 polypeptide sequences (e.g., human MHC class II sequences). In an embodiment, neither the dimerization sequence nor the multimerization sequence of the framework polypeptide comprises a class II MHC peptide sequence having at least 90% (e.g., 95% or 98%) sequence identity to at least 15 (e.g., at least 20, 30, 40, 50, 60 or 70) contiguous aas of a MHC class II polypeptide in any of FIGS. 4 to 18B.

Another group of MAPPs, those having epitope presenting complexes, comprise: a multimerizing framework polypeptide having, from N-terminus to C-terminus, a dimerization sequence and multimerization sequence; and a dimerization polypeptide comprising a counterpart dimerization sequence complementary to the dimerization sequence of the framework polypeptide and dimerizing therewith through covalent and/or non-covalent interactions to form a heterodimer; wherein at least one (e.g., one, or both) of a dimerization polypeptides and/or at least one (e.g., one or both) of the framework polypeptide comprise a presenting complex 1st sequence located on the N-terminal side of their dimerization sequence. A presenting complex 2nd sequence is associated with the presenting complex 1st sequence (e.g., non-covalently or covalently such as by one or two interchain disulfide bonds) to form a presenting complex. In such a MAPP each of the presenting complex 1st sequence and its associated presenting complex 2nd sequence are comprised of one or more MHC polypeptide sequences, with one of the sequences further comprising the peptide epitope. The peptide epitope may be located (i) at or within 10 aa, 15 aa, 20 aa, or 25 aa of the N-terminus of the presenting complex 1st sequence or presenting complex 2nd sequence, or (ii) in a polypeptide located at the N-terminus of the presenting complex 1^{st} sequence or presenting complex 2^{nd} sequence, with the polypeptide comprising, from N-terminus to C-terminus, a MOD, one or more optional linkers, and the peptide epitope. Optionally, at least one (e.g., one, two or each) of the framework polypeptide, dimerization peptide, or the peptides of a presenting complex comprise one or more independently selected MODs located at their N-terminus or C-terminus (or on the N-terminal or C-terminal side of the dimerization sequences).

MAPPs of the present disclosure may be constructed such that neither the dimerization sequence nor the multimerization sequence of the framework polypeptide comprises a class II MHC peptide sequence having at least 90% (e.g., 95% or 98%) sequence identity to at least 15 (e.g., at least 20, 30, 40, 50, 60 or 70) contiguous aas of a MHC class II polypeptide in any of FIGS. 4 to 18B.

As discussed above, a dimerization sequence of a framework polypeptide may interact with dimerization peptides to form heterodimers. The multimerization sequence of the framework polypeptide may associate with another framework polypeptide multimerization sequence forming a duplex (or higher order structure, such as a triplex, quadraplex or pentaplex) of the heterodimers. Where the multimerization sequences are interspecific (e.g., a knob-in-hole Fc peptide pair), and at least one heterodimer comprises an interspecific dimerization and counterpart dimerization pair, two different heterodimers may be formed. When the different heterodimers are combined to form a duplex MAPP, any one or more component (e.g., MODs) may differ (e.g., in type or location) between the two heterodimers.

C. MAPP Components

1. Framework Polypeptides and Dimerization Polypeptides

As may be understood from the preceding sections, framework polypeptides serve as the structural basis or skeleton of MAPPs, permitting the organization of other elements in the MAPP complex. Framework peptides interact with other peptides through binding interactions, principally at dimerization and multimerization sequences. Interactions at dimerization sequences permit association of non-framework peptides (e.g., dimerization peptides) with framework peptides. In contrast, multimerization sequences are involved in the interaction of two or more framework peptides.

The framework polypeptide(s) of MAPPs comprise at least one multimerization sequence, and at least one independently selected dimerization sequence that is not identical to, or of the same type (e.g., not both leucine zipper variants) as the multimerization sequence. By utilizing different types of sequences for the interactions at multimerization and dimerization sequences, it becomes possible to control the interactions of the framework polypeptide with other framework polypeptides and with dimerization polypeptides. In an embodiment, framework polypeptides comprise one multimerization sequence and one dimerization sequence. In an embodiment, framework polypeptides comprise at least one multimerization sequence and at least two independently selected dimerization sequences. Framework peptides may contain peptide sequences (e.g., linker sequences and/or MOD sequences) between any of the elements of the framework polypeptide or at the ends of the framework polypeptide including the multimerization sequences and dimerization sequences.

In addition to providing for the structural organization of MAPPs through their multimerization and dimerization sequences, framework peptides, and particularly their N- and C-termini, may also serve as locations for placement of elements such as MOD sequences, an epitope, presenting sequences, and/or a presenting complex 1^{st} sequence (one polypeptide of an epitope presenting complexes, see FIG. 1B). When placed at the N- and/or C-termini of a framework polypeptide, such polypeptide elements are part of the framework polypeptide (e.g., a single translation product formed in a cell).

Within a MAPP, all of the dimerization sequence may be non-interspecific (such as leucine zipper pairs) while the multimerization sequences is either interspecific or non-interspecific (see e.g., structures A & B of FIGS. 9 and 10). For example, in a duplex MAPP with first and second framework polypeptides, the multimerization sequences may be a non-interspecific (e.g., IgFc (e.g., CH2, CH3 domains) or leucine zippers) or the multimerization sequences may be an interspecific knob-in-hole sequence pair; with the dimerization sequences of the first and second framework polypeptide as a non-interspecific leucine zipper polypeptides. Where an Fc polypeptide is employed it may be, for example, from an IgA, IgD, IgE, IgG, or IgM, which may be a human polypeptide sequence, a humanized polypeptide sequence, a Fc region polypeptide of a synthetic heavy chain constant region, or a consensus heavy chain constant region.

Within a MAPP, all of the dimerization sequences may be interspecific, while the multimerization sequences are not interspecific (see e.g., FIG. 23 A). For example, in a duplex MAPP with first and second framework polypeptides, the multimerization sequences may be an IgFc sequence, with the a ZW1 sequence or its counterpart employed as the dimerization sequence of the first framework polypeptide and an Ig CH1 domain or its counterpart Ig C_{L} κ sequence as the dimerization sequence of the second framework polypeptide.

All of the dimerization sequences or all of the dimerization and multimerization sequences, in a MAPP may differ in that they bind only specific binding partners present in the MAPP (e.g., each are part of a different interspecific sequence pair). For example, in a duplex MAPP with first and second framework polypeptides, the multimerization sequences may be a pair of knob-in-hole IgFc sequences, with the a ZW1 sequence or its counterpart employed as the dimerization sequence of the first framework polypeptide, and a Ig CH1 or its counterpart Ig C_{L} sequence as the dimerization sequence of the second framework polypeptide.

2. Multimerization and Dimerization Polypeptide Sequences

Amino acid sequences that permit polypeptides to interact may be utilized as dimerization sequences or counterpart dimerization sequences when they are involved in the formation of dimers between a framework polypeptide and a dimerization polypeptide. The same type of aa sequences may be utilized as multimerization sequences when they are used to form duplex or higher order structures (trimers, tetramers, pentamer, etc.) between framework polypeptides. In any given MAPP, sequences that can interact with each other are not utilized as both dimerization and multimerization sequences. Stated another way, the same aa sequence pair may serve as either dimerization or multimerization sequences depending on whether they: bring together two or more framework peptides, in which case they are multimerization sequences; or they bring together a dimerization and multimerization sequence, in which case they are designated as dimerization sequences.

Where dimerization or multimerization sequences employ identical sequences that pair or multimerize (e.g., some leucine zipper sequences), they can form symmetrical pairs or multimers (e.g., homodimers) as shown in FIG. 19 structure A. In contrast, where dimerization or multimerization sequences that pair are not identical and require a specific complementary counterpart sequence to form a dimer, they are interspecific binding sequences and can form asymmetric pairs. Both immunoglobulin (e.g., IgFc) and non-immunoglobulin polypeptides can be interspecific or non-interspecific in nature. For example, both Fos/Jun binding pairs and Ig CH1 polypeptide sequences and light chain constant region C_{L} sequences form interspecific binding pairs. Natural Ig Fc regions tend to be non-interspecific, but, as discussed below, can be made to form interspecific pairs (e.g., KiH and KiHs-s pairs). Coiled-coil sequences, including leucine zipper sequences, can be either interspecific leucine zipper or non-interspecific leucine zipper sequences. See e.g., Zeng et al., (1997) PNAS (USA) 94:3673-3678; and Li et al., (2012), Nature Comms. 3:662.

Interspecific binding sequences may in some instances form some amount of homodimers, but preferentially dimerize by binding more strongly with their counterpart interspecific binding sequence. Accordingly, specific heterodimers tend to be formed when an interspecific dimerization sequence and its counterpart interspecific binding sequence are incorporated into a pair of polypeptides. By way of example, where an interspecific dimerization sequence and its counterpart are incorporated into a pair of polypeptides, they may selectively form greater than 70%, 80%, 90%, 95%, 98% or 99% heterodimers when an equimolar mixture of the polypeptides are combined (for example in PBS buffer at 20° C.). The remainder of the polypeptides may be present as monomers or homodimers, which may be separated from the heterodimer. See, for example, FIG. 19, structure B, with an interspecific multimerization sequence and structure C with two different interspecific dimerization sequences. Moreover, because interspecific sequences are selective for their counterpart sequence, they can limit the interaction with other proteins expressed by cells (e.g., in culture or in a subject) particularly where the interspecific sequences are not naturally occurring or are variants of naturally occurring protein sequences.

Sequence are considered orthogonal to other sequences when they do not form complexes (bind) with each other's counterpart sequences. See FIG. 19 structure D where the MAPP comprises an interspecific multimerization sequence and two independently selected interspecific dimerization sequences, all of which are orthogonal to each other. Any of the MAPPS described herein may have two or more (e.g., three, four or more) orthogonal dimerization sequences. In an embodiment, MAPPs with multimerizing framework peptides may have orthogonal multimerization and dimerization domains (where the dimerization domains may or may not be orthogonal to each other).

Some sequences permitting polypeptides to interact with sufficient affinity to be used as dimerization and/or multimerization sequences are provided for example in U.S. Patent Publication No. 2003/0138440. The sequences may be of relatively compact size (e.g., such as less than about 300, 250, 225, 200, 175, 150, 125, 100, 75, 60, 50, 40, or 30 aa). In an embodiment, at least one (e.g., at least two or all) of the dimerization and/or multimerization sequences are less than 300 aa. In an embodiment, at least one (e.g., at least two or all) of the dimerization and/or multimerization sequences are less than 200 aa. In an embodiment, at least one (e.g., at least two or all) of the dimerization and/or multimerization sequences are less than 100 aa. In an embodiment, at least one (e.g., at least two or all) of the dimerization and/or multimerization sequences are less than 75 aa. In another embodiment, at least one (e.g., at least two or all) of the dimerization and/or multimerization sequences are less than are less than 50 aa. In an embodiment, at least one (e.g., at least two or all) of the dimerization and/or multimerization sequences are less than 30 aa.

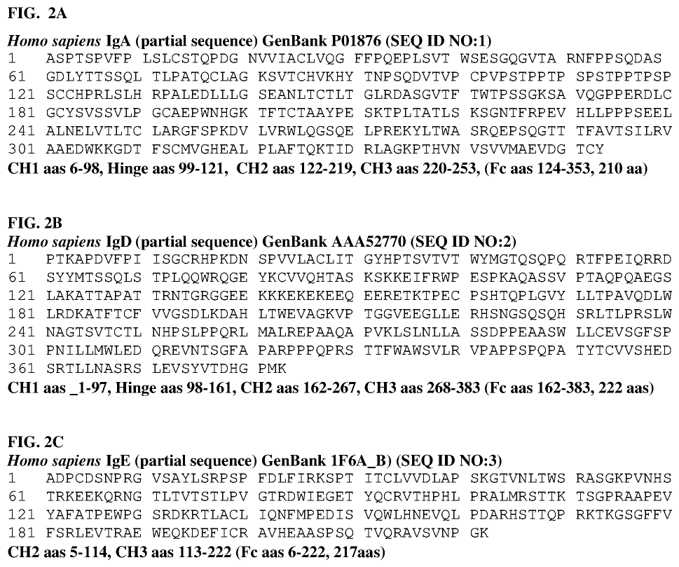

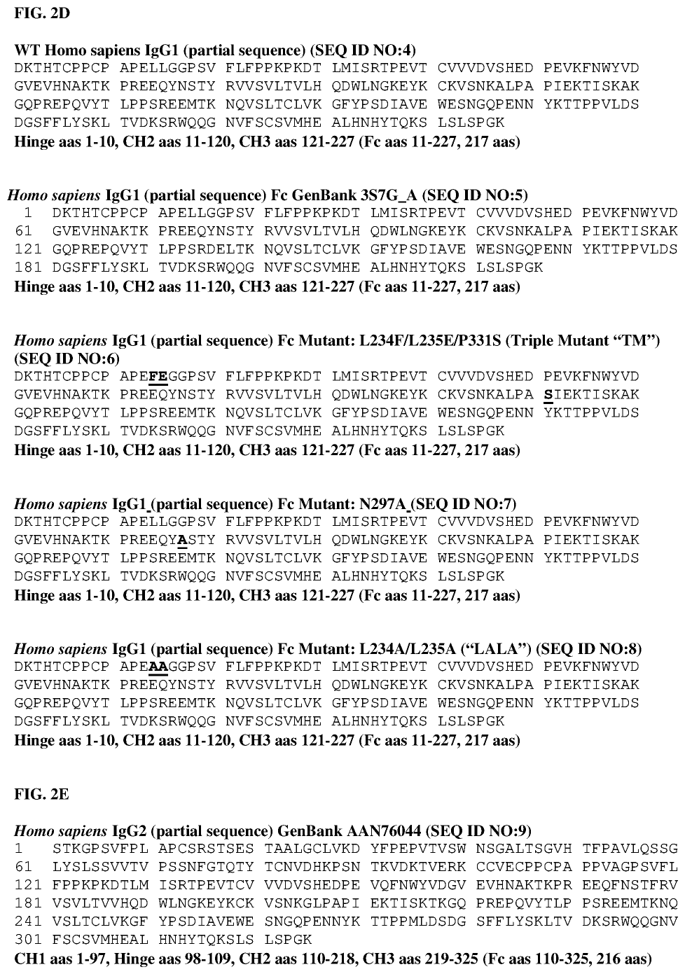

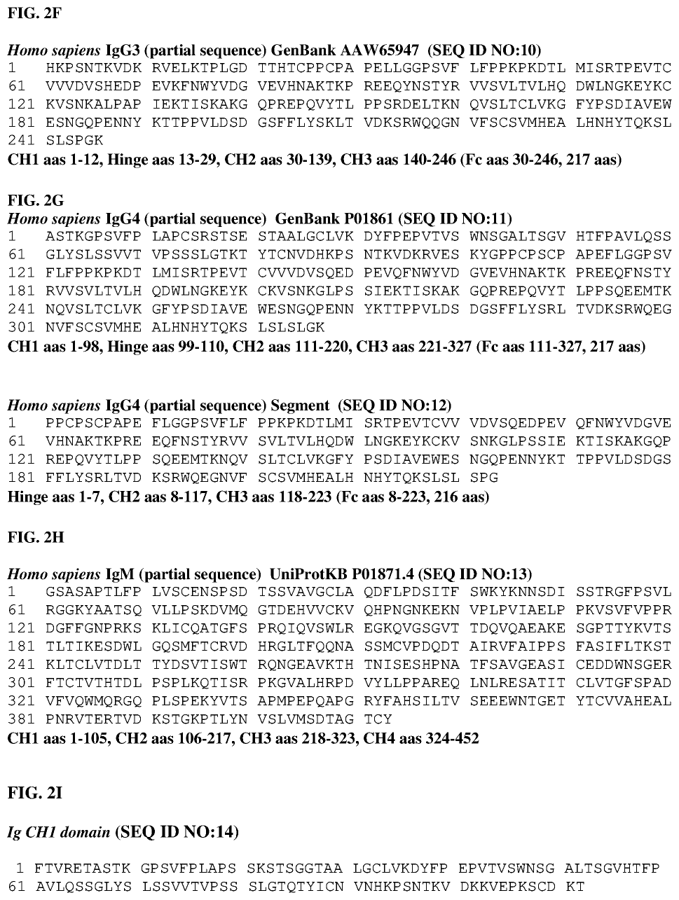

Dimerization/multimerization sequences include but are not limited to: immunoglobulin heavy chain constant region (Ig Fc) polypeptide sequences (e.g., sequences comprising CH2-CH3 regions of immunoglobulins such as those provided in FIGS. 2A-2H and SEQ ID NOs: 1 to 13); polypeptides of the collectin family (e.g., ACRP30 or ACRP30-like proteins) that contain collagen domains consisting of collagen repeats Gly-Xaa-Yaa and/or Gly-Xaa-Pro (which may be repeated from 10-40 times); coiled-coil domains; leucine-zipper domains; interspecific Ig Fc heavy chain constant regions (such as knob-in-hole sequences described in more detail below); Fos/Jun binding pairs; immunoglobulin heavy chain constant region (CH2-CH3) sequences, and; Ig CH1 and light chain constant region CL sequences (Ig CH1/CL pairs such as a Ig CH1 sequence paired with a Ig C_{L} κ or λ light chain constant region sequence).

Framework and/or dimerization polypeptides of a MAPP may comprise an immunoglobulin heavy chain constant region (e.g., CH2-CH3 domains) polypeptide sequence that functions as a dimerization or multimerization sequence. Where the framework polypeptide comprises an IgFc multimerization sequence, and a CH1 dimerization sequence it may comprise all or part a native or variant immunoglobulin sequence set forth in any of FIGS. 2A to 2H that comprise the CH1, CH2 and CH3 domains and any hinge sequences that may be present. An Ig Fc sequence, or any one or more of the CH1, CH2, and CH3 domains, may have at least about 70%, at least about 80%, at least about 90%, at least about 95%, at least about 98%, at least about 99%, or 100% aa sequence identity to an aa sequence of an Fc region depicted in FIGS. 2A-2H. In particular, the terminal lysine provided in some of the sequences provided in FIGS. 2A-2H (e.g., the IgG sequences in FIGS. 2D, 2E, 2F, and 2G) may be removed during cellular processing of the MAPPs and may not be present on some or all of the MAPP molecules as expressed. See, e.g., van den Bremer et al. (2015) mAbs 7:4; and Sissolak et al. (2019) J. Industrial Microbiol. & Biotechnol. 46:1167. Alternatively, in preparing MAPPs, the nucleotides encoding a C-terminal lysine may simply be omitted from any of the sequences provided in FIGS. 4A-4H in which a C-terminal lysine occurs.

Such immunoglobulin sequences can covalently link the polypeptides of MAPP complex together by forming one or two interchain disulfide bonds, thereby stabilizing MAPPs, particularly where a pair of interspecific Ig sequence such as knob-in-hole polypeptide pairs are employed. Where an Fc polypeptide sequence, alone or in combination with a CH1 polypeptide sequence, is employed as a multimerization or dimerization sequence it may be, for example, from an IgA, IgD, IgE, IgG, or IgM, which may be a human polypeptide sequence, a humanized polypeptide sequence, a Fc region polypeptide of a synthetic heavy chain constant region, or a consensus heavy chain constant region. As discussed below, the Ig Fc region can further contain substitutions that can substantially remove the ability of the Ig Fc to effect complement-dependent cytotoxicity (CDC) or antibody-dependent cell cytotoxicity (ADCC). Accordingly, framework and/or dimerization polypeptides, and in particular Ig Fc sequences used as multimerization or dimerization sequences, may comprise substitutions that reduce or substantially eliminate ADCC and/or CDC responses.

Framework and/or dimerization polypeptides of a MAPP may comprise a sequence that has at least about 70%, at least about 80%, at least about 90%, at least about 95%, at least about 98%, at least about 99%, or 100% aa sequence identity to at least 150 contiguous aas (at least 175, at least 200, at least 225, at least 250, at least 275, at least 300, at least 325, or at least 350 contiguous aas), or all aas, of the IgA Fc sequence depicted in FIG. 2A (SEQ ID NO:1). Framework and/or dimerization polypeptides of a MAPP may comprise a sequence that has at least about 70%, at least about 80%, at least about 90%, at least about 95%, at least about 98%, at least about 99%, or 100% aa sequence identity to at least 150 contiguous aas (at least 175, at least 200, at least 225, at least 250, at least 275, at least 300, at least 325, or at least 350 contiguous aas), or all aas, of the IgD Fc sequence depicted in FIG. 2B (SEQ ID NO:2). Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 contiguous aas (at least 150, at least 175, or at least 200 contiguous aas), or all aas, of the IgE Fc sequence depicted in FIG. 2C (SEQ ID NO:3).

A MAPP may comprise one or more IgG Fc sequences as dimerization and/or multimerization sequences. The Fc polypeptide of a MAPP can be a human IgG1 Fc, a human IgG2 Fc, a human IgG3 Fc, a human IgG4 Fc, etc. In some cases, the Fc sequence has at least about 70%, at least about 80%, at least about 90%, at least about 95%, at least about 98%, at least about 99%, or 100% aa sequence identity to an aa sequence of an Fc region depicted in FIG. 2D-2G. Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 contiguous aas (e.g., at least 150, at least 175, at least 200, or at least 220 contiguous aas), or all aas, of the wt. IgG1 Fc polypeptide sequence depicted in FIG. 2D (SEQ ID NO:4). Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 (e.g., at least 150, at least 175, at least 200, or at least 225) contiguous aas, or all aas, of the IgG2 Fc polypeptide sequence depicted in FIG. 2E (SEQ ID NO:9). Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 (e.g., at least 150, at least 175, at least 200, at least 225, or at least 240) contiguous aas, or all aas, of the IgG3 Fc sequence depicted in FIG. 2F (SEQ ID NO:10). Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 (e.g., at least 150, at least 175, at least 200, or at least 220) contiguous aas, or all aas, of the IgG4 Fc sequence depicted in FIG. 2G (SEQ ID NO:11 or 12).

Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 (at least 150, at least 175, at least 200, at least 225, or at least 250) contiguous aas, or all aas, of the IgM Fc polypeptide sequence depicted in FIG. 2H (SEQ ID NO:13).

Framework and/or dimerization polypeptides of a MAPP comprising immunoglobulin sequences (e.g., depicted in FIGS. 2A-2H) can be covalently linked together by formation of at least one or at least two interchain disulfide bonds between cysteines that are adjacent to the immunoglobulin hinge regions. Such disulfide bonds can stabilize the interaction of framework and dimerization polypeptide heterodimers, or, for example, duplexes of such heterodimers when the disulfide bonds are between framework multimerization sequences.

A framework or dimerization polypeptide may comprise an aa sequence having 100% aa sequence identity to the wt. human IgG1 Fc polypeptide depicted in FIG. 2D. A framework or dimerization polypeptide may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 2D, that includes a substitution of N297 (N77 as numbered in FIG. 2D, SEQ ID NO:7) with an aa other than asparagine. In one case, N297 is substituted by alanine, (N297A). Substitutions at N297 lead to the removal of carbohydrate modifications and result antibody sequences with reduced complement component 1q (“C1q”) binding compared to the wt. protein, and accordingly a reduction in CDC. K322 (e.g., K322A) substitutions shows a substantial reduction in reduction in FcγR binding affinity and ADCC, with the C1q binding and CDC functions substantially or completely eliminated. Hezareh et al., (2001) J. Virol. 75:12161-168.

Amino acid L234 and other aas in the lower hinge region (e.g., aas 234 to 239, such as L235, G236, G237, P238, S239) which correspond to aas 14-19 of SEQ ID NO:8) of IgG are involved in binding to the Fc gamma receptor (FcγR), and accordingly, mutations at that location reduce binding to the receptor (relative to the wt. protein) and result in a reduction in antibody-dependent cellular cytotoxicity (or alternatively antibody-dependent cell-mediated cytotoxicity, “ADCC”). Hezareh et al., (2001) have demonstrated that the double mutant (L234A, L235A) does not effectively bind either FcγR or C1q, and both ADCC and CDC functions were substantially or completely abolished. A framework or dimerization polypeptide with a substitution in the lower hinge region may comprise an aa sequence having at least about 70% (e.g., at least about 80%, 90%, 95%, 98%, or 99%) aa sequence identity to at least 125 contiguous aas (e.g., at least 150, at least 175, at least 200, or at least 210 contiguous aas), or all aas, of the wt. human IgG1 Fc polypeptide depicted in FIG. 2D, that includes a substitution of L234 (L14 of the aa sequence depicted in FIG. 2D) with an aa other than leucine.

A framework or dimerization polypeptide with a substitution in the lower hinge region may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 2D, that includes a substitution of L235 (L15 of the aa sequence depicted in FIG. 2D) with an aa other than leucine. In some cases, the framework and/or dimerization polypeptide present in a MAPP with substitutions in the lower hinge region includes L234A and L235A (“LALA”) substitutions (the positions corresponding to positions 14 and 15 of the wt. aa sequence depicted in FIG. 2D; see, e.g., SEQ ID NO:8).

A framework or dimerization polypeptide with a substitution in the lower hinge region may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 2D, that includes a substitution of P331 (P111 of the aa sequence depicted in FIG. 2D) with an aa other than proline. Substitutions at P331, like those at N297, lead to reduced binding to C1q relative to the wt. protein, and thus a reduction in complement dependent cytotoxicity (CDC). In one embodiment, the substitution is a P331S substitution. In another embodiment, the substitution is a P331A substitution.

A framework or dimerization polypeptide may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 2D, and include substitutions of D270, K322, and/or P329 (corresponding to D50, K102, and P109 of SEQ ID NO:4 in FIG. 2D) that reduce binding to C1q protein relative to the wt protein.

A framework or dimerization polypeptide may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 2D, including substitutions at L234 and/or L235 (L14 and/or L15 of the aa sequence depicted in FIG. 2D) with aas other than leucine such as L234A and L235A, and a substitution of P331 (P111 of the aa sequence depicted in FIG. 2D) with an aa other than proline such as P331S. In one instance, a framework or dimerization polypeptide present in a MAPP comprises the “Triple Mutant” aa sequence (SEQ ID NO:6) depicted in FIG. 2D (human IgG1 Fc) having L234F, L235E, and P331S substitutions (corresponding to aa positions 14, 15, and 111 of the aa sequence depicted in FIG. 2D).

Where an asymmetric pairing between two polypeptides of a MAPP is desired, a framework or dimerization polypeptide present in a MAPP may comprise, consist essentially of, or consist of an interspecific binding sequence. Interspecific binding sequences favor formation of heterodimers with their cognate polypeptide sequence (i.e., the interspecific sequence and its counterpart interspecific sequence), particularly those based on immunoglobulin Fc (Ig Fc) sequence variants. Such interspecific polypeptide sequences include (KiH) and (KiHs-s), HA-TF, ZW-1, 7.8.60, DD-KK, EW-RVT, EW-RVTs-s, and A107 sequences. One interspecific binding pair comprises a T366Y and Y407T mutant pair in the CH3 domain interface of IgG1, or the corresponding residues of other immunoglobulins. See Ridgway et al., Protein Engineering 9:7, 617-621 (1996). A second interspecific binding pair involves the formation of a knob by a T366W substitution, and a hole by the triple substitutions T366S, L368A and Y407V on the complementary Ig Fc sequence. See Xu et al. mAbs 7:1, 231-242 (2015). Another interspecific binding pair has a first Fc polypeptide with Y349C, T366S, L368A, and Y407V substitutions and a second Ig Fc polypeptide with S354C, and T366W substitutions (disulfide bonds can form between the Y349C and the S354C). See e.g., Brinkmann and Konthermann, mAbs 9:2, 182-212 (2015). Ig Fc polypeptide sequences, either with or without knob-in-hole modifications, can be stabilized by the formation ofdisulfide bonds between the Ig Fc polypeptides (e.g., the hinge region disulfide bonds). Several interspecific binding sequences based upon immunoglobulin sequences are summarized in the table that follows, with cross reference to the numbering of the aa positions as they appear in the wt. IgG1 sequence (SEQ ID NO:4) set forth in FIG. 2D shown in brackets “{ }”.

TABLE 1
Interspecific immunoglobulin sequences and their cognate counterpart interspecific sequences

| | Substitutions in the first | Substitutions in the second | |
|---|---|---|---|
| Interspecific | interspecific polypeptide | (counterpart) interspecific | |
| Pair Name | sequence | polypeptide sequence | Comments |
| KiH | T366W | T366S/L368A/Y407V | Hydrophobic/steric |
| | {T146W} | {T146S/L148A/Y187V} | complementarity |
| KiHs-s | T366W/S354C* | T366S/L368A/Y407V/Y349C | KiH + inter-CH3 |
| | {T146W/S134C*} | {T146S/L148A/Y187V/Y129C} | domain S—S bond |
| HA-TF | S364H/F405A | Y349T/T394F | Hydrophobic/steric |
| | {S144H/F185A} | {Y129T/T174F} | complementarity |
| ZW1 | T350V/L351Y/F405A/Y407V | T350V/T366L/K392L/T394W | Hydrophobic/steric |
| | {T130V/L131Y/F185A/Y187V} | {T130V/T146L/K172L/T174W} | complementarity |
| 7.8.60 | K360D/D399M/Y407A | E345R/Q347R/T366V/K409V | Hydrophobic/steric |
| | {K140D/D179M/Y187A} | {E125R/Q127R/T146V/K189V} | complementarity + |
| | | | electrostatic |
| | | | complementarity |
| DD-KK | K409D/K392D | D399K/E356K | Electrostatic |
| | {K189D/K172D} | {D179K/E136K} | complementarity |
| EW-RVT | K360E/K409W | Q347R/D399V/F405T | Hydrophobic/steric |
| | {K140E/K189W} | {Q127R/D179V/F185T} | complementarity & |
| | | | long-range electro- |
| | | | static interaction |
| EW-RVTs-s | K360E/K409W/Y349C* | Q347R/D399V/F405T/S354C | EW-RVT + inter- |
| | {K140E/K189W/Y129C*} | {Q127R/D179V/F185T/S134C} | CH3 domain S—S |
| | | | bond |
| A107 | K370E/K409W | E357N/D399V/F405T | Hydrophobic/steric |
| | {K150E/K189W} | {E137N/D179V/F185T} | complementarity + |
| | | | hydrogen bonding |
| | | | complementarity |

Table 1 modified from Ha et al., Frontiers in Immunol. 7: 1-16 (2016).
*aa forms a stabilizing disulfide bond.

In addition to the interspecific pairs of sequences in Table 1, framework and/or dimerization polypeptides may include interspecific “SEED” sequences having 45 residues derived from IgA in an IgG1 CH3 domain of the interspecific sequence, and 57 residues derived from IgG1 in the IgA CH3 in its counterpart interspecific sequence. See Ha et al., Frontiers in Immunol. 7:1-16 (2016).

A framework or dimerization polypeptide found in a MAPP may comprise an interspecific binding sequence or its counterpart interspecific binding sequence selected from the group consisting of: KiH; KiHs-s; HA-TF; ZW-1; 7.8.60; DD-KK; EW-RVT; EW-RVTs-s; A107; or SEED sequences.

A MAPP may comprise a framework or dimerization polypeptide comprising an IgG1 KiH or KiHs-s sequence with a T146W sequence substitution, and its counterpart interspecific KiH or KiHs-s binding partner polypeptide comprises an IgG1 sequence having T146S, L148A, and Y187V sequence substitutions, where the framework and/or dimerization polypeptides comprises a sequence having at least 80%, at least 90%. at least 95%, or at least 97% sequence identity to at least 100 (e.g., at least 125, 150, 170, 180, 190, 200, 210, 220, or all 227) contiguous aas of the wt. IgG1 of FIG. 2D. One or both of the framework, or both of dimerization polypeptides optionally comprising substitutions at one of more of: L234 and L235 (e.g., L234A/L235A “LALA” or L234F/L235E); N297 (e.g., N297A); P331 (e.g., P331S); L351 (e.g., L351K); T366 (e.g., T366S); P395 (e.g., P395V); F405 (e.g., F405R); Y407 (e.g., Y407A); and K409 (e.g., K409Y). Those substitutions appear at: L14 and L15 (e.g., L14A/L15A “LALA” or L14F/L15E); N77 (e.g., N77A); P111 (e.g., P111S) L131 (e.g., L131K); T146 (e.g., T146S); P175 (e.g., P175V); F185 (e.g., F185R); Y187 (e.g., Y187A); and K189 (e.g., K189Y) in the wt. IgG1 sequence of FIG. 2D.

A MAPP may comprise a framework or dimerization polypeptide comprising an IgG1 sequence with a T146W KiH sequence substitution, and its counterpart interspecific binding partner polypeptide comprises an IgG1 sequence having T146S, L148A, and Y187V KiH sequence substitutions, where the framework and/or dimerization polypeptides comprise a sequence having at least 80%, at least 90%. at least 95%, or at least 97% sequence identity to at least 100 (e.g., at least 125, 150, 170, 180, 190, 200, 210, 220, or all 227) contiguous aas of the wt. IgG1 of FIG. 2D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a T146W and S134C KiHs-s substitutions, and its counterpart interspecific binding partner polypeptide comprises an IgG1 sequence having T146S, L148A, Y187V and Y129C KiHs-s substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a S144H and F185A HA-TF substitutions, and its counterpart interspecific binding partner polypeptide comprises an IgG1 sequence having Y129T and T174F HA-TF substitutions, where the framework and/or dimerization polypeptides comprise a sequence having at least 80%, at least 90%. at least 95%, or at least 97% sequence identity to at least 100 (e.g., at least 125, 150, 170, 180, 190, 200, 210, 220, or all 227) contiguous aas of the wt. IgG1 of FIG. 2D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a T130V, L131Y, F185A, and Y187V ZW1 substitutions, and its counterpart interspecific binding partner polypeptide comprises an IgG1 sequence having T130V, T146L, K172L, and T174W ZW1 substitutions, where the framework and/or dimerization polypeptides comprise a sequence having at least 80%, at least 90%. at least 95%, or at least 97% sequence identity to at least 100 (e.g., at least 125, 150, 170, 180, 190, 200, 210, 220, or all 227) contiguous aas of the wt. IgG1 of FIG. 2D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a K140D, D179M, and Y187A 7.8.60 substitutions, and its counterpart interspecific binding partner polypeptide comprises an IgG1 sequence having T130V E125R, Q127R, T146V, and K189V 7.8.60 substitutions, where the framework and/or dimerization polypeptides comprise a sequence having at least 80%, at least 90%. at least 95%, or at least 97% sequence identity to at least 100 (e.g., at least 125, 150, 170, 180, 190, 200, 210, 220, or all 227) contiguous aas of the wt. IgG1 of FIG. 2Ds

A MAPP may comprise a framework or dimerization polypeptide comprising an IgG1 sequence with a K189D, and K172D DD-KK substitutions, and its counterpart interspecific binding partner polypeptide comprises an IgG1 sequence having T130V D179K and E136K DD-KK substitutions, where the framework and/or dimerization polypeptides comprise a sequence having at least 80%, at least 90%. at least 95%, or at least 97% sequence identity to at least 100 (e.g., at least 125, 150, 170, 180, 190, 200, 210, 220, or all 227) contiguous aas of the wt. IgG1 of FIG. 2D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a K140E and K189W EW-RVT substitutions, its counterpart interspecific binding partner polypeptide comprises an IgG1 sequence having T130V Q127R, D179V, and F185T EW-RVT substitutions, where the framework and/or dimerization polypeptides comprise a sequence having at least 80%, at least 90%. at least 95%, or at least 97% sequence identity to at least 100 (e.g., at least 125, 150, 170, 180, 190, 200, 210, 220, or all 227) contiguous aas of the wt. IgG1 of FIG. 2D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a K140E, K189W, and Y129C EW-RVTs-s substitutions, its counterpart interspecific binding partner polypeptide comprises an IgG1 sequence having T130V Q127R, D179V, F185T, and S134C EW-RVTs-s substitutions, where the framework and/or dimerization polypeptides comprise a sequence having at least 80%, at least 90%. at least 95%, or at least 97% sequence identity to at least 100 (e.g., at least 125, 150, 170, 180, 190, 200, 210, 220, or all 227) contiguous aas of the wt. IgG1 of FIG. 2D. One or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a K150E and K189W A107 substitutions, its counterpart interspecific binding partner polypeptide comprises an IgG1 sequence having T130V E137N, D179V, and F185T A107 substitutions, where the framework and/or dimerization polypeptides comprise a sequence having at least 80%, at least 90%. at least 95%, or at least 97% sequence identity to at least 100 (e.g., at least 125, 150, 170, 180, 190, 200, 210, 220, or all 227) contiguous aas of the wt. IgG1 of FIG. 2D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

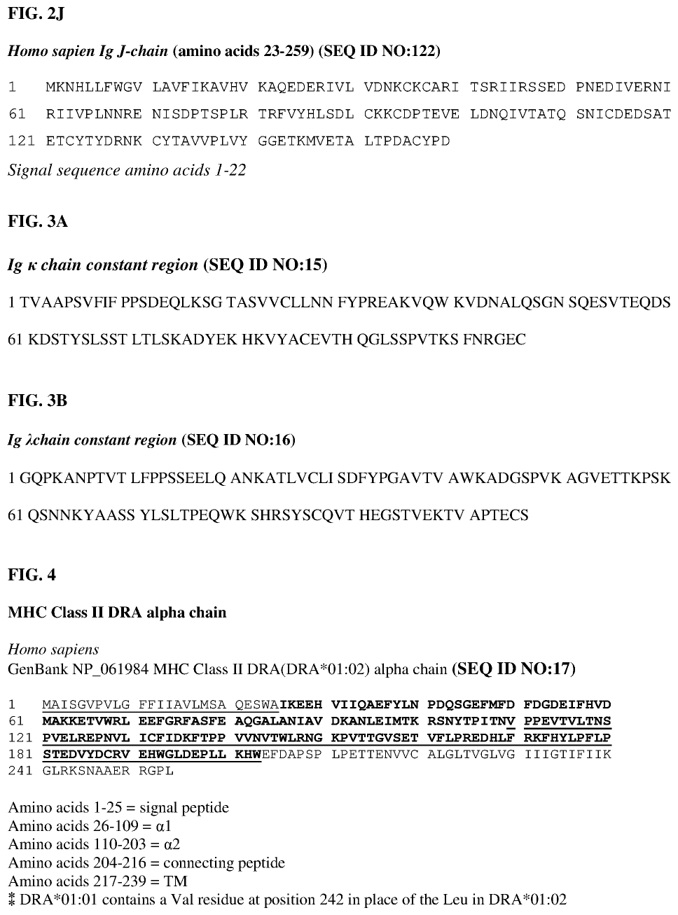

As an alternative to the use of immunoglobulin CH2 and CH3 heavy chain constant regions as dimerization or multimerization sequences, immunoglobulin light chain constant regions (See FIGS. 3A and 3B) can be paired with Ig CH1 sequences (See FIG. 2I) as multimerization or dimerization sequences and their counterpart sequences of a framework polypeptide.

A MAPP framework or dimerization polypeptide may comprise an Ig CH1 domain (e.g., the polypeptide of FIG. 2I), and the sequence with which it will form a complex (its counterpart binding partner) comprises an Ig κ chain constant region sequence, where the framework or dimerization polypeptide comprise a sequence having at least 80%, 85%, 90%. 95%, 98%, 99%, or 100% sequence identity to at least 70, at least 80, at least 90, at least 100, or at least 110 contiguous aas of SEQ ID NOs: 14 and/or 15 respectively. See FIGS. 2I and 3A. The Ig CH1 and Ig κ sequences may be modified to increase their affinity for each other, and accordingly the stability of any heterodimer formed utilizing them as a dimerization or multimerization sequences. Among the substitutions that increase the stability of CH1-Ig κ heterodimers are those identified as the MD13 combination in Chen et al., MAbs, 8(4):761-774 (2016). In the MD13 combination two substitutions are introduced into to each of the IgCH1 and Ig κ sequences. The Ig CH1 sequence is modified to contain S64E and S66V substitutions (S70E and S72V of the sequence shown in FIG. 2I). The Ig κ sequence is modified to contain S69L and T71S substitutions (S68L and T70S of the sequence shown in FIG. 3A).

A framework or dimerization polypeptide of a MAPP may comprise an Ig CH1 domain (e.g., the polypeptide of FIG. 2I SEQ ID NO:14), and its counterpart sequence comprises an Ig λ chain constant region sequence such as is shown in FIG. 3B (SEQ ID NO:16), where the framework or dimerization polypeptide comprises a sequence having at least 80%, 85%, 90%. 95%, 98%, 99%, or 100% sequence identity to at least 70 (e.g., at least 80, at least 90, or at least 100) contiguous aas of the sequences shown in FIG. 3B.

Framework and/or dimerization polypeptides of a MAPP may each comprise a leucine zipper polypeptide as a dimerization or multimerization sequence. The leucine zipper polypeptides bind to one another to form dimer (e.g., homodimer). Non-limiting examples of leucine-zipper polypeptides include a peptide comprising any one of the following aa sequences: RMKQIEDKIEEILSKIYHIENEIARIKKLIGER (SEQ ID NO:106); LSSIEKKQEEQTS-WLIWISNELTLIRNELAQS (SEQ ID NO:107); LSSIEKKLEEITSQLIQISNELTLIRNELAQ (SEQ ID NO:108; LSSIEKKLEEITSQLIQIRNELTLIRNELAQ (SEQ ID NO:109); LSSIEKKLEEITSQLQQIRNELTLIRNELAQ (SEQ ID NO:110); LSSLEKKLEEL-TSQLIQLRNELTLLRNELAQ (SEQ ID NO:111); ISSLEKKIEELTSQIQQLRNEITLLRNEIAQ (SEQ ID NO:112). In some cases, a leucine zipper polypeptide comprises the following aa sequence:

(SEQ ID NO: 113)
LEIEAAFLERENTALETRVAELRQRVQRLRNRVSQYRTRYGPLGGGK.

Additional leucine-zipper polypeptides are known in the art, a number of which are suitable for use as multimerization or dimerization sequences.

The framework and/or dimerization polypeptides of a MAPP may comprise a coiled-coil polypeptide that forms a dimer. Non-limiting examples of coiled-coil polypeptides include, for example, a peptide of any one of the following aa sequences: LKSVENRLAVVENQLKTVIEELKTVKDLLSN (SEQ ID NO:114); LARIEEKLKTIKAQLSEIASTLNMIREQLAQ (SEQ ID NO:115); VSRLEEKVKT-LKSQVTELASTVSLLREQVAQ (SEQ ID NO:116); IQSEKKIEDISSLIGQIQSEITLIRNEIAQ (SEQ ID NO:117); and LMSLEKKLEELTQTLMQLQNELSMLKNELAQ (SEQ ID NO:118).

A MAPP may comprise a pair of two framework polypeptides and/or a framework and dimerization polypeptide that each have an aa sequence comprising at least one cysteine residue that can form a disulfide bond permitting homodimerization or heterodimerization of those polypeptides stabilized by disulfide bond between the cysteine residues. Examples of such aa sequences include: VDLEGSTSN-GRQCAGIRL (SEQ ID NO:119); EDDVTTTEELAPALVPPPKGTCAGWMA (SEQ ID NO:120); and GHDQETTTQGPGVLLPLPKGACTGQMA (SEQ ID NO:121).

Some aa sequences suitable as multimerization (oligomerization) sequences permit formation of MAPPs capable of forming structures greater than duplexes of a heterodimers comprising a framework and dimerization polypeptide. In some instances, triplexes, tetraplexes, pentaplexes may be formed. Such aa sequences include, but are not limited to, IgM constant regions (see e.g., FIG. 2H) which forms hexamer, or pentamers (particularly when combined with a mature j-chain peptide lacking a signal sequence such as that provided in FIG. 2J (SEQ ID NO:122). Collagen domains, which form trimers, can also be employed. Collagen domains may comprise the three aa sequence Gly-Xaa-Xaa and/or GlyXaaYaa, where Xaa and Yaa are independently any aa, with the sequence appear or are repeated multiple times (e.g., from 10 to 40 times, such as 10-20, 20-30, or 30-40 times). In such sequences, Xaa and Yaa are frequently proline and hydroxyproline respectively in greater than 25%, 50%, 75%, 80% 90% or 95% of the Gly-Xaa-Yaa occurrences, or in each of the Gly-Xaa-Yaa occurrences. In some cases, a collagen domain comprises the sequence Gly-Xaa-Pro repeated from 10 to 40 times, such as 10-20, 20-30, or 30-40 times. A collagen oligomerization peptide can comprise the following aa sequence:

(SEQ ID NO: 123)
VTAFSNMDDMLQKAHLVIEGTFIYLRDSTEFFIRVRDGWKKLQLGELIP
IPADSPPPPALSSNP.

Suitable framework polypeptides (e.g., those with an Ig Fc multimerization sequence) will, in some cases, be half-life extending polypeptides. Thus, in some cases, a suitable framework polypeptide increases the in vivo half-life (e.g., the serum half-life) of the MAPPs, compared to a control MAPP having a framework polypeptide with a different aa sequence. For example, in some cases, a framework polypeptide increases the in vivo half-life (e.g., the serum half-life in a mammal such as a human) of the MAPP, compared to a control MAPP having a framework polypeptide with a different aa sequence. The half-life may be extended by at least about 10%, at least about 15%, at least about 25%, at least about 50%, at least about 100%, at least about 2-fold, at least about 5-fold, at least about 10-fold, at least about 25-fold, at least about 50-fold, at least about 100-fold, or more than 100-fold. As an example, in some cases, an Ig Fc polypeptide sequence (e.g., utilized as a multimerization sequence to form a duplex of MAPP heterodimers comprising a framework and dimerization polypeptide) increases the stability and/or in vivo half-life (e.g., the serum half-life) of a MAPP duplex compared to a control MAPP duplex lacking the Ig Fc polypeptide sequence by at least about 10%, at least about 15%, at least about 25%, at least about 50%, at least about 100%, at least about 2-fold, at least about 2.5-fold, at least about 5-fold, at least about 10-fold, at least about 25-fold, at least about 50-fold, at least about 100-fold, or more than 100-fold.

3. Presenting Sequence and Presenting Complexes

As discussed in more detail below, class II MHC polypeptides, include two types of polypeptide chains, α-chain and β-chain. More specifically, MHC class II α-chain polypeptides include α1 and α2 domains, and β-chain polypeptides include β1 and β2 domains. Presenting sequences and presenting complexes comprise MHC class II polypeptides sufficient to bind and present an epitope to a TCR. Presenting sequences and complexes may also comprise additional protein (peptide) elements including one or more independently selected MODs and/or one or more independently selected linkers (e.g., linkers placed between various domains). As discussed herein, unless stated otherwise, neither presenting sequences nor presenting complexes comprise an MHC transmembrane domain (or intracellular domain such as a cytoplasmic tail) sufficient to anchor MAPP molecules (e.g., more than 50% of the MAPP molecules) in a mammalian cell membrane (e.g., a CHO cell membrane) when expressed therein.

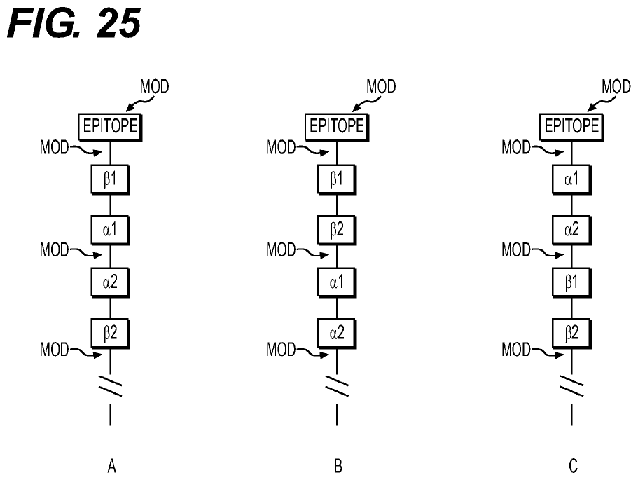

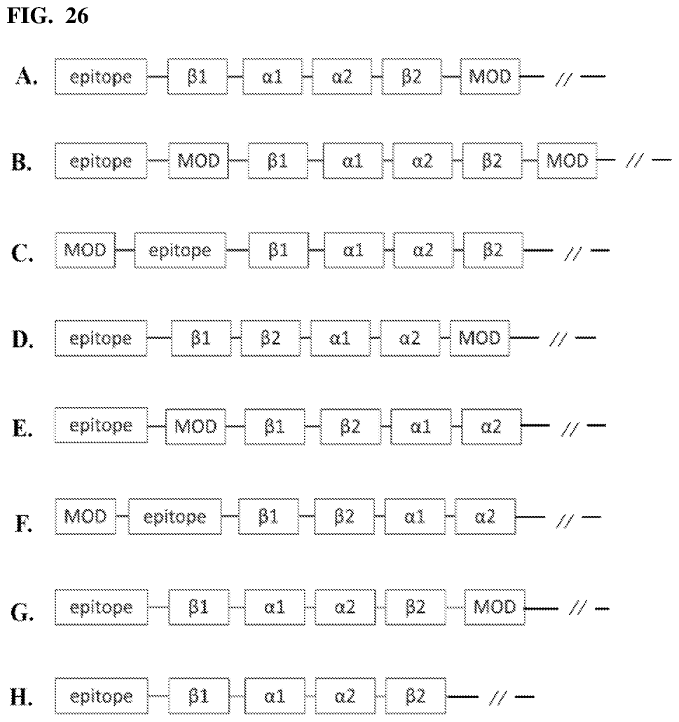

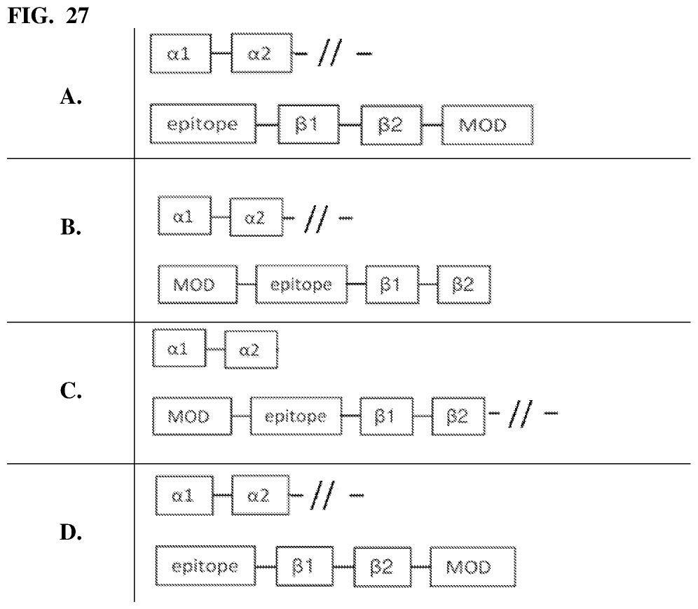

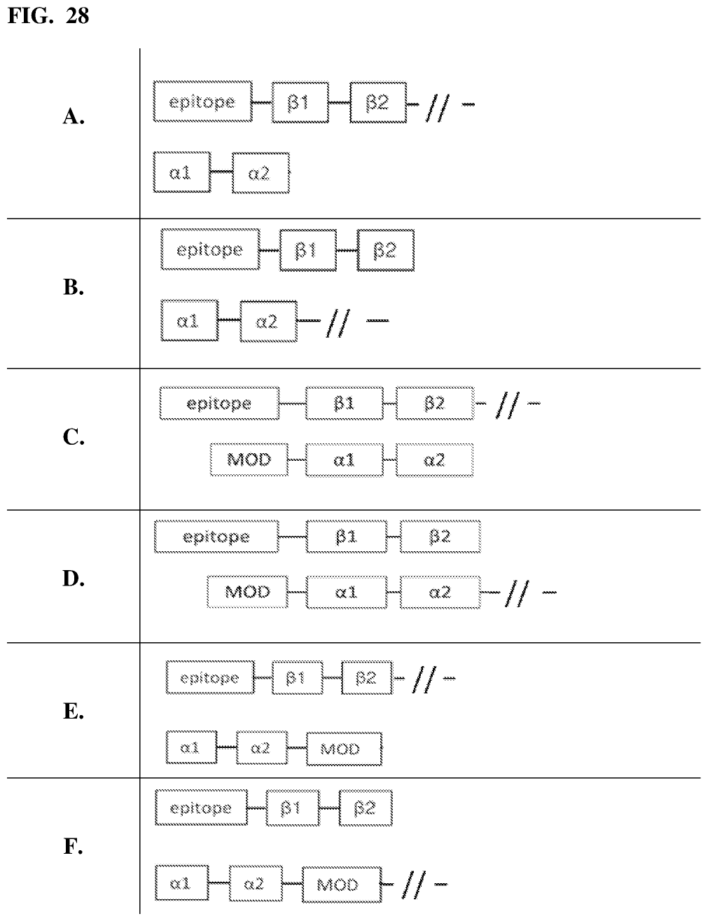

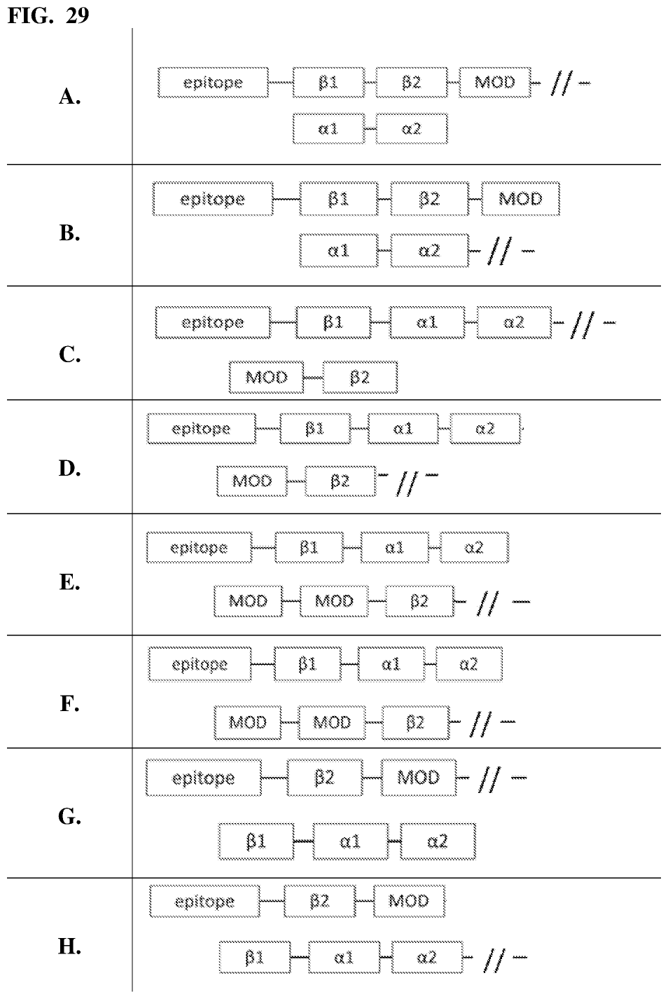

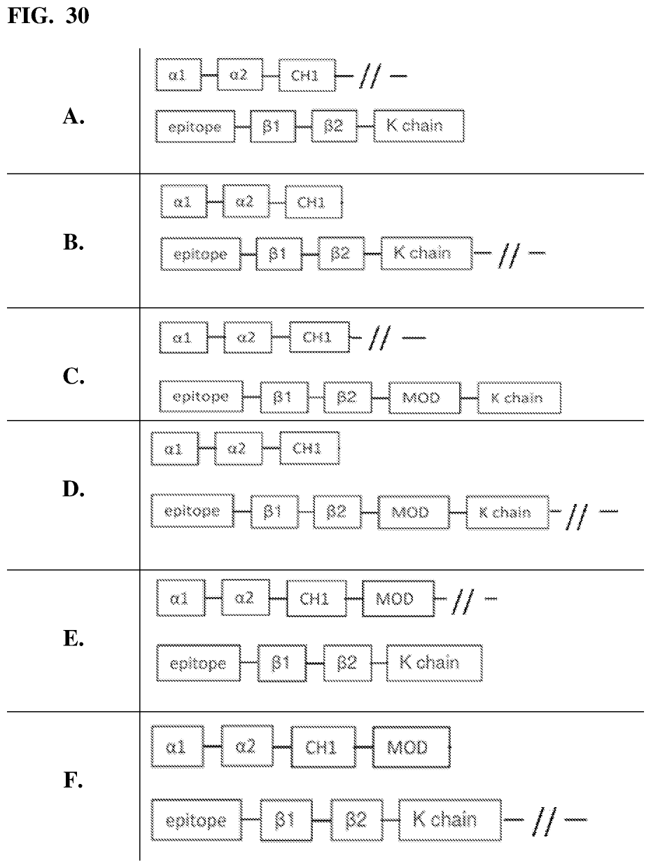

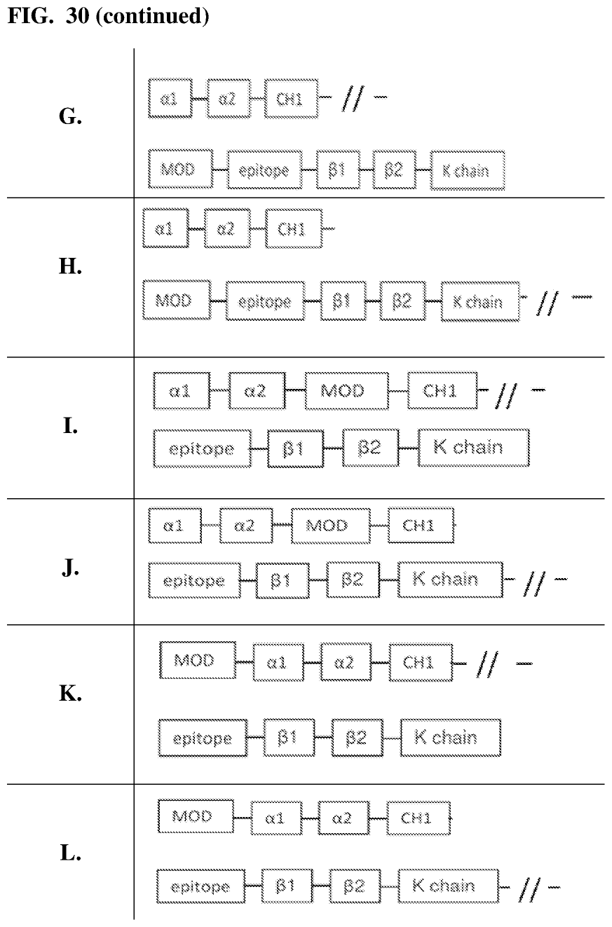

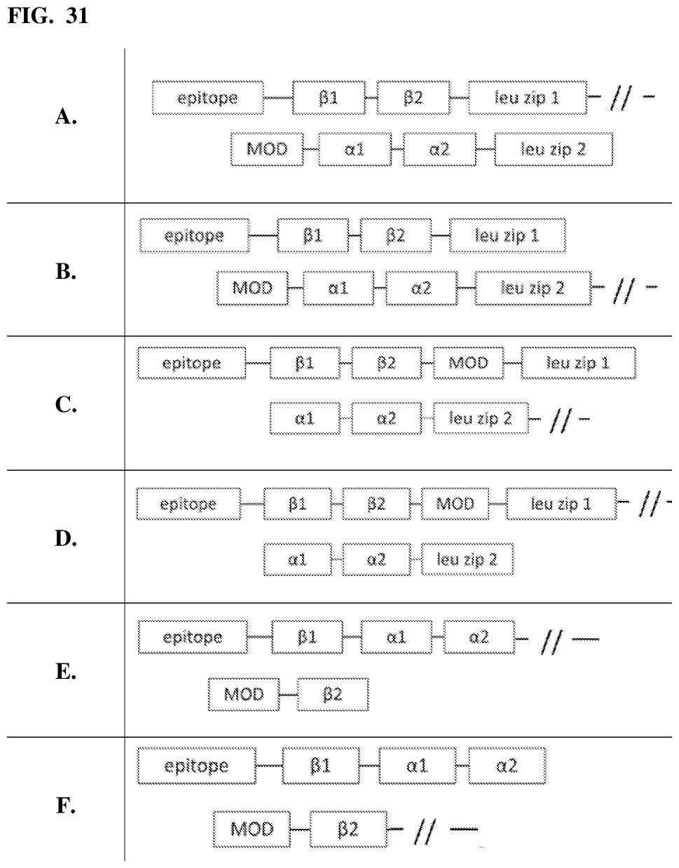

Conceptually, each of the presenting sequences and presenting complexes may be considered a “soluble MHC” that is fully capable of binding and presenting a peptide epitope. Unless stated otherwise, in presenting sequences all of the MHC α1, α2, β1, and β2 domain sequences, as well as the epitope polypeptide, are present in a single polypeptide chain (single linear sequence of aas produced by translation). See, e.g., FIGS. 25 and 26.

Where the MHC α1, α2, β1, and β2 domain sequences are divided among two or more polypeptide chains, the “soluble MHC” is termed a presenting complex. The presenting complex has one chain that is part of a framework peptide or dimerization peptide, referred to as a “presenting complex 1st sequence.” The second chain of the presenting complex is termed the “presenting complex 2nd sequence.” The presenting complex 2nd sequence may be associated non-covalently with the MHC components present in the presenting complex 1st sequence (through binding interactions between MHC-Class II α1, α2, β1, and β2 domain components as in FIGS. 27 to 29), in addition, one or more disulfide bonds between the presenting complex 1st sequence and the presenting complex 2nd sequence. Alternatively, the presenting complex 2nd sequence may be associated non-covalently with the MHC components present in the presenting complex 1st sequence through binding interactions between MHC-Class II α1, α2, β1, and β2 domain components and/or through binding sequences (e.g., such as interspecific binding sequences) as in FIGS. 30, 31 structures A-E, and 32, in the presence or absence of one or more disulfide bonds between the presenting complex 1st sequence and the presenting complex 2nd sequence.

In some cases, a MAPP comprises one or more presenting sequence of having all of the Class II components required for binding and presenting the epitope of interest to a TCR; e.g., the α1, α2, β1, and β2 domain and epitope in a single polypeptide sequence In some cases MAPPs comprise presenting complexes with all of the Class II components required for binding and presenting the epitope of interest to a TCR, and the peptide epitope is part of the presenting complex 1^{st} sequence or the presenting complex 2^{nd} sequence.

As noted above, presenting sequences and complexes typically will comprise a peptide epitope that is part of a polypeptide chain. It is possible, however, to make MAPPS that comprise the MHC components, but which do not comprise a peptide epitope that is part of a polypeptide chain. In such embodiments, the epitope, which is non-covalently loaded into the MHC pocket, may be a separate peptide (e.g., phosphopeptide, lipopeptide, glycosylated peptide, etc.) or non-peptide epitope, and may be subject to dissociation from the MAPPs.

4. MHC Class II Polypeptides

As noted above, the epitope containing MAPPs include MHC class II polypeptides of various species, including human MHC polypeptides (HLA polypeptides), rodent (e.g., mouse, rat, etc.) MHC polypeptides, and MHC polypeptides' of other mammalian species (e.g., lagomorphs, non-human primates, canines, felines, ungulates (e.g., equines, bovines, ovines, caprines, etc.)), and the like.

For the purpose of this disclosure the term “MHC polypeptide” is meant to include class II MHC polypeptides, including the α- and β-chains or portions thereof. More specifically, MHC class II polypeptides include the α1 and α2 domains of class II MHC α chains, and the β1 and β2 domains of class II MHC β chains, which represent all or most of the extracellular class II protein required for presentation of an epitope. In an embodiment, both the α and β class II MHC polypeptide sequences in a MAPP are of human origin.

MAPPs and their higher order complexes (e.g., duplex MAPPs) are intended to be soluble in aqueous media under physiological conditions (e.g., soluble in human blood plasma at therapeutic levels).

Unless expressly stated otherwise, as noted above, the MAPPs described herein are not intended to include membrane anchoring domains (such as transmembrane regions of MHC Class II α and β chains) or a part thereof sufficient to anchor MAPP molecules (e.g., more than 50% of the MAPP molecules), or a peptide thereof, in the membrane of a cell (e.g., a eukaryotic cell such as a mammalian cell such as a Chinese Hamster Ovary or “CHO” cell) in which the MAPP is expressed. Similarly, unless expressly stated otherwise, the MAPPs described herein do not include the leader and/or intracellular portions (e.g., cytoplasmic tails) that may be present in some naturally-occurring MHC Class II proteins.

MAPPs of the present disclosure comprise class II MHC polypeptides. Naturally occurring class II MHC polypeptides comprise an α chain and a β chain (e.g., HLA α- and β-chains). MHC Class II polypeptides include MHC Class II DP α and β polypeptides, DM α and β polypeptides, DO α and β polypeptides, DQ α and β polypeptides, and DR α and β polypeptides. As used herein, the term “Class II MHC polypeptide” refers to a Class II MHC α chain polypeptide, a Class II MHC β chain polypeptide, or only a portion of a Class II MHC α and/or β chain polypeptide, or combinations of the foregoing. For example, the term “Class II MHC polypeptide” as used herein can be a polypeptide that includes: i) only the α1 domain of a Class II MHC α chain; ii) only the α2 domain of a Class II MHC α chain; iii) only the α1 domain and an α2 domain of a Class II MHC α chain; iv) only the β1 domain of a Class II MHC β chain; v) only the β2 domain of a Class II MHC β chain; vi) only the β1 domain and the β2 domain of a Class II MHC β chain; vii) the α1 domain of a Class II MHC α chain, the β1 domain of a Class II MHC β chain, and the β2 domain of a Class II MHC; and the like.

The human MHC or HLA locus is highly polymorphic in nature, and thus as used herein, the term “Class II MHC polypeptide” includes allelic forms of any known Class II MHC polypeptide. See, e.g., the HLA Nomenclature site run by the Anthony Nolan Research Institute, available on the world wide web at hla.alleles.org/nomenclature/index.html, which indicates that there are numerous DRA alleles, DRB1 alleles, DRB3 alleles, DRB4 alleles, DRB5 alleles, DRB6 alleles, DRB7 alleles, DRB9 alleles, DQA1 alleles, DQB1 alleles, DPA1, DPB1 alleles, DMA alleles, DMB alleles, DOA alleles and DOB alleles.

In some cases, a MAPP comprises a Class II MHC α chain, without the leader, transmembrane, and intracellular portions (e.g., cytoplasmic tails) that may be present in a naturally-occurring Class II MHC α chain. Thus, in some cases, a MAPP comprises only the α1 and α2 portions of a Class II MHC α chain; and does not include the leader, transmembrane, and intracellular portions (e.g., cytoplasmic tails) that may be present in a naturally-occurring Class II MHC α chain.

In some cases, a MAPP comprises a Class II MHC β chain, without the leader, transmembrane, and intracellular portions (e.g., cytoplasmic tails) that may be present in a naturally-occurring Class II MHC β chain. Thus, in some cases, a MAPP comprises only the β1 and β2 portions of a Class II MHC β chain; and does not include the leader, transmembrane, and intracellular portions (e.g., cytoplasmic tails) that may be present in a naturally-occurring Class II MHC β chain.

(i) MHC Class II Alpha Chains

MHC Class II alpha chains comprise an α1 domain and an α2 domain. In some cases, the α1 and α2 domains present in an antigen-presenting cell are from the same MHC Class II α chain polypeptide. In some cases, the α1 and α2 domains present in an antigen-presenting cell are from two different MHC Class II α chain polypeptides.

MHC Class II alpha chains suitable for inclusion in a presenting sequence or complex of a MAPP may lack a signal peptide. An MHC Class II alpha chain suitable for inclusion in a MAPP can have a length of from about 60 aas to about 200 aas; for example, an MHC Class II alpha chain suitable for inclusion in a MAPP can have a length of from about from about 60 amino acids to about 80 amino acids, 80 aas to about 100 aas, from about 100 aas to about 140 aas, from about 140 aas to about 170 aas, from about 170 aas to about 200 aas. An MHC Class II α1 domain suitable for inclusion in a MAPP can have a length of from about 30 aas to about 95 aas; for example, an MHC Class II α1 domain suitable for inclusion in a MAPP of the present disclosure can have a length of from about 30 aas to about 50 aas, from about 50 aas to about 70 aas, or from about 70 aas to about 95 aas. In an embodiment a MHC Class II α1 domain of a MAPP is from about 70 aas to about 95 aas. An MHC Class II α2 domain suitable for inclusion in a MAPP can have a length of from about 30 aas to about 95 aas; for example, an MHC Class II α2 domain suitable for inclusion in a MAPP can have a length of from about 30 aas to about 50 aas, from about 50 aas to about 70 aas, or from about 70 aas to about 95 aas. In an embodiment, an MHC Class II α2 domain of a MAPP is from about 70 aas to about 95 aas.

(a) DRA Polypeptides

A suitable MHC Class II DRA polypeptide for inclusion in a MAPP may have at least 60%, at least 70%, at least 80%, at least 90%, at least 95%, at least 98%, at least 99%, or 100% aa sequence identity with at least 150, at least 160, or at least 170 contiguous amino acids of the aa sequence from aa 26 to aa 203 of the DRA aa sequence depicted in FIG. 4 or a naturally occurring allelic variant thereof. In some cases, the DRA polypeptide has a length of about 178 aas (e.g., 175, 176, 177, 178, 179, or 180 aas).

As used herein, the term “DRA polypeptide” includes allelic variants, e.g., naturally occurring allelic variants. Thus, in some cases, a suitable DRA polypeptide comprises aas 26-203 of DRA*01:02:01 (see FIG. 4), or an allelic variant thereof. In some cases, the allelic variant is the DRA*01:01 polypeptide (e.g., from the DRA*01:01:01:01 allele) that differs from DRA*01:02 by having a valine in place of the leucine at position 242 (see FIG. 4).

A suitable DRA for inclusion in a MAPP polypeptide can have at least 70%, at least 80%, at least 90%, at least 95%, at least 98%, at least 99%, or 100%, aa sequence identity with at least 160, at least 170, or at least 180 contiguous aas of the sequence from aa 26 to aa 216 of the DRA*01:02 sequence depicted in FIG. 4. A “DRA polypeptide” includes allelic variants, e.g., naturally occurring allelic variants.

Thus, in some cases, a suitable DRA polypeptide comprises the following amino acid sequence: IKEEH VIIQAEFYLN PDQSGEFMFD FDGDEIFHVD MAKKETVWRL EEFGRFASFE AQGALANIAV DKANLEIMTK RSNYTPITNV PPEVTVLTNS PVELREPNVL ICFIDKFTPP VVNVTWLRNG KPVTTGVSET VFLPREDHLF RKFHYLPFLPSTEDVYDCRV EHWGLDEPLL KHW (SEQ ID NO: 125, amino acids 26-203 of DRA*01:02, see FIG. 4), or an allelic variant thereof. In some cases, the allelic variant is the DRA*01:01 allelic variant that differs from DRA*01:02 polypeptide by having a valine in place of the leucine at position 242 of the sequence in FIG. 4. In some cases, a DRA polypeptide suitable for inclusion in a MAPP comprises an amino acid substitution, relative to a wild-type DRA polypeptide, where the amino acid substitution replaces an amino acid (other than a Cys) with a Cys (e.g., for forming a disulfide bond stabilizing the MAPP).

In some cases, a MAPP comprises a variant DRA polypeptide that comprises a non-naturally occurring Cys residue (e.g., for forming a disulfide bond stabilizing the MAPP). For example, in some cases, a MAPP comprises a variant DRA polypeptide that comprises an amino acid substitution selected from E3C, E4C, F12C, G28C, D29C, I72C, K75C, T80C, P81C, I82C, T93C, N94C, and S95C.

A suitable DRA α1 domain for inclusion in a MAPP polypeptide, including naturally occurring allelic variants thereof, may comprise an aa sequence having at least 70%, at least 80%, at least 90%, at least 95%, at least 98%, at least 99%, or 100% aa sequence identity to the following aa sequence: VIIQAEFYLN PDQSGEFMFD FDGDEIFHVD MAKKETVWRL EEFGRFASFE AQGALANIAV DKANLEIMTK RSNYTPITN (SEQ ID NO:124); and can have a length of about 84 aas (e.g., 80, 81, 82, 83, 84, 85, or 86 aas).

A suitable DRA α2 domain for inclusion in a MAPP polypeptide, including naturally occurring allelic variants thereof, may comprise an aa sequence having at least 70%, at least 80%, at least 90%, at least 95%, at least 98%, at least 99%, or 100% aa sequence identity to the following aa sequence: V PPEVTVLTNSPVELREPNVL ICFIDKFTPP VVNVTWLRNG KPVTTGVSET VFLPREDHLF RKFHYLPFLP STEDVYDCRV EHWGLDEPLL KHW (SEQ ID NO:126); and can have a length of about 94 aas (e.g., 90, 91, 92, 93, 94, 95, 96, 97, or 98 aas).

(b) DMA Polypeptides

In some cases, a suitable MHC Class II α chain polypeptide is a DMA polypeptide. A DMA polypeptide can have at least 60%, at least 70%, at least 80%, at least 90%, at least 95%, at least 98%, at least 99%, or 100% aa sequence identity with aas 27-217 of the DMA aa sequence depicted in FIG. 9, including-naturally occurring allelic variants thereof. In some cases, the DMA polypeptide has a length of about 191 aas (e.g., 188, 189, 190, 191, 192, or 193 aas).

A “DMA polypeptide” includes allelic variants, e.g., naturally occurring allelic variants. Thus, in some cases, a suitable DMA polypeptide comprises aas 27-217 of DMA*01:01:01 (see FIG. 9), or an allelic variant thereof.

A suitable DMA α1 domain, including-naturally occurring allelic variants thereof, may comprise an aa sequence having at least 70%, at least 80%, at least 90%, at least 95%, at least 98%, at least 99%, or 100% aa sequence identity to the following aa sequence: VPEA PTPMWPDDLQ NHTFLHTVYC QDGSPSVGLS EAYDEDQLFF FDFSQNTRVP RLPEFADWAQ EQGDAPAILF DKEFCEWMIQ QIGPKLDGKI PVSR (SEQ ID NO:127); and can have a length of about 98 aas (e.g., 94, 95, 96, 97, 98, 99, 100, or 101 aas).

A suitable DMA α2 domain, including-naturally occurring allelic variants thereof, may comprise an aa sequence having at least 70%, at least 80%, at least 90%, at least 95%, at least 98%, at least 99%, or 100% aa sequence identity to the following aa sequence: GFPIAE VFTLKPLEFG KPNTLVCFVS NLFPPMLTVN WQHHSVPVEG FGPTFVSAVD GLSFQAFSYL NFTPEPSDIF SCIVTHEIDR YTAIAYW (SEQ ID NO:128); and can have a length of about 93 aas (e.g., 90, 91, 92, 93, 94, 95, 96, or 97 aas).

(c) DOA Polypeptides

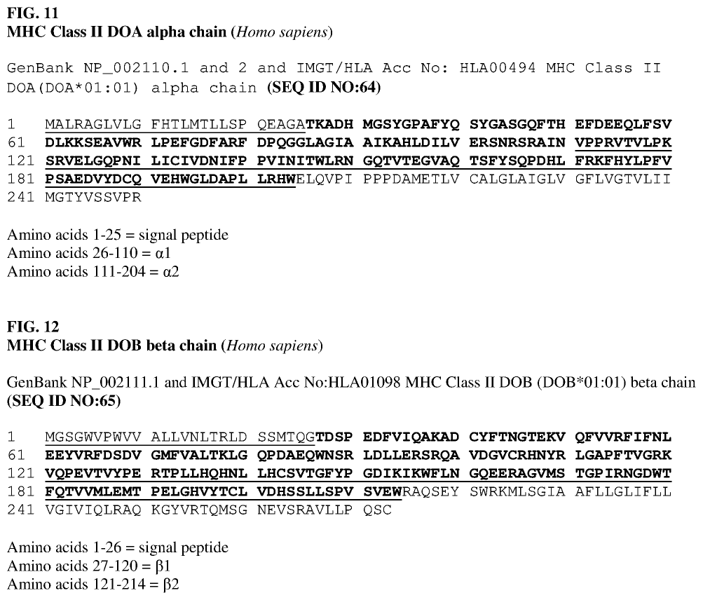

In some cases, a suitable MHC Class II α chain polypeptide is a DOA polypeptide. A DOA polypeptide can have at least 60%, at least 70%, at least 80%, at least 90%, at least 95%, at least 98%, at least 99%, or 100% aa sequence identity with aas 26-204 of the DOA aa sequence depicted in FIG. 11. In some cases, the DOA polypeptide has a length of about 179 aas (e.g., 175, 176, 177, 178, 179, 180, 181, or 182 aas).

A “DOA polypeptide” includes allelic variants, e.g., naturally occurring allelic variants. Thus, in some cases, a suitable DOA polypeptide comprises aas 26-204 of DOA*01:01:01:01 (see FIG. 11), or an allelic variant thereof. In some cases, the allelic variant may be the DOA*01:02 by having an arginine in place of the cysteine (R80C) at position 80 or the DOA*01:03 variant having a valine in place of the leucine at position 74 (L74V) relative to DOA*01:01:01:01.

A suitable DOA α1 domain, including-naturally occurring allelic variants thereof, may comprise an aa sequence having at least 70%, at least 80%, at least 90%, at least 95%, at least 98%, at least 99%, or 100% aa sequence identity to the following aa sequence: TKADH MGSYGPAFYQ SYGASGQFTH EFDEEQLFSV DLKKSEAVWR LPEFGDFARF DPQGGLAGIA AIKAHLDILV ERSNRSRAIN (SEQ ID NO:129); and can have a length of about 85 aas (e.g., 83, 84, 85, 86, 87, or 88 aas). Suitable α1 domain sequences may incorporate the L74V and/or R80C substitutions found in DOA*01:02 and DOA*01:03 (the aas corresponding to L74 and R 80 are shown italicized and bolded).

A suitable DOA α2 domain, including-naturally occurring allelic variants thereof, may comprise an aa sequence having at least 70%, at least 80%, at least 90%, at least 95%, at least 98%, at least 99%, or 100% aa sequence identity to the following aa sequence: VPPRVTVLPK SRVELGQPNI LICIVDNIFP PVINITWLRN GQTVTEGVAQ TSFYSQPDHL FRKFHYLPFV PSAEDVYDCQ VEHWGLDAPL LRHW (SEQ ID NO:130); and can have a length of about 94 aas (e.g., 91, 92, 93, 94, 95, 96, or 97 aas).

(d) DPA1 Polypeptides

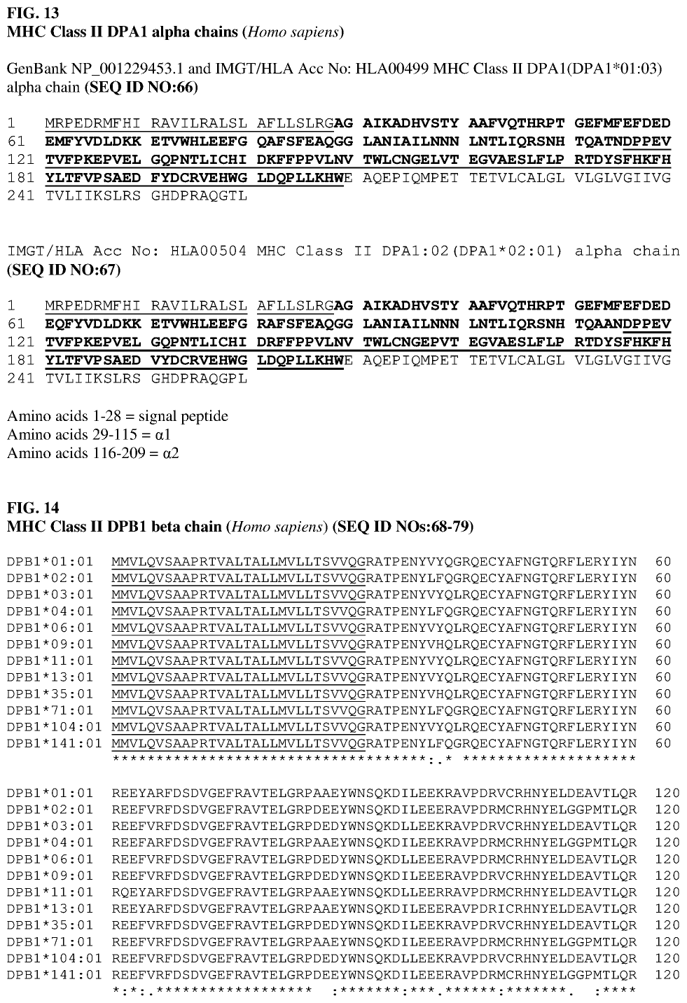

In some cases, a suitable MHC Class II α chain polypeptide is a DPA1 polypeptide. A DPA1 polypeptide can have at least 60%, at least 70%, at least 80%, at least 90%, at least 95%, at least 98%, at least 99%, or 100% aa sequence identity with aas 29-209 of the DPA1 aa sequence depicted in FIG. 13. In some cases, the DPA1 polypeptide has a length of about 181 aas (e.g., 178, 179, 180, 181, 182, 183, or 184 aas).

A “DPA1 polypeptide” includes allelic variants, e.g., naturally occurring allelic variants. Thus, in some cases, a suitable DPA1 polypeptide comprises aas 29-209 of DPA1*01:03:01:01 (see FIG. 13), or an allelic variant thereof.

A suitable DPA1 α1 domain, including-naturally occurring allelic variants thereof, may comprise an aa sequence having at least 70%, at least 80%, at least 90%, at least 95%, at least 98%, at least 99%, or 100% aa sequence identity to the following aa sequence: AIKADHVSTY AAFVQTHRPT GEFMFEFDED EMFYVDLDKK ETVWHLEEFG QAFSFEAQGG LANIAILNNN LNTLIQRSNH TQATN (SEQ ID NO:131); and can have a length of about 87 aas (e.g., 84, 85, 86, 87, 88, or 89 aas).

A suitable DPA1 α2 domain, including-naturally occurring allelic variants thereof, may comprise an aa sequence having at least 70%, at least 80%, at least 90%, at least 95%, at least 98%, at least 99%, or 100% aa sequence identity to the following aa sequence: DPPEV TVFPKEPVEL GQPNTLICHI DKFFPPVLNV TWLCNGELVT EGVAESLFLP RTDYSFHKFH YLTFVPSAED FYDCRVEHWG LDQPLLKHW (SEQ ID NO:132); and can have a length of about 97 aas (e.g., 91, 92, 93, 94, 95, 96, or 97 aas).

Another DPA1 polypeptide comprises aas 29-209 of DPA1*02:01:01:01 (see FIG. 13), or a variant thereof having at least 70%, at least 80%, at least 90%, at least 95%, at least 98%, at least 99%, or 100% aa sequence identity. A suitable DPA1 α1 domain, including-naturally occurring allelic variants thereof, may comprise an aa sequence having at least 70%, at least 80%, at least 90%, at least 95%, at least 98%, at least 99%, or 100% aa sequence identity to aas 29-115 of DPA1*02:01:01:01, SEQ ID NO:67; and can have a length of about 87 aas (e.g., 84, 85, 86, 87, 88, or 89 aas). A suitable DPA1 α2 domain, including-naturally occurring allelic variants thereof, may comprise an aa sequence having at least 70%, at least 80%, at least 90%, at least 95%, at least 98%, at least 99%, or 100% aa sequence identity to aas 116 to 209 of DPA1*02:01:01:01, SEQ ID NO:67; and can have a length of about 97 aas (e.g., 91, 92, 93, 94, 95, 96, or 97 aas).

(e) DQA1 Polypeptides

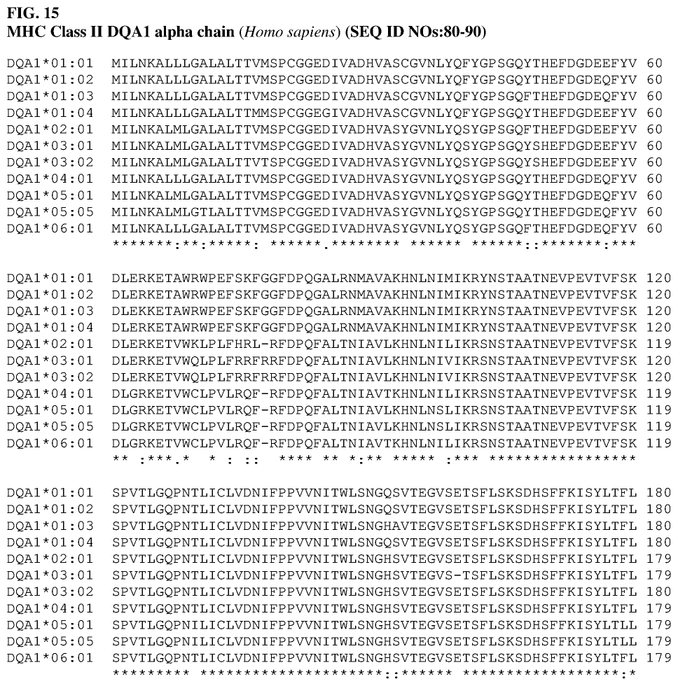

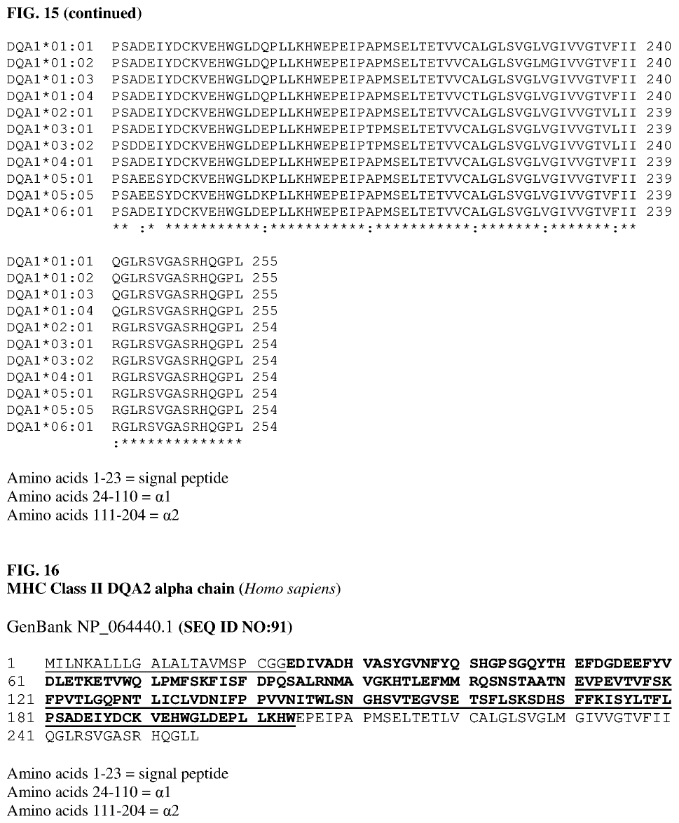

In some cases, a suitable MHC Class II α chain polypeptide is a DQA1 polypeptide. A suitable DQA1 polypeptide, including-naturally occurring allelic variants thereof, may comprise an aa sequence having at least 60%, at least 70%, at least 80%, at least 90%, at least 95%, at least 98%, at least 99%, or 100% aa sequence identity with aas 24-204 of any of the DQA1 aa sequences depicted in FIG. 15. In some cases, the DQA1 polypeptide has a length of about 181 aas (e.g., 177, 178, 179, 180, 181, 182, or 183 aas). In an embodiment, a DQA1 α chain polypeptide can have at least 60%, at least 70%, at least 80%, at least 90%, at least 95%, at least 98%, at least 99%, or 100% aa sequence identity with aas 24-204 of the DQA1*01:01 α chain aa sequence in FIG. 15, ImMunoGeneTics (“IMGT”)/HLA Acc No: HLA00601. In an embodiment, a DQA1 α chain polypeptide can have at least 60%, at least 70%, at least 80%, at least 90%, at least 95%, at least 98%, at least 99%, or 100% aa sequence identity with aas 24-204 of the DQA1*01:02 α chain aa sequence in FIG. 15, IMGT/HLA Acc No: HLA00603, GenBank NP_002113. In an embodiment, a DQA1 α chain polypeptide can have at least 60%, at least 70%, at least 80%, at least 90%, at least 95%, at least 98%, at least 99%, or 100% aa sequence identity with aas 24-204 of the DQA1*02:01 α chain aa sequence in FIG. 15, IMGT/HLA Acc No:HLA00607. In an embodiment, a DQA1 α chain polypeptide can have at least 60%, at least 70%, at least 80%, at least 90%, at least 95%, at least 98%, at least 99%, or 100% aa sequence identity with aas 24-204 of the DQA1*03:01: α chain aa sequence in FIG. 15, IMGT/HLA Acc No:HLA00609. In an embodiment, a DQA1 α chain polypeptide can have at least 60%, at least 70%, at least 80%, at least 90%, at least 95%, at least 98%, at least 99%, or 100% aa sequence identity with aas 24-204 of the DQA1*04:01 α chain aa sequence in FIG. 15, IMGT/HLA Acc No:HLA00612. In an embodiment, a DQA1 α chain polypeptide can have at least 60%, at least 70%, at least 80%, at least 90%, at least 95%, at least 98%, at least 99%, or 100% aa sequence identity with aas 24-204 of the DQA1*05:01 α chain aa sequence in FIG. 15, IMGT/HLA Acc No:HLA00613. In an embodiment, a DQA1 α chain polypeptide can have at least 60%, at least 70%, at least 80%, at least 90%, at least 95%, at least 98%, at least 99%, or 100% aa sequence identity with aas 24-204 of the DQA1*06:01 α chain aa sequence in FIG. 15, IMGT/HLA Acc No:HLA00620.

A “DQA1 polypeptide” includes allelic variants, e.g., naturally occurring allelic variants. Thus, in some cases, a suitable DQA1 polypeptide comprises the following aa sequence: EDIVADH VASCGVNLYQ FYGPSGQYTH EFDGDEQFYV DLERKETAWR WPEFSKFGGF DPQGALRNMA VAKHNLNIMI KRYNSTAATN EVPEVTVFSK SPVTLGQPNT LICLVDNIFP PVVNITWLSN GQSVTEGVSE TSFLSKSDHS FFKISYLTFL PSADEIYDCK VEHWGLDQPL LKHW (SEQ ID NO:133), or an allelic variant thereof.

A suitable DQA1 α1 domain, including-naturally occurring allelic variants thereof, may comprise an aa sequence having at least 70%, at least 80%, at least 90%, at least 95%, at least 98%, at least 99%, or 100% aa sequence identity to the following aa sequence: EDIVADH VASCGVNLYQ FYGPSGQYTH EFDGDEQFYV DLERKETAWR WPEFSKFGGF DPQGALRNMA VAKHNLNIMI KRYNSTAATN (SEQ ID NO:134); and can have a length of about 87 aas (e.g., 84, 85, 86, 87, 88, or 89 aas).

A suitable DQA1 α2 domain, including-naturally occurring allelic variants thereof, may comprise an aa sequence having at least 70%, at least 80%, at least 90%, at least 95%, at least 98%, at least 99%, or 100% aa sequence identity to the following aa sequence: EVPEVTVFSK SPVTLGQPNT LICLVDNIFP PVVNITWLSN GQSVTEGVSE TSFLSKSDHS FFKISYLTFL PSADEIYDCK VEHWGLDQPL LKHW (SEQ ID NO:135); and can have a length of about 94 aas (e.g., 91, 92, 93, 94, 95, 96, or 97 aas).

(f) DQA2 Polypeptides

In some cases, a suitable MHC Class II α chain polypeptide is a DQA2 polypeptide. A DQA2 polypeptide can have at least 60%, at least 70%, at least 80%, at least 90%, at least 95%, at least 98%, at least 99%, or 100% aa sequence identity with aas 24-204 of the DQA2 aa sequence depicted in FIG. 16. In some cases, the DQA2 polypeptide has a length of about 181 aas (e.g., 177, 178, 179, 180, 181, 182, or 183 aas).

A “DQA2 polypeptide” includes allelic variants, e.g., naturally occurring allelic variants. Thus, in some cases, a suitable DQA2 polypeptide comprises the following aa sequence: EDIVADH VASYGVNFYQ SHGPSGQYTH EFDGDEEFYV DLETKETVWQ LPMFSKFISF DPQSALRNMA VGKHTLEFMM RQSNSTAATN EVPEVTVFSK FPVTLGQPNT LICLVDNIFP PVVNITWLSN GHSVTEGVSE TSFLSKSDHS FFKISYLTFL PSADEIYDCK VEHWGLDEPL LKHW (SEQ ID NO:136), or an allelic variant thereof.

A suitable DQA2 α1 domain, including-naturally occurring allelic variants thereof, may comprise an aa sequence having at least 70%, at least 80%, at least 90%, at least 95%, at least 98%, at least 99%, or 100% aa sequence identity to the following aa sequence: EDIVADH VASYGVNFYQ SHGPSGQYTH EFDGDEEFYV DLETKETVWQ LPMFSKFISF DPQSALRNMA VGKHTLEFMM RQSNSTAATN (SEQ ID NO:137); and can have a length of about 87 aas (e.g., 84, 85, 86, 87, 88, or 89 aas).

A suitable DQA2 α2 domain, including-naturally occurring allelic variants thereof, may comprise an aa sequence having at least 70%, at least 80%, at least 90%, at least 95%, at least 98%, at least 99%, or 100% aa sequence identity to the following aa sequence: EVPEVTVFSK FPVTLGQPNT LICLVDNIFP PVVNITWLSN GHSVTEGVSE TSFLSKSDHS FFKISYLTFL PSADEIYDCK VEHWGLDEPL LKHW (SEQ ID NO:138); and can have a length of about 94 aas (e.g., 91, 92, 93, 94, 95, 96, or 97 aas).

(ii) MHC Class II Beta Chains

MHC Class II beta chains comprise a β1 domain and a β2 domain. In some cases, the β1 and β2 domains present in an antigen-presenting cell are from the same MHC Class II β chain polypeptide. In some cases, the β1 and β2 domains present in an antigen-presenting cell are from two different MHC Class II β chain polypeptides.

MHC Class II beta chains suitable for inclusion in a MAPP (e.g., a higher order MAPP construct such as a duplex MAPP) lack a signal peptide. An MHC Class II beta chain suitable for inclusion in a MAPP can have a length of from about 60 aas to about 210 aas; for example, an MHC Class II beta chain suitable for inclusion in a MAPP can have a length of from about 60 aas to about 90 aas, from about 90 aas to about 120 aas, from about 120 aas to about 150 aas, from about 150 aas to about 180 aas, from about 180 aas to 210 aas. An MHC Class II β1 domain suitable for inclusion in a MAPP can have a length of from about 30 aas to about 105 aas; for example, an MHC Class II β1 domain suitable for inclusion in a MAPP can have a length of from about 30 aas to about 50 aas, from about 50 aas to about 70 aas, from about 70 aas to about 90 aas, from about 90 aas to about 105 aas. An MHC Class II β2 domain suitable for inclusion in a MAPP can have a length of from about 30 aas to about 105 aas; for example, an MHC Class II β2 domain suitable for inclusion in a MAPP can have a length of from about 30 aas to about 50 aas, from about 50 aas to about 70 aas, from about 70 aas to about 90 aas, from about 90 aas to about 105 aas.

An MHC class II β chain polypeptide suitable for inclusion in a MAPP may comprises an aa substitution, relative to a wild-type MHC class II β chain polypeptide, where the aa substitution replaces an aa (other than a Cys) with a Cys (e.g., for forming a disulfide bond stabilizing the MAPP). For example, in some cases, the MHC class II β chain polypeptide is a variant DRB1 MHC class II polypeptide that comprises an aa substitution selected from the group consisting of P5C, F7C, Q10C, N19C, G20C, H33C, G151C, D152C, and W153C. In some cases, the MHC class II β chain polypeptide is a variant DRB1 polypeptide comprising an aa sequence having at least 60%, at least 70%, at least 80%, at least 90%, at least 95%, at least 98%, or at least 99%, aa sequence identity to the following mature DRB1 aa sequence lacking the signal peptide: GDTRPRFLEQVKHECHFFNGTERVRFLDRYFYHQEEYVRFDSDVGEYRAVTELGRPDAEYWNS QKDLLEQKRAAVDTYCRHNYGVGESFTVQRRVYPEVTVYPAKTQPLQHHNLLVCSVNGFYPA SIEVRWFRNGQEEKTGVVSTGLIQNGDWTFQTLVMLETVPRSGEVYTCQVEHPSLTSPLTVEWR ARSESAQSKM (SEQ ID NO:139), and comprising an cysteine substitution at one or more (e.g., two or more) aas selected from the group consisting of P5C, F7C, Q10C, N19C, G20C, H33C, G151C, D152C, and W153C. In some cases, the MHC Class II β chain polypeptide is a variant of a mature DRB3 polypeptide, mature DRB4 polypeptide, or mature DRB5 polypeptide (lacking their signal sequences) comprising a cysteine substitution at one or more (e.g., two or more) of positions 5, 7, 10, 19, 20, 33, 151, 152, and 153 (e.g., PSC, F7C, Q10C, N19C, G20C, N33C, G151C, D152C, and/or W153C substitutions).

(a) DRB1 Polypeptides

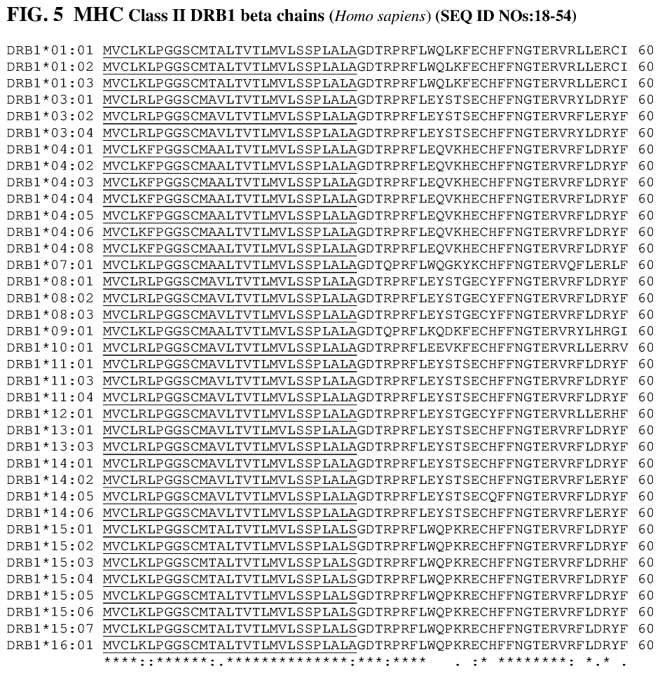

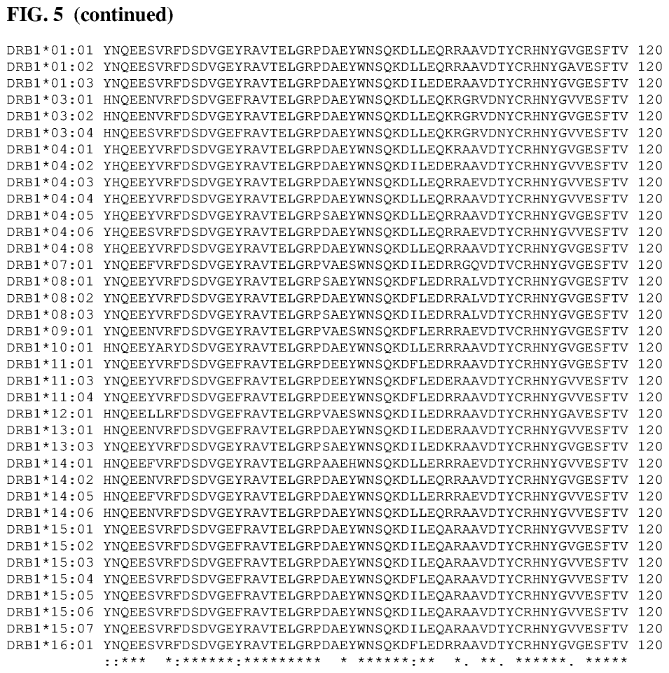

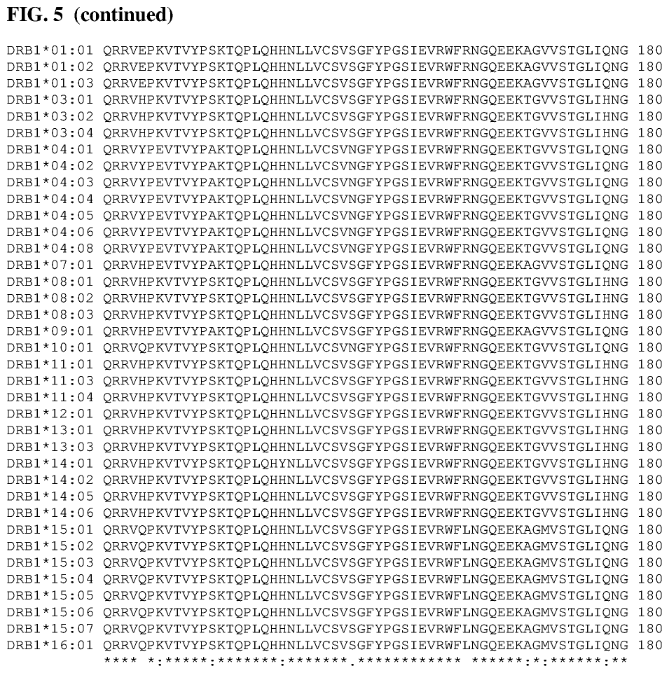

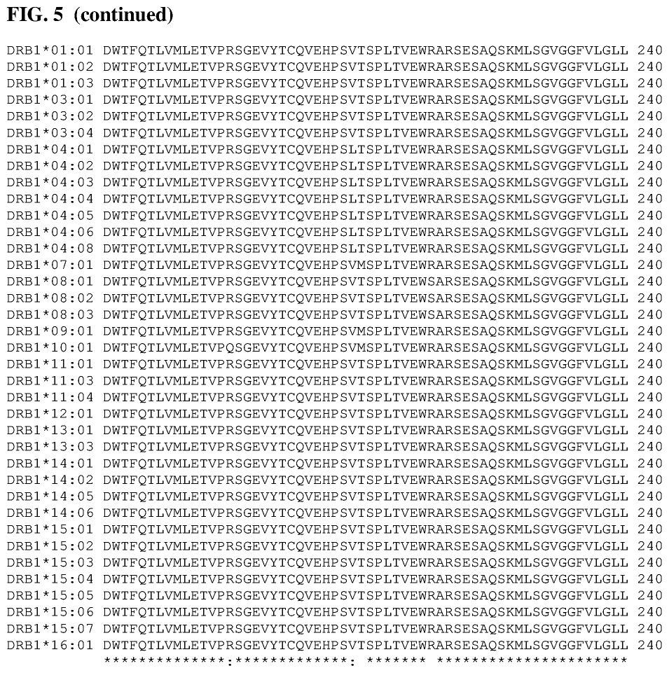

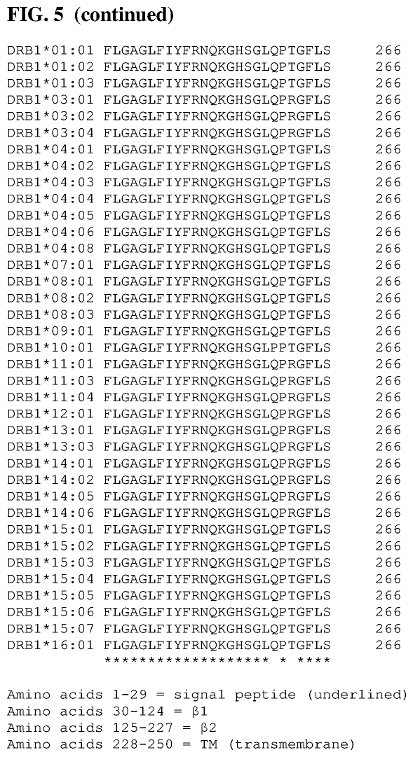

In some cases, a suitable MHC Class II β chain polypeptide is a DRB1 polypeptide. In an embodiment, a DRB1 polypeptide can have at least 60%, at least 70%, at least 80%, at least 90%, at least 95%, at least 98%, at least 99%, or 100%, aa sequence identity with at least 170, at least 180, or at least 190, contiguous aas of the sequence from aa 30 to aa 227 of any DRB1 aa sequence depicted in FIG. 5, including naturally occurring allelic variants. FIG. 5 displays the DRB1 precursor proteins in which aas 1-29 are the signal sequence (underlined), 30-124 form the β 1 region (bolded), 125-227 for the β2 region (bolded and underlined), and 228-250 the transmembrane region. In some cases, a DRB1 polypeptide suitable for inclusion in a MAPP comprises an aa substitution, relative to a wild-type DRB1 polypeptide, where the aa substitution replaces an aa (other than a Cys) with a Cys.

A suitable MHC Class II β chain polypeptide suitable for incorporation into a MAPP may be a DRB1 polypeptide. A DRB1 polypeptide may have at least 60%, at least 70%, at least 80%, at least 90%, at least 95%, at least 98%, at least 99%, or 100% aa sequence identity with at least 170, at least 180, or at least 190, contiguous aas of the sequence from aa 30 to aa 227 of a DRB1 sequence provided in FIG. 5, including one of the following DRB1 polypeptides:
(i) the DRB1-1 (DRB1*01:01) beta chain aa sequence Swiss-Prot/UniProt reference (“sp”) P04229.2 in FIG. 5;
(ii) the DRB1-3 (DRB1*03:01) beta chain aa sequence sp P01912.2 in FIG. 5;
(iii) the DRB1-4 (DRB1*04:01) beta chain aa sequence sp P13760.1 in FIG. 5;
(iv) the DRB1-7 (DRB1*07:01) beta chain aa sequence sp P13761.1 in FIG. 5;
(v) the DRB1-8 (DRB1*08:01) beta chain aa sequence sp Q30134.2 in FIG. 5;
(vi) the DRB1-9 (DRB1*09:01) beta chain aa sequence sp Q9TQE0.1 in FIG. 5;
(vii) the DRB1-10 (DRB1*10:01) beta chain aa sequence sp Q30167.2 in FIG. 5;
(viii) the DRB1-11 (DRB1*11:01) beta chain aa sequence sp P20039.1 in FIG. 5;
(ix) the DRB1-12 (DRB1*12:01) beta chain aa sequence sp Q951E3.1 in FIG. 5;
(x) the DRB1-13 (DRB1*13:01) beta chain aa sequence sp Q5Y7A7.1 in FIG. 5;
(xi) the DRB1-14 (DRB1*14:01) beta chain aa sequence sp Q9GIY3.1 in FIG. 5;
(xii) the DRB1-15 (DRB1*15:01) beta chain aa sequence sp P01911 in FIG. 5; and
(xiii) the DRB1-16 (DRB1*16:01) beta chain aa sequence sp Q29974.1 in FIG. 5.

As use herein “DRB1 polypeptide” includes allelic variants, e.g., naturally occurring allelic variants. Thus, in some cases, a suitable DRB1 polypeptide comprises aas 31-227 of DRB1*04:01 (DRB1-4) provided in FIG. 5 (SEQ ID NO:24) or an allelic variant thereof.

Another suitable DRB1 polypeptide may comprise a sequence having at least 70%, at least 80%, at least 90%, at least 95%, at least 98%, at least 99%, or 100% aa sequence identity to at least 170, at least 180, or at least 190 contiguous aas of the following DRB1*04:01 aa sequence: GDTRPRFLEQVKHECHFFNGTERVRFLDRYFYHQEEYVRFDSDVGEYRAVTELGRPDAEYWNS QKDLLEQKRAAVDTYCRHNYGVGESFTVQRRVYPEVTVYPAKTQPLQHHNLLVCSVNGFYPA SIEVRWFRNGQEEKTGVVSTGLIQNGDWTFQTLVMLETVPRSGEVYTCQVEHPSLTSPLTVEWR ARSESAQSKM (SEQ ID NO:139), which may bear one or more cysteine substitutions. In an embodiment the cysteine substitution is a P5C substitution. In an embodiment the cysteine substitution is a G151C substitution. In an embodiment the cysteine substitution is a W153C substitution.

A suitable DRB1 β1 domain, including-naturally occurring allelic variants thereof, may comprise an aa sequence having at least 70%, at least 80%, at least 90%, at least 95%, at least 98%, at least 99%, or 100% aa sequence identity to the following aa sequence: DTRPRFLEQVKHECHFFNGTERVRFLDRYFYHQEEYVRFDSDVGEYRAVTELGRPDAEYWNSQ KDLLEQKRAAVDTYCRHNYGVGESFTVQRRV (SEQ ID NO:140); and can have a length of about 95 aas (including, e.g., 92, 93, 94, 95, 96, 97, or 98 aas).

A suitable DRB1 β1 domain can comprise the following amino acid sequence: GDTRCRFLEQVKHECHFFNGTERVRFLDRYFYHQEEYVRFDSDVGEYRAVTELGRPDAEYWNS QKDLLEQKRAAVDTYCRHNYGVGESFTVQRRV (SEQ ID NO:141), where P5 is substituted with a Cys (shown in bold and italics text).

A suitable DRB1 β2 domain, including-naturally occurring allelic variants thereof, may comprise an aa sequence having at least 70%, at least 80%, at least 90%, at least 95%, at least 98%, at least 99%, or 100% aa sequence identity to the following aa sequence: YPEVTVYPAKTQPLQHHNLLVCSVNGFYPGSIEVRWFRNGQEEKTGVVSTGLIQNGDWTFQTLV MLETVPRSGEVYTCQVEHPSLTSPLTVEWRARSESAQSK (SEQ ID NO:142); and can have a length of about 103 aas (including, e.g., 100, 101, 102, 103, 104, 105, or 106 aas).

A suitable DRB1 β2 domain can comprise the following amino acid sequence: YPEVTVYPAKTQPLQHHNLLVCSVNGFYPASIEVRWFRNGQEEKTGVVSTGLIQNGDCTFQTLV MLETVPRSGEVYTCQVEHPSLTSPLTVEWRARSESAQSKM (SEQ ID NO:143), where W153 is substituted with a Cys (shown in bold and italics text).

(b) DRB3 Polypeptides

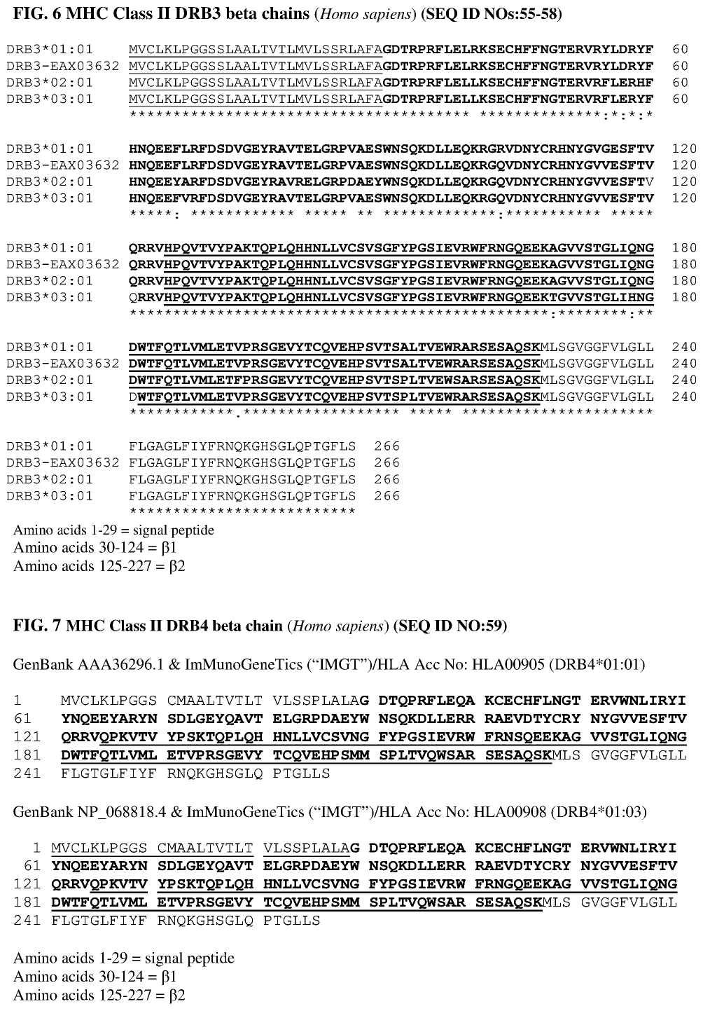

In some cases, a suitable MHC Class II β chain polypeptide is a DRB3 polypeptide. In an embodiment, a DRB3 polypeptide can have at least 60%, at least 70%, at least 80%, at least 90%, at least 95%, at least 98%, at least 99%, or 100% aa sequence identity with aas 30-227 of any DRB3 aa sequence depicted in FIG. 6, which displays the DRB3 precursor proteins in which aas 1-29 are the signal sequence (underlined), 30-124 form the β 1 region (shown bolded), 125-227 form the β2 region, and 228-250, the transmembrane region. A DRB3 β chain polypeptide suitable for incorporation into a MAPP may have at least 60%, at least 70%, at least 80%, at least 90%, at least 95%, at least 98%, at least 99%, or 100% aa sequence identity with aas 30-227 of one of the following DRB3 polypeptides:

(i) the DRB1-3 (DRB3*01:01) beta chain aa sequence GenBank NP_072049.1 in FIG. 6;

(ii) the DRB1-3 beta chain aa sequence in GenBank accession EAX03632.1 in FIG. 6;

(iii) the DRB1-3 (DRB3*02:01) beta chain aa sequence GenBank CAA23781.1 in FIG. 6; and

(iv) the DRB1-3 (DRB3*03:01) beta chain aa sequence GenBank AAN15205.1 in FIG. 6. ADRB3 polypeptide suitable for inclusion in a MAPP may comprise an aa substitution, relative to a wild-type DRB3 polypeptide, where the aa substitution replaces an aa (other than a Cys) with a Cys (e.g., for forming a disulfide bond stabilizing the MAPP).

As used herein, the term “DRB3 polypeptide” includes allelic variants, e.g., naturally occurring allelic variants. Thus, in some cases, a suitable DRB3 polypeptide comprises aas 30 to 227 of DRB3*01:01 provided in FIG. 6 (SEQ ID NO:55), or an allelic variant thereof. Thus, in some cases, a suitable DRB3 polypeptide comprises a sequence having at least 80%, at least 90%, at least 95%, at least 98%, at least 99%, or 100% sequence identity to at least 170, at least 180, or at least 190 contiguous aas of the following sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRVHPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:144), or an allelic variant thereof. In some cases, a DRB3 polypeptide suitable for inclusion in a MAPP comprises an aa substitution, relative to a wild-type DRB3 polypeptide, where the aa substitution replaces an aa (other than a Cys) with a Cys. Thus, e.g., in some cases, the MHC class II β chain polypeptide is a variant DRB3 MHC class II polypeptide that comprises a non-naturally occurring Cys at an aa selected from the group consisting of P5C, F7C, L10C, N19C, G20C, N33C, G151C, D152C, and W153C (of a mature DRB3 polypeptide (lacking the N-terminal signal peptide MVCLKLPGGSSLAALTVTLMVLSSRLAFA (SEQ ID NO:145) depicted in FIG. 6).

A suitable DRB3 β1 domain, including-naturally occurring allelic variants thereof, may comprise an aa sequence having at least 70%, at least 80%, at least 90%, at least 95%, at least 98%, at least 99%, or 100% aa sequence identity to the following aa sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRV (SEQ ID NO:146); and can have a length of about 95 aas (e.g., 93, 94, 95, 96, 97, or 98 aas). A suitable DRB3 β1 domain can comprise the following aa sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRV (SEQ ID NO:146), or a naturally-occurring allelic variant A suitable DRB3 β2 domain, including-naturally occurring allelic variants thereof, may comprise an aa sequence having at least 70%, at least 80%, at least 90%, at least 95%, at least 98%, at least 99%, or 100% aa sequence identity to the following aa sequence: HPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:147); and can have a length of about 103 aas (e.g., 100, 101, 102, 103, 104, or 105 aas). A suitable DRB3 β2 domain can comprise the following aa sequence: HPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:147), or a naturally-occurring allelic variant thereof.

(c) DRB4 Polypeptides

In some cases, a suitable MHC Class II β chain polypeptide is a DRB4 polypeptide. A DRB4 polypeptide can have at least 60%, at least 70%, at least 80%, at least 90%, at least 95%, at least 98%, at least 99%, or 100% aa sequence identity with aas 30-227 of a DRB4 aa sequence depicted in FIG. 7. In some cases, the DRB4 polypeptide has a length of about 198 aas (including e.g., 195, 196, 197, 198, 199, 200, 201, or 202 aas). In some cases, a DRB4 polypeptide suitable for inclusion in a MAPP comprises an amino acid substitution, relative to a wild-type DRB4 polypeptide, where the amino acid substitution replaces an amino acid (other than a Cys) with a Cys.

As used herein, the term “DRB4 polypeptide” includes allelic variants, e.g., naturally occurring allelic variants. Thus, in some cases, a suitable DRB4 polypeptide comprises aas 30 to 227 of DRB4*01:03 (SEQ ID NO:60) provided in FIG. 7, or an allelic variant thereof. In some cases, a DRB4 polypeptide suitable for inclusion in a MAPP comprises an amino acid substitution, relative to a wild-type DRB4 polypeptide, where the amino acid substitution replaces an amino acid (other than a Cys) with a Cys. Thus, e.g., in some cases, the MHC class II β chain polypeptide is a variant DRB4 MHC class II polypeptide that comprises a non-naturally occurring Cys residue; e.g., where the variant DRB4 MHC class II polypeptide comprises an amino acid substitution selected from the group consisting of P15C, F17C, Q20C, N29C, G30C, N43C, G161C, D162C, and W163C of a mature DRB4 polypeptide (lacking the N-terminal signal peptide MVCLKLPGGSCMAALTVTL (SEQ ID NO:148) depicted in FIG. 7).

A suitable DRB4 β1 domain, including-naturally occurring allelic variants thereof, may comprise an aa sequence having at least 70%, at least 80%, at least 90%, at least 95%, at least 98%, at least 99%, or 100% aa sequence identity to the following aa sequence: T VLSSPLALAG DTQPRFLEQA KCECHFLNGT ERVWNLIRYI YNQEEYARYN SDLGEYQAVT ELGRPDAEYW NSQKDLLERR RAEVDTYCRY NYGVVESFTV QRRV (SEQ ID NO:149); and can have a length of about 95 aas (e.g., 93, 94, 95, 96, 97, or 98 aas).

A suitable DRB4 β2 domain, including-naturally occurring allelic variants thereof, may comprise an aa sequence having at least 70%, at least 80%, at least 90%, at least 95%, at least 98%, at least 99%, or 100% aa sequence identity to the following aa sequence: QPKVTV YPSKTQPLQH HNLLVCSVNG FYPGSIEVRW FRNGQEEKAG VVSTGLIQNG DWTFQTLVML ETVPRSGEVY TCQVEHPSMM SPLTVQWSAR SESAQSK (SEQ ID NO:150); and can have a length of about 103 aas (e.g., 100, 101, 102, 103, 104, or 105 aas).

(d) DRB5 Polypeptides

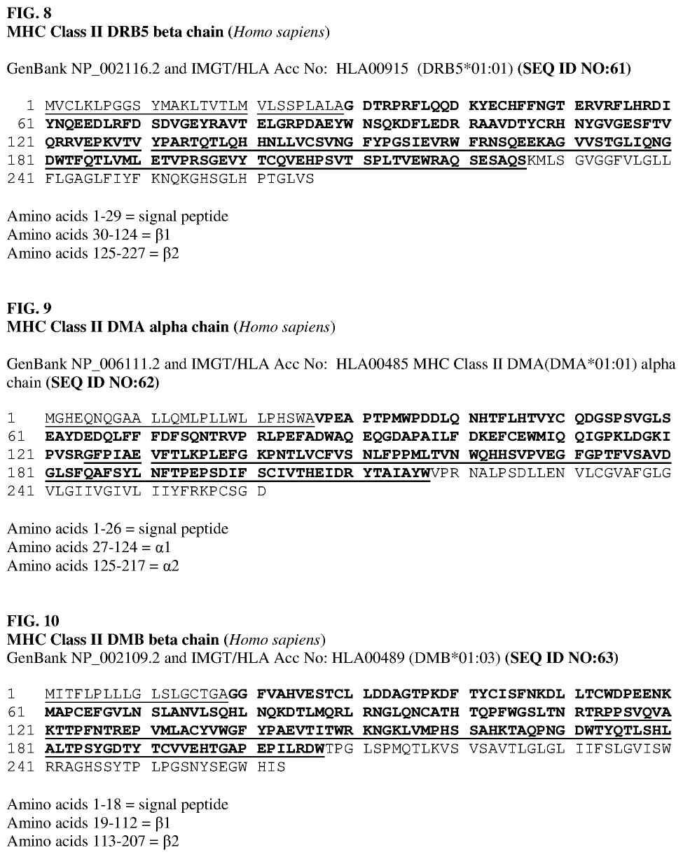

A suitable MHC Class II β chain polypeptide for inclusion in a MAPP is a DRB5 polypeptide. A DRB5 polypeptide can have at least 60%, at least 70%, at least 80%, at least 90%, at least 95%, at least 98%, at least 99%, or 100% aa sequence identity with aas 30-227 of the DRB5 aa sequence depicted in FIG. 8. In some cases, the DRB5 polypeptide has a length of about 198 aas (including, e.g., 195, 196, 197, 198, 199, 200, 201, or 202 aas). In some cases, a DRB5 polypeptide suitable for inclusion in a MAPP comprises an aa substitution, relative to a wild-type DRB5 polypeptide, where the aa substitution replaces an aa (other than a Cys) with a Cys (e.g., for forming a disulfide bond stabilizing the MAPP).

As used herein, the term “DRB5 polypeptide” includes allelic variants, e.g., naturally occurring allelic variants. Thus, in some cases, a suitable DRB4 polypeptide comprises aas 30 to 227 of DRB5*01:01 (SEQ ID NO:61) provided in FIG. 8, or an allelic variant thereof.

A suitable DRB5 β1 domain, including-naturally occurring allelic variants thereof, may comprise an aa sequence having at least 70%, at least 80%, at least 90%, at least 95%, at least 98%, at least 99%, or 100% aa sequence identity to the following aa sequence: M VLSSPLALAG DTRPRFLQQD KYECHFFNGT ERVRFLHRDI YNQEEDLRFD SDVGEYRAVT ELGRPDAEYW NSQKDFLEDR RAAVDTYCRH NYGVGESFTV QRRV (SEQ ID NO:151); and can have a length of about 95 aas (e.g., 93, 94, 95, 96, 97, or 98 aas).

A suitable DRB5 β2 domain, including-naturally occurring allelic variants thereof, may comprise an aa sequence having at least 70%, at least 80%, at least 90%, at least 95%, at least 98%, at least 99%, or 100% aa sequence identity to the following aa sequence: EPKVTV YPARTQTLQH HNLLVCSVNG FYPGSIEVRW FRNSQEEKAG VVSTGLIQNG DWTFQTLVML ETVPRSGEVY TCQVEHPSVT SPLTVEWRAQ SESAQS (SEQ ID NO:152); and can have a length of about 103 aas (e.g., 100, 101, 102, 103, 104, or 105 aas).

(e) DMB Polypeptides

In some cases, a suitable MHC Class II β chain polypeptide is a DMB polypeptide. A DMB polypeptide can have at least 60%, at least 70%, at least 80%, at least 90%, at least 95%, at least 98%, at least 99%, or 100%, aa sequence identity with aas 19-207 of the DMB aa sequence depicted in FIG. 10. In some cases, the DMB polypeptide has a length of about 189 aas (including, e.g., 187, 188, 189, 190, or 191 aas).

As used herein, the term “DMB polypeptide” includes allelic variants, e.g., naturally occurring allelic variants. Thus, in some cases, a suitable DMB polypeptide comprises aas 19 to 207 of DMB*01:03 (SEQ ID NO:63) provided in FIG. 10 (SEQ ID NO:63), or an allelic variant thereof. A suitable DMB β1 domain, including-naturally occurring allelic variants thereof, may comprise an aa sequence having at least 70%, at least 80%, at least 90%, at least 95%, at least 98%, at least 99%, or 100%, aa sequence identity to the following aa sequence: GG FVAHVESTCL LDDAGTPKDF TYCISFNKDL LTCWDPEENK MAPCEFGVLN SLANVLSQHL NQKDTLMQRL RNGLQNCATH TQPFWGSLTN RT (SEQ ID NO:153); and can have a length of about 94 aas (including, e.g., 92, 93, 94, 95, 96, or 97 aas).

A suitable DMB β2 domain, including-naturally occurring allelic variants thereof, may comprise an aa sequence having at least 70%, at least 80%, at least 90%, at least 95%, at least 98%, at least 99%, or 100%, aa sequence identity to the following aa sequence: RPPSVQVA KTTPFNTREP VMLACYVWGF YPAEVTITWR KNGKLVMPHS SAHKTAQPNG DWTYQTLSHL ALTPSYGDTY TCVVEHTGAP EPILRDW (SEQ ID NO:154); and can have a length of about 95 aas (including, e.g., 93, 94, 95, 96, 97, or 98 aas).

(f) DOB Polypeptides

In some cases, a suitable MHC Class II β chain polypeptide is a DOB polypeptide. A DOB polypeptide can have at least 60%, at least 70%, at least 80%, at least 90%, at least 95%, at least 98%, at least 99%, or 100%, aa sequence identity with aas 27-214 of the DOB aa sequence depicted in FIG. 12. In some cases, the DOB polypeptide has a length of about 188 aas (e.g., 186, 187, 188, 189, or 190 aas).

As used herein, the term “DOB polypeptide” includes allelic variants, e.g., naturally occurring allelic variants. Thus, in some cases, a suitable DOB polypeptide comprises aas 27-214 of DOB*01:01 (SEQ ID NO:65) provided in FIG. 12, or an allelic variant thereof.

A suitable DOB β1 domain, including-naturally occurring allelic variants thereof, may comprise an aa sequence having at least 70%, at least 80%, at least 90%, at least 95%, at least 98%, at least 99%, or 100%, aa sequence identity to the following aa sequence: TDSP EDFVIQAKAD CYFTNGTEKV QFVVRFIFNL EEYVRFDSDV GMFVALTKLG QPDAEQWNSR LDLLERSRQA VDGVCRHNYR LGAPFTVGRK (SEQ ID NO:155); and can have a length of about 94 aas (including, e.g., 92, 93, 94, 95, 96, or 97 aas).

A suitable DOB β2 domain, including-naturally occurring allelic variants thereof, may comprise an aa sequence having at least 70%, at least 80%, at least 90%, at least 95%, at least 98%, at least 99%, or 100%, aa sequence identity to the following aa sequence: VQPEVTVYPE RTPLLHQHNL LHCSVTGFYP GDIKIKWFLN GQEERAGVMS TGPIRNGDWT FQTVVMLEMT PELGHVYTCL VDHSSLLSPV SVEW (SEQ ID NO:156); and can have a length of about 94 aas (including, e.g., 92, 93, 94, 95, 96, or 97 aas).

(g) DPB1 Polypeptides

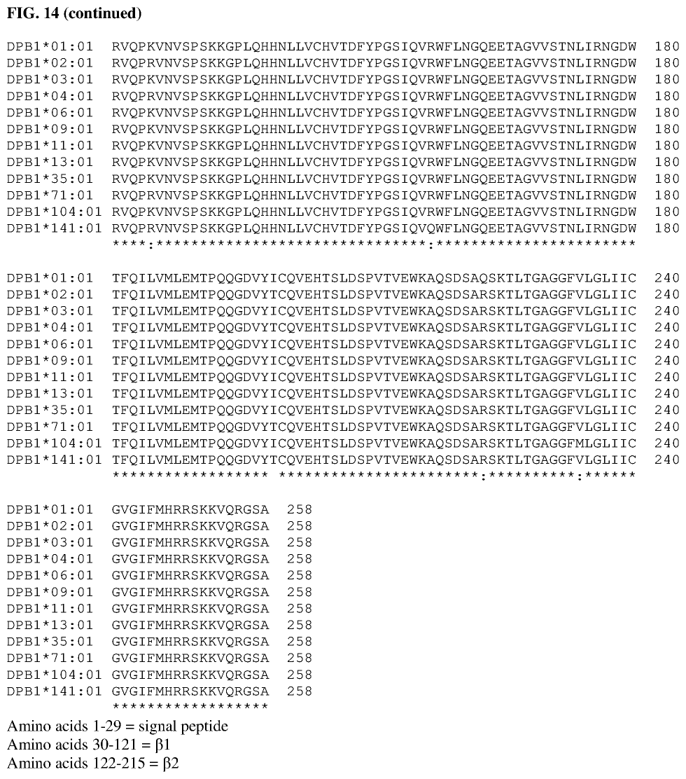

In some cases, a suitable MHC Class II β chain polypeptide is a DPB1 polypeptide. A DPB1 polypeptide can have at least 60%, at least 70%, at least 80%, at least 90%, at least 95%, at least 98%, at least 99%, or 100%, aa sequence identity with aas 30-215 of any of the DPB1 aa sequences depicted in FIG. 14 including naturally occurring allelic variants. FIG. 14 displays the DPB1 precursor proteins in which aas 1-29 are the signal sequence (underlined), 30-121 form the β1 region, and 122-215 form the β2 region. In some cases, a DPB1 polypeptide suitable for inclusion in a MAPP comprises an aa substitution, relative to a wild-type DPB1 polypeptide, where the aa substitution replaces an aa (other than a Cys) with a Cys (e.g., for forming a disulfide bond stabilizing the MAPP).

A suitable MHC Class II β chain polypeptide for inclusion in a MAPP includes a DPB1 polypeptide. In some cases, the DPB1 polypeptide has a length of about 186 aas (including, e.g., 184, 185, 186, 187, or 188 aas). In an embodiment, a DPB1 can have at least 60%, at least 70%, at least 80%, at least 90%, at least 95%, at least 98%, at least 99%, or 100%, aa sequence identity with aas 30-215 of a DPB1 sequence provided in FIG. 14, including one of the following DPB1 polypeptides:
(i) the DPB1*01:01 β chain aa sequence of IMGT/HLA Acc No: HLA00514 in FIG. 14;
(ii) the DPB1*02:01 β chain aa sequence of IMGT/HLA Acc No: HLA00517 in FIG. 14;
(iii) the DPB1*03:01 β chain aa sequence of IMGT/HLA Acc No: HLA00520 in FIG. 14;
(iv) the DPB1*04:01 β chain aa sequence of IMGT/HLA Acc No: HLA00521, GenBank NP_002112.3 in FIG. 14;
(v) the DPB1*06:01 β chain aa sequence of IMGT/HLA Acc No: HLA00524 in FIG. 14;
(vi) the DPB1*11:01 β chain aa sequence of IMGT/HLA Acc No: HLA00528 in FIG. 14;
(vii) the DPB1*71:01 β chain aa sequence of IMGT/HLA Acc No:HLA00590 in FIG. 14;
(viii) the DPB1*104:01 β chain aa sequence IMGT/HLA Acc No: HLA02046 in FIG. 14; and
(ix) the DPB1*141:01 beta chain aa sequence in FIG. 14, IMGT/HLA Acc No: HLA10364.

As used herein “DPB1 polypeptide” includes allelic variants, e.g., naturally occurring allelic variants. Thus, in some cases, a suitable DPB1 polypeptide comprises the following aa sequence: R ATPENYLFQG RQECYAFNGT QRFLERYIYN REEFARFDSD VGEFRAVTEL GRPAAEYWNS QKDILEEKRA VPDRMCRHNY ELGGPMTLQR RVQPRVNVSP SKKGPLQHHN LLVCHVTDFY PGSIQVRWFL NGQEETAGVV STNLIRNGDW TFQILVMLEM TPQQGDVYTC QVEHTSLDSP VTVEW (SEQ ID NO:157), or an allelic variant thereof.

A suitable DPB1 β1 domain, including-naturally occurring allelic variants thereof, may comprise an aa sequence having at least 70%, at least 80%, at least 90%, at least 95%, at least 98%, at least 99%, or 100%, aa sequence identity to the following aa sequence: R ATPENYLFQG RQECYAFNGT QRFLERYIYN REEFARFDSD VGEFRAVTEL GRPAAEYWNS QKDILEEKRA VPDRMCRHNY ELGGPMTLQR R (SEQ ID NO:158); and can have a length of about 92 aas (including, e.g., 90, 91, 92, 93, or 94 aas).

A suitable DPB1 β2 domain, including-naturally occurring allelic variants thereof, may comprise an aa sequence having at least 70%, at least 80%, at least 90%, at least 95%, at least 98%, at least 99%, or 100%, aa sequence identity to the following aa sequence: VQPRVNVSP SKKGPLQHHN LLVCHVTDFY PGSIQVRWFL NGQEETAGVV STNLIRNGDW TFQILVMLEM TPQQGDVYTC QVEHTSLDSP VTVEW (SEQ ID NO:159); and can have a length of about 94 aas (including, e.g., 92, 93, 94, 95, 96, or 97 aas).

(h) DQB1 Polypeptides

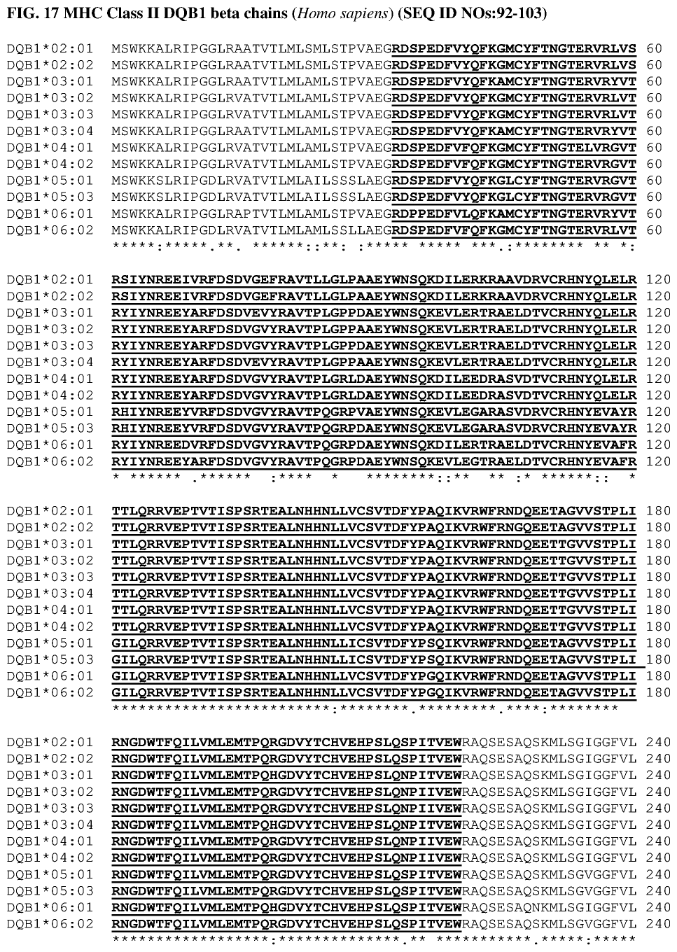

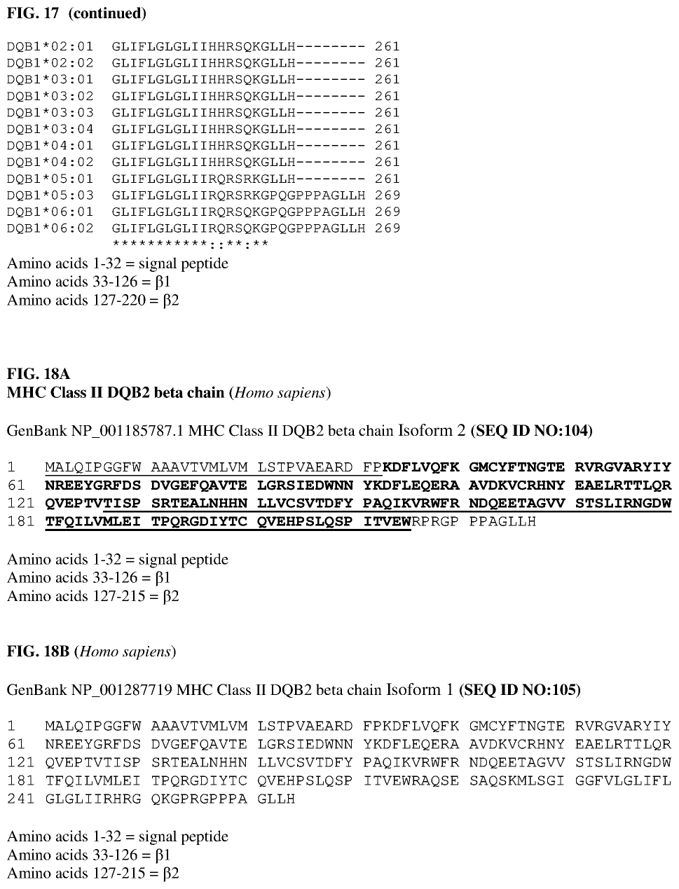

In some cases, a suitable MHC Class II β chain polypeptide is a DQB1 polypeptide. A DQB1 polypeptide can have at least 60%, at least 70%, at least 80%, at least 90%, at least 95%, at least 98%, at least 99%, or 100%, aa sequence identity with aas 33-220 of the DQB1 aa sequence depicted in FIG. 17. In some cases, the DQB1 polypeptide has a length of about 188 aas (e.g., 186, 187, 188, 190, 191, or 192 aas).

As used herein “DQB1 polypeptide” includes allelic variants, e.g., naturally occurring allelic variants. Thus, in some cases, a suitable DQB1 polypeptide comprises aas 33-220 of DQB1*06:02 provided in FIG. 17 (SEQ ID NO:103), or an allelic variant thereof.

A suitable DQB1 β1 domain, including-naturally occurring allelic variants thereof, may comprise an aa sequence having at least 70%, at least 80%, at least 90%, at least 95%, at least 98%, at least 99%, or 100%, aa sequence identity to the following aa sequence: RDSPEDFV FQFKGMCYFT NGTERVRLVT RYIYNREEYA RFDSDVGVYR AVTPQGRPDA EYWNSQKEVL EGTRAELDTV CRHNYEVAFR GILQRR (SEQ ID NO:160); and can have a length of about 94 aas (including e.g., 92, 93, 94, 95, or 96 aas).

A suitable DQB1 β2 domain, including-naturally occurring allelic variants thereof, may comprise an aa sequence having at least 70%, at least 80%, at least 90%, at least 95%, at least 98%, at least 99%, or 100%, aa sequence identity to the following aa sequence: VEPT VTISPSRTEA LNHHNLLVCS VTDFYPGQIK VRWFRNDQEE TAGVVSTPLI RNGDWTFQIL VMLEMTPQRG DVYTCHVEHP SLQSPITVEW (SEQ ID NO:161); and can have a length of about 94 aas (including e.g., 92, 93, 94, 95, or 96 aas).

(i) DQB2 Polypeptides

In some cases, a suitable MHC Class II β chain polypeptide is a DQB2 polypeptide. A DQB2 polypeptide can have at least 60%, at least 70%, at least 80%, at least 90%, at least 95%, at least 98%, at least 99%, or 100%, aa sequence identity with aas 33-215 of the DQB2 aa sequence depicted in FIG. 18A or FIG. 18B. In some cases, the DQB2 polypeptide has a length of about 182 aas (e.g., 175, 176, 177, 178, 179, 180, 181, or 182 aas).

As used herein “DQB2 polypeptide” includes allelic variants, e.g., naturally occurring allelic variants. Thus, in some cases, a suitable DQB2 polypeptide comprises the following aa sequence: DFLVQFK GMCYFTNGTE RVRGVARYIY NREEYGRFDS DVGEFQAVTE LGRSIEDWNN YKDFLEQERA AVDKVCRHNY EAELRTTLQR QVEPTVTISP SRTEALNHHN LLVCSVTDFY PAQIKVRWFR NDQEETAGVV STSLIRNGDW TFQILVMLEI TPQRGDIYTC QVEHPSLQSP ITVEW (SEQ ID NO:162), or an allelic variant thereof.

A suitable DQB2 β1 domain, including-naturally occurring allelic variants thereof, may comprise an aa sequence having at least 70%, at least 80%, at least 90%, at least 95%, at least 98%, at least 99%, or 100%, aa sequence identity to the following aa sequence: DFLVQFK GMCYFTNGTE RVRGVARYIY NREEYGRFDS DVGEFQAVTE LGRSIEDWNN YKDFLEQERA AVDKVCRHNY EAELRTTLQR QVEPTV (SEQ ID NO:163); and can have a length of about 94 aas (including e.g., 92 93, 94, 95, 96, or 97 aas).

A suitable DQB2 β2 domain, including-naturally occurring allelic variants thereof, may comprise an aa sequence having at least 70%, at least 80%, at least 90%, at least 95%, at least 98%, at least 99%, or 100%, aa sequence identity to the following aa sequence: TISP SRTEALNHHN LLVCSVTDFY PAQIKVRWFR NDQEETAGVV STSLIRNGDW TFQILVMLEI TPQRGDIYTC QVEHPSLQSP ITVEW (SEQ ID NO:164); and can have a length of about 94 aas (including e.g., 92, 93, 94, 95, 96, or 97 aas).

(iii) MHC Class II Disease Risk-Associated Alleles and Haplotypes

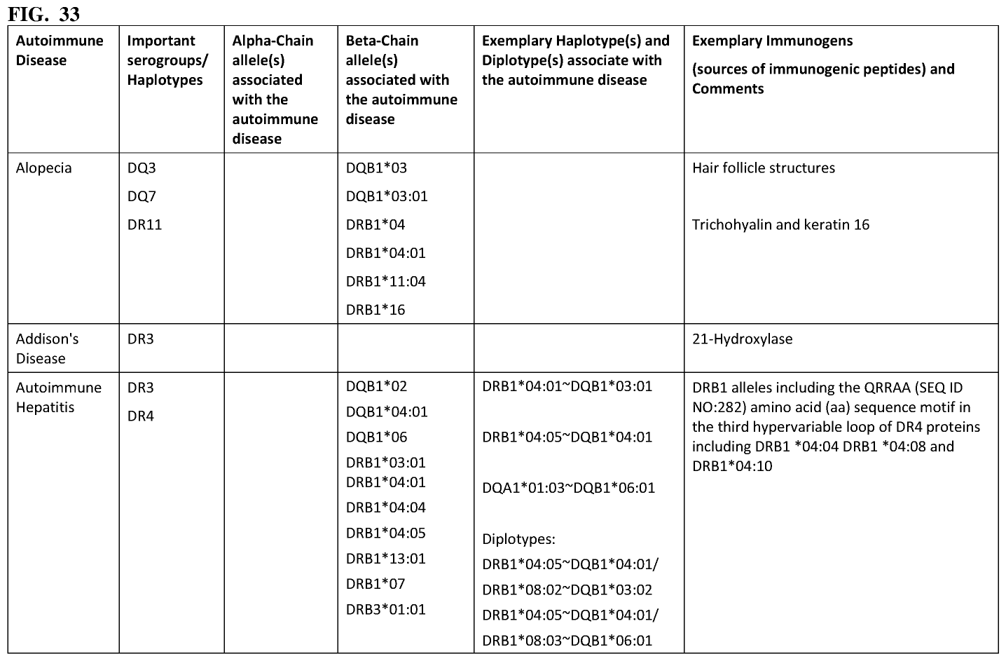

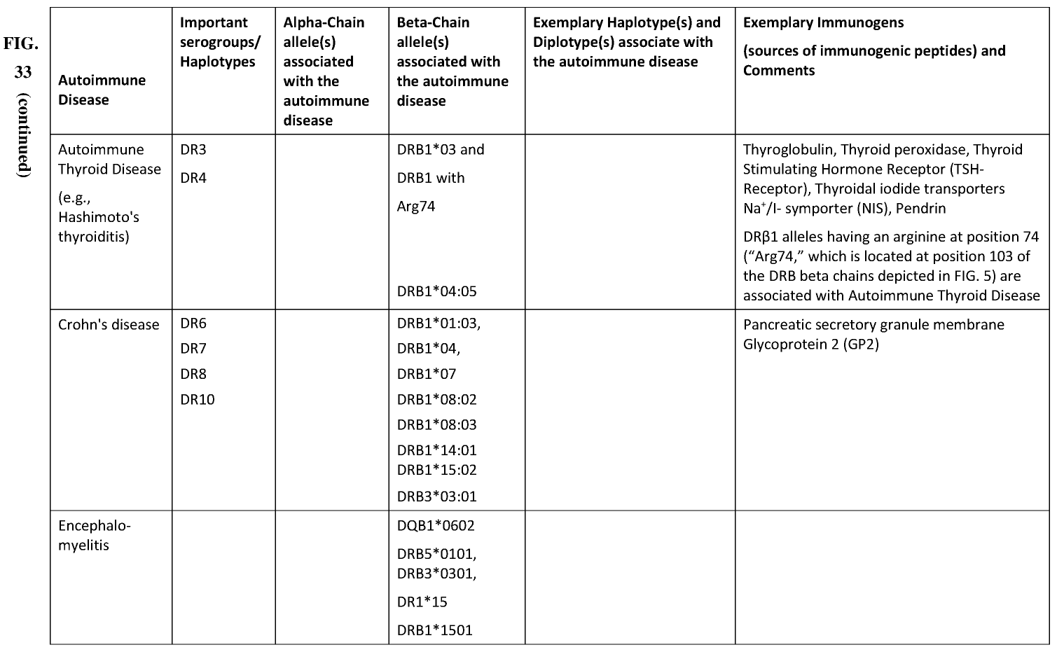

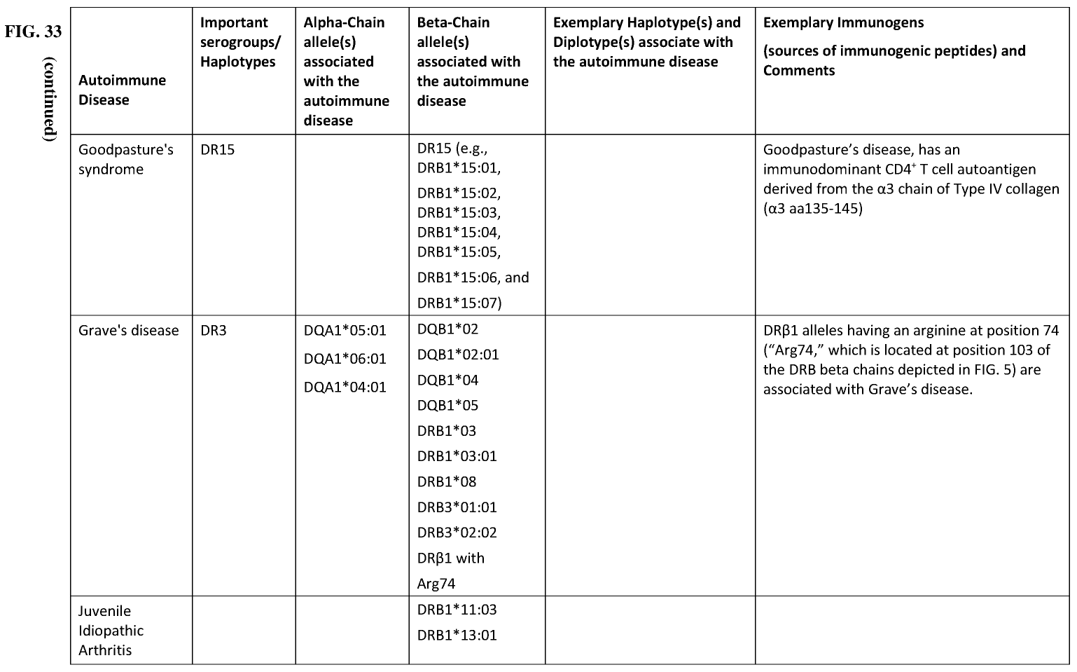

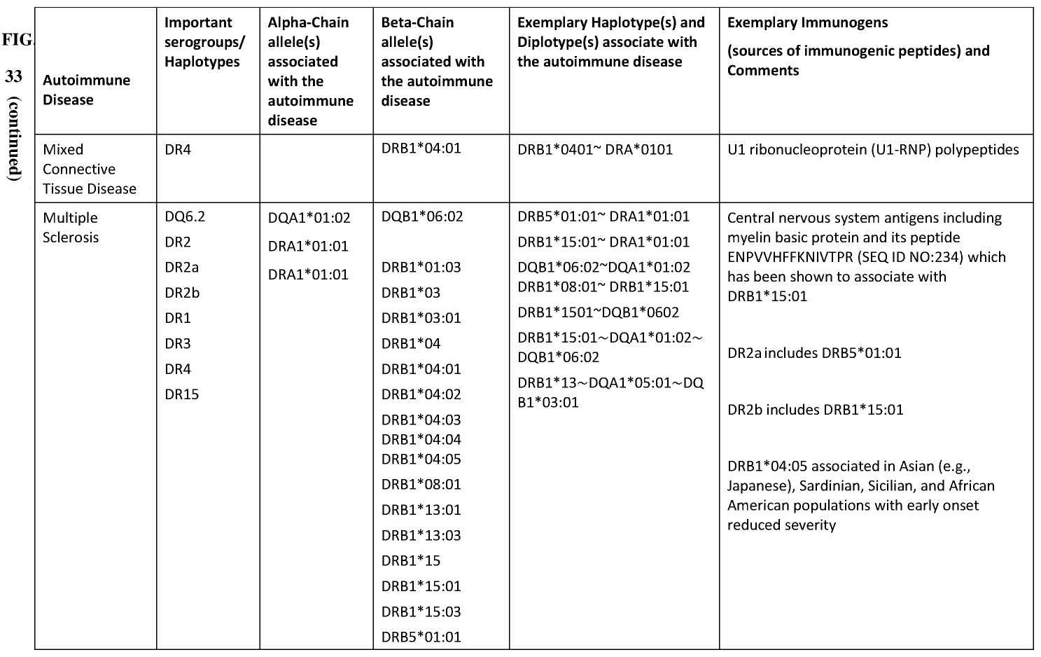

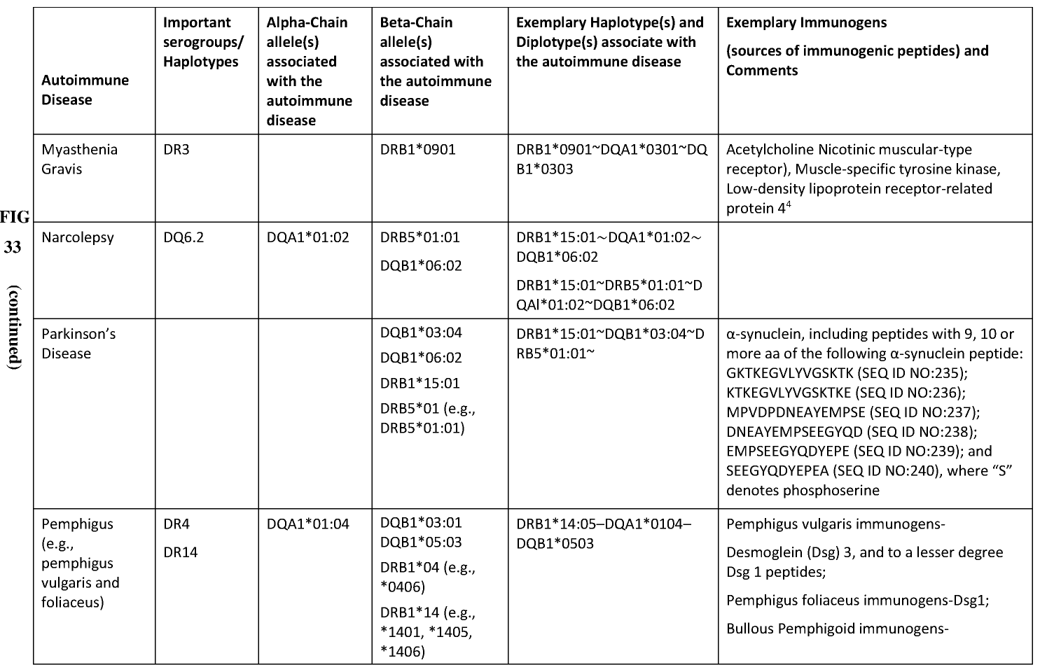

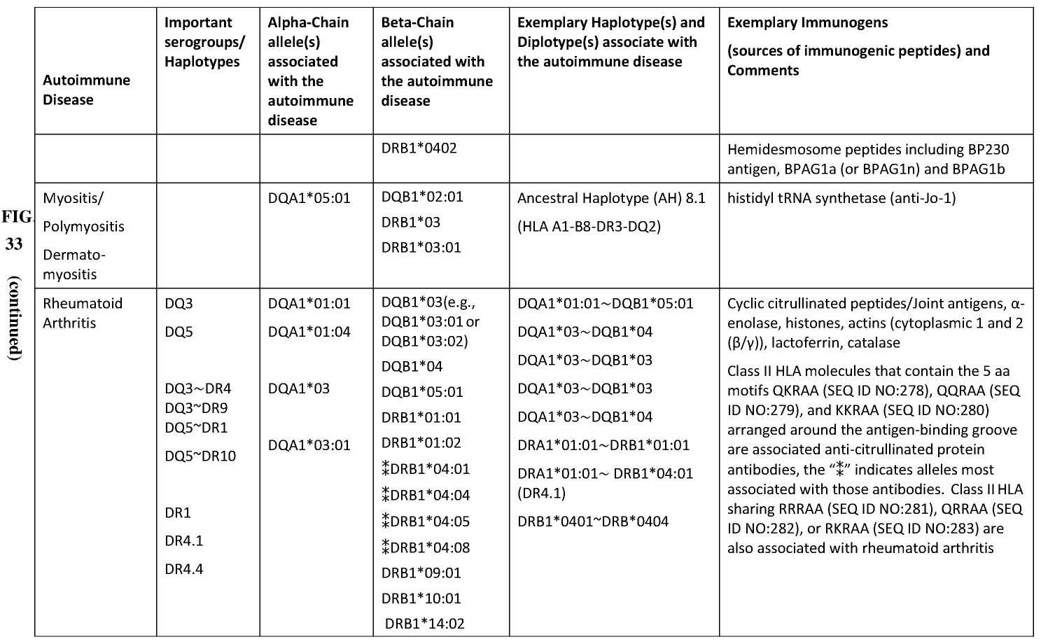

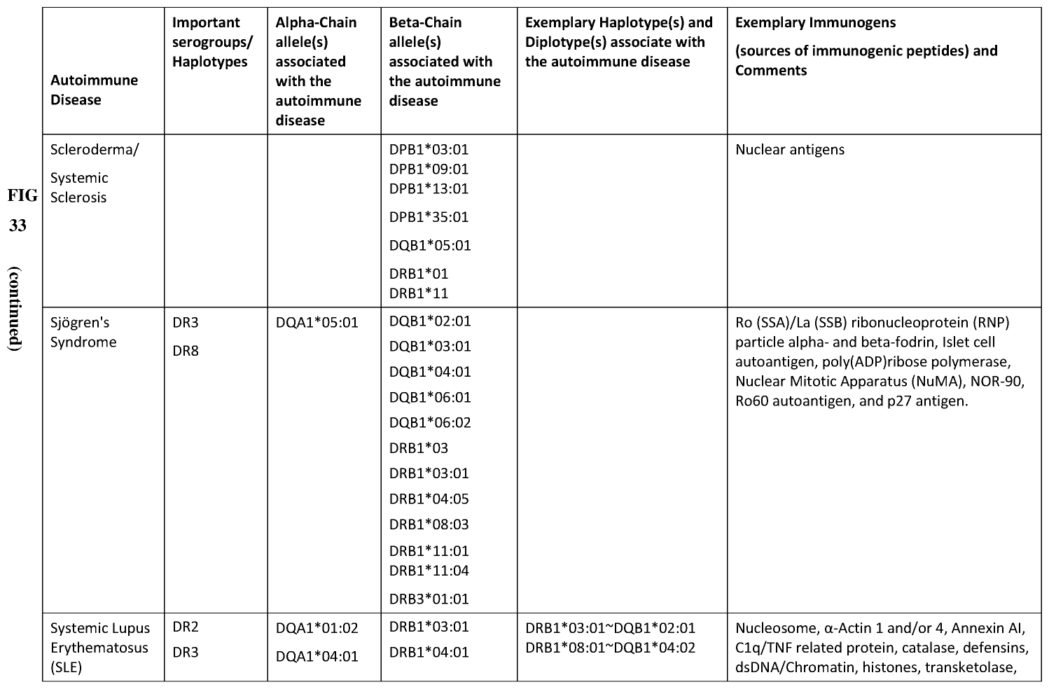

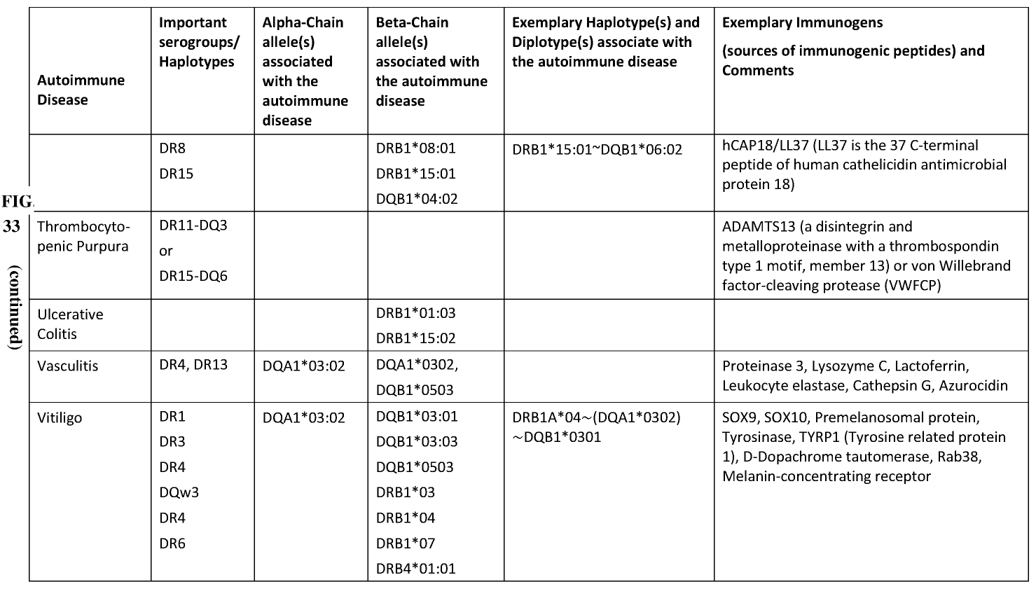

Certain alleles and haplotypes of MHC Class II have been associated with disease, e.g., increased risk of developing a particular disease. See, e.g., Erlich et al. (2008) Diabetes 57:1084; Gough and Simmonds (2007) Curr. Genomics 8:453; Mitchell et al. (2007) Robbins Basic Pathology Philadelphia: Saunders, 8^{th} ed.; Margaritte-Jeannin et al. (2004) Tissue Antigens 63:562; and Kurko et al. (2013) Clin. Rev. Allergy Immunol. 45:170. A number of those diseases and their associated alleles and/or haplotypes are described in WO 2020/181273 assigned to Cue Biopharma and references cited therein. Some HLA haplotypes and alleles associated with increased risk that an individual expressing such HLA haplotypes and/or alleles will develop a given autoimmune disease are set forth in the table provided in FIG. 33. That table also provides a listing of the molecules associated with the disease (e.g., autoantigens such as proteins and peptides) that can act as epitopes or a source of epitopes. A MAPP of the present disclosure that is directed to the treatment of a specific disease can include any of the disease associated HLA haplotypes and/or alleles and the corresponding epitopes set out in FIG. 33. The peptide epitope can be, for example, a peptide of from 4 aas to about 25 aas in length of any of the autoantigens set out in the table.

The following are notes to the table provided in FIG. 33: 1) AH8.1 (e.g., HLA A1-B8-DR3-DQ2 haplotype); 2) DQ3 alleles include DQB1*03 alleles such as DQB1*03:01 to DQB1*03:05 proteins; 3) DQ5 alleles include DQB1*05 alleles such as DQB1*05:01 to DQB1*05:04 and may be associated with DQA1*01:01; 4) DR2 alleles include DRB1*15:01-15:04 and DRB1*16:01-16:06; 5) DR3 haplotypes include: DRB1*03:01, DRB1*03:02, DRB1*03:03, and DRB1*03:04; 6) DR4 haplotypes include: DRB1*04:01 through DRB1*04:13; AH=ancestral haplotype; 7) Simmonds et al., Am. J. Hum. Genet. 76:157-163, (2005), see Table 1, HLAs with odds ratios greater than 1.5 include the following DRB1, DAB1 and DQA1 alleles: DRB1*:−03:01 to −03:05, −10:01, −08:01 to 11, −16:01 to 16:06, −11:01 to −11; 21, −01:01 to −01:04, −04:01 to −04:22, and −15:01 to −15:05; DQB1*: −02, −04, −03:01, −03:04, −05, −06:01 to 06:09, and −03:02; and HLA−DQA1*: −05:01 to −05:02, −06:01, −04:01, −01:01, −01:02, −01:04, −01:03, −03:11, and −03:12; 8) Li et al., Mol Med Rep.; 17(5): 6533-6541 (2018) noting epitopes from auto antigens including: SMD1 (NCBI Accession: CAE11897.1); SMD2 (NCBI Accession: AAC13776.1); SMD3 (NCBI Accession: AAA57034.1); Proliferating cell nuclear antigen (PCNA) (NCBI Accession: NP_872590.1); Acidic ribosomal phosphoprotein (P1) (NCBI Accession: AAA36471.1); Acidic ribosomal phosphoprotein (P2) (NCBI Accession: AAA36472.1); snRNP-B/B′ (NCBI Accession: P14678.2); U1-snRNP-C(NCBI Accession: NP_003084.1); U1-snRNP-A (NCBI Accession: NP_004587.1); Nucleolin (NCBI Accession: AAA59954.1); Acidic ribosomal phosphoprotein (P0) (NCBI Accession: AAA36470.1); DNA topoisomerasel (truncated) (NCBI Accession: NP_003277.1); DNA topoisomerase 1 (full length) (NCBI Accession: NP_003277.1); and U1-SnRNP 68/70 KDa (NCBI Accession: P08621.2).

(a) Individual Disease Risk-Associated Alleles

The association a number of HLA alleles with one or more autoimmune diseases is described in, for example, FIG. 33. The sequences of the disease-associated alleles are provided in the figures accompanying this disclosure (e.g., DRB1 alleles are provided in FIG. 5). Where diseases associations are made to groups of alleles (e.g., DRB1*03), the sequences of additional alleles may be obtained from standard references including those provided by the U.S. National Center for Biotechnology Information (NCBI) and at hla.alleles.org/nomenclature/index.html.

An exemplary association between various diseases states and particular HLA alleles include the association of the alleles of the HLA-DR3 with early-age onset myasthenia gravis, Hashimoto's thyroiditis, autoimmune hepatitis, primary Sjögren's syndrome, and SLE. Other exemplary associations include: DRB1*0301 (“DRB1*03:01” in FIG. 5) association with an increased of developing early onset Grave's disease and/or type 1 autoimmune hepatitis; DRB1*04:01 association with an increased risk of developing multiple sclerosis and/or rheumatoid arthritis.

DRB1*04:02 association with increased risk of developing idiopathic Pemphigus vulgaris, and/or SLE (e.g., SLE-associated anti-cardiolipin, SLE-associated anti-β2 glycoprotein I).

DRB1*0403 association with increased risk of developing SLE (e.g., increased risk of developing SLE-associated anti-cardiolipin antibodies and/or SLE-associated anti-β2 glycoprotein I antibodies); DRB1*04:05 association with increased risk of developing rheumatoid arthritis and/or autoimmune hepatitis; and DRB1*04:06 association. with increased risk of developing anti-caspase-8 autoantibodies (e.g., in silicosis-systemic sclerosis (SSc)-systemic lupus erythematosus (SLE)).

Certain DQB1 alleles are also associated with increased risk that an individual expressing such an allele will develop an autoimmune disease. For example, DQB1*0301, and DQB1*0602 are associated with an increased risk of developing MS and/or a more severe MS phenotype (e.g., more severe inflammatory and neurodegenerative damage).

(iv) Disulfide Bonds and the Presenting Sequences and Presenting Complexes

Disulfide bonds involving an MHC peptide sequence may be included in a presenting sequence or complex of a MAPP. The disulfide bonds may increase the stability (e.g., thermal stability) and/or assist in positioning a peptide epitope in the binding pocket/groove of the MHC formed by its α and β chain sequences. The disulfide bonds may be between two MHC peptide sequences (e.g., a cysteine located in an α chain and a cysteine located in a β chain sequence). Disulfide bonds, and particularly disulfide bonds made to position a peptide epitope may be between two MHC peptide sequences or, alternatively, between a MHC peptide sequence and a linker attaching the peptide epitope and an MHC sequence (e.g., the linker between the epitope and β1 domain sequence in FIG. 15 structures A and B). Disulfide bonds for the stabilization and/or positioning epitope may be made using cysteines found within the MHC sequences and/or cysteines that have been added/engineered into one or more MHC sequences using the techniques of molecular biology. As discussed above, the α chain may include cysteines at positions 3, 4, 12, 28, 29, 72, 75, 80, 81, 82, 93, 94, and 95 of the mature α chain (lacking its signal sequence). For the DRA polypeptides cysteines substitutions include those at E3C, E4C, F12C, G28C, D29C, I72C, K75C, T80C, P81C, I82C, T93C, N94C, and S95C (see FIG. 4). The β chain may include cysteine at positions 5, 7, 10, 19, 20, 33, 151, 152, and 153 of the mature β chain (lacking its signal sequence). For the DB1 polypeptides cysteines substitutions include those at positions P5C, F7C, Q10C (may be Y10C or E10C for some DRB1 alleles), N19C, G20C, H33C (may be N33C for some DRB1alleles), G151C, D152C, and W153C.

Stabilizing disulfide bonds between α and β chain sequences include those between the α and β chain positions set forth in Table 3, which also provides the specific cysteine substitutions for HLA DRA*01:02 and DRB*0401 sequences. The stabilizing disulfide bonds between the MHC (e.g., HLA) α and β chains may be incorporated into any of the MAPP structures described herein. For example, such disulfide bonds may be incorporated into presenting sequences such as those shown in FIG. 15 and the presenting sequences shown in the MAPPs of FIG. 14. Stabilizing disulfide bonds may, for example be incorporated into a presenting sequence having, in order from the N- to C-terminus β1, β2, α1, and α2 domains (see e.g., FIG. 15 structure B).

TABLE 3

| | | DRA*01:02 | DRB*0401 |
|---|---|---|---|
| α chain | β chain | α chain | β chain |
| position | position | substitution | substitution |
| 3 | 19 | E3C | N19C |
| 3 | 20 | E3C | G20C |
| 4 | 19 | E4C | N19C |
| 4 | 20 | E4C | G20C |
| 28 | 151 | G28C | G151C |
| 28 | 152 | G28C | D152C |
| 28 | 153 | G28C | W153 |
| 29 | 151 | D29C | G151C |
| 29 | 152 | D29C | D152C |
| 29 | 153 | D29C | W153 |
| 80 | 33 | T80C | H33C |
| 81 | 33 | P81C | H33C |
| 82 | 33 | I82C | H33C |
| 93 | 156 | T93C | Q156C |
| 93 | 153 | T93C | W153C |
| 94 | 156 | N94C | Q156C |
| 94 | 120 | N94C | N120C |
| 95 | 156 | S95C | Q156C |
| 95 | 120 | S95C | N120C |

Disulfide bonds between the MHC α and β chain sequences that assist in positioning the peptide epitope and/or stabilizing the structure of the presenting sequence or complex are formed between a first aa and second aa of the MAPP. The first aa is either (i) an aa position proximate to the point where a peptide epitope (or a peptide epitope and linker) are attached to an MHC peptide sequence or (ii) is an aa (a cysteine) in a linker attached to the peptide epitope, while the second aa is position elsewhere in the MHC peptide sequence. By way of example, where a presenting sequence comprises from N-terminus to C-terminus a peptide epitope, β1 domain, β2 domain, α1 domain, and α2 domain aa sequences, a cysteine substituted within the first ten amino acids (e.g., aas 5-10) of the β1 domain can serve as a first aa and provide a point to anchor the peptide epitope and/or stabilize the MAPP when bonded to a with second cysteine located in, for example, the α1 domain, or α2 domain of the presenting sequence. Some examples of disulfide bonds between the MHC α and β chain sequences that assist in positioning the peptide epitope and/or stabilizing the structure of a presenting sequence or complex include those set forth in Table 4.

TABLE 4

| | | DRA*01:02 | DRB*0401 |
|---|---|---|---|
| α chain | β chain | α chain | β chain |
| position | position | substitution | substitution |
| 12 | 7 | F12C | F7C |
| 12 | 10 | F12C | Q10C |
| 80 | 5 | T80C | P5C |
| 80 | 7 | T80C | F7C |
| 81 | 5 | P81C | P5C |
| 81 | 7 | P81C | F7C |
| 82 | 5 | I82C | P5C |
| 82 | 7 | I82C | F7C |

Thus, for example, when a presenting sequence of complex comprises in the N-terminal to C-terminal direction a peptide epitope bound to a Rβ1 domain, then a disulfide bond between a cysteine substituted at one of position 5-7 of the β chain, and a cysteine at one of aa positions 80-82 of the α chain may be use for positioning the peptide epitope or stabilizing the structure of a presenting sequence. By way of example a disulfide bond between a β chain P5C substitution and an α chain P81C substitution may be used for positioning of the peptide epitope and or stabilization of a presenting sequence. The same type of disulfide bonding is applicable to presenting complexes, and both presenting complexes and presenting sequences may have additional disulfide bonds (e.g., as in Table 3) for stabilization.

Where a cysteine residue in a linker attached to the peptide epitope is employed to position the peptide epitope and/or stabilize the structure of a presenting sequence or complex, the cysteine is typically located at an aa proximate to the point where the linker and peptide epitope meet. For example, where the MAPP comprises an epitope place on the N-terminal side of a linker peptide sequence the cysteine may be within about 6 aas of the position were the linker and peptide epitope meet, that is to say at one of amino acids 1-5 (aa1, aa2, aa3, aa4, or aa5) of a MAPP comprising the construct epitope-aa1-aa2,aa3-aa4-aa5-(remainder of the linker! MAPP). Where the linker comprises repeats of the sequence GGGGS (SEQ ID NO:203), aa1 to aa5 are G1, G2, G3, G4, and S5, and the linker substitutions may be referred to as, for example a “G2C.” This is exemplified by SEQ ID NO:165, that has four repeats of GGGGS in which the aa at position 2 of the linker (aa2), is a glycine substituted by a cysteine: GCGGSGGGGSGGGGSGGGGS (SEQ ID NO:165). Examples of cysteine containing linkers suitable for forming disulfide bonds with a cysteine in an MHC peptide (e.g., an α chain peptide sequence such a DRA peptide) in a presenting sequence or complex comprising an epitope placed on the N-terminal side of a linker bound to an MHC β chain such as a DRB polypeptide (i.e., the MAPP comprises the structure epitope-aa1-aa2-aa3-aa4-aa5-remainder of linker if present]-β1 domain such as a DRB β1 domain) are set forth in Table 5. Also provided in Table 5 is the location for a cysteine substituted in a DRA peptide (see e.g., FIG. 4) that will form the disulfide bond for positioning the peptide epitope and/or stabilizing the structure of a presenting sequence or complex.

TABLE 5
Exemplary Linker disulfide bonds for MAPPS comprising the structure epitope-aa1-
aa2-aa3-aa4-aa5-(remainder of linker if present)-β1 domain e.g., of a DRB peptide

| | | | α chain position, |
|---|---|---|---|
| Cysteine | Cysteine substitution in the | Cysteine substitution in a | (DRA Cysteine |
| position | general linker structure | GGGGS containing linker | substitution) |
| aa1 | epitope-C-aa2-aa3-aa4-aa5- | epitope-CGGGS-(remainder | 72 (I72C) |
| | (remainder of linker)-DRB | of linker)-DRB | |
| | | (i.e., a linker G1C substitution) | |
| aa2 | epitope-aa1-C-aa3-aa4-aa5- | epitope-GCGGS-(remainder | 72 (I72C) |
| | (remainder of linker)-DRB | of linker)-DRB | |
| | | (i.e., a linker G2C substitution) | |
| aa3 | epitope-aa1-aa2-C-aa4-aa5- | epitope-GGCGS-(remainder | 72 (I72C) |
| | (remainder of linker)-DRB | of linker)-DRB | |
| | | (i.e., a linker G3C substitution) | |
| aa4 | epitope-aa1-aa2-aa3-C-aa5- | epitope-GGGCS-(remainder | 72 (I72C) |
| | (remainder of linker)-DRB | of linker)-DRB | |
| | | (i.e., a linker G4C substitution) | |
| aa5 | epitope-aa1-aa2-aa3 -aa4-C- | epitope-GCGGGC-(remainder | 72 (I72C) |
| | (remainder of linker)-DRB | of linker)-DRB | |
| | | (i.e., a linker S5C substitution) | |
| aa1 | epitope-C-aa2-aa3-aa4-aa5- | epitope-CGGGS-(remainder | 75 (K75C) |
| | (remainder of linker)-DRB | of linker)-DRB | |
| aa2 | epitope-aa1-C-aa3-aa4-aa5- | epitope-GCGGS-(remainder | 75 (K75C) |
| | (remainder of linker)-DRB | of linker)-DRB | |
| aa3 | epitope-aa1-aa2-C-aa4-aa5- | epitope-GGCGS-(remainder | 75 (K75C) |
| | (remainder of linker)-DRB | of linker)-DRB | |
| aa4 | epitope-aa1-aa2-aa3-C-aa5- | epitope-GGGCS-(remainder | 75 (K75C) |
| | (remainder of linker)-DRB | of linker)-DRB | |
| aa5 | epitope-aa1-aa2-aa3 -aa4-C- | epitope-GCGGGC-(remainder | 75 (K75C) |
| | (remainder of linker)-DRB | of linker)-DRB | |

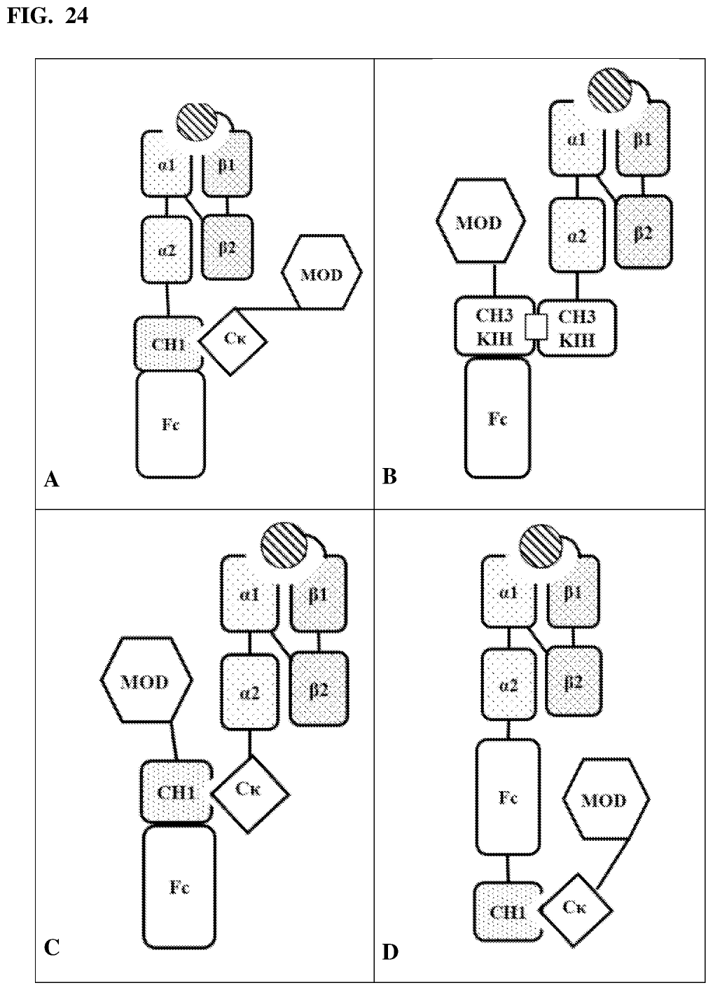

MAPPs with presenting sequences or complexes comprising an epitope-linker-DRB structure recited in Table 5 (see, e.g.: FIG. 24; FIG. 25 structures A and B; FIG. 26 structures A, D, F and H; FIG. 27; FIG. 28, and FIG. 29 A-F) may have for example a disulfide bond for positioning the peptide epitope and/or stabilizing the structure of a presenting sequence or complex. The disulfide may be formed between linker aa2 (e.g., a G2C) and a cysteine at DRA aa 72 (e.g., I72C). The disulfide may be formed between linker aa2 (e.g., a G2C) and a cysteine at DRA aa 72 (e.g., K75C).

Where a disulfide bond is formed between the linker and an MHC polypeptide of a presenting sequence or presenting complex, the presenting sequence or presenting complexe may have additional disulfide bonds (e.g., as in Table 3) for stabilization.

5. Immunomodulatory Polypeptides (“MODs”)

A MAPP may comprise one or more immunomodulatory polypeptides or “MODs”. MODs that are suitable for inclusion in a MAPP include, but are not limited to, IL-1, IL-2, IL-4, IL-6, IL-7, IL-10, IL-12, IL-15, IL-17, IL-21, IL-23, CD7, CD30L, CD40, CD70, CD80, (B7-1), CD83, CD86 (B7-2), HVEM (CD270), ILT3 (immunoglobulin-like transcript 3), ILT4 (immunoglobulin-like transcript 4), Fas ligand (FasL), ICAM (intercellular adhesion molecule), ICOS-L (inducible costimulatory ligand), JAGI (CD339), lymphotoxin beta receptor, 3/TR6, OX40L (CD252), PD-L1, PD-L2, TGF-β1, TGF-β2, TGF-β3, 4-iBBL, and fragments of any thereof, such as ectodomain fragments, capable of engaging and signaling through their cognate receptor. Some MOD polypeptides suitable for inclusion in a MAPP of the present disclosure, and their “co-MODS (“co-immunomodulatory polypeptides” or cognate costimulatory receptors) include polypeptide sequences with T cell modulatory activity from the protein pairs recited in the following table:

Exemplary Pairs of MODs and Co-MODs

| | |
|---|---|
| a) | 4-1BBL (MOD) and 4-1BB (Co-MOD); |
| b) | PD-L1 (MOD) and PD1 (Co-MOD); |
| c) | IL-2 (MOD) and IL-2 receptor (Co-MOD); |
| d) | CD80 (MOD) and CD28 (Co-MOD); |
| e) | CD86 (MOD) and CD28 (Co-MOD); |
| f) | OX40L (CD252) (MOD) and OX40 (CD134) |
| | (Co-MOD); |
| g) | Fas ligand (MOD) and Fas (Co-MOD); |
| h) | ICOS-L (MOD) and ICOS (Co-MOD); |
| i) | ICAM (MOD) and LFA-1 (Co-MOD); |
| j) | CD30L (MOD) and CD30 (Co-MOD); |
| k) | CD40 (MOD) and CD40L (Co-MOD); |
| l) | CD83 (MOD) and CD83L (Co-MOD); |
| m) | HVEM (CD270) (MOD) and CD160 (Co- |
| | MOD); |
| n) | JAG1 (CD339) (MOD) and Notch (Co- |
| | MOD); |
| o) | JAG1 (CD339) (MOD) and CD46 (Co- |
| | MOD); |
| p) | CD70 (MOD) and CD27 (Co-MOD); |
| q) | CD80 (MOD) and CTLA4 (Co-MOD); |
| r) | CD86 (MOD) and CTLA4 (Co-MOD); |
| s) | PD-L1(MOD) and CD-80 (Co-MOD); and |
| t) | TGF-β1, TGF-β2, and/or TGF-β3 (MODs) |
| | and TGF-β Receptor (e.g., TGFBR1 |
| | and/or TGFBR2) (Co-MOD) |

In some cases, the MOD is selected from an IL-2 polypeptide, a 4-1BBL polypeptide, a B7-1 polypeptide; a B7-2 polypeptide, an ICOS-L polypeptide, an OX-40L polypeptide, a CD80 polypeptide, a CD86 polypeptide, a PD-L1 polypeptide, a FasL polypeptide, a TGFβ polypeptide, and a PD-L2 polypeptide. In some cases, the MAPP or duplex MAPP comprises two different MODs, such as an IL-2 MOD or IL-2 variant MOD polypeptide and either a CD80 or CD86 MOD polypeptide. In another instance, the MAPP or duplex MAPP comprises an IL-2 MOD or IL-2 variant MOD polypeptide and a PD-L1 MOD polypeptide. In some case MODs, which may be the same or different, are present in a MAPP or duplex MAPP in tandem. When MODs are presented in tandem, their sequences are immediately adjacent to each other on a single polypeptide, either without any intervening sequence or separated by only a linker polypeptide (e.g., no MHC sequences or epitope sequences intervene). The MOD polypeptide may comprise all or part of the extracellular portion of a full-length MOD. Thus, for example, the MOD can in some cases exclude one or more of a signal peptide, a transmembrane domain, and an intracellular domain normally found in a naturally-occurring MOD. Unless stated otherwise, a MOD present in a MAPP or duplex MAPP does not comprise the signal peptide, intracellular domain, or a sufficient portion of the transmembrane domain to anchor a substantial amount (e.g., more than 5% or 10%) of a MAPP or duplex MAPP into a mammalian cell membrane.

In some cases, a MOD suitable for inclusion in a MAPP comprises all or a portion of (e.g., an extracellular portion of) the aa sequence of a naturally-occurring MOD. In other instances, a MOD suitable for inclusion in a MAPP is a variant MOD that comprises at least one aa substitution compared to the aa sequence of a naturally-occurring MOD. In some instances, a variant MOD exhibits a binding affinity for a co-MOD that is lower than the affinity of a corresponding naturally-occurring MOD (e.g., a MOD not comprising the aa substitution(s) present in the variant) for the co-MOD. Suitable variations in MOD polypeptide sequence that alter affinity may be identified by scanning (making aa substitution e.g., alanine substitutions or “alanine scanning” or charged residue changes) along the length of a peptide and testing its affinity. Once key aa positions altering affinity are identified those positions can be subject to a vertical scan in which the effect of one or more aa substitutions other than alanine are tested.

a. MODs and Variant MODs with Reduced Affinity

A MOD can comprise a wild-type amino acid sequence, or can comprise one or more amino acid substitutions, insertions, and/or deletions relative to a wild-type amino acid sequence. The immunomodulatory polypeptide can comprise only the extracellular portion of a full-length immunomodulatory polypeptide. Alternatively, a MOD can comprise all or a portion of (e.g., an extracellular portion of) the amino acid sequence of a naturally-occurring MOD polypeptide.

Variant MODs comprise at least one amino acid substitution, addition and/or deletion as compared to the amino acid sequence of a naturally-occurring immunomodulatory polypeptide. As noted above, in some instances a variant MOD exhibits a binding affinity for a co-MOD that is lower than the affinity of a corresponding naturally-occurring MOD (e.g., an immunomodulatory polypeptide not comprising the amino acid substitution(s) present in the variant) for the co-MOD.

MOD polypeptides and variants, including reduced affinity variants, of proteins such as PD-L1, CD80, CD86, 4-1BBL and IL-2 are described in the published literature, e.g., published PCT application WO2020132138A1, the disclosure of which as it pertains to immunomodulatory polypeptides and specific variant immunomodulatory polypeptides of PD-L1, CD80, CD86, 4-1BBL, IL-2 are expressly incorporated herein by reference, including specifically paragraphs [00260]-[00455] of WO2020132138A1.

Suitable immunomodulatory domains that exhibit reduced affinity for a co-immunomodulatory domain can have from 1 aa to 20 aa differences from a wild-type immunomodulatory domain. For example, in some cases, a variant MOD present in a MAPP may include a single aa substitution compared to a corresponding reference (e.g., wild-type) MOD. A variant MOD present in a MAPP may include 2 aa substitutions compared to a corresponding reference (e.g., wild-type) MOD. A variant MOD present in a MAPP may include 3 or 4 aa substitutions compared to a corresponding reference (e.g., wild-type) MOD. A variant MOD present in a MAPP may include 5 or 6 aa substitutions compared to a corresponding reference (e.g., wild-type) MOD. A variant MOD present in a MAPP may include 7, 8, 9 or 10 aa substitutions compared to a corresponding reference (e.g., wild-type) MOD. A variant MOD present in a MAPP may include 11-15 or 15-20 aa substitutions compared to a corresponding reference (e.g., wild-type) MOD.

As discussed above, a variant MOD suitable for inclusion in a MAPP may exhibit reduced affinity for a cognate co-MOD, compared to the affinity of a corresponding wild-type MOD for the cognate co-MOD. In some cases, a variant MOD present in a MAPP has a binding affinity for a cognate co-MOD that is from 100 nM to 100 μM. For example, in some cases, a variant MOD present in a MAPP has a binding affinity for a cognate co-MOD that is from about 100 nM to about 200 nM, from about 200 nM to about 300 nM, from about 300 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about 10 μM to about 20 μM, from about 20 μM to about 30 μM, from about 30 μM to about 50 μM, from about 50 μM to about 75 μM, or from about 75 μM to about 100 μM.

Binding affinity between a MOD polypeptide sequence and its cognate co-MOD polypeptide can be determined by bio-layer interferometry (BLI) using the purified MOD polypeptide sequence and purified cognate co-MOD polypeptide, following the procedure set forth in published PCT Application WO 2020/132138 A1.

b. IL-2 and its Variants

As one non-limiting example, a MOD or variant MOD present in a MAPP is an IL-2 or variant IL-2 polypeptide. In some cases, a variant MOD present in a MAPP is a variant IL-2 polypeptide. Wild-type IL-2 binds to an IL-2 receptor (IL-2R). A wild-type IL-2 aa sequence can be as follows:

APTSSSTKKT QLQLEHLLLD LQMILNGINN YKNPKLTRML TFKFYMPKKA TELKHLQCLE
EELKPLEEVL NLAQSKNFHL RPRDLISNIN VIVLELKGSE TTFMCEYADE TATIVEFLNR
WITFCQSIIS TLT (aa 21-153 of UniProt P60568, SEQ ID NO: 166).

Wild-type IL2 binds to an IL2 receptor (IL2R) on the surface of a cell. An IL2 receptor is in some cases a heterotrimeric polypeptide comprising an alpha chain (IL-2Rα; also referred to as CD25), a beta chain (IL-2Rβ; also referred to as CD122) and a gamma chain (IL-2Rγ; also referred to as CD132). Amino acid sequences of human IL-2Rα, IL2Rβ, and IL-2Rγ are provided in the accompanying sequence listing as SEQ ID NO:167, SEQ ID NO:168 and SEQ ID NO:169, respectively, and are also provided in, for example, U.S. Patent Pub. No. 20200407416.

In some cases, a variant IL-2 polypeptide exhibits reduced binding affinity to one or more of the IL-2Rα, IL2Rβ, and/or IL-2Rγ chains of human IL-2R, compared to the binding affinity of an IL-2 polypeptide comprising the aa sequence set forth in SEQ ID NO:166. For example, in some cases, a variant IL-2 polypeptide binds to one or more of the IL-2Rα, IL2Rβ, and/or IL-2Rγ chains of human IL-2R with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of an IL-2 polypeptide comprising the aa sequence set forth in SEQ ID NO:166 for the α, β, and/or γ chains of IL-2R (e.g., an IL-2R comprising polypeptides comprising the aa sequence set forth in SEQ ID NOs:167-169), when assayed under the same conditions.

For example, IL-2 variants with a substitution of phenylalanine at position 42 (e.g., with an alanine), exhibit substantially reduced binding to the IL-2Rα chain, in which case the variant may reduce the activation of Tregs. IL-2 variants with a substitution of histidine at position 16 (e.g., with an alanine) exhibit reduced binding to the IL2Rβ chain, thereby reducing the likelihood of a MAPP binding to non-target T cells by virtue of off-target binding of the IL-2 MOD. Some IL-2 variants, e.g., those with substitutions of the F42 and H16 amino acids, exhibit substantially reduced binding to the IL-2Rα chain and also reduced binding to the IL2Rβ chain. See, e.g., Quayle, et al., Clin Cancer Res; 26(8) Apr. 15, 2020.

In some cases, a variant IL-2 polypeptide has a single aa substitution compared to the IL-2 aa sequence set forth in SEQ ID NO:166. In some cases, a variant IL-2 polypeptide has from 2 to 10 aa substitutions compared to the IL-2 aa sequence set forth in SEQ ID NO:166. In some cases, a variant IL-2 polypeptide has 2, 3, 4, 5, 6, 7, 8, 9 or 10 aa substitutions compared to the IL-2 aa sequence set forth in SEQ ID NO:166. In some cases, a variant IL-2 polypeptide has 2 or 3 aa substitutions compared to the IL-2 aa sequence set forth in SEQ ID NO:166.

Suitable variant IL-2 polypeptide sequences include polypeptide sequences comprising an aa sequence having at least 80% (e.g., at least 85%, at least 90%, at least 95%, at least 98%, at least 99%, or 100%) aa sequence identity to at least 80 (e.g., 90, 100, 110, 120, 130 or 133) contiguous aas of SEQ ID NO:166. Potential amino acids where substitutions may be introduced include one or more of the following positions:
(i) position 15, where the aa is other than E (e.g., A);
(ii) position 16, where the aa is other than H (e.g., A, T, N, C, Q, M, V or W);
(iii) position 20 is an aa other than D (e.g., A);
(iv) position 42, where the aa is other than F (e.g., A, M, P, S, T, Y, V or H);
(v) position 45, where the aa is other than Y (e.g., A);
(vi) position 88, where the aa is other than N (e.g., A or R);
(vii) position 126, where the aa is other than Q (e.g., A);

Combinations of the above substitutions include (H16X, F42X), (D20X, F42X), (E15X, D20X, F42X), (an H16X, D20X, F42X), (H16X, F42X, R88X), (H16X, F42X, Q126X), (D20X, F42X, Q126X), (D20X, F42X, and Y4X), (H16X, D20X, F42X, and Y45X), (D20X, F42X, Y45X, Q126X), (H16X, D20X, F42X, Y45X, Q126X), where X is the substituted aa, optionally chosen from the following: positions 15, 20, 45, 126—A; position 16—A or T, or also N, C, Q, M, V or W; position 42—A, or also M, P, S, T, Y, V or H; position 88—A or R.

IL-2 variants include polypeptides having at least 90% or at least 95% (e.g., at least 95%, 98%, or 99%) aa sequence identity to at least 80 (e.g., at least 90, 100, 110, 120, or 130) contiguous aas of SEQ ID NO:166, wherein the aa at position 16 is an aa other than H. In one case, the position of H16 is substituted by Asn, Cys, Gln, Met, Val, or Trp. In one case, the position of H16 is substituted by Ala. In another case, the position of H16 is substituted by Thr. Additionally, or alternatively, IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:166, wherein the aa at position 42 is an aa other than F. In one case, the position of F42 is substituted by Met, Pro, Ser, Thr, Trp, Tyr, Val, or His. In one case, the position of F42 is substituted by Ala.

IL-2 variants include polypeptides comprising an aa sequence comprising all or part of human IL-2 polypeptide having a substitution at position H16 and/or F42 (e.g., H16A and/or F42A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:166, wherein the aa at position 16 is an aa other than H and the aa at position 42 is other than F. In one case, the position of H16 is substituted by Ala or Thr and the position of F42 is substituted by Ala or Thr. In one case, the position of H16 is substituted by Ala and the position of F42 is substituted by Ala (an H16A and F42A variant). In a second case, the position of H16 is substituted by Thr and the position of F42 is substituted by Ala (an H16T and F42A variant). In a third case, the position of H16 is substituted by Ala and the position of F42 is substituted by Thr (an H16A and F42T variant). In a fourth case, the position of H16 is substituted by Thr and the position of F42 is substituted Thr Ala (an H16T and F42T variant). As noted above, such variants will exhibit reduced binding to both the human IL-2Rα chain and IL2Rβ chain.

In any of the wild-type or variant IL-2 sequences provided herein, the cysteine at position 125 may be substituted with an aa other than cystine, such as alanine (a C125A substitution). In addition to any stability provided by the substitution, it may be employed where, for example, an additional peptide is to be conjugated to a cysteine residue elsewhere in a MAPP, thereby avoiding competition from the C125 of the IL-2 MOD sequence.

c. Fas Ligand (FasL) and its Variants

In some cases, a wild-type and/or a variant Fas Ligand (FasL) polypeptide sequence is present as a MOD in a MAPP. FasL is a homomeric type-II transmembrane protein in the tumor necrosis factor (TNF) family. FasL signals by trimerization of the Fas receptor in a target cell, which forms a death-inducing complex leading to apoptosis of the target cell. Soluble FasL results from matrix metalloproteinase-7 (MMP-7) cleavage of membrane-bound FasL at a conserved site. In an embodiment, a wt. Homo sapiens FasL protein has the sequence MQQPFNYPYP QIYWVDSSAS SPWAPPGTVL PCPTSVPRRP GQRRPPPPPP PPPLPPPPPP PPLPPLPLPP LKKRGNHSTG LCLLVMFFMV LVALVGLGLG MFQLFHLQKE LAELRESTSQ MHTASSLEKQ IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMM SYCTTGQMWA RSSYLGAVFN LTSADHLYVN VSELSLVNFE ESQTFFGLYK L, (SEQ ID NO:170), NCBI Ref. Seq. NP_000630.1, UniProtKB—P48023 where residues 1-80 are cytoplasmic, 81-102 are the transmembrane domain and aas 103-281 are extracellular (ectodomain). In some cases, a FasL polypeptide suitable for inclusion in a MAPP comprises an aa sequence having at least 60%, at least 70%, at least 80%, at least 90%, at least 95%, at least 98%, at least 99%, or 100%, aa sequence identity to a contiguous stretch of at least 150 aas, at least 170, at least 180 aas, at least 200 aas, at least 225 aas, at least 250 aas, at least 270 aas, at least 280, or all aas of the aa sequence of SEQ ID NO:170).

A Fas recptor can have the sequence MLGIWTLLPL VLTSVARLSS KSVNAQVTDI NSKGLELRKT VTTVETQNLE GLHHDGQFCH KPCPPGERKA RDCTVNGDEP DCVPCQEGKE YTDKAHFSSK CRRCRLCDEG HGLEVEINCT RTQNTKCRCK PNFFCNSTVC EHCDPCTKCE HGIIKECTLT SNTKCKEEGS RSNLGWLCLL LLPIPLIVWV KRKEVQKTCR KHRKENQGSH ESPTLNPETV AINLSDVDLS KYITTIAGVM TLSQVKGFVR KNGVNEAKID EIKNDNVQDT AEQKVQLLRN WHQLHGKKEA YDTLIKDLKK ANLCTLAEKI QTIILKDITS DSENSNFRNE IQSLV, (SEQ ID NO:171) NCBI Reference Sequence: NP_000034.1, UniProtKB—P25445, where aas 26-173 form the ectodomain (extracellular domain), aas 174-190 form the transmembrane domain, and 191-335 the cytoplasmic domain. The ectodomain may be used to determine binding affinity with FasL.

A FasL polypeptide suitable for inclusion in a MAPP may comprise an aa sequence having at least 60%, at least 70%, at least 80%, at least 90%, at least 95%, at least 98%, at least 99%, or 100%, aa sequence identity to the following aa sequence: IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMMSYCTTGQMWA RSSYLGAVFN LTSADHLYVN VSELSLVNFE ESQTFFGLYK (SEQ ID NO:172); and has a length of about 150 aas, including 148, 149, 150, 151, or 152 aas.

In some cases, a FasL polypeptide suitable for inclusion in a MAPP comprises an aa sequence having at least 60%, at least 70%, at least 80%, at least 90%, at least 95%, at least 98%, at least 99%, or 100%, aa sequence identity to a contiguous stretch of at least 50 aas, at least 160 aas, at least 170, at least 175, or all of the aas the following aa sequence: QLFHLQKE LAELRESTSQ MHTASSLEKQ IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMM SYCTTGQMWA RSSYLGAVFN LTSADHLYVN VSELSLVNFE ESQTFFGLYK L (SEQ ID NO:173). Suitable variant FasL polypeptide sequences include polypeptide sequences with at least 80%, at least 85%, at least 90%, at least 95%, at least 98%, or at least 99% aa sequence identity to at least 140 contiguous aa (e.g., at least 150, at least 160, at least 170, or at least 175 contiguous aa) of SEQ ID NO:173) (e.g., which have at least one aa substitution, deletion or insertion).

In some cases, a variant FasL polypeptide (e.g., comprising a variant of SEQ ID NO:172 or SEQ ID NO:173) exhibits reduced binding affinity to a mature Fas receptor sequence (e.g., a FasL receptor comprising all or part of the polypeptide set forth in SEQ ID NO:171, such as its ectodomain), compared to the binding affinity of an FasL polypeptide comprising the aa sequence set forth in SEQ ID NO:172 or SEQ ID NO:173. For example, in some cases, a variant FasL polypeptide (e.g., comprising a variant of SEQ ID NO:173) binds an Fas receptor (e.g., comprising all or part of the polypeptides set forth in SEQ ID NO: 171, such as its ectodomains), with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of an FasL polypeptide comprising the aa sequence set forth in SEQ ID NO:170 or 173.

d. PD-L1 and its Variants

As one non-limiting example, a MOD or variant MOD present in a MAPP is a PD-L1 or variant PD-L1 polypeptide. Wild-type PD-L1 binds to PD1.

A wild-type human PD-L1 polypeptide can comprise the following aa sequence: MRIFAVFIFM TYWHLLNAFT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKICLT LSPST (SEQ ID NO:174); where aas 1-18 form the signal sequence, aas 19-127 form the Ig-like V-type or IgV domain, and 133-225 for the Ig-like C2 type domain.

A wild-type human PD-L1 ectodomain aa sequence can comprise the following aa sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:175); where aas 1-109 form the Ig-like V-type or “IgV” domain, and aas 115-207 for the Ig-like C2 type domain.

A wild-type human PD-L1 ectodomain aa sequence can also comprise the following aa sequence:FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELP LAHPPNER LNVSIKI (SEQID NO:176); where aas 1-109 form the Ig-like V-type or “IgV” domain, and aas 115-207 for the Ig-like C2 type domain. See e.g., NCBI Accession and version 3BIK_A, which includes an N-terminal alanine as its first aa.

A wild-type PD-L1 IgV domain, suitable for use as a MOD may comprise aa 18 and aas IgV aas 19-127 of SEQ ID NO:174, and a carboxyl terminal stabilization sequences, such as for instance the last seven aas (bolded and italicized) of the sequence: A FTVTVPKDLY VVEYGSNMTI ECKFPVEKQL DLAALIVYWE MEDKNIIQFV HGEEDLKTQH SSYRQRARLL KDQLSLGNAAI [symbol] TDVKLQD AGVYRCMISY GGADYKRITV KVNAPY [symbol] (SEQ ID NO:177). Where the carboxyl stabilizing sequence comprises a histidine (e.g., a histidine approximately 5 residues to the C-terminal side of the Tyr (Y) appearing as aa 117 of SEQ ID NO:177) to about aa 122, the histidine may form a stabilizing electrostatic bond with the backbone amide at aas 82 and 83 (bolded and italicized in SEQ ID NO:174 (Q107 and L106 of SEQ ID NO:174). As an alternative, a stabilizing disulfide bond may be formed by substituting one of aas 82 or 83) (Q107 and L106 of SEQ ID NO:174) and one of aa residues 121, 122, or 123 (equivalent to aa positions 139-141 of SEQ ID NO:174).

A wild-type PD-1 polypeptide can comprise the following aa sequence: PGWFLDSPDR PWNPPTFSPA LLVVTEGDNA TFTCSFSNTS ESFVLNWYRM SPSNQTDKLA AFPEDRSQPG QDCRFRVTQL PNGRDFHMSV VRARRNDSGT YLCGAISLAP KAQIKESLRA ELRVTERRAE VPTAHPSPSP RPAGQFQTLV VGVVGGLLGS LVLLVWVLAV ICSRAARGTI GARRTGQPLK EDPSAVPVFS VDYGELDFQW REKTPEPPVP CVPEQTEYAT IVFPSGMGTS SPARRGSADG PRSAQPLRPE DGHCSWPL (SEQ ID NO:178).

In some cases, a variant PD-L1 polypeptide (e.g., a variant of SEQ ID NO:175 or PD-L1's IgV domain) exhibits reduced binding affinity to PD-1 (e.g., a PD-1 polypeptide comprising the aa sequence set forth in SEQ ID NO:178), compared to the binding affinity of a PD-L1 polypeptide comprising the aa sequence set forth in SEQ ID NO:174 or SEQ ID NO:175. For example, in some cases, a variant PD-L1 polypeptide binds PD-1 (e.g., a PD-1 polypeptide comprising the aa sequence set forth in SEQ ID NO:178) with a binding affinity that is at least 10% less, at least 20% less, at least 30% less, at least 40% less, at least 50% less, at least 60% less, at least 70% less, at least 80% less, at least 90% less, at least 95% less, or more than 95% less than the binding affinity of a PD-L1 polypeptide comprising the aa sequence set forth in SEQ ID NO:174 or SEQ ID NO:175.

e. TGF-β and its Variants

In some cases, at least one of the one or more MOD polypeptides present in a MAPP comprises the aa sequence of a wild-type TGF-β polypeptide. In other instances, at least one of the one or more MOD polypeptides present in a MAPP is a variant TGF-β polypeptide. Wild-type TGF-β and variant TGF-β polypeptides bind to TGF receptor.

As noted above, in some cases, the MOD polypeptide present in a MAPP is a TGF-β polypeptide. The aa sequences of TGF-β polypeptides are known in the art. In some cases, the MOD polypeptide present in a MAPP is a TGF-β1 polypeptide. MOD polypeptide present in a MAPP is a TGF-β2 polypeptide. MOD polypeptide present in a MAPP is a TGF-β3 polypeptide. A suitable TGF-β polypeptide can comprise an aa sequence having at least 60%, at least 70%, at least 80%, at least 90%, at least 95%, at least 98%, at least 99%, or 100%, aa sequence identity to the mature form of a human TGF-β1 polypeptide, a human TGF-β2 polypeptide, or a human TGF-β3 polypeptide. A suitable TGF-β polypeptide can have a length of from about 100 aas to about 125 aas; for example, a suitable TGF-β polypeptide can have a length of from about 100 aas to about 105 aas, from about 105 aas to about 110 aas, from about 110 aas to about 115 aas, from about 115 aas to about 120 aas, or from about 120 aas to about 125 aas.

A suitable TGF-β1 polypeptide can comprise an aa sequence having at least 60%, at least 70%, at least 80%, at least 90%, at least 95%, at least 98%, at least 99%, or 100%, aa sequence identity to the following TGF-β1 aa sequence: AL DTNYCFSSTE KNCCVRQLYI DFRKDLGWKW IHEPKGYHAN FCLGPCPYIW SLDTQYSKVL ALYNQHNPGA SAAPCCVPQA LEPLPIVYYV GRKPKVEQLS NMIVRSCKCS (SEQ ID NO:179); or the foregoing sequence comprising a C77S substitution; where the TGF-β1 polypeptide has a length of about 112 aas.

A suitable TGF-β2 polypeptide can comprise an aa sequence having at least 60%, at least 70%, at least 80%, at least 90%, at least 95%, at least 98%, at least 99%, or 100%, aa sequence identity to the following TGF-β2 aa sequence: ALDAAYCF RNVQDNCCLR PLYIDFKRDL GWKWIHEPKG YNANFCAGAC PYLWSSDTQH SRVLSLYNTI NPEASASPCC VSQDLEPLTI LYYIGKTPKI EQLSNMIVKS CKCS (SEQ ID NO:180); or the foregoing sequence comprising a C77S substitution, where the TGF-β2 polypeptide has a length of about 112 aas.

A suitable TGF-β3 polypeptide can comprise an aa sequence having at least 60%, at least 70%, at least 80%, at least 90%, at least 95%, at least 98%, at least 99%, or 100% aa sequence identity to the following TGF-β3 aa sequence: ALDTNYCFRN LEENCCVRPL YIDFRQDLGW KWVHEPKGYY ANFCSGPCPY LRSADTTHST VLGLYNTLNP EASASPCCVP QDLEPLTILY YVGRTPKVEQ LSNMVVKSCK CS (SEQ ID NO:181); or the foregoing sequence comprising a C77S substitution, where the TGF-β3 polypeptide has a length of about 112 aas.

f. CD80 and its Variants

In some cases, a wild-type and/or a variant CD80 MOD polypeptide sequence is present as a MOD in a MAPP of the present disclosure. Wild-type CD80 and variant CD80 MOD polypeptides bind to CD28 which acts as their receptor.

A wild-type aa sequence of the ectodomain of human CD80 can be as follows: VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:182). See NCBI Reference Sequence: NP_005182.1. The aa sequence of the IgV domain of a wild-type human CD80 can be as follows: VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSV, (SEQ ID NO:183), which is aas 1-104 of SEQ ID NO:182.

A wild-type CD28 aa sequence can be as follows: MLRLLLALNL FPSIQVTGNK ILVKQSPMLV AYDNAVNLSC KYSYNLFSRE FRASLHKGLD SAVEVCVVYG NYSQQLQVYS KTGFNCDGKL GNESVTFYLQ NLYVNQTDIY FCKIEVMYPP PYLDNEKSNG TIIHVKGKHL CPSPLFPGPS KPFWVLVVVG GVLACYSLLV TVAFIIFWVR SKRSRLLHSD YMNMTPRRPG PTRKHYQPYA PPRDFAAYRS (SEQ ID NO:184).

A wild-type CD28 aa sequence can be as follows: MLRLLLALNL FPSIQVTGNK ILVKQSPMLV AYDNAVNLSW KHLCPSPLFP GPSKPFWVLV VVGGVLACYS LLVTVAFIIF WVRSKRSRLL HSDYMNMTPR RPGPTRKHYQ PYAPPRDFAA YRS (SEQ ID NO:185)

A wild-type CD28 aa sequence can be as follows: MLRLLLALNL FPSIQVTGKH LCPSPLFPGP SKPFWVLVVV GGVLACYSLL VTVAFIIFWV RSKRSRLLHS DYMNMTPRRP GPTRKHYQPY APPRDFAAYR S (SEQ ID NO:186).

Variant CD80 polypeptides suitable as a MOD in a MAPP of the present discosure may exhibit reduced binding affinity to CD28, compared to the binding affinity of a CD80 polypeptide comprising the aa sequence set forth in SEQ ID NO:182, or the IgV domain sequence SEQ ID NO:183, for CD28. A variant CD80 MOD polypeptide may bind CD28 with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of a CD80 polypeptide comprising the aa sequence set forth in SEQ ID NO:182 for CD28 (e.g., a CD28 polypeptide comprising the aa sequence set forth in one of SEQ ID NO:184, SEQ ID NO:185, or SEQ ID NO:186).

CD80 ectodomain variants suitable for use as a MOD in a MAPP include those polypeptides with at least one aa substitution having at least 90%, at least 95%, at least 98%, or at least 99% aa sequence identity to SEQ ID NO:182, or the IgV domain sequence SEQ ID NO:183.

CD80 ectodomain variants suitable for use as a MOD in a MAPP include those polypeptides having at least 90%, at least 95%, at least 98%, or at least 99% aa sequence identity to SEQ ID NO:182, or the IgV domain sequence SEQ ID NO:183, and which have at least one (e.g., at least two, or at least three) aa substitutions.

CD80 ectodomain variants suitable for use as a MOD in a MAPP include those polypeptides having at least 90% (e.g., at least 95%, 98%, or 99%) aa sequence identity to at least 80 (e.g., at least 90, 100, 104, 120, 150, 180, 200, or 208) contiguous aas of SEQ ID NO:182, or least 80 (e.g., at least 90, 100, or 104) contiguous aas of the IgV domain sequence of SEQ ID NO:183.

g. CD86 and its Variants

In some cases, a wild-type and/or a variant CD86 MOD polypeptide sequence is present as a MOD in a MAPP of the present disclosure. Wild-type CD86 and variant CD86 MOD polypeptides bind to CD28 which acts as their receptor as discussed for CD80 MOD polypeptides.

A wild-type aa sequence of the ectodomain of human CD86 can be as follows:

(SEQ ID NO: 187)
APLKIQAYFNETADLPCQFANSQNQSLSELVVFWQDQENLVLNEVYLGK
EKFDSVHSKYMNRTSFDSDSWTLRLHNLQIKDKGLYQCIIHHKKPTGMI
RIHQMNSELSVLANFSQPEIVPISNITENVYINLTCSSIHGYPEPKKMS
VLLRTKNSTIEYDGIMQKSQDNVTELYDVSISLSVSFPDVTSNMTIFCI
LETDKTRLLSSPFSIELEDPQPPPDHIP.

The aa sequence of the IgV domain of a wild-type human CD86 can be as follows:

(SEQ ID NO: 188)
APLKIQAYFNETADLPCQFANSQNQSLSELVVFWQDQENLVLNEVYLGK
EKFDSVHSKYMNRTSFDSDSWTLRLHNLQIKDKGLYQCIIHHKKPTGMI
RIHQMNSELSVL.

Variant CD86 polypeptides suitable as a MOD in a MAPP may exhibits reduced binding affinity to CD28, compared to the binding affinity of a CD86 polypeptide comprising the aa sequence set forth in SEQ ID NO:187 or SEQ ID NO:188 for CD28. A variant CD86 MOD polypeptide may bind CD28 with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of a CD86 polypeptide comprising the aa sequence set forth in SEQ ID NO:187 or SEQ ID NO:188 for CD28 (e.g., a CD28 polypeptide comprising the aa sequence set forth in one of SEQ ID NO:184, SEQ ID NO:185, or SEQ ID NO:186).

CD86 ectodomain variants suitable for use as a MOD in a MAPP include those polypeptides with at least one aa substitution having at least 90%, at least 95%, at least 98%, or at least 99% aa sequence identity to SEQ ID NO:187, or the IgV domain sequence SEQ ID NO:188.

CD86 ectodomain variants suitable for use as a MOD in a MAPP include those polypeptides having at least 90%, at least 95%, at least 98%, or at least 99% aa sequence identity to SEQ ID NO:187, or the IgV domain sequence SEQ ID NO:188, and which have at least one (e.g., at least two, or at least three) aa substitutions.

CD86 ectodomain variants suitable for use as a MOD in a MAPP include those polypeptides with at least one aa substitution having at least 90%, at least 95%, at least 98%, or at least 99% aa sequence identity to at least 80 (e.g., at least 90, 100, or 109, 120, 150, 180, 200, or 224) contiguous aas of SEQ ID NO:187, or at least 80 (e.g., at least 90, 100, 104) contiguous aas of the IgV domain sequence SEQ ID NO:188.

h. 4-1BBL and its Variants

In some cases, a wild-type and/or a variant 4-1BBL MOD polypeptide sequence is present as a MOD in a MAPP of the present disclosure. Wild-type 4-1BBL binds to 4-1BB (CD137). A wild-type 4-1BBL aa sequence can be as follows: MEYASDASLD PEAPWPPAPR ARACRVLPWA LVAGLLLLLL LAAACAVFLA CPWAVSGARA SPGSAASPRL REGPELSPDD PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:189). NCBI Reference Sequence: NP_003802.1, where aas 29-49 are a transmembrane region.

In some cases, a variant 4-1BBL polypeptide is a variant of the tumor necrosis factor (TNF) homology domain (THD) of human 4-1BBL. A wild-type aa sequence of the THD of human 4-1BBL can comprise, e.g., one of SEQ ID NOs:190-192, as follows:

(SEQ ID NO: 190)
PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE;
(SEQ ID NO: 191)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE;
and
(SEQ ID NO: 192)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPA.

A wild-type 4-1BB aa sequence can be as follows: MGNSCYNIVA TLLLVLNFER TRSLQDPCSN CPAGTFCDNN RNQICSPCPP NSFSSAGGQR TCDICRQCKG VFRTRKECSS TSNAECDCTP GFHCLGAGCS MCEQDCKQGQ ELTKKGCKDC CFGTFNDQKR GICRPWTNCS LDGKSVLVNG TKERDVVCGP SPADLSPGAS SVTPPAPARE PGHSPQIISF FLALTSTALL FLLFFLTLRF SVVKRGRKKL LYIFKQPFMR PVQTTQEEDG CSCRFPEEEE GGCEL (SEQ ID NO:193).

A variant 4-1BBL polypeptide exhibits reduced binding affinity to 4-1BB, compared to the binding affinity of a 4-1BBL polypeptide comprising the aa sequence set forth in one of SEQ ID NOs:190-192. For example, a variant 4-1BBL polypeptide may bind 4-1BB with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of a 4-1BBL polypeptide comprising the aa sequence set forth in one of SEQ ID NOs:190-192 for a 4-1BB polypeptide (e.g., a 4-1BB polypeptide comprising the aa sequence set forth in SEQ ID NO:193), when assayed under the same conditions.

4-1BBL variants suitable for use as a MOD in a MAPP include those polypeptides with at least one aa substitution having at least 90%, at least 95%, at least 98%, or at least 99% aa sequence identity to one of SEQ ID NOs:190, 191 or 192.

4-1BBL variants suitable for inclusion in a MAPP include those with at least one aa substitution (e.g., two, three, or four substitutions) include those having at least 90%, at least 95%, at least 98%, or at least 99% aa sequence identity to at least 140 (e.g., at least 160, 175, 180, or 181) contiguous aas of SEQ ID NO:190.

6. Linkers

As noted above, a MAPP can include a linker sequence (aa, peptide, or polypeptide linker sequence) or “linker” interposed between any two elements of a MAPP, e.g., an epitope and an MHC polypeptide; between an MHC polypeptide and an Ig Fc polypeptide; between a first MHC polypeptide and a second MHC polypeptide; etc. Although termed “linkers,” sequences employed for linkers may also be placed at the N- and/or C-terminus of a MAPP polypeptide to, for example, stabilize the MAPP polypeptide or protect it from proteolytic degradation.

Suitable polypeptide linkers (also referred to as “spacers”) are known in the art and can be readily selected and can be of any of a number of suitable lengths, e.g., from 2 to 50 aa in length, e.g., from 2 aa to 10 aa, from 10aa to 20 aa, 20 aa to 30 aa, from 30 aa to 40aa, from 40aa to 50aa, or longer than 50aa. In embodiments, a suitable linker can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 aa in length. Linkers can be generally classified into three groups, i.e., flexible, rigid and cleavable. See, e.g., Chen et al. (2013) Adv. Drug Deliv. Rev. 65:1357; and Klein et al. (2014) Protein Engineering, Design & Selection 27:325. Unless stated otherwise, the linkers employed in the MAPPs of this disclosure are not the cleavable linkers generally known in the art.

Polypeptide linkers in the MAPP may include, for example, polypeptides that comprise, consist essentially of, or consists of: i) Gly and Ser; ii) Ala and Ser; iii) Gly, Ala, and Ser; iv) Gly, Ser, and Cys (e.g., a single Cys residue); v) Ala, Ser, and Cys (e.g., a single Cys residue); and vi) Gly, Ala, Ser, and Cys (e.g., a single Cys residue). Exemplary linkers may comprise glycine polymers, glycine-serine polymers, glycine-alanine polymers; alanine-serine polymers (including, for example polymers comprising the sequences GSGGS (SEQ ID NO:194) or GGGS (SEQ ID NO:195), any of which may be repeated from 1 to 10 times (e.g., repeated 1, 2, 3, 4, 5, 6, 7, 8, 9, or 10 times); and other flexible linkers known in the art. Glycine and glycine-serine polymers can both be used; both Gly and Ser are relatively unstructured and therefore can serve as a neutral tether between components. Glycine polymers access significantly more phi-psi space than even alanine, and are much less restricted than residues with longer side chains (see Scheraga, Rev. Computational Chem. 11173-142 (1992)). Exemplary linkers may also comprise an aa sequence comprising, but not limited to, GGSG (SEQ ID NO:196), GGSGG (SEQ ID NO:197), GSGSG (SEQ ID NO:198), GSGGG (SEQ ID NO:199), GGGSG (SEQ ID NO:200), GSSSG (SEQ ID NO:201), any which may be repeated from 1 to 10 times (e.g., repeated 1, 2, 3, 4, 5, 6, 7, 8, 9, or 10 times), or combinations thereof, and the like. Linkers can also comprise the sequence Gly(Ser)₄ (SEQ ID NO:202) or (Gly)₄Ser (SEQ ID NO:203), either of which may be repeated from 1 to 10 times (e.g., 1, 2, 3, 4, 5, 6, 7, 8, 9, or 10 times). In one embodiment the linker comprises the aa sequence AAAGG (SEQ ID NO:204), which may be repeated from 1 to 10 times.

Rigid polypeptide linkers comprise a sequence of amino acids that effectively separates protein domains by maintaining a substantially fixed distance/spatial separation between the domains, thereby reducing or substantially eliminating unfavorable interactions between such domains. Rigid polypeptide linkers thus may be employed where it is desired to minimize the interaction between the domains of the MAPP. Rigid peptide linkers include peptide linkers rich in proline, and peptide linkers having an inflexible helical structure, such as an α-helical structure. Examples of rigid peptide linkers include, e.g., (EAAAK)n (SEQ ID NO:205), A(EAAAK)nA (SEQ ID NO:206), A(EAAAK)nALEA(EAAAK)nA (SEQ ID NO:207), (Lys-Pro)n, (Glu-Pro)n, (Thr-Pro-Arg)n, and (Ala-Pro)n where n is an integer from 1 to 20 (e.g., n is 1, 2, 3, 4, 5, 6, 7, 8, 9, 10, 11, 12, 13, 14, 15, 16, 17, 18, 19, or 20). Non-limiting examples of suitable rigid linkers comprising EAAAK (SEQ ID NO:208) include EAAAK (SEQ ID NO:208), (EAAAK)₂ (SEQ ID NO:209), (EAAAK)₃ (SEQ ID NO:210), A(EAAAK)₄ALEA(EAAAK)₄A (SEQ ID NO:211), and AEAAAKEAAAKA (SEQ ID NO:212). Non-limiting examples of suitable rigid linkers comprising (AP)n include PAPAP (SEQ ID NO:213; also referred to herein as “(AP)2”); APAPAPAP (SEQ ID NO:214; also referred to herein as “(AP)4”); APAPAPAPAPAP (SEQ ID NO:215; also referred to herein as “(AP)6”); APAPAPAPAPAPAPAP (SEQ ID NO:216; also referred to herein as “(AP)8”); and APAPAPAPAPAPAPAPAPAP (SEQ ID NO:217; also referred to herein as “(AP)10”). Non-limiting examples of suitable rigid linkers comprising (KP)n include KPKP (SEQ ID NO:218; also referred to herein as “(KP)2”); KPKPKPKP (SEQ ID NO:219; also referred to herein as “(KP)4”); KPKPKPKPKPKP (SEQ ID NO:220; also referred to herein as “(KP)6”); KPKPKPKPKPKPKPKP (SEQ ID NO:221; also referred to herein as “(KP)8”); and KPKPKPKPKPKPKPKPKPKP (SEQ ID NO:222; also referred to herein as “(KP)10”). Non-limiting examples of suitable rigid linkers comprising (EP)n include EPEP (SEQ ID NO:223; also referred to herein as “(EP)2”); EPEPEPEP (SEQ ID NO:224; also referred to herein as “(EP)4”); EPEPEPEPEPEP (SEQ ID NO:225; also referred to herein as “(EP)6”); EPEPEPEPEPEPEPEP (SEQ ID NO:226; also referred to herein as “(EP)8”); and EPEPEPEPEPEPEPEPEPEP (SEQ ID NO:227; also referred to herein as “(EP)10”).

As with other linker sequences, rigid peptide linkers may be interposed between any two elements of a MAPP. Rigid peptide linkers find particular use in joining MOD polypeptide sequences to other elements of a MAPP. In particular, rigid peptide linkers may be employed to link a MOD polypeptide sequence to the carboxy terminus of frame work polypeptides (position 3 and/or 3′) or a dimerization polypeptide (positions 5 and/or 5′) of a duplex MAPP. For example, a MOD polypeptide comprising an immunoglobulin CH2CH3 multimerization sequence may comprise a rigid peptide linker and a MOD (e.g., a wild type variant IL-2 or PD-L1 MOD) at position 3 and/or 3′ (See e.g., FIGS. 19-23). Rigid peptide linkers may also be used to link a MOD polypeptide to the N-terminus of a MAPP polypeptide (e.g., the N-terminus of a dimerization and/or framework polypeptide at positions 1 and/or 1′).

In some cases, a linker polypeptide, present in a polypeptide of a MAPP includes a cysteine residue that can form a disulfide bond with a cysteine residue present in another polypeptide of the MAPP. In some cases, for example, the linker comprises an aa sequence selected from (CGGGS), (GCGGS), (GGCGS), (GGGCS), and (GGGGC) with the rest of the linker comprised of Gly and Ser residues (e.g., GGGGS units that may be repeated from 1 to 10 times, such as 1, 2, 3, 4, 5, 6, 7, 8, 9, or 10 times). Cysteine containing linkers may also be selected from the sequences Q [symbol] GASGGGGSGGGGS (SEQ ID NO:228), G [symbol] GGSGGGGSGGGGSGGGGS (SEQ ID NO:165), and G [symbol] GGSGGGGSGGGGS (SEQ ID NO:229).

Accordingly, the linker to which an epitope is attached may be from about 5 to about 50 aas in length. The linker to which an epitope may be attached may, for example be from about 5 to about 50 aas in length and comprise more than 50% Gly and Ser residues with one cysteine residue. The linker to which an epitope may be attached may be from about 5 to about 50 aas in length and comprise more than 50% (Gly)₄S repeats with one optional cysteine residue. The linker to which an epitope may be attached may be a (Gly)₄S sequence repeated from 3 to 8 (e.g., 3 to 7) times, optionally having one aa replaced by a cysteine residue.

7. Epitopes

A variety of peptide epitopes (also referred to herein as “epitopes” or “epitope peptides”) may be present in a MAPP or higher order complexes of MAPPs (such as duplex MAPPs of the present disclosure), and presentable to a TCR on the surface of a T cell.

A peptide epitope present in a MAPP (e.g., a duplex MAPP) is designed to be specifically bound by a target T cell that has a T cell receptor (“TCR”) that is specific for the epitope and which specifically binds the peptide epitope of the MAPP. An epitope-specific T cell thus binds a peptide epitope having a reference aa sequence, but substantially does not bind an epitope that differs from the reference aa sequence. For example, an epitope-specific T cell binds a peptide epitope having a reference aa sequence, and binds an epitope that differs from the reference aa sequence, if at all, with an affinity that is less than 10⁻⁶ M, less than 10⁻⁵ M, or less than 10⁻⁴ M. An epitope-specific T cell can bind a peptide epitope for which it is specific with an affinity of at least 10⁻⁷ M, at least 10⁻⁸ M, at least 10⁻⁹ M, or at least 10⁻¹⁰ M.

a. Peptide Epitopes in MAPPs with Class II MHC Presenting Sequences and Presenting Complexes

Among the epitopes that may be bound and presented to a TCR by a MAPP with class II MHC presenting sequences or Class II MHC presenting complexes are epitope presenting peptides (or simply epitopes) derived from a variety of self and non-self antigens, depending upon the nature of the MAPP and its desired use. Self and non-self antigens include, but are not limited to, cancer antigens, allergens, and antigens derived from infectious agents (e.g., bacteria, viruses etc.) may be incorporated into MAPPs for the treatment or prophylaxis of, for example, cancers, allergies, and viral or bacterial diseases.

Epitopes associated with graft versus host disease (“GVHD”) or host versus graft disease (“HVGD”) may also be incorporated into MAPPs for the treatment of those conditions. Self antigens may be incorporated into MAPPs for the treatment or prophylaxis of, for example, cancers and autoimmune disorders.

A peptide epitope can have a length of from about 4 aas to about 25 aas (aa), e.g., the epitope can have a length of from 5 aa to 10 aa, from 10 aa to 15 aa, from 15 aa to 20 aa, or from 20 aa to 25 aa. For example, a TID epitope present in a MAPP can have a length of 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa. In some cases, a T1D peptide epitope present in a MAPP has a length of from 10 aa to 20 aa, e.g., 10 aa, 11 aa, 12 aa, 13 aa, 14 aa, 15 aa, 16 aa, 17 aa, 18 aa, 19 aa and 20 aa

(i) Cancer Epitopes

Suitable epitope-presenting peptides include, but are not limited to, epitope-presenting peptides present in a cancer-associated antigen. Cancer-associated antigens include, but are not limited to: α-folate receptor; acid phosphatase; AFP (alpha-fetoprotein polypeptide) (e.g., GenBank NP_001125); AKAP-4 (A-kinase anchoring protein-4) (e.g., GenBank NP_003877); ALK (anaplastic lymphoma kinase polypeptide) (e.g., GenBank NP_004295); androgen receptor polypeptide (e.g., GenBank NP_000035); B7H3 (B7 homolog 3 (also known as CD276) polypeptide (e.g., GenBank NP_001019907, XP_947368, XP_950958, XP 950960, XP_950962, XP 950963, XP 950965, and XP_950967); bcr-abl (bcr-abl polypeptide) (e.g., GenBank AAB60388); BORIS (BORIS polypeptide (also known as CCCTC-binding factor or CTCF)) (e.g., GenBank NP_001255969); CAIX (carbonic anhydrase IX) polypeptide; carbonic anhydrase IX (e.g., GenBank EAW58359); CD19; CD20; CD22; CD30; CD33; CD44v7/8; CEA (carcinoembryonic antigen) polypeptide (e.g., GenBank AAA51967); cyclin B1 polypeptide (e.g., GenBank CA099273); CYP1B1 (cytochrome P450 1B1 polypeptide) (e.g., GenBank AAM50512) EGFRvIII (epidermal growth factor receptor vIII polypeptide) (e.g., GenBank NP_001333870); EGP-2 (epithelial glycoprotein-2); EGP-40 (epithelial glycoprotein-40); EpCAM (epithelial cell adhesion molecule polypeptide) (e.g., GenBank NP_002345); Eph2A (EphA2 polypeptide) (e.g., GenBank NP_004422); ERG (TMPRSS2 ETS fusion polypeptide) (e.g., GenBank ACA81385); ETV6 (ETV6-AML polypeptide) (e.g., GenBank NP_001978); FAP (fibroblast activation protein) polypeptide (e.g., GenBank NP_004451); FBP (folate binding protein); fetal acetylcholine receptor; FOSL (Fos-related antigen-1) polypeptide (e.g., GenBank NP_005429); ganglioside antigen GD2; gp100 (e.g., Gp100 polypeptide) (e.g., GenBank AAC60634); h5T4 (oncofetal antigen); HER-2/neu (HER-2/neu polypeptide) (e.g., GenBank AA167147); HMW-MAA (high molecular weight melanoma associated antigen) (e.g., GenBank NP_001888); HPV E6 (human papillomavirus E6 polypeptide) (e.g., GenBank AAD33252); HPV E7 (HPV E7 polypeptide) (e.g., GenBank AHG99480); HPV16E7/11-19 (YMLDLQPET; SEQ ID NO:230); HPV16E7/11-20 (YMLDLQPETT; SEQ ID NO:231); HPV16E7/82-90 (LLMGTLGIV; SEQ ID NO:232); HPV16E7/86-93 (TLGIVCPI; SEQ ID NO:233); hTERT (hTERT polypeptide) (e.g., GenBank BAC11010); IL-13R-a2; kappa light chain; L1 cell adhesion molecule; LCK (lymphocyte cell-specific protein-tyrosine kinase) polypeptide (e.g., GenBank NP_001036236); LeY; LGMN1 (legumain polypeptide (also known as asparaginyl endopeptidase)) (e.g., GenBank NP_001008530); LMP2 (LMP2 polypeptide) (e.g., GenBank CAA47024); MAD-CT-1 (MAD-CT-1 polypeptide) (e.g., GenBank NP_005893, NP_056215); MAD-CT-2 (MAD-CT-2 polypeptide) (e.g., GenBank NP_001138574); MAGE (melanoma-associated antigen); MAGE A1 (melanoma-associated antigen A1 polypeptide) (e.g., GenBank NP_004979); MAGE-A3 (melanoma-associated antigen A3) (e.g., GenBank AAH11744); melan/MART1 (melanoma antigen recognized by T cells) polypeptide (e.g., GenBank NP_005502); melanoma antigen family A; mesothelin polypeptide (e.g., GenBank AAH09272); ML-IAP (melanoma inhibitor of apoptosis polypeptide) (e.g., GenBank AAH14475); MUC1 (MUCI polypeptide) (e.g., GenBank CAA56734); mutant p53 polypeptide; MYCN (N-myc proto-oncogene polypeptide) (e.g., GenBank NP_001280157); NA17 polypeptide; NKG2D ligands; NY-BR1 (breast cancer antigen NY-BR-1 polypeptide (also referred to as ankyrin repeat domain-containing protein 30A)) (e.g., GenBank NP_443723); NY-ESO-1 (NY-ESO-1 polypeptide) (e.g., GenBank CAA05908); OY-TES1 (testis antigen; also known as acrosin binding protein) polypeptide (e.g., GenBank NP_115878); p53 (p53 polypeptide) (e.g., GenBank BAC16799); PAGE4 (P antigen family member 4 polypeptide (e.g., GenBank NP_001305806); PAP (prostate polypeptide) (e.g., GenBank AAH16344); PAX3 (paired-box-3) polypeptide (e.g., GenBank AAI01301); PAX5 (paired box-5) polypeptide (e.g., GenBank NP_057953); PDGFO (platelet derived growth factor receptor beta) polypeptide (e.g., GenBank NP_002600); PLACI (placenta-specific protein 1 polypeptide) (e.g., GenBank AAG22596); PR1 (proteinase3 polypeptide); PSA (prostate specific antigen) polypeptide (e.g., GenBank CAD54617); PSCA (prostate stem cell antigen) polypeptide (e.g., GenBank AAH65183); PSMA (a folate hydrolase polypeptide, prostate-specific membrane antigen) (e.g., GenBank AAH25672); Ras (Ras polypeptide) (e.g., GenBank NP_001123914); regulator of G-protein signaling (RGS5) polypeptide; RhoC (Ras homolog gene family member C polypeptide) (e.g., GenBank AAH52808); sarcoma translocation breakpoints polypeptide; SART3 (squamous cell carcinoma antigen recognized by T cells) polypeptide (e.g., GenBank NP_055521); sperm protein 17 polypeptide (e.g., GenBank AAK20878); SSX (synovial sarcoma X breakpoint) polypeptide (e.g., GenBank NP_001265620); SSX2 (synovial sarcoma X breakpoint 2) polypeptide (e.g., GenBank CAA60111); Surviving (survivin polypeptide) (e.g., GenBank AAC51660); TAG-72 (tumor-associate glycoprotein-72); TIE-2 (tyrosine kinase with Ig and EGF homology domains-2 (also known as angiopoietin-1 receptor)) polypeptide (e.g., GenBank NP_000450); TRP-2 (tyrosinase-related protein-2 polypeptide) (e.g., GenBank AAC60627); Tyrosinase (tyrosinase polypeptide) (e.g., GenBank AAB60319); VEGFR2 (vascular endothelial growth factor receptor-2) polypeptide (e.g., GenBank NP_002244); Wilms tumor-1 (WTi) polypeptide; WT-1 (e.g., GenBank NP_000369); and XAGE1 (X antigen family member 1) polypeptide (e.g., GenBank NP_001091073; XP_001125834; XP_001125856; and XP_001125872). See; e.g., Ramos et al. (2013) J. Immunother. 36:66; Cheever et al. (2009) Clin. Cancer Res. 15:5323, and references cited therein; Wagner et al. (2003) J. Cell. Sci. 116:1653; Matsui et al. (1990) Oncogene 5:249; Zhang et al. (1996) Nature 383:168 Cancer-associated antigen peptide epitopes may be a peptide fragment of any of the foregoing proteins from about 4 aas to about 25 aas (e.g., 4 aas (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa) in length.

In some cases, the epitope is HPV16 E7/82-90 (LLMGTLGIV; SEQ ID NO:232). In some cases, the epitope is HPV16 E7/86-93 (TLGIVCPI; SEQ ID NO:233). In some cases, the epitope is HPV16 E7/11-20 (YMLDLQPETT; SEQ ID NO:231). In some cases, the epitope is HPV16 E7/11-19 (YMLDLQPET; SEQ ID NO:230). See, e.g., Ressing et al. ((1995) J. Immunol. 154:5934) for additional suitable HPV epitopes. In some cases, the epitope is a hepatitis B virus (HBV) epitope.

(ii) Self Epitopes

In some cases, the peptide epitope of a MAPP is an epitope associated with or present in a “self”-antigen (an autoantigen). Antigens associated with autoimmune disease can be autoantigens associated with autoimmune diseases such as Addison disease (autoimmune adrenalitis, Morbus Addison), alopecia areata, Addison's anemia (Morbus Biermer), autoimmune hemolytic anemia (AIHA), autoimmune hemolytic anemia (AIHA) of the cold type (cold hemagglutinin disease, cold autoimmune hemolytic anemia (AIHA) (cold agglutinin disease), (CHAD)), autoimmune hemolytic anemia (AIHA) of the warm type (warm AIHA, warm autoimmune hemolytic anemia (AIHA)), autoimmune hemolytic Donath-Landsteiner anemia (paroxysmal cold hemoglobinuria), antiphospholipid syndrome (APS), atherosclerosis, autoimmune arthritis, arteriitis temporalis, Takayasu arteriitis (Takayasu's disease, aortic arch disease), temporal arteriitis/giant cell arteriitis, autoimmune chronic gastritis, autoimmune infertility, autoimmune inner ear disease (AIED), Basedow's disease (Morbus Basedow), Bechterew's disease (Morbus Bechterew, ankylosing spondylitis, spondylitis ankylosans), Behcet's syndrome (Morbus Behcet), bowel disease including autoimmune inflammatory bowel disease (including colitis ulcerosa (Morbus Crohn, Crohn's disease), autoimmune cardiomyopathy, idiopathic dilated cardiomyopathy (DCM), chronic fatigue immune dysfunction syndrome (CFIDS), chronic inflammatory demyelinating polyneuropathy (CIDP), chronic polyarthritis, Churg-Strauss syndrome, cicatricial pemphigoid, Cogan syndrome, CREST syndrome (syndrome with Calcinosis cutis, Raynaud phenomenon, motility disorders of the esophagus, sklerodaktylia and teleangiectasia), Crohn's disease (Morbus Crohn, colitis ulcerosa), dermatitis herpetiformis during, dermatologic autoimmune diseases, dermatomyositis, essential mixed cryoglobulinemia, essential mixed cryoglobulinemia, fibromyalgia, fibromyositis, Goodpasture syndrome (anti-GBM mediated glomerulonephritis), Guillain-Barre syndrome (GBM, Polyradikuloneuritis), hematologic autoimmune diseases, Hashimoto thyroiditis, hemophilia, acquired hemophilia, autoimmune hepatitis, idiopathic pulmonary fibrosis (IPF), idiopathic thrombocytopenic purpura, Immuno-thrombocytopenic purpura (Morbus Werlhof, ITP), IgA nephropathy, autoimmune infertility, juvenile rheumatoid arthritis (Morbus Still, Still syndrome), Lambert-Eaton syndrome, systemic lupus erythematosus (SLE), lupus erythematosus (discoid form), Lyme arthritis (Lyme disease, borrelia arthritis), Meniere's disease (Morbus Meniere); mixed connective tissue disease (MCTD), multiple sclerosis (MS, encephalomyelitis disseminate, Charcot's disease), myasthenia gravis (myasthenia, MG), myositis, polymyositis, neural autoimmune diseases, Pemphigus vulgaris, bullous pemphigoid, polyglandular (autoimmune) syndrome (PGA syndrome, Schmidt's syndrome), polymyalgia rheumatica, primary agammaglobulinemia, primary autoimmune cholangitis, progressive systemic sclerosis (PSS), rheumatoid arthritis (RA, chronic polyarthritis, rheumatic disease of the joints, rheumatic fever), sarcoidosis (Morbus Boeck, Besnier-Boeck-Schaumann disease), stiff-man syndrome, Sclerodermia, Scleroderma, Sjögren's syndrome, autoimmune uveitis, and Wegner's disease (Morbus Wegner, Wegner's granulomatosis).

In some cases, a peptide epitope present in a MAPP is a peptide associated with Addison's disease, alopecia areata, ankylosing spondylitis, autoimmune encephalomyelitis, autoimmune hemolytic anemia, autoimmune hepatitis, autoimmune-associated infertility, autoimmune thrombocytopenic purpura, bullous pemphigoid, Crohn's disease, Goodpasture's syndrome, glomerulonephritis (e.g., crescentic glomerulonephritis, proliferative glomerulonephritis), Grave's disease, Hashimoto's thyroiditis, mixed connective tissue disease, multiple sclerosis, myasthenia gravis (MG), Pemphigus (e.g., Pemphigus vulgaris), pernicious anemia, polymyositis, psoriasis, psoriatic arthritis, rheumatoid arthritis, scleroderma, Sjögren's syndrome, systemic lupus erythematosus (SLE), vasculitis, or vitiligo.

Autoantigens include, e.g., aggrecan, alanyl-tRNA synthetase (PL-12), alpha beta crystallin, alpha fodrin (Sptan 1), alpha-actinin, α1 antichymotrypsin, α1 antitrypsin, α1 microglobulin, aldolase, aminoacyl-tRNA synthetase, an amyloid, an annexin, an apolipoprotein, aquaporin, bactericidal/permeability-increasing protein (BPI), β-globin precursor BP1, β-actin, β-lactoglobulin A, β-2-glycoprotein I, β2-microglobulin, a blood group antigen, C reactive protein (CRP), calmodulin, calreticulin, cardiolipin, catalase, cathepsin B, a centromere protein, chondroitin sulfate, chromatin, collagen, a complement component, cytochrome C, cytochrome P450 2D6, cytokeratin, decorin, dermatan sulfate, DNA topoisomerase I, elastin, Epstein-Barr nuclear antigen 1 (EBNA1), elastin, entactin, an extractable nuclear antigen, Factor I, Factor P, Factor B, Factor D, Factor H, Factor X, fibrinogen, fibronectin, formiminotransferase cyclodeaminase (LC-1), gp210 nuclear envelope protein, GP2 (major zymogen granule membrane glycoprotein), a glutenin, glycoprotein gpIIb/IIIa, glial fibrillary acidic protein (GFAP), glycated albumin, glyceraldehyde 3-phosphate dehydrogenase (GAPDH), haptoglobin A2, heat shock proteins, hemocyanin, heparin, a histone, histidyl-tRNA synthetase (Jo-1), a hordein, hyaluronidase, immunoglobulins, an integrin, interstitial retinol-binding protein 3, intrinsic factor, Ku (p70/p80), lactate dehydrogenase, laminin, liver cytosol antigen type 1 (LC1), liver/kidney microsomal antigen 1 (LKM1), lysozyme, melanoma differentiation-associated protein 5 (MDAS), Mi-2 (chromodomain helicase DNA binding protein 4), a mitochondrial protein, muscarinic receptors, myelin-associated glycoprotein, myosin, myelin basic protein, myelin proteolipid protein, myelin oligodendrocyte glycoprotein, myeloperoxidase (MPO), rheumatoid factor (IgM anti-IgG), neuron-specific enolase, nicotinic acetylcholine receptor A chain, nucleolin, a nucleoporin, nucleosome antigen, PM/Scl100, PM/Scl 75, pancreatic β-cell antigen, pepsinogen, peroxiredoxin 1, phosphoglucose isomerase, phospholipids, phosphatidyl inositol, platelet derived growth factors, polymerase beta (POLB), potassium channel KIR4.1, proliferating cell nuclear antigen (PCNA), proteinase-3, proteolipid protein, proteoglycan, prothrombin, recoverin, rhodopsin, ribonuclease, a ribonucleoprotein, ribosomes, a ribosomal phosphoprotein, RNA, an Sm protein, Sp100 nuclear protein, SRP54 (signal recognition particle 54 kDa), a selectin, smooth muscle proteins, sphingomyelin, streptococcal antigens, superoxide dismutase, synovial joint proteins, T1F1 gamma collagen, threonyl-tRNA synthetase (PL-7), tissue transglutaminase, thyroid peroxidase, thyroglobulin, thyroid stimulating hormone receptor, transferrin, triosephosphate isomerase, tubulin, tumor necrosis factor-alpha, topoisomerase, U1-dnRNP 68/70 kDa, U1-snRNP A, U1-snRNP C, U-snRNP B/B′, ubiquitin, vascular endothelial growth factor, vimentin, and vitronectin.

For the purposes of this disclosure, the epitopes that form part of the MAPPs are not associated with celiac disease or type I diabetes (T1D). In other words, autoantigens (or the self epitopes they present) associated with celiac or TID are not included in a MAPP of the present disclosure. Epitopes associated with type 1 diabetes (T1D) include, e.g., those derived from preproinsulin, proinsulin, insulin, insulin B chain, insulin A chain, 65 kDa isoform of glutamic acid decarboxylase (GAD65), 67 kDa isoform of glutamic acid decarboxylase (GAD67), tyrosine phosphatase (IA-2), heat-shock protein HSP65, islet-specific glucose-6-phosphatase catalytic subunit related protein (IGRP), islet antigen 2 (IA2), zinc transporter (ZnT8), and antigenic peptides thereof. See, e.g., Mallone et al. (2011) Clin. Dev. Immunol. 2011:513210; and U.S. Patent Publication No. 2017/0045529. Epitopes/antigens associated with celiac disease include celiac-associated epitopes derived from e.g., tissue transglutaminase, gliadins, glutenins, secalins, hordeins, and avenins. Examples of secalins include rye secalins. Examples of hordeins include barley hordeins. Examples of glutenins include wheat glutenins. See, e.g., U.S. 2016/0279233. An antigen “associated with” a particular autoimmune disorder is an antigen that is a target of autoantibodies and/or autoreactive T cells present in individuals with that autoimmune disorder, where such autoantibodies and/or autoreactive T cells mediate a pathological state associated with the autoimmune disorder. The present disclosure does not encompass methods of preparing protein constructs comprising antigens/epitopes associated with celiac or T1D, compositions comprising such proteins constructs or nucleic acids encoding such proteins, or the treatment of T1D and/or celiac disease.

Autoantigens associated with alopecia areata (autoimmune alopecia) include, e.g., hair follicle keratinocyte polypeptides, melanogenesis-associated autoantigens, and melanocyte polypeptides. An example of a melanocyte autoantigen is tyrosinase. Autoantigens associated with autoimmune alopecia also include trichohyalin (Leung et al. (2010) J. Proteome Res. 9:5153) and keratin 16. A suitable epitope-presenting peptide for inclusion in a MAPP can be an epitope-presenting peptide of from 4 aas to about 25 aas in length of a hair follicle keratinocyte polypeptide, a melanocyte polypeptide, a melanogenesis-associated polypeptide, tyrosinase, trichohyalin, or keratin 16. Autoantigens associated with Addison's disease include, e.g., 21-hydroxylase. A suitable epitope-presenting peptide for inclusion in a MAPP can be an epitope-presenting peptide of from 4 aas to about 25 aas in length of 21-hydroxylase.

Autoantigens associated with autoimmune thyroiditis (Hashimoto's thyroiditis) include, e.g., thyroglobulin, thyroid peroxidase, thyroid Stimulating Hormone Receptor (TSH-Receptor), thyroidal iodide transporters Na+/I− symporter (NIS), pendrin, and the like. A suitable epitope-presenting peptide for inclusion in a MAPP can be an epitope-presenting peptide of from 4 aas to about 25 aas in length of any one of the aforementioned Hashimoto's thyroiditis-associated polypeptides.

Autoantigens associated with Crohn's disease include, e.g., pancreatic secretory granule membrane glycoprotein-2 (GP2). A suitable epitope-presenting peptide for inclusion in a MAPP can be an epitope-presenting peptide of from 4 aas to about 25 aas in length of GP2.

Autoantigens associated with Goodpasture's disease include, e.g., the α3 chain of type IV collagen, e.g., aas 135-145 of the α3 chain of type IV collagen. Penades et al. (1995) Eur. J. Biochem. 229:754; Kalluri et al. (1994) Proc. Natl. Acad. Sci. USA 91:6201. A suitable epitope-presenting peptide for inclusion in a MAPP can be an epitope-presenting peptide of from 4 aas to about 25 aas in length of the α3 chain of type IV collagen.

Autoantigens associated with Grave's disease include, for example, thyroglobulin, thyroid peroxidase, and thyrotropin receptor (TSH-R). A suitable epitope-presenting peptide for inclusion in a MAPP can be an epitope-presenting peptide of from 4 aas to about 25 aas in length of any one of the aforementioned Grave's disease-associated antigens.

Autoantigens associated with mixed connective tissue disease include, e.g., U1 ribonucleoprotein (U1-RNP) polypeptide (also known as snRNP70). Sato et al. (2010) Mol. Cell. Biochem. 106:55. A suitable epitope-presenting peptide for inclusion in a MAPP can be an epitope-presenting peptide of from 4 aas to about 25 aas in length of U1-RNP polypeptide.

Autoantigens associated with multiple sclerosis include, e.g., myelin basic protein, myelin oligodendrocyte glycoprotein, and myelin proteolipid protein. A suitable epitope-presenting peptide for inclusion in a MAPP can be an epitope-presenting peptide of from 4 aas to about 25 aas in length of any one of the aforementioned multiple sclerosis-associated antigens. As one non-limiting example, the peptide epitope can comprise the aa sequence ENPVVHFFKNIVTPR (SEQ ID NO:234). In some cases, a MAPP comprises a DRB1*15:01 MHC class II β chain; and a peptide epitope of the aa sequence ENPVVHFFKNIVTPR (SEQ ID NO:234).

Autoantigens associated with myasthenia gravis include, e.g., acetylcholine receptor (AchR; see, e.g., Lindstrom (2000) Muscle & Nerve 23:453), muscle-specific tyrosine kinase, and low-density lipoprotein receptor-related protein-4. A suitable epitope-presenting peptide for inclusion in a MAPP can be an epitope-presenting peptide of from 4 aas to about 25 aas in length of any one of the aforementioned myasthenia gravis-associated antigens. In some cases, a suitable epitope-presenting peptide for inclusion in a MAPP is an epitope-presenting peptide of from 4 aas to about 25 aas in length of an AchR.

Autoantigens associated with Parkinson's disease include, e.g., α-synuclein. A suitable epitope-presenting peptide for inclusion in a MAPP can be an epitope-presenting peptide of from 4 aas to about 25 aas in length of α-synuclein. For example, a suitable epitope-presenting peptide for inclusion in a MAPP includes a peptide of from 5 aas to the entire length of any one of the following: GKTKEGVLYVGSKTK (SEQ ID NO:235); KTKEGVLYVGSKTKE (SEQ ID NO:236); MPVDPDNEAYEMPSE (SEQ ID NO:237); DNEAYEMPSEEGYQD (SEQ ID NO:238); EMPSEEGYQDYEPE (SEQ ID NO:239); and SEEGYQDYEPEA (SEQ ID NO:240) where “S” denotes phosphoserine.

Autoantigens associated with Pemphigus (e.g., Pemphigus vulgaris, Pemphigus foliaceus, bullous pemphigoid) include Pemphigus vulgaris immunogens such as desmosomal cadherin desmoglein 3 (Dsg3); Pemphigus foliaceus immunogens such as Dsg1; bullous pemphigoid immunogens such as hemidesmosome peptides including BP230 antigen, GPAG1a, and BPAG1b. See, e.g., Cirillo et al. (2007) Immunology 121:377. Autoantigens associated with bullous pemphigoid include bullous pemphigoid antigen 1 (BPAG1; also known as BP230 or dystonin), bullous pemphigoid antigen 2 (BPAG2; also known as BP180 or type XVII collagen), and subunits of human integrins α-5 and β-4. A suitable epitope-presenting peptide for inclusion in a MAPP can be an epitope-presenting peptide of from 4 aas to about 25 aas in length of any of the aforementioned Pemphigus-associated antigens.

Autoantigens associated with myositis (e.g., polymyositis; dermatomyositis) include, e.g., histidyl tRNA synthetase. A suitable epitope-presenting peptide for inclusion in a MAPP can be an epitope-presenting peptide of from 4 aas to about 25 aas in length of histidyl tRNA synthetase.

Autoantigens associated with rheumatoid arthritis include, e.g., collagen, vimentin, aggrecan, fibrinogen, cyclic citrullinated peptides, α-enolase, histone polypeptides, lactoferrin, catalase, actinin, and actins (cytoplasmic 1 and 2 (β/γ). A suitable epitope-presenting peptide for inclusion in a MAPP can be an epitope-presenting peptide of from 4 aas to about 25 aas in length of any one of the aforementioned rheumatoid arthritis-associated antigens.

Autoantigens associated with scleroderma include nuclear antigens. A suitable epitope-presenting peptide for inclusion in a MAPP can be an epitope-presenting peptide of from 4 aas to about 25 aas in length of a nuclear antigen associated with scleroderma.

Autoantigens associated with Sjögren's syndrome include, e.g., Ro/La ribonucleoprotein (RNP) complex, alpha-fodrin, beta-fodrin, islet cell autoantigen, poly(ADP)ribose polymerase (PARP), nuclear mitotic apparatus (NuMA), NOR-90, Ro60 kD autoantigen, Ro52 antigen, La antigen (see, e.g., GenBank Accession No. NP_001281074.1), and p27 antigen. A suitable epitope-presenting peptide for inclusion in a MAPP can be an epitope-presenting peptide of from 4 aas to about 25 aas in length of any one of the aforementioned Sjögren's syndrome-associated antigens.

Autoantigens associated with systemic lupus erythematosus (SLE) include, e.g., Ro60 autoantigen, low-density lipoproteins, Sm antigens of the U-1 small nuclear ribonucleoprotein complex (B/B′, D1, D2, D3, E, F, G), α-actin 1, α-actin 4, annexin A1, C1q/tumor necrosis factor-related protein, catalase, defensins, chromatin, histone proteins, transketolase, hCAP18/LL37, and ribonucleoproteins (RNPs). A suitable epitope-presenting peptide for inclusion in a MAPP can be an epitope-presenting peptide of from 4 aas to about 25 aas in length of any one of the aforementioned SLE-associated antigens.

Autoantigens associated with thrombocytopenia purpura include ADAMTS13 (a disintegrin and metalloproteinase with a thrombospondin type 1 motif, member 13), and von Willebrand factor-cleaving protease (VWFCP). A suitable epitope-presenting peptide for inclusion in a MAPP can be an epitope-presenting peptide of from 4 aas to about 25 aas in length of an ADAMTS13 polypeptide or a VWFCP polypeptide.

Autoantigens associated with vasculitis include proteinase-3, lysozyme C, lactoferrin, leukocyte elastase, cathepsin G, and azurocidin. A suitable epitope-presenting peptide for inclusion in a MAPP can be an epitope-presenting peptide of from 4 aas to about 25 aas in length of any of the aforementioned vasculitis-associated antigens.

Autoantigens associated with vitiligo include SOX9, SOX10, PMEL (Premelanosomal protein), tyrosinase, TYRP1 (Tyrosine related protein 1), DDT (D-Dopachrome tautomerase), Rab38, and MCHR1 (Melanin-concentrating receptor. A suitable epitope-presenting peptide for inclusion in a MAPP can be an epitope-presenting peptide of from 4 aas to about 25 aas in length of any one of the aforementioned vitiligo-associated polypeptides.

Autoantigens associated with autoimmune uveitis include, for example, interphotoreceptor retinoid-binding protein (IRBP). A suitable epitope-presenting peptide for inclusion in a MAPP can be an epitope-presenting peptide of from 4 aas to about 25 aas in length IRBP. A suitable epitope-presenting peptide for inclusion in a MAPP can be an epitope-presenting peptide of from 4 aas to about 25 aas in length of any one of the aforementioned antigens.

Autoantigens associated with autoimmune polyendocrine syndrome include, e.g., 17-alpha hydroxylase, histidine decarboxylase, tryptophan hydroxylase, and tyrosine hydroxylase. A suitable epitope-presenting peptide for inclusion in a MAPP can be an epitope-presenting peptide of from 4 aas to about 25 aas in length of any one of the aforementioned autoimmune polyendocrine syndrome-associated antigens.

Autoantigens associated with psoriasis include ADAMTS15. See, e.g., Prinz (2017) Autoimmunity Reviews 16:970. A suitable epitope-presenting peptide for inclusion in a MAPP can be an epitope-presenting peptide of from 4 aas to about 25 aas in length of an ADAMTS15 polypeptide.

(iii) Allergens

In some cases, the peptide presented in the context of a MAPP comprises class II MHC presenting sequence(s) or complexe(s) is an allergen. Allergens are too numerous to recite, but by way of example, allergens include, but are not limited to, peanuts and tree nuts, plant pollens, latex, and the like. Allergens also include proteins from hymenoptera proteins (e.g., allergens in bee and wasp venoms such as phospholipase A2, melittin, “antigen 5” found in wasp venom, and hyaluronidases).

Peptide presenting epitopes to peanut allergens, such as the Ara h 1 to 13 proteins that come from seven protein families, include those in Ara h 1 (e.g., PGQFEDFF (SEQ ID NO:241), YLQGFSRN (SEQ ID NO:242), FNAEFNEIRR (SEQ ID NO:243), QEERGQRR (SEQ ID NO:244), DITNPINLRE (SEQ ID NO:245), NNFGKLFEVK (SEQ ID NO:246), GNLELV (SEQ ID NO:247), RRYTARLKEG (SEQ ID NO:248), ELHLLGFGIN (SEQ ID NO:249), HRIFLAGDKD (SEQ ID NO:250), IDQIEKQAKD (SEQ ID NO:251), KDLAFPGSGE (SEQ ID NO:252), KESHFVSARP (SEQ ID NO:253), NEGVIVKVSKEHVEELTKHAKSVSK (SEQ ID NO:254)), Ara h 2 (e.g., HASARQQWEL (SEQ ID NO:255), QWELQGDRRC (SEQ ID NO:256), DRRCQSQLER (SEQ ID NO:257), LRPCEQHLMQ (SEQ ID NO:258), KIQRDEDSYE (SEQ ID NO:259), YERDPYSPSQ (SEQ ID NO:260), SQDPYSPSPY (SEQ ID NO:261), DRLQGRQQEQ (SEQ ID NO:262), KRELRNLPQQ (SEQ ID NO:263), QRCDLDVESG (SEQ ID NO:264)), and Ara h 3 (e.g., IETWNPNNQEFECAG (SEQ ID NO:265), GNIFSGFTPEFLAQA (SEQ ID NO:266), VTVRGGLRILSPDRK (SEQ ID NO:267), DEDEYEYDEEDRRRG (SEQ ID NO:268)). See, e.g., Zhou et al, (2013) Intl. J. of Food Sci. 2013: 8 pages article ID 909140.

8. Additional Polypeptides

A polypeptide chain of a MAPP (e.g., a dimerization or framework polypeptide) may include one or more polypeptides in addition to those described above. Suitable additional polypeptides include epitope tags and affinity domains. The one or more additional polypeptides can be included at the N-terminus of a polypeptide chain of a MAPP of the present disclosure, at the C-terminus of a polypeptide chain of a MAPP of the present disclosure, or within (internal to) a polypeptide chain of a MAPP of the present disclosure.

a. Affinity Tags, Epitope Tags and Affinity Domains

Suitable affinity/epitope tags include, but are not limited to, hemagglutinin (HA; e.g., YPYDVPDYA (SEQ ID NO:269); FLAG (e.g., DYKDDDDK (SEQ ID NO:270); c-myc (e.g., EQKLISEEDL; SEQ ID NO:271), and the like.

Affinity domains include peptide sequences that can interact with a binding partner, e.g., such as one immobilized on a solid support, useful for identification or purification. DNA sequences encoding multiple consecutive single aas, such as histidine, when fused to the expressed protein, may be used for one-step purification of the recombinant protein by high affinity binding to a resin column, such as nickel sepharose. Exemplary affinity domains include HisX5 (HHHHH) (SEQ ID NO:272), HisX6 (HHHHHH) (SEQ ID NO:273), C-myc (EQKLISEEDL) (SEQ ID NO:271), Flag (DYKDDDDK) (SEQ ID NO:270), StrepTag (WSHPQFEK) (SEQ ID NO:277, hemagglutinin, e.g., HA Tag (YPYDVPDYA) (SEQ ID NO:269), glutathione-S-transferase (GST), thioredoxin, cellulose binding domain, RYIRS (SEQ ID NO:274), Phe-His-His-Thr (SEQ ID NO:275), chitin binding domain, S-peptide, T7 peptide, SH2 domain, C-end RNA tag, WEAAAREACCRECCARA (SEQ ID NO:276), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, calretinin, inteins, biotin, streptavidin, MyoD, Id, leucine zipper sequences, and maltose binding protein.

b. Targeting Sequences

MAPPs may include, as part of any one or more framework and/or any one or more dimerization polypeptide, a targeting polypeptide or “targeting sequence.” Targeting sequences serve to bind or “localize” MAPPs to cells and/or tissues displaying the protein (or other molecule) to which the targeting sequence binds. Targeting sequences may be located, for example at or near the carboxyl terminal end of a framework or dimerization peptide (e.g., in place of a C-terminal MOD in FIG. 1A or 1B or at position 3, 3′, 5 and/or 5′ of the MAPP in any of FIG. 1A, 1B or 19-23). In an embodiment the targeting sequence may be located at position 3 and/or 3′. Targeting sequences serve to bind or “localize” MAPPs to cells and tissue displaying the protein (or other molecule) which the targeting sequence binds. In some cases, a targeting sequence is an antibody or antigen binding fragment thereof (e.g., an scFv or a nanobody such as a heavy chain nanobody or a light chain nanobody). In some cases, a targeting sequence is a single-chain T cell receptor (scTCR). Targeting sequences may be translated as part of the MAPP (e.g., part of the framework polypeptide) or incorporated by covalent attachment (e.g., using a crosslinker) of a targeting sequence, where the targeting sequence effectively becomes a payload-like molecule attached to the MAPP. Targeting sequences may also be non-covalently bound to a MAPP. For example, a MAPP having a biotin labeled framework polypeptide may be non-covalently attached to an avidin labeled targeting antibody or Fab directed to, for example, a cancer antigen). A bispecific antibody (e.g., a bispecific IgG or humanized antibody) having a first antigen binding site directed to a part of the MAPP (e.g., the framework polypeptide) may also be employed to non-covalently attach a MAPP to a targeting sequence (the second bispecific antibody binding site) directed to a cell or tissue target (e.g., a cancer antigen).

In some instances, a targeting sequence present in a MAPP targets an antigen of an infecting organism and/or of an infected cell. Targeting polypeptides may be directed to proteins/epitopes of infectious agents including, but not limited to, viruses, bacteria, fungi, protozoans, and helminths, including those proteins/epitopes of infectious agents that are expressed on cell surfaces. By way of example, cells infected with HPV may express E6 or E7 proteins (described herein as cancer associated epitope) or portions thereof to which the targeting sequence may be directed. A targeting sequence may also be a Cancer Targeting Polypeptide, or “CTP” that is specific for a cancer associated antigen (“CAA”), such as an antigen associated with a non-solid cancer (e.g., a leukemia) and/or solid tumor-associated antigen. In one instance, the targeting sequence is specific for a cancer-associated peptide/HLA (pHLA) complex on the surface of a cancer cell, where the peptide can be a cancer-associated peptide (e.g., a peptide fragment of a cancer-associated antigen). MAPPs can also be targeted to specific tissues or cell types by using targeting sequences directed toward molecules expressed selectively by cells of the desired tissue.

9. Payloads—Drug And Other Conjugates

A polypeptide chain of a MAPP can comprise a payload such as a therapeutic (e.g., a small molecule drug or therapeutic) a label (e.g., a fluorescent label or radio label), or other biologically active agent that is linked (e.g., covalently attached) to the polypeptide chain. For example, where a MAPP comprises an Fc polypeptide, the Fc polypeptide may comprise a covalently linked payload such as an agent that treats a cancer, infectious disease, or an autoimmune disease, potentates the action of the MAPP, or is an agent that relieves a symptom of such diseases.

A payload can be linked directly or indirectly to a polypeptide chain of a MAPP (e.g., to an Ig Fc polypeptide in the MAPP). Direct linkage can involve linkage to an aa side chain without an intervening linker. Indirect linkage can be linkage via a cross-linker, such as a bifunctional cross-linker. A payload can be linked to a MAPP by any acceptable chemical linkage including, but not limited to a thioether bond, an amide bond, a carbamate bond, a disulfide bond, or an ether bond, including those formed by reaction with a crosslinking agent.

Crosslinkers (crosslinking agents) include cleavable cross-linkers and non-cleavable cross-linkers. The cross-linkers may be homobifunctional or heterobifunctional cross-linkers. In some cases, the cross-linker is a protease-cleavable cross-linker. Suitable cross-linkers may include as moieties, for example, peptides (e.g., from 2 to 10 aas in length; e.g., 2, 3, 4, 5, 6, 7, 8, 9, or 10 aas in length), alkyl chains, poly(ethylene glycol), disulfide groups, thioether groups, acid labile groups, photolabile groups, peptidase labile groups, and esterase labile groups. Non-limiting example of suitable cross-linkers are: N-succinimidyl-[(N-maleimidopropionamido)-tetraethyleneglycol]ester (NHS-PEG4-maleimide); N-succinimidyl 4-(2-pyridyldithio)butanoate (SPDB); N-succinimidyl 4-(2-pyridyldithio)2-sulfobutanoate (sulfo-SPDB); N-succinimidyl 4-(2-pyridyldithio) pentanoate (SPP); N-succinimidyl-4-(N-maleimidomethyl)-cyclohexane-1-carboxy-(6-amidocaproate) (LC-SMCC); κ-maleimidoundecanoic acid N-succinimidyl ester (KMUA); γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); N-(α-maleimidoacetoxy)-succinimide ester (AMAS); succinimidyl-6-(β-maleimidopropionamide)hexanoate (SMPH); N-succinimidyl 4-(p-maleimidophenyl)butyrate (SMPB); N-(p-maleimidophenyl)isocyanate (PMPI); N-succinimidyl 4(2-pyridylthio)pentanoate (SPP); N-succinimidyl(4-iodo-acetyl)aminobenzoate (SIAB); 6-maleimidocaproyl (MC); maleimidopropanoyl (MP); p-aminobenzyloxycarbonyl (PAB); N-succinimidyl 4-(maleimidomethyl)cyclohexanecarboxylate (SMCC); N-succinimidyl-4-(N-maleimidomethyl)-cyclohexane-1-carboxy-(6-amidocaproate), a “long chain” analog of SMCC (LC-SMCC); 3-maleimidopropanoic acid N-succinimidyl ester (BMPS); N-succinimidyl iodoacetate (SIA); N-succinimidyl bromoacetate (SBA); and N-succinimidyl 3-(bromoacetamido)propionate (SBAP).

MAPP payload conjugates may be formed by reaction of a MAPP polypeptide (e.g., an IgFc polypeptide) with a cross-linking reagent to introduce 1-10 reactive groups. The polypeptide is then reacted with the molecule to be conjugated (e.g., a thiol-containing payload drug, label or agent) to produce a MAPP-payload conjugate. For example, where a MAPP comprises an IgFc polypeptide, the conjugate can be of the form (A)-(L)-(C), where (A) is the polypeptide chain comprising the IgFc polypeptide; where (L), if present, is a cross-linker; and where (C) is a payload. (L), if present, links (A) to (C). In some cases, the MAPP includes an IgFc polypeptide that comprises one or more (e.g., 2, 3, 4, 5, or more than 5) molecules of a payload. Introducing payloads into a MAPP using an excess of cross-linking agents can result in multiple molecules of payload being incorporated into the MAPP.

Suitable payloads (e.g., drugs) include virtually any small molecule (e.g., less than 2,000 Daltons in molecular weight) approved by the U.S. Food and Drug Administration, and/or listed in the 2020 U.S. Pharmacopeia or National Formulary. In an embodiment, those drugs are less than 1,000 molecular weight. Suitable drugs include antibiotics, chemotherapeutic (antineoplastic), anti-fungal, or anti-helminth agents and the like (e.g., sulfasalazine, azathioprine, cyclophosphamide, leflunomide; methotrexate, antimalarials, D-penicillamine, cyclosporine). Suitable chemotherapeutics may be alkylating agents, cytoskeletal disruptors (taxanes), epothilone, histone deacetylase inhibitors, topoisomerase I inhibitors, topoisomerase II inhibitors, kinase inhibitors, nucleotide analog or precursor analogs, peptide antineoplastic antibiotics (e.g., bleomycin or actinomycin), platinum-based agents, retinoids, or vinca alkaloids. Suitable drugs also include non-steroidal anti-inflammatory drugs and glucocorticoids, and the like.

D. Nucleic Acids

The present disclosure provides a nucleic acid comprising a nucleotide sequence encoding one or more polypeptides of a MAPP of the present disclosure. In some cases, the nucleic acid is a recombinant expression vector; thus, the present disclosure provides a recombinant expression vector comprising a nucleotide sequence encoding a MAPP of the present disclosure.

1. Nucleic Acids Encoding a MAPP or MAPP Forming a Higher Order Complex, Such as a Duplex MAPP, that Comprises at Least One Dimerization Sequence and a Multimerization Sequence

The present disclosure provides nucleic acids comprising a nucleotide sequence encoding a MAPP having a framework polypeptide that comprises at least one dimerization sequence and at least one multimerization sequence that permits two molecules of the framework polypeptide to form dimers or higher order complexes. The nucleic acids may additionally comprise a nucleotide sequence encoding a dimerization peptide. Where the MAPP comprises a presenting sequence the nucleic acids encoding either or both of the framework polypeptide and/or dimerization peptide may include a sequence encoding a presenting sequence. Where the MAPP comprises a presenting complex, the nucleic acids encoding either or both of the framework polypeptide and/or dimerization peptide may further comprise sequences encoding a presenting complex 1^{st} sequence and/or a presenting complex 2^{nd} sequence. Where desired, he nucleic acid sequences encoding a MAPP may also exclude a sequence encoding a peptide epitope. The nucleotide sequence(s) comprising any of the MAPP polypeptides can be operably linked to a transcription control element(s), e.g., a promoter. It will be apparent that individual polypeptides of a MAPP (e.g., a framework polypeptide and dimerization polypeptide) may be encoded on a single nucleic acid (e.g., under the control of separate promoters), or alternatively, may be located on two or more separate nucleic acids (e.g., plasmids).

2. Recombinant Expression Vectors

The present disclosure provides recombinant expression vectors comprising nucleic acids encoding one or more polypeptides of a MAPP or its higher order complexes. In some cases, the recombinant expression vector is a non-viral vector. In some cases, the recombinant expression vector is a viral construct, such as a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g., viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol. 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available.

Depending on the host/vector system utilized, any of a number of suitable transcription and translation control elements, including constitutive and inducible promoters, transcription enhancer elements, transcription terminators, etc. may be used in the expression vector (see, e.g., Bitter et al. (1987) Methods in Enzymology, 153:516-544).

In some cases, a nucleotide sequence encoding one or more polypeptides of a MAPP is operably linked to a control element, e.g., a transcriptional control element, such as a promoter. The transcriptional control element may be functional in either a eukaryotic cell, e.g., a mammalian cell such as a human, hamster, or mouse cell; or a prokaryotic cell (e.g., bacterial). In some cases, a nucleotide sequence encoding a DNA-targeting RNA and/or a site-directed modifying polypeptide is operably linked to multiple control elements that allow expression of the nucleotide sequence encoding a DNA-targeting RNA and/or a site-directed modifying polypeptide in both prokaryotic and eukaryotic cells.

Non-limiting examples of suitable eukaryotic promoters (promoters functional in a eukaryotic cell) include the cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

E. Genetically Modified Host Cells

The present disclosure provides a genetically modified host cell, where the host cell is genetically modified with a nucleic acid(s) that encode, or encode and express, MAPP proteins or higher order complexes of MAPPs (e.g., duplex MAPPs).

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2),™), CHO cells (e.g., ATCC Nos. CRL9618, CCL61, CRL-9618™, CCL-61™ CRL9096), 293 cells (e.g., ATCC No. CRL-1573),™), Vero cells, NIH 3T3 cells (e.g., ATCC No. CRL-1658), Huh-7 cells, BHK cells (e.g., ATCC No. CCL10), CCL-10T^{M}), PC12 cells (ATCC No. CRL1721), CRL-1721™), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like. In some cases, the host cell is a mammalian cell that has been genetically modified such that it does not synthesize endogenous MHC Class II heavy chains (MHC-H).

Genetically modified host cells can be used to produce a MAPP and higher order complexes of MAPPs. For example, a genetically modified host cell can be used to produce a duplex MAPP. For example, an expression vector(s) comprising nucleotide sequences encoding the MAPP polypeptide(s) is/are introduced into a host cell, generating a genetically modified host cell, which genetically modified host cell produces the polypeptide(s) (e.g., as an excreted soluble protein).

F. Methods of Producing MAPPs

The present disclosure provides methods of producing the MAPPs (e.g., duplex MAPPs) described herein. The methods generally involve culturing, in a culture medium, a host cell that is genetically modified with a recombinant expression vector(s) comprising a nucleotide sequence(s) encoding the MAPP (e.g., a genetically modified host cell of the present disclosure); and isolating the MAPP from the genetically modified host cell and/or the culture medium. As noted above, in some cases, the individual polypeptide chains of a MAPP are encoded in separate nucleic acids (e.g., recombinant expression vectors). In some cases, all polypeptide chains of a MAPP are encoded in a single recombinant expression vector.

Isolation of the MAPP from the host cell employed for expression (e.g., from a lysate of the expression host cell) and/or the culture medium in which the host cell is cultured, can be carried out using standard methods of protein purification. For example, a lysate of the host cell may be prepared, and the MAPP purified from the lysate using high performance liquid chromatography (HPLC), exclusion chromatography (e.g., size exclusion chromatography), gel electrophoresis, affinity chromatography, or other purification technique. Alternatively, where the MAPP is secreted from the expression host cell into the culture medium, the MAPP can be purified from the culture medium using HPLC, exclusion chromatography, gel electrophoresis, affinity chromatography, or other purification technique. In some cases, the MAPP is purified, e.g., a composition is generated that comprises at least 80% by weight, at least about 85% by weight, at least about 95% by weight, or at least about 99.5% by weight, of the MAPP in relation to contaminants related to the method of preparation of the product and its purification. The percentages can be based upon total protein.

In some cases, e.g., where the expressed MAPP comprises an affinity tag or affinity domain, the MAPP can be purified using an immobilized binding partner of the affinity tag. For example, where a MAPP comprises an Ig Fc polypeptide, the MAPP can be isolated from genetically modified mammalian host cell and/or from culture medium comprising the MAPP by affinity chromatography, e.g., on a Protein A column, a Protein G column, or the like. An example of a suitable mammalian cell is a CHO cell; e.g., an Expi-CHO-S™ cell (e.g., ThermoFisher Scientific, Catalog #A29127).

The polypeptides of the MAPP will self-assemble into heterodimers, and where applicable, spontaneously form disulfide bonds between, for example, framework polypeptides, or framework and dimerization polypeptides. As also noted above, when both framework polypeptides include Ig Fc polypeptides, disulfide bonds will spontaneously form between the respective Ig Fc polypeptides to covalently link the two heterodimers of framework and dimerization polypeptides to one another to form a covalently linked duplex MAPP.

G. Compositions

1. Compositions Comprising a MAPP

The present disclosure provides compositions, including pharmaceutical compositions, comprising a MAPP and/or higher order complexes of MAPPs (e.g., duplex MAPPs). Pharmaceutical composition can comprise, in addition to a MAPP, one or more known carriers, excipients, diluents, buffers, salts, surfactants (e.g., non-ionic surfactants), amino acids (e.g., arginine), etc., a variety of which are known in the art and need not be discussed in detail herein. For example, see “Remington: The Science and Practice of Pharmacy”, 19^{th} Ed. (1995), or latest edition, Mack Publishing Co.

In some cases, a subject pharmaceutical composition will be suitable for administration to a subject, e.g., will be sterile and/or substantially free of pyrogens. For example, in some embodiments, a subject pharmaceutical composition will be suitable for administration to a human subject, e.g., where the composition is sterile and is substantially free of detectable pyrogens and/or other toxins, or such detectable pyrogens and/or other toxins are below a permissible limit.

The compositions may, for example, be in the form of aqueous or other solutions, powders, granules, tablets, pills, suppositories, capsules, suspensions, sprays, and the like. The composition may be formulated according to the various routes of administration described below.

Where a MAPP or higher order MAPP complex (e.g., duplex MAPP) is administered as an injectable (e.g., subcutaneously, intraperitoneally, intramuscularly, intralymphatically, and/or intravenously) directly into a tissue, a formulation can be provided as a ready-to-use dosage form, or as non-aqueous form (e.g., a reconstitutable storage-stable powder) or an aqueous form, such as liquid composed of pharmaceutically acceptable carriers and excipients. MAPPs may also be provided so as to enhance serum half-life of the subject protein following administration. For example, the protein may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

In some cases, a MAPP composition comprises: a) a MAPP higher order MAPP complex (e.g., a duplex MAPP) of the present disclosure; and b) saline (e.g., 0.9% NaCl). In some cases, the composition is sterile and/or substantially pyrogen free. In some cases, the composition is suitable for administration to a human subject, e.g., where the composition is sterile and is free of detectable pyrogens and/or other toxins. Thus, the present disclosure provides a composition comprising: a) a MAPP or higher order MAPP complex (e.g., duplex MAPP) of the present disclosure; and b) saline (e.g., 0.9% NaCl), where the composition is sterile and is substantially free of detectable pyrogens and/or other toxins, or such detectable pyrogens and/or other toxins are below a permissible limit.

Other examples of components suitable for inclusion in formulations suitable for parenteral administration include isotonic sterile injection solutions, anti-oxidants, bacteriostats, and solutes that render the formulation isotonic with the blood of the intended recipient, suspending agents, solubilizers, thickening agents, stabilizers, and preservatives. A pharmaceutical composition can be present in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The concentration of a MAPP in a formulation can vary widely. For example, a MAPP or higher order MAPP complex (e.g., duplex MAPP) may be present from less than about 0.1% (usually at least about 2%) to as much as 20% to 50% or more by weight (e.g., from 1% to 10%, 5% to 15%, 10% to 20% by weight, or 20-50% by weight) by weight. The concentration will usually be selected primarily based on fluid volumes, viscosities, and patient-based factors in accordance with the particular mode of administration selected and the patient's needs.

The present disclosure provides a container comprising a composition of the present disclosure, e.g., a liquid composition. The container can be, e.g., a syringe, an ampoule, and the like. In some cases, the container is sterile. In some cases, both the container and the composition are sterile.

2. Compositions Comprising a Nucleic Acid or a Recombinant Expression Vector

The present disclosure provides compositions (e.g., pharmaceutical compositions) comprising a nucleic acid or a recombinant expression vector that comprise one or more nucleic acid sequences encoding any one or more MAPP polypeptides (or each of the polypeptides of a MAPP). As discussed above, a wide variety of pharmaceutically acceptable excipients is known in the art and need not be discussed in detail herein.

A nucleic acid or a recombinant expression vector composition can include one or more nucleic acids or one or more recombinant expression vectors comprising a nucleic acid (e.g., DNA or RNA) sequences encoding a MAPP polypeptide or all polypeptides of a MAPP. Such compositions may further include one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative.

A pharmaceutically acceptable formulation may comprise a nucleic acid or recombinant expression vector encoding one or more polypeptides of a MAPP (e.g., in an amount of from about 0.001% to about 90% (w/w)). In some cases, such pharmaceutical compositions will be suitable for administration to a subject, e.g., will be sterile and/or substantially free of pyrogens. For example, in some embodiments, a the pharmaceutical composition will be suitable for administration to a human subject, e.g., where the composition is sterile and is substantially free of detectable pyrogens and/or other toxins, or such detectable pyrogens and/or other toxins are below a permissible limit.

A composition comprising a nucleic acid or a recombinant expression vector encoding one or more polypeptides of a MAPP, including pharmaceutically acceptable formulations, may be:
(i) admixed, encapsulated, conjugated or otherwise associated with other compounds or mixtures of compounds (e.g., liposomes or receptor-targeted molecules), or combined in a formulation with one or more components that assist in uptake, distribution and/or absorption of the nucleic acids or vectors;
(ii) formulated into dosage forms including, but not limited to, tablets, capsules, gel capsules, liquid syrups, soft gels, or suspensions in aqueous, non-aqueous or mixed media; and
(iii) formulated as a liposomal formulation. As used herein, the term “liposome” means a vesicle composed of amphiphilic lipids.

The compositions comprising a nucleic acid or a recombinant expression vector described herein may include penetration enhancers to effect the efficient delivery of nucleic acids or expression vectors. Penetration enhancers may be classified as belonging to one of five broad categories, i.e., surfactants, fatty acids, bile salts, chelating agents, and non-chelating non-surfactants. Penetration enhancers and their uses are further described in, for example, U.S. Pat. No. 6,287,860, which is incorporated for its discussion penetration enhancers.

H. Methods

MAPPs and higher order MAPP complexes (e.g., duplex MAPP) are useful for modulating an activity of a T cell. Thus, the present disclosure provides methods of modulating an activity of a T cell, the methods generally involving contacting a target T cell with a MAPP or a higher order MAPP complex (e.g., duplex MAPP) of the present disclosure.

1. Methods of Modulating T Cell Activity

The present disclosure provides a method of selectively modulating the activity of a T cell that is specific for an epitope presented by a MAPP, the method comprising contacting, in vitro or in vivo, the T cell with a MAPP, where contacting the T cell with a MAPP or higher order MAPP, such as a duplex MAPP, presenting the epitope selectively modulates the activity of the epitope-specific T cell. The contacting may occur in vivo, typically in a human, but potentially in another animal such as a rat, mouse, dog, cat, pig, horse, or primate. In some cases, the contacting occurs in vitro.

In some cases, a MAPP reduces activity of an autoreactive T cell and/or an autoreactive B cell. In some cases, a MAPP increases the number and/or activity of a regulator T cell (Treg), resulting in reduced activity of an autoreactive T cell and/or an autoreactive B cell.

In some cases, a MAPP is contacted with an epitope-specific CD4⁺ T cell. In some cases, the epitope-specific T cell is a CD4⁺ CD8⁺ (double positive) T cell (see e.g., Boher et al Front. Immunol., 29 Mar. 2019 on the www at: doi.org/10.3389/fimmu.2019.00622 and Matsuzaki et al. J. Immuno. Therapy of Cancer 7: Article number: 7 (2019)). In some cases the epitope-specific T cell is a NK-T cell (see, e.g., Nakamura et al. J, Immunol. 2003 Aug. 1; 171(3):1266-71). In some cases, the epitope-specific T cell is a T (Treg). The contacting may result in modulating the activity of a T cell, which can result in, but is not limited to: (i) proliferation and maintenance regulatory T cells (e.g., when IL-2 MOD polypeptides are present); and (ii) proliferation and differentiation of effector and memory T cells (e.g., when IL-2 and a B7 MODs such as CD86 are present).

In some cases, a MAPP (e.g., particularly where the MAPP comprises class II MHC polypeptides) is contacted with an epitope-specific CD4⁺ T cell. In some cases, the CD4⁺ T cell is a ThI that produces, among other things, interferon gamma, and which may be stimulated to defend against intracellular bacteria, viruses and cancers, or is a target for inhibition in autoimmunity (e.g., inMS). In some cases, the CD4⁺ T cell is a Th2 cell that produces, among other things, IL-4. Th2 cells may be stimulated to aid in the differentiation of antibody production by B cells and inhibited to suppress autoimmune diseases, and asthma and other allergic diseases. In some cases, the CD4⁺ T cell is a Th17 cell that produces, among other things, IL-17, and which may be stimulated to assist a mammalian subject defend against gut pathogens (e.g., at the mucosal barrier), and which may be inhibited to suppress rheumatoid arthritis or psoriasis. In some cases, the CD4⁺ T cell is a Th9 cell that produces, among other things, IL-9, and which may be stimulated to assist a mammalian subject defend against, for example, helminths, and which may be inhibited to suppress its actions in autoimmue conditions such as multiple sclerosis. In some cases, the CD4⁺ T cell is a Tfh cell that produces, among other things, IL-21 and IL-4, and which may be stimulated to assist B cell produce antibody, and which may be inhibited to suppress autoimmune diseases such as asthma and allergies.

In some cases, the T cell being contacted with a MAPP is a regulatory T cell (Treg) that is CD4⁺, FOXP3⁺, and CD25⁺. Tregs can suppress autoreactive T cells. In some cases, a method activates Tregs, thereby reducing autoreactive T cell activity.

The present disclosure provides a method of increasing proliferation of Tregs, the method comprising contacting Tregs with a MAPP of the present disclosure, where the contacting increases proliferation of Tregs. The present disclosure provides a method of increasing the number of epitope specific Tregs in an individual, the method comprising administering to the individual a MAPP of the present disclosure, where the administering results in an increase in the number of Tregs specific to the epitope presented by the MAPP in the individual. For example, the number of Tregs can be increased by at least 5%, at least 10%, at least 15%, at least 20%, at least 25%, at least 30%, at least 40%, at least 50%, at least 60%, at least 70%, at least 80%, at least 90%, at least 2-fold, at least 2.5-fold, at least 5-fold, at least 10-fold, or more than 10-fold.

In some cases, the cell being contacted with a MAPP is a helper T cell, where contacting the helper T cell with a MAPP results in activation of the helper T cell. In some cases, activation of the helper T cell results in an increase in the activity and/or number of CD8⁺ cytotoxic T cells. In some cases (e.g., where a MAPP comprises a cancer epitope), a MAPP activates a CD8⁺ T cell response, e.g., a CD8⁺ T cell response to a cancer cell.

2. Methods of Detecting an Antigen-Specific T Cell

The present disclosure provides a method of detecting an antigen-specific T-cell. The methods comprise contacting a T cell with a MAPP either lacking a MOD or bearing a MOD with minimal affinity for its co-MOD; and detecting binding of the MAPP to the T cell. Binding of such a MAPP to the T cell indicates that the T cell is specific for the epitope present in the MAPP. In some cases, the MAPP comprises a detectable label. Suitable detectable labels include, but are not limited to, a radioisotope, a fluorescent polypeptide, or an enzyme that generates a fluorescent product, and an enzyme that generates a colored product. Where the MAPP comprises a detectable label, binding of the MAPP to the T cell is detected by detecting the detectable label.

In some cases, an MAPP comprises a detectable label suitable for use in in vivo imaging, e.g., suitable for use in positron emission tomography (PET), single photon emission tomography (SPECT), near infrared (NIR) optical imaging, x-ray imaging, computer-assisted tomography (CAT), or magnetic resonance imaging (MRI), or other in vivo imaging method. Examples of suitable labels for in vivo imaging include gadolinium chelates (e.g., gadolinium chelates with DTPA (diethylenetriamine penta-acetic acid), DTPA-bismethylamide (BMA), DOTA (dodecane tetraacetic acid), or HP-DO3A (1,4,7-tris(carboxymethyl)-10-(2′-hydroxypropyl)-1,4,7,10-tetraazacyclododecane)), iron chelates, magnesium chelates, manganese chelates, copper chelates, chromium chelates, iodine-based materials, and radionuclides.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), and the like. Other examples of fluorescent proteins include mHoneydew, mBanana, mOrange, dTomato, tdTomato, mTangerine, mStrawberry, mCherry, mGrapel, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes that may be employed as lables include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

In some cases, binding of the MAPP to the T cell is detected using a detectably labeled antibody specific for the MAPP. An antibody specific for the MAPP can comprise a detectable label such as a radioisotope, a fluorescent polypeptide, or an enzyme that generates a fluorescent product, or an enzyme that generates a colored product.

In some cases, the T cell being detected is present in a sample comprising a plurality of T cells. For example, a T cell being detected can be present in a sample comprising from 10 to 10⁹ T cells, e.g., from 10 to 10², from 10² to 10⁴, from 10⁴ to 10⁶, from 10⁶ to 10⁷, from 10⁷ to 10⁸, or from 10⁸ to 10⁹, or more than 10⁹, T cells.

3. Treatment Methods

The present disclosure provides treatment methods, the methods comprising administering to the individual a composition comprising an amount of a MAPP or higher order MAPP complex (e.g., duplex MAPP) of the present disclosure, or one or more nucleic acids or expression vectors encoding a MAPP that may assemble into a higher order complex (e.g., duplex MAPP), effective to selectively modulate the activity of an epitope-specific T cell in an individual and to treat the individual. In some cases, a treatment method comprises administering to an individual in need thereof a composition comprising one or more recombinant expression vectors comprising nucleotide sequences encoding a MAPP (e.g., a MAPP that may assemble into a higher order MAPP complex) of the present disclosure. In some cases, a treatment method comprises administering to an individual in need thereof one or more mRNA molecules comprising nucleotide sequences encoding a MAPP of the present disclosure. In some cases, a treatment method comprises administering to an individual in need thereof a MAPP or higher order MAPP complex (e.g., duplex MAPP) of the present disclosure. The conditions that can be treated include autoimmune disorders other than, or in addition to, T1D and/or celiac disease.

The present disclosure provides a method of selectively modulating the activity of an epitope-specific T cell in an individual, the method comprising administering to the individual an effective amount of a MAPP or higher order MAPP complex (e.g., duplex MAPP) of the present disclosure, or one or more nucleic acids (e.g., expression vectors; mRNA; etc.) comprising nucleotide sequences encoding the MAPP which may assemble into a higher order complex, where the MAPP or its complex selectively modulates the activity of the epitope-specific T cell in the individual. Selectively modulating the activity of an epitope-specific T cell can treat a disease or disorder in the individual. Thus, the present disclosure provides a treatment method comprising administering to an individual in need thereof an effective amount of a MAPP (e.g., a duplex MAPP of the present disclosure) sufficient to effect treatment of a disease or disorder other than, or in addition to, T1D and/or celiac disease.

In some cases, a MAPP comprises an inhibitory MOD polypeptide sequence, and the MAPP inhibits activity of the epitope-specific T cell (e.g., effector functions or proliferation). In some cases, the epitope is an epitope of an autoantigen (self-epitope), and a MAPP selectively inhibits the activity of a T cell specific for the epitope of the autoantigen.

The present disclosure provides a method of treating an autoimmune disorder in an individual, the method comprising administering to the individual a pharmaceutical composition comprising an effective amount of a MAPP or higher order MAPP complex such as a duplex MAPP of the present disclosure, or one or more nucleic acids comprising nucleotide sequences encoding the MAPP (which may assemble into a higher order complex such as an duplex MAPP), wherein the MAPP (e.g., a MAPP comprising an epitope of an autoantigen), and wherein the MAPP or higher order MAPP complex (e.g., duplex MAPP) comprises an inhibitory MOD (e.g., FasL and/or PDL1). In some cases an “effective amount” of a MAPP or higher order MAPP complex is an amount that, when administered in one or more doses to an individual in need thereof, reduces the number of self-reactive (i.e., reactive with an epitope of an autoantigen) CD4+ and/or CD8+ T cells by at least 10%, at least 15%, at least 20%, at least 25%, at least 30%, at least 40%, at least 50%, at least 60%, at least 70%, at least 80%, at least 90%, or at least 95%, compared to number of self-reactive T cells in the individual before administration of the MAPP, or in the absence of MAPP administration. In some cases, an “effective amount” of a MAPP is an amount that, when administered in one or more doses to an individual in need thereof, reduces production of Th1 cytokines (e.g., IL-2, IL-10, and TNF-alpha/beta) in the individual. In some cases, an “effective amount” of a MAPP is an amount that, when administered in one or more doses to an individual in need thereof, reduces production of Th2 cytokines (e.g., IL-4, IL-5, and IL-13) in the individual or a tissue of an individual. In some cases, an “effective amount” of a MAPP is an amount that, when administered in one or more doses to an individual in need thereof, reduces production of Th17 cytokines (e.g., IL-17A, IL-17F, and IL-22) in the individual. In some cases an “effective amount” of a MAPP is an amount that, when administered in one or more doses to an individual in need thereof, ameliorates one or more symptoms associated with an autoimmune disease in the individual. In some instances, the MAPP reduces the number or activity of CD4⁺ self-reactive T cells (i.e., the number of CD4+ T cells reactive with a TlD-associated antigen), which in turn leads to a reduction in CD8⁺ self-reactive T cells. In some instances, the MAPP increases the number or activity (e.g., IL-10 and/or TGF-β production) of CD4⁺ Tregs specific for an epitope presented by the MAPP, which in turn may reduce the number or activity of CD4⁺ self-reactive T cells, B cells. and/or CD8⁺ T self-reactive T cells specific for that epitope.

In some cases, the MOD is an activating polypeptide, and the MAPP with its associated epitope activates an epitope-specific T cell that recognizes a cancer or pathogen specific antigen (e.g., a viral or bacterial antigen). In some cases, the T cells are T-helper cells (CD4⁺ cells) or NK-T cells which may also indirectly influence CD 8⁺ Treg cells (see e.g., Nakamura et al., J. Immnnol. 171(3) 1266-1271 (2003) and cytotoxic T cells (CD8⁺ cells). In some cases, a MAPP with its associated epitope increases the activity of a T cell specific for a cancer cell expressing the epitope (e.g., T-helper cells and/or NK-T cells). Activation of CD4⁺ T cells can include increasing proliferation of CD4⁺ T cells and/or inducing or enhancing the release of cytokines by CD4⁺ T cells. Activation of NK-T cells and/or CD8⁺ cells can include: increasing proliferation of NK-T cells and/or CD8⁺ cells; and/or inducing release of cytokines such as interferon γ by NK-T cells and/or CD8⁺ cells.

In some cases, e.g., where MAPP comprises an inhibitory MOD (e.g., FasL, and the like), the MAPP may reduce the proliferation and/or activity of a Treg (e.g., FoxP3⁺, CD4⁺ Treg cells).

As noted above, in some cases, in carrying out a subject treatment method, a MAPP (e.g., a duplex MAPP or other higher order MAPP complex) is administered to an individual in need thereof, as the polypeptide per se. In other instances, in carrying out a subject treatment method, one or more nucleic acids comprising nucleotide sequences encoding a MAPP is/are administering to an individual in need thereof. Thus, in other instances, one or more nucleic acids of the present disclosure, e.g., one or more recombinant expression vectors of the present disclosure, is/are administered to an individual in need thereof.

A MAPP or higher order MAPP complex (e.g., duplex MAPP), or one or more nucleic acids encoding such molecules may be administered alone or with one or more additional therapeutic agents or drugs. The therapeutic agents may be administered before, during, or subsequent to MAPP or higher order MAPP complex (e.g., duplex MAPP) or nucleic acids encoding such molecules. When the additional therapeutic agents are administered with a composition or formulation comprising a MAPP or higher order MAPP complex (e.g., duplex MAPP) or nucleic acids encoding such molecules, the therapeutic agent may be administered concurrently with the MAPP. Alternatively, the therapeutic agents may be co-administered with the MAPP as part of a formulation or composition comprising the MAPP or higher order MAPP complex (e.g., duplex MAPP).

Suitable therapeutic agents or drugs that may be administered with a MAPP or higher order MAPP complex include virtually any therapeutic agent, including small molecule therapeutics (e.g., less than 2,000 Daltons in molecular weight) approved by the U.S. Food and Drug Administration, and/or listed in the 2020 U.S. Pharmacopeia or National Formulary. In an embodiment, those therapeutic agents or drugs are less than 1,000 molecular weight. Suitable drugs include antibiotics, chemotherapeutic (antineoplastic), anti-fungal, or anti-helminth agents and the like (e.g., sulfasalazine, azathioprine, cyclophosphamide, leflunomide; methotrexate, antimalarials, D-penicillamine, cyclosporine). Suitable chemotherapeutics may be alkylating agents, cytoskeletal disruptors (taxanes), epothilones, histone deacetylase inhibitors, topoisomerase I inhibitors, topoisomerase II inhibitors, kinase inhibitors, nucleotide analog or precursor analogs, peptide antineoplastic antibiotics (e.g., bleomycin or actinomycin), platinum-based agents, retinoids, or vinca alkaloids. Suitable drugs also include non-steroidal anti-inflammatory drugs and glucocorticoids, and the like.

In an embodiment, a suitable therapeutic agent that may be administered with a MAPP or higher order MAPP complex comprises an anti-TGF-β antibody, such as Metelimumab (CAT192) directed against TGF-β1 and/or Fresolimub directed against TGF-β1 and TGF-β2, or a TGF-β trap (e.g., Cablivi® caplacizumab-yhdp). Such antibodies would, as a generality, not be administered in conjunction with a MAPP or higher order MAPP complex (e.g., a duplexed MAPP) that comprise a sequence to which the antibodies bind such as a TGF-β1 or TGF-β2 MOD.

In an embodiment, a suitable therapeutic agent that may be administered with a MAPP or higher order MAPP complex comprises one or more antibodies directed against: B lymphocyte antigens (e.g., ibritumomab tiuxetan, obinutuzumab, ofatumumab, rituximab to CD20, brentuximab vedotin directed against CD30, and alemtuzumab to CD52); EGFR (e.g., cetuximab, panitumumab, and necitumumab); VEGF (e.g., bevacizumab); VEGFR2 (e.g., ramucirumab); HER2 (e.g., pertuzumab, trastuzumab, and ado-trastuzumab); PD-1 (e.g., nivolumab and pembrolizumab targeting a check point inhibition); RANKL (e.g., denosumab); CTLA-4 (e.g., ipilimumab targeting check point inhibition); IL-6 (e.g., siltuximab); disialoganglioside (GD2), (e.g., dinutuximab) disialoganglioside (GD2); CD38 (e.g., daratumumab); SLAMF7 (Elotuzumab); both EpCAM and CD3 (e.g., catumaxomab); or both CD19 and CD3 (blinatumomab). Such antibodies would, as a generality, not be administered in conjunction with a MAPP or higher order MAPP complex (e.g., a duplexed MAPP) that comprise a sequence to which any of the administered antibodies bind, or which may block the action of a MOD present in the administered MAPP.

In an embodiment, a suitable therapeutic agent that may be administered with a MAPP or higher order MAPP complex comprises an antibiotic, anti-fungal, and/or anti-helminth agent.

In an embodiment, a suitable therapeutic agent that may be administered with a MAPP or higher order MAPP complex comprises one or more chemotherapeutic agents. Such therapeutic agents may be selected from: alkylating agents, cytoskeletal disruptors (taxanes), epothilones, histone deacetylase inhibitors, topoisomerase I inhibitors, topoisomerase II inhibitors, kinase inhibitors, nucleotide analog or precursor analogs, peptide antineoplastic antibiotics (e.g., bleomycin or actinomycin), platinum-based agents, retinoids, or vinca alkaloids and their derivatives. In an embodiment, the chemotherapeutic agents are selected from actinomycin all-trans retinoic acid, azacytidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.

The present disclosure provides treatment methods, the methods comprising administering to the individual an amount of a MAPP or higher order MAPP complex of the present disclosure, or one or more nucleic acids or expression vectors encoding the MAPP, effective to selectively modulate the activity of an epitope-specific T cell in an individual and to treat the individual. In some cases, a treatment method comprises administering to an individual in need thereof one or more recombinant expression vectors comprising nucleotide sequences encoding a MAPP or higher order MAPP complex of the present disclosure. In some cases, a treatment method comprises administering to an individual in need thereof one or more mRNA molecules comprising nucleotide sequences encoding a MAPP or higher order MAPP complex of the present disclosure. In some cases, a treatment method comprises administering to an individual in need thereof a MAPP or higher order MAPP complex of the present disclosure.

The present disclosure also provides treatment methods, the methods comprising administering to the individual a pharmaceutical composition comprising an effective amount of a MAPP of the present disclosure, or one or more nucleic acids or expression vectors encoding the MAPP optionally bearing an immunoglobulin sequence that can support complement-dependent cytotoxicity (CDC) or antibody-dependent cell cytotoxicity (ADCC). Such MAPPs can selectively engage with an epitope-specific T cell in an individual in order to treat the individual, (e.g., by depleting epitope-specific T cells by inducing cell lysis through activation of CDC, and/or ADCC).

The present disclosure provides a method of selectively modulating the activity of an epitope-specific T cell in an individual, the method comprising administering to the individual an effective amount of a MAPP or higher order MAPP complex of the present disclosure, or one or more nucleic acids (e.g., expression vectors; mRNA; etc.) comprising nucleotide sequences encoding the MAPP or higher order MAPP complex, which selectively modulates the activity of the epitope-specific T cell in the individual. Selectively modulating the activity of an epitope-specific T cell can treat a disease or disorder in the individual. Thus, the present disclosure provides a treatment method comprising administering to an individual in need thereof an effective amount of a MAPP or higher order MAPP complex in order to treat a disease or disorder other than, or in addition to, TID and/or celiac disease.

In some cases, the MOD is an inhibitory polypeptide, and a MAPP or higher order MAPP complex inhibits activity of the epitope-specific T cell. In some cases, the epitope is an epitope of an autoantigen, and a MAPP or higher order MAPP complex selectively inhibits the activity of a T cell specific for the epitope of the autoantigen.

The present disclosure provides a method of treating an autoimmune disorder in an individual, the method comprising administering to the individual an effective amount of a MAPP or higher order MAPP complex that comprises an epitope of an autoantigen and an inhibitory MOD (or one or more nucleic acids comprising nucleotide sequences encoding those molecules. In some cases, an “effective amount” of a MAPP or higher order MAPP complex is an amount that, when administered in one or more doses to an individual in need thereof, reduces the number of self-reactive T cells specific to the epitope presented by the MAPP or higher order MAPP complex by at least 10%, at least 15%, at least 20%, at least 25%, at least 30%, at least 40%, at least 50%, at least 60%, at least 70%, at least 80%, at least 90%, or at least 95%, compared to number of those self-reactive T cells in the individual before or in the absence of administration of the MAPP or higher order MAPP complex. In some cases, an “effective amount” of a MAPP or higher order MAPP complex is an amount that, when administered in one or more doses to an individual in need thereof, reduces production of Th2 cytokines in the individual. In some cases, an “effective amount” of a MAPP or higher order MAPP complex is an amount that, when administered in one or more doses to an individual in need thereof, ameliorates one or more symptoms associated with an autoimmune disease in the individual. In some instances, the MAPP or higher order MAPP complex reduces the number of CD4⁺ self-reactive T cells specific to the epitope presented by those molecules, which may lead to a reduction in antibody production and which may in turn may lead to a reduction in CD8⁺ self-reactive T cells. In some instances, a MAPP or higher order MAPP complex increases the number of CD4⁺ Tregs specific to the epitope presented by those molecules, which in turn reduces the number of CD4⁺ self-reactive T cells and may subsequently reduce the production of antibodies.

In some cases, the MOD is an activating polypeptide, and the MAPP with its associated epitope activates an epitope-specific T cell that recognizes a cancer or pathogen specific antigen (e.g., a viral or bacterial antigen). In some cases, the T cells are CD 4⁺ T-helper cells or NK-T cells. In some cases MAPP or higher order MAPP complex with its associated epitope increases the activity of a T cell specific for a cancer cell expressing the epitope (e.g., CD 4⁺ T-helper cells (CD4⁺ cells) and/or NK-T cells). Activation of CD4⁺ T cells can include increasing proliferation of CD4⁺ T cells and/or inducing or enhancing release cytokines by CD4⁺ T cells. Activation of NK-T cells can include: increasing proliferation of NK-T cells and/or inducing release of cytokines such as interferon γ by NK-T cells.

In some cases where a MAPP or higher order MAPP complex comprises an inhibitory MOD (e.g., PD-L1, FasL, and the like) it may reduce the proliferation and/or activity of a regulatory T (Treg) cell (e.g., FoxP3⁺, CD4⁺ T cells) specific to the epitope presented by the MAPP or higher order MAPP complex.

As noted above, in some cases, in carrying out a subject treatment method, a MAPP is administered to an individual in need thereof, as the polypeptide per se. In other instances, in carrying out a subject treatment method, one or more nucleic acids comprising nucleotide sequences encoding a MAPP is/are administering to an individual in need thereof. Thus, in other instances, one or more nucleic acids of the present disclosure, e.g., one or more recombinant expression vectors of the present disclosure, is/are administered to an individual in need thereof.

4. Methods of Selectively Delivering a MOD

The present disclosure provides a method of delivering a MOD polypeptide such as IL-2, 4-1BBL, Fas-L, PD-L1, TGF-β, or a reduced-affinity variant of any thereof (e.g., PD-L1 and/or an IL-2 variant disclosed herein) to a selected T cell or a selected T cell population, e.g., in a manner such that a TCR specific for a given epitope is targeted. The present disclosure thus provides a method of delivering a MOD polypeptide such as a PD-L1 polypeptide, or a reduced-affinity variant of a naturally occurring MOD polypeptide such as a PD-L1 variant, selectively to a target T cell bearing a TCR specific for the peptide epitope sequence present in a MAPP or higher order MAPP complex (e.g., duplex MAPP). The method comprises contacting a population of T cells with a MAPP or higher order MAPP complex (e.g., duplex MAPP). The population of T cells can be a mixed population that comprises: i) the target T cell with a TCR specific to a target epitope; and ii) non-target T cells that are not specific for the target epitope presented by the peptide epitope (e.g., T cells that are specific for an epitope(s) other than the epitope to which the epitope-specific T cell binds). The epitope-specific T cell is specific for the peptide epitope present in the MAPP or higher order MAPP complex, and binds to the peptide MHC complex provided by the MAPP or higher order MAPP complex. Contacting the population of T cells with the MAPP or higher order MAPP complex delivers the MOD polypeptide (e.g., PD-L1 or a reduced-affinity variant of PD-L1) selectively to the T cell(s) that are specific for the epitope present in the MAPP or higher order complex.

Thus, the present disclosure provides a method of delivering a MOD polypeptide such as PD-L1, or a reduced-affinity variant of a naturally occurring MOD polypeptide such as a PD-L1 variant disclosed herein, or a combination of both, selectively to a target T cell selective for the epitope presented by the peptide epitope as presented by the MAPP. Similarly, the disclosure provides a method of delivering an IL-2, MOD polypeptide or a reduced-affinity variant of a naturally occurring IL-2 MOD polypeptide such as disclosed herein, or a combination of both, to a target T cell that is selective for the epitope presented by the peptide epitope as presented by the MAPP. In some cases, the IL-2 MOD bears a substitution at position H16 and/or F42 (e.g., H16 and F42 such as H16A and F42A) (see supra SEQ ID NO:166).

For example, a MAPP or higher order MAPP complex (e.g., duplex MAPP) is contacted with a population of T cells comprising: i) target T cells that are specific for the epitope present in the MAPP or a higher order MAPP complex; and ii) non-target T cells, e.g., a T cells that are specific for a second epitope(s) that is not the epitope present in the MAPP or a higher order MAPP complex. Contacting the population results in substantially selective delivery of the MOD polypeptide(s) (e.g., naturally-occurring or variant MOD polypeptide) to the target T cell. Less than 50%, less than 40%, less than 30%, less than 25%, less than 20%, less than 15%, less than 10%, less than 5%, or less than 4%, 3%, 2% or 1%, of the MAPP or higher order MAPP complex (e.g., duplex MAPP) may bind to non-target T cells and, as a result, the MOD polypeptide (e.g., PD-L1 or PD-L1 variant) is selectively delivered to target T cell (and accordingly, not effectively delivered to the non-target T cells).

The population of T cells to which a MOD and/or variant MOD is selectively delivered may be in vivo. In some cases, the population of T cells to which a MOD and/or variant MOD is selectively delivered is in vitro.

In some cases, the population of T cells to which a MOD and/or variant MOD is selectively delivered is in vitro. For example, a mixed population of T cells is obtained from an individual, and is contacted with a MAPP or higher order MAPP complex (e.g., duplex MAPP) in vitro. Such contacting, which can comprise single or multiple exposures of the T cells to a defined dose(s) and/or exposure schedule(s) in the context of in vitro cell culture, can be used to determine whether the mixed population of T cells includes T cells that are specific for the epitope presented by the MAPP or higher order MAPP complex. The presence of T cells that are specific for the epitope of the MAPP or higher order MAPP complex can be determined by assaying a sample comprising a mixed population of T cells, which population of T cells comprises T cells that are not specific for the epitope (non-target T cells) and may comprise T cells that are specific for the epitope (target T cells). Known assays can be used to detect the desired modulation of the target T cells, thereby providing an in vitro assay that can determine whether a particular MAPP or higher order MAPP complex possesses an epitope that binds to T cells present in the individual, and thus whether the MAPP or higher order complex has potential use as a therapeutic composition for that individual. Suitable known assays for detection of the desired modulation (e.g., activation/proliferation or inhibition/suppression) of target T cells include, e.g., flow cytometric characterization of T cell phenotype, numbers, and/or antigen specificity. Such an assay to detect the presence of epitope-specific T cells, e.g., a companion diagnostic, can further include additional assays (e.g., effector cytokine ELISpot assays) and/or appropriate controls (e.g., antigen-specific and antigen-nonspecific multimeric peptide-HLA staining reagents) to determine whether the MAPP or higher order MAPP complex is selectively binding, modulating (activating or inhibiting), and/or expanding the target T cells. Thus, for example, the present disclosure provides a method of detecting, in a mixed population of T cells obtained from an individual, the presence of a target T cell that binds an epitope of interest, the method comprising: a) contacting in vitro the mixed population of T cells with a MAPP or higher order MAPP complex comprising an epitope of the present disclosure; and b) detecting modulation (activation or inhibition) and/or proliferation of T cells in response to said contacting, wherein modulation of and/or proliferation of T cells indicates the presence of the target T cell. Alternatively, and/or in addition, if activation and/or expansion (proliferation) of the desired T cell population is obtained using a MAPP or higher order MAPP complex (e.g., a duplex MAPP), then all or a portion of the population of T cells comprising the activated/expanded T cells can be administered back to the individual as a therapy.

In some instances, the population of T cells is in vivo in an individual. In such instances, a method of the present disclosure for selectively delivering a MOD polypeptide (e.g., PD-L1 or a reduced-affinity PD-L1) to an epitope-specific T cell comprises administering the MAPP or higher order MAPP complex (e.g., duplex MAPP) to the individual.

In some instances, the epitope-specific T cell to which a MOD polypeptide sequence (e.g., wild type or reduced affinity IL-2 and/or PD-L1 MOD) is being selectively delivered is referred to herein is a target regulatory T cell (Treg) that may inhibit or suppresses activity of an autoreactive T cell.

I. Dosages

A suitable dosage can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. A MAPP (whether as a single heterodimer or, as described above, as a higher order complex such as a duplex MAPP) may be administered in amounts between 1 ng/kg body weight and 20 mg/kg body weight per dose; for example from 0.1 μg/kg body weight to 1.0 mg/kg body weight, from 0.1 mg/kg body weight to 0.5 mg/kg body weight, from 0.5 mg/kg body weight to 1 mg/kg body weight, from 1.0 mg/kg body weight to 5 mg/kg body weight, from 5 mg/kg body weight to 10 mg/kg body weight, from 10 mg/kg body weight to 15 mg/kg body weight, and from 15 mg/kg body weight to 20 mg/kg body weight. Doses below 0.1 mg/kg body weight or above 20 mg/kg are envisioned, especially considering the aforementioned factors. Amounts thus include from about 0.1 mg/kg body weight to about 0.5 mg/kg body weight, from about 0.5 mg/kg body weight to about 1 mg/kg body weight, from about 1.0 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, and above about 20 mg/kg body weight.

Those of skill will readily appreciate that dose levels can vary as a function of the MAPP or higher order MAPP complex (e.g., duplex MAPP), the severity of the symptoms and the susceptibility of the subject to side effects. Preferred dosages for a given compound are readily determinable by those of skill in the art by a variety of means.

In some cases, multiple doses of a MAPP or higher order MAPP complex (e.g., duplex MAPP) are administered. The frequency of administration of a MAPP or higher order MAPP complex (e.g., duplex MAPP) can vary depending on any of a variety of factors, e.g., severity of the symptoms, patient response, etc. For example, in some cases, a MAPP or higher order MAPP complex (e.g., duplex MAPP) is administered once per month, less frequently than once per month, e.g., once every 6 weeks, once every two months, once every three months, or more frequently than once per month, e.g., twice per month, three times per month, every other week (qow), one every three weeks, once every four weeks, once per week (qw), twice per week (biw), three times per week (tiw), four times per week, five times per week, six times per week, every other day (qod), daily (qd), twice a day (qid), or three times a day (tid).

The duration of administration of a MAPP, e.g., the period of time over which a MAPP is administered, can vary, depending on any of a variety of factors, e.g., patient response, etc. For example, a MAPP can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more, including continued administration for the patient's life.

Where treatment is of a finite duration, following successful treatment, it may be desirable to have the patient undergo periodic maintenance therapy to prevent the recurrence of the disease state, wherein a MAPP is administered in maintenance doses, ranging from those recited above, i.e., 0.1 mg/kg body weight to about 0.5 mg/kg body weight, from about 0.5 mg/kg body weight to about 1 mg/kg body weight, from about 1.0 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, and above about 20 mg/kg body weight. The periodic maintenance therapy can be once per month, once every two months, once every three months, once every four months, once every five months, once every six months, or less frequently than once every six months.

J. Routes of Administration

A MAPP or higher order MAPP complex (e.g., a duplex MAPP), or a nucleic acid or recombinant expression vectors comprising nucleic acids encoding one or more polypeptides of a MAPP or its higher order complexes, is administered to an individual using any available method and route suitable for drug delivery, including in vivo and in vitro methods, as well as systemic and localized routes of administration. A MAPP or higher order MAPP complex can be administered to a host using any available conventional methods and routes suitable for delivery of conventional drugs, including systemic or localized routes. In general, routes of administration contemplated for use in a method include, but are not necessarily limited to, enteral, parenteral, and inhalational routes.

Conventional and pharmaceutically acceptable routes of administration include intramuscular, intratracheal, subcutaneous, intradermal, topical application, intravenous, intraarterial, intralymphatic, rectal, nasal, oral, intratumoral, peritumoral, and other enteral and parenteral routes of administration. Of these, intravenous, intramuscular and subcutaneous may be more commonly employed. MAPPS and their higher order complexes, nucleic acids and expression vectors encoding them may be administered, for example, intravenously. Routes of administration may be combined, if desired, or adjusted depending upon, for example, the MAPP or higher order MAPP complex (e.g., duplex MAPP) and/or the desired effect. A MAPP or higher order MAPP complex can be administered in a single dose or in multiple doses.

A MAPP or higher order MAPP complex (e.g., a duplex MAPP), or a nucleic acid or recombinant expression vectors comprising nucleic acids encoding one or more polypeptides of a MAPP or its higher order complexes may also be contacted with cells in vitro. The cells subject to such in vitro treatment and/or their progeny, may then be administered to a patient or subject (e.g., the subject from which the cells treated in vitro were obtained.

K. Subjects Suitable for Treatment

Subjects suitable for treatment include those with a cancer, infectious diseases (e.g., including those with viral, bacterial, and/or mycoplasma causative agents), allergic reactions, and/or autoimmune diseases other than, or in addition to, celiac disease and/or TID.

Subjects suitable for treatment who have a cancer include, but are not limited to, individuals who have been provided other treatments for the cancer but who failed to respond to the treatment. Cancers that can be treated with a method include, but are not limited to, those displaying any of the cancer epitopes recited herein including, but not limited to peptide epitopes of AFP, WT-1, HPV and HBV.

Subjects suitable for treatment may also include individuals who have an infectious disease include, but are not limited to, individuals who have been provided other treatments for the infectious disease but who failed to respond to the treatment. Infectious diseases that can be treated with a method include, but are not limited to, those having an infectious agent recited herein including, but not limited to, EBV, HPV and HBV epitopes.

Subjects suitable for treatment also include individuals who have an allergy include, but are not limited to, individuals who have been provided other treatments for the allergy but who failed to respond to the treatment. Allergic conditions that can be treated with a method include, but are not limited to, those resulting from exposure to nuts (e.g., tree and/or peanuts), pollen, and insect venoms (e.g., bee and/or wasp venom antigens).

Subjects suitable for treatment include those with an autoimmune disease or a genetic disposition to develop an autoimmune disease including a family history of an autoimmune disease (e.g., a grandparent, parent, or sibling with the autoimmune disease. The genetic disposition to certain autoimmune disease, and the serotype/haplotype of some individuals either with or predisposed to those diseases is set forth in for example, FIG. 33. Subjects suitable for treatment who have an autoimmune disease include, but are not limited to, individuals who have been provided other treatments for the autoimmune disease but who failed to respond to the treatment. Autoimmune diseases that can be treated with a method include, but are not limited to, Addison's disease, alopecia areata, ankylosing spondylitis, autoimmune encephalomyelitis, autoimmune hemolytic anemia, autoimmune hepatitis, autoimmune-associated infertility, autoimmune thrombocytopenic purpura, bullous pemphigoid, Crohn's disease, Goodpasture's syndrome, glomerulonephritis (e.g., crescentic glomerulonephritis, proliferative glomerulonephritis), Grave's disease, Hashimoto's thyroiditis, mixed connective tissue disease, multiple sclerosis, myasthenia gravis (MG), Pemphigus (e.g., Pemphigus vulgaris), pernicious anemia, polymyositis, psoriasis, psoriatic arthritis, rheumatoid arthritis, scleroderma, Sjögren's syndrome, systemic lupus erythematosus (SLE), vasculitis, and vitiligo. See e.g., FIG. 33

### V. CERTAIN ASPECTS

Certain aspects, including embodiments/aspects of the present subject matter described above, may be beneficial alone or in combination, with one or more other aspects recited hereinbelow. In addition, while the present subject matter has been disclosed with reference to certain aspects recited below and in the claims, numerous modifications, alterations, and changes to the described aspects/embodiments are possible without departing from the sphere and scope of the present disclosure.

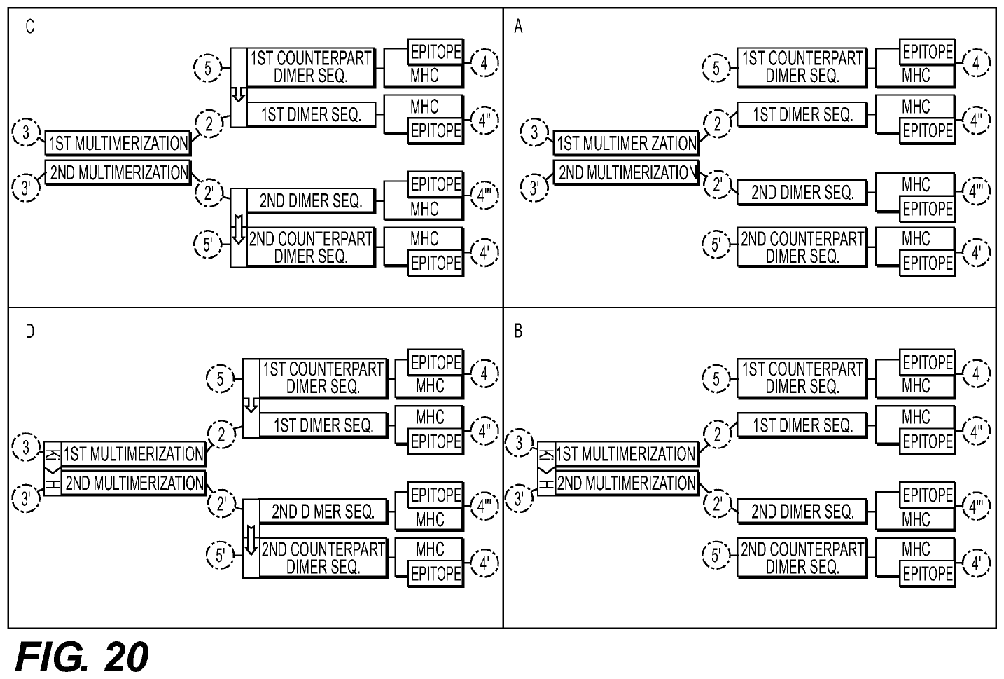

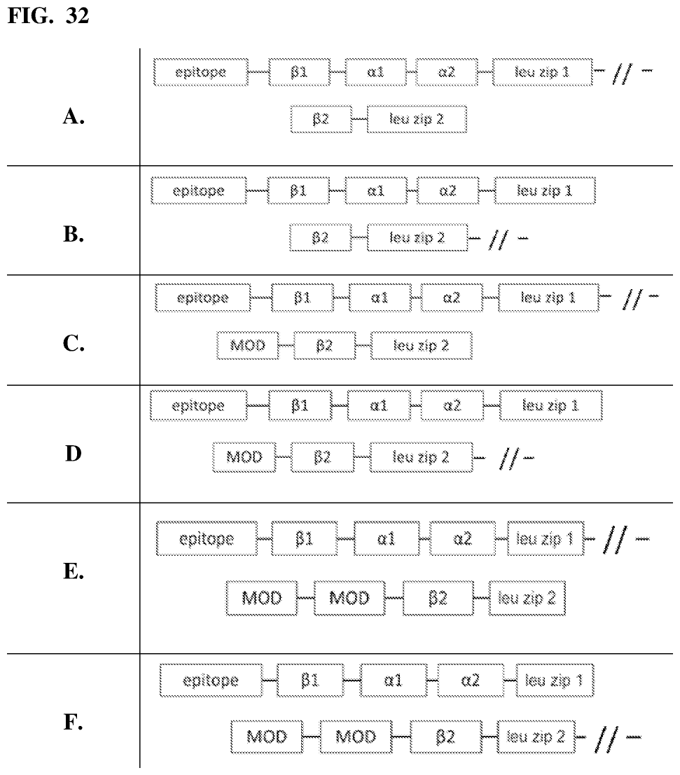

Accordingly, it is intended that the present disclosure not be limited to the described embodiments, aspects, and claims, but that it has the full scope defined by the language of this disclosure and equivalents thereof.
1. A multimeric antigen-presenting polypeptide complex (MAPP) comprising:
   (i) a framework polypeptide comprising (e.g., from N-terminus to C-terminus) a dimerization sequence and a multimerization sequence;
   (ii) a dimerization polypeptide comprising a counterpart dimerization sequence complementary to the dimerization sequence of the framework polypeptide, and dimerizing therewith through covalent (e.g., disulfide bonds) and/or non-covalent interactions to form a MAPP heterodimer; and
   (iii) at least one (e.g., at least two) presenting sequence and/or presenting complex,
   wherein each presenting sequence comprises a peptide epitope, and MHC class II α1, α2, β1, and β2 domain polypeptide sequences;
   wherein each presenting complex comprises a presenting complex 1^{st} sequence and a presenting complex 2^{nd} sequence that together comprise an epitope and MHC class II α1, α2, β1, and β2 domain polypeptide sequences, where the epitope is part of the presenting complex 1^{st} sequence or presenting complex 2^{nd} sequence along with at least one of the α1, α2, β1, and β2 polypeptide sequences;
   wherein at least one or both of the dimerization polypeptide and/or the framework polypeptide (e.g., either the framework polypeptide, dimerization polypeptide, or both polypeptides) comprises a presenting sequence or a presenting complex 1st sequence (e.g., located on the N-terminal side of the framework polypeptide's dimerization sequence, or the N-terminal side of the dimerization polypeptide' counterpart dimerization sequence);
   wherein optionally at least one (e.g., one, two, or more) of the framework polypeptide, or dimerization peptide (including the presenting sequence(s) or presenting complex(s)) comprises one, two, three or more independently selected MOD and/or variant MOD polypeptide sequences (e.g., located at the N-terminus or C-terminus of the dimerization polypeptide or framework polypeptide, and/or on the C-terminal side of the dimerization sequences);
   wherein the framework polypeptide, dimerization polypeptide, presenting sequence, presenting complex 1^{st} sequence and/or presenting complex 2^{nd} sequence optionally comprise one or more independently selected linker sequences. (See, e.g., FIGS. 1A and 1B).
      It is understood that the dimerization sequence and multimerization sequences are different polypeptide sequences and do not bind in any substantial manner to each other, e.g., the framework polypeptides do not, to any substantial extent, form hair pin structures, self-polymerize, or self-aggregate. Similarly, this aspect may be subject to the proviso that neither the dimerization sequence nor the multimerization sequence of the framework polypeptide comprises an MHC-Class II polypeptide sequence having at least 85% (e.g., 90%, 95% or 98%) sequence identity to at least 20 (e.g., at least 30, 40, 50, 60 or 70) contiguous aas of a MHC-Class II polypeptide in any of FIGS. 4 through 18B. It is also understood that none of the α1, α2, β1 and β2 domain polypeptide sequences include a transmembrane domain, or a portion thereof, that will anchor the MAPP in a cell membrane.
2. The MAPP of aspect 1, wherein the MHC class II α1 and α2 domain polypeptide sequences comprise human class II α1 and α2 domain polypeptide sequences selected from HLA DR alpha (DRA), DM alpha (DMA), DO alpha (DOA), DP alpha 1 (DPA1), DQ alpha 1 (DQA1), and DQ alpha 2 (DQA2) α1 and α2 domain polypeptide sequences.
3. The MAPP of aspect 1 or 2, wherein the MHC class II β1 and β2 domain polypeptide sequences comprises human class β1 and β2 domain polypeptide sequences selected from a HLA DR beta 1 (DRB1), DR beta 3 (DRB3), DR beta 4 (DRB4), DR beta 5 (DRB5), DM beta (DMB), DO beta (DOB), DP beta 1 (DPB1), DQ beta 1 (DQB1), and DQ beta 2 (DQB2) β1 and β2 domain polypeptide sequences.
4. The MAPP of any preceding aspect, wherein at least one presenting sequence or presenting complex (e.g., at least two, at least three, or all presenting sequences and/or complexes) comprises:
   an α1 and α2 domain polypeptide sequences each having at least 90% (e.g., at least 95% or 98%) or 100% sequence identity to all or at least about 50 (e.g., at least about 60, 70, 80, 85, or 90) contiguous aas of an HLA DR alpha (DRA), DM alpha (DMA), DO alpha (DOA), DP alpha 1 (DPA1), DQ alpha 1 (DQA1), or DQ alpha 2 (DQA2) α1 and/or α2 domain polypeptide sequence provided in any of FIG. 4, 9, 11, 13, 15, or 16; and
   a β1 and β2 domain polypeptide sequences each having at least 90% (e.g., at least 95% or 98%) or 100% sequence identity to all or at least about 50 (e.g., at least about 60, 70, 80, 85, or 90) contiguous aas of an HLA DR beta 1 (DRB1), DR beta 3 (DRB3), DR beta 4 (DRB4), DR beta 5 (DRB5), DM beta (DMB), DO beta (DOB), DP beta 1 (DPB1), DQ beta 1 (DQB1), or DQ beta 2 (DQB2) β1 and β2 domain polypeptide sequences provided in any of FIG. 5, 6, 7, 8, 10, 12, 14, 17 or 18.
5. The MAPP of aspect 4, wherein at least one presenting sequence or presenting complex (e.g., at least two or all presenting sequences and/or complexes) comprises:
   a α1 and α2 domain polypeptide sequences each having at least 90% (e.g., at least 95% or 98%) or 100% sequence identity to all or at least about 50 (e.g., at least about 60, 70, 80, 85, or 90) contiguous aas of a HLA DR alpha (DRA) α1 and/or α2 domain polypeptide sequence provided in FIG. 4; and
   a β1 and β2 domain polypeptide sequences each having at least 90% (e.g., at least 95% or 98%) or 100% sequence identity to all or at least about 50 (e.g., at least about 60, 70, 80, 85, or 90) contiguous aas of a HLA DR beta 1 (DRB1), DR beta 3 (DRB3), DR beta 4 (DRB4), or DR beta 5 (DRB5) β1 and/or β2 domain polypeptide sequences provided in any one of FIG. 5, 6, 7, or 8.
6. The MAPP of aspect 4, wherein at least one presenting sequence or presenting complex (e.g., at least two or all presenting sequences and/or complexes) comprises:
   a α1 and α2 domain polypeptide sequences each having at least 90% (e.g., at least 95% or 98%) or 100% sequence identity to all or at least about 50 (e.g., at least about 60, 70, 80, 85, or 90) contiguous aas of a HLA DR alpha (DRA) α1 and/or α2 domain polypeptide sequence provided in FIG. 4; and
   a β1 and β2 domain polypeptide sequences each having at least 90% (e.g., at least 95% or 98%) or 100% sequence identity to all or at least about 50 (e.g., at least about 60, 70, 80, 85, or 90) contiguous aas of a HLA DR beta 1 (DRB1) β1 and/or β2 domain polypeptide sequences provided in FIG. 5.
7. The MAPP of aspect 4, wherein at least one presenting sequence or presenting complex (e.g., at least two or all presenting sequences and/or complexes) comprises:
   a α1 and α2 domain polypeptide sequences each having at least 90% (e.g., at least 95% or 98%) or 100% sequence identity to all or at least about 50 (e.g., at least about 60, 70, 80, 85, or 90) contiguous aas of a HLA DR alpha (DRA) α1 and/or α2 domain polypeptide sequence provided in FIG. 4; and
   a β1 and β2 domain polypeptide sequences each having at least 90% (e.g., at least 95% or 98%) or 100% sequence identity to all or at least about 50 (e.g., at least about 60, 70, 80, 85, or 90) contiguous aas of a HLA DR beta 3 (DRB3), DR beta 4 (DRB4), and DR beta 5 (DRB5) β1 and/or β2 domain polypeptide sequences provided in any of FIG. 6, 7, or 8.
8. The MAPP of aspect 4, wherein at least one presenting sequence or presenting complex (e.g., at least two or all presenting sequences and/or complexes) comprises:
   a α1 and α2 domain polypeptide sequences each having at least 90% (e.g., at least 95% or 98%) or 100% sequence identity to all or at least about 50 (e.g., at least about 60, 70, 80, 85, or 90) contiguous aas of a HLA DM alpha (DMA) α1 and/or α2 domain polypeptide sequence provided of FIG. 9; and
   a β1 and β2 domain polypeptide sequences each having at least 90% (e.g., at least 95% or 98%) or 100% sequence identity to all or at least about 50 (e.g., at least about 60, 70, 80, 85, or 90) contiguous aas of a HLA DM beta (DMB) β1 and/or β2 domain polypeptide sequences provided in FIG. 10.
9. The MAPP of aspect 4, wherein at least one presenting sequence or presenting complex (e.g., at least two or all presenting sequences and/or complexes) comprises:
   a α1 and α2 domain polypeptide sequences each having at least 90% (e.g., at least 95% or 98%) or 100% sequence identity to all or at least about 50 (e.g., at least about 60, 70, 80, 85, or 90) contiguous aas of a HLA DO alpha (DOA) α1 and/or α2 domain polypeptide sequence provided in FIG. 11; and
   a β1 and/or β2 domain polypeptide sequences each having at least 90% (e.g., at least 95% or 98%) or 100% sequence identity to all or at least about 50 (e.g., at least about 60, 70, 80, 85, or 90) contiguous aas of a HLA DO beta (DOB) β1 and/or β2 domain polypeptide sequences provided in FIG. 12.
10. The MAPP of aspect 4, wherein at least one presenting sequence or presenting complex (e.g., at least two or all presenting sequences and/or complexes) comprises:
   a α1 and α2 domain polypeptide sequences each having at least 90% (e.g., at least 95% or 98%) or 100% sequence identity to all or at least about 50 (e.g., at least about 60, 70, 80, 85, or 90) contiguous aas of a HLA DP alpha 1 (DPA1) α1 and/or α2 domain polypeptide sequence provided in 13; and
   a β1 and β2 domain polypeptide sequences each having at least 90% (e.g., at least 95% or 98%) or 100% sequence identity to all or at least about 50 (e.g., at least about 60, 70, 80, 85, or 90) contiguous aas of a HLA DP beta 1 (DPB1) β1 and/or β2 domain polypeptide sequences provided in FIG. 14.
11. The MAPP of aspect 4, wherein at least one presenting sequence or presenting complex (e.g., at least two or all presenting sequences and/or complexes) comprises:
   a α1 and α2 domain polypeptide sequences each having at least 90% (e.g., at least 95% or 98%) or 100% sequence identity to all or at least about 50 (e.g., at least about 60, 70, 80, 85, or 90) contiguous aas of a HLA DQ alpha 1 (DQA1) α1 and/or α2 domain polypeptide sequence provided in FIG. 15; and
   a β1 and β2 domain polypeptide sequences each having at least 90% (e.g., at least 95% or 98%) or 100% sequence identity to all or at least about 50 (e.g., at least about 60, 70, 80, 85, or 90) contiguous aas of a HLA DQ beta 1 (DQB1) β1 and/or β2 domain polypeptide sequences provided in FIG. 17.
12. The MAPP of aspect 4, wherein at least one presenting sequence or presenting complex (e.g., at least two or all presenting sequences and/or complexes) comprises:
   a α1 and α2 domain polypeptide sequences each having at least 90% (e.g., at least 95% or 98%) or 100% sequence identity to all or at least about 50 (e.g., at least about 60, 70, 80, 85, or 90) contiguous aas of a HLA DQ alpha 2 (DQA2) α1 and/or α2 domain polypeptide sequence provided in FIG. 16; and
   a β1 and β2 domain polypeptide sequences each having at least 90% (e.g., at least 95% or 98%) or 100% sequence identity to all or at least about 50 (e.g., at least about 60, 70, 80, 85, or 90) contiguous aas of a HLA DQ beta 2 (DQB2) β1 and/or β2 domain polypeptide sequences provided in FIG. 18A or 18B.
13. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*01:01 (see FIG. 5), wherein the at least 90% sequence identity may be at least 95%.
14. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*01:02 (see FIG. 5), wherein the at least 90% sequence identity may be at least 95%.
15. The MAPP of any of aspects 1-6 wherein the MAPP comprise an MHC (HLA) aa sequence having at least 90% (e.g., at least 95% or at least 98%) or 100% aa sequence identity to the β1 and/or β2 domains of DRB1*01:03 (see FIG. 5), wherein the at least 90% sequence identity may be at least 95%.
16. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*03:01 (see FIG. 5), wherein the at least 90% sequence identity may be at least 95%.
17. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*03:02 (see FIG. 5), wherein the at least 90% sequence identity may be at least 95%.
18. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*03:04 (see FIG. 5), wherein the at least 90% sequence identity may be at least 95%.
19. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*04:01 (see FIG. 5), wherein the at least 90% sequence identity may be at least 95%.
20. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*04:02, DRB1*04:03, or DRB1*04:04 (see FIG. 5), wherein the at least 90% sequence identity may be at least 95%.
21. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*04:05 (see FIG. 5), wherein the at least 90% sequence identity may be at least 95%.
22. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*04:06 or DRB1*04:08 (see FIG. 5), wherein the at least 90% sequence identity may be at least 95%.
23. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*08:01 (see FIG. 5), wherein the at least 90% sequence identity may be at least 95%.
24. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*08:02 or DRB1*08:03 (see FIG. 5), wherein the at least 90% sequence identity may be at least 95%.
25. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*09:01 (see FIG. 5), wherein the at least 90% sequence identity may be at least 95%.
26. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*10:01 (see FIG. 5), wherein the at least 90% sequence identity may be at least 95%.
27. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*11:01 or DRB1*11:04 (see FIG. 5), wherein the at least 90% sequence identity may be at least 95%.
28. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*11:03 (see FIG. 5), wherein the at least 90% sequence identity may be at least 95%.
29. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*13:01 or DRB1*13:03 (see FIG. 5), wherein the at least 90% sequence identity may be at least 95%.
30. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*14:01 or DRB1*14:02 (see FIG. 5), wherein the at least 90% sequence identity may be at least 95%.
31. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*14:03, DRB1*14:04, DRB1*14:05, or DRB1*14:06 (see FIG. 5), wherein the at least 90% sequence identity may be at least 95%.
32. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*15:01 (see FIG. 5), wherein the at least 90% sequence identity may be at least 95%.
33. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*15:02 (see FIG. 5), wherein the at least 90% sequence identity may be at least 95%.
34. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*15:03 (see FIG. 5), wherein the at least 90% sequence identity may be at least 95%.
35. The MAPP of any of aspects 1-6, wherein the MAPP comprise an MHC (HLA) aa sequence having at least 90% (e.g., at least 95% or at least 98%) or 100% aa sequence identity to the β1 and/or β2 domains of DRB1*15:04, DRB1*15:05, DRB1*15:06, or DRB1*15:07 (see FIG. 5), wherein the at least 90% sequence identity may be at least 95%.
36. The MAPP of any of aspects 1-4 and 7, wherein the MAPP comprise an MHC (HLA) aa sequence having at least 90% (e.g., at least 95% or at least 98%) or 100% aa sequence identity to the β1 and/or β2 domains of DRB3*03:01 (see FIG. 6), wherein the at least 90% sequence identity may be at least 95%.
37. The MAPP of any of aspects 1-4 and 7, wherein the MAPP comprise an MHC (HLA) aa sequence having at least 90% (e.g., at least 95% or at least 98%) or 100% aa sequence identity to the β1 and/or β2 domains of DRB4*01:01 or DRB4*01:03 (see FIG. 7), wherein the at least 90% sequence identity may be at least 95%.
38. The MAPP of any of aspects 1-4 and 7, wherein the MAPP comprise an MHC (HLA) aa sequence having at least 90% (e.g., at least 95% or at least 98%) or 100% aa sequence identity to the β1 and/or β2 domains of DRB5*01:01 (see FIG. 8), wherein the at least 90% sequence identity may be at least 95%.
39. The MAPP of any of aspects 1-4 and 11, wherein the MAPP comprise an MHC (HLA) aa sequence having at least 90% (e.g., at least 95% or at least 98%) or 100% aa sequence identity to the β1 and/or β2 domains of DQB1*02:01 or DQB1*02:02 (see FIG. 17), wherein the at least 90% sequence identity may be at least 95%.
40. The MAPP of any of aspects 1-4 and 11, wherein the MAPP comprise an MHC (HLA) aa sequence having at least 90% (e.g., at least 95% or at least 98%) or 100% aa sequence identity to the β1 and/or β2 domains of DQB1*03:01, DQB1*03:02, DQB1*03:03 or DQB1*03:04, (see FIG. 17), wherein the at least 90% sequence identity may be at least 95%.
41. The MAPP of any of aspects 1-4 and 11, wherein the MAPP comprise an MHC (HLA) aa sequence having at least 90% (e.g., at least 95% or at least 98%) or 100% aa sequence identity to the β1 and/or β2 domains of DQB1*04:01 or DQB1*04:02, (see FIG. 17), wherein the at least 90% sequence identity may be at least 95%.
42. The MAPP of any of aspects 1-4 and 11, wherein the MAPP comprise an MHC (HLA) aa sequence having at least 90% (e.g., at least 95% or at least 98%) or 100% aa sequence identity to the β1 and/or β2 domains of DQB1*05:01 or DQB1*05:03, (see FIG. 17), wherein the at least 90% sequence identity may be at least 95%.
43. The MAPP of any of aspects 1-4 and 11, wherein the MAPP comprise an MHC (HLA) aa sequence having at least 90% (e.g., at least 95% or at least 98%) or 100% aa sequence identity to the β1 and/or β2 domains of DQB1*06:01 or DQB1*06:02, (see FIG. 17), wherein the at least 90% sequence identity may be at least 95%.
44. The MAPP of any of aspects 1-4 and 10, wherein the MAPP comprise an MHC (HLA) aa sequence having at least 90% (e.g., at least 95% or at least 98%) or 100% aa sequence identity to the β1 and/or β2 domains of DPB1*03:01 or DPB1*09:01, (see FIG. 14), wherein the at least 90% sequence identity may be at least 95%.
45. The MAPP of any of aspects 1-4 and 10, wherein the MAPP comprise an MHC (HLA) aa sequence having at least 90% (e.g., at least 95% or at least 98%) or 100% aa sequence identity to the β1 and/or β2 domains of DPB1*13:01 or DPB1*35:01, (see FIG. 14), wherein the at least 90% sequence identity may be at least 95%.
46. The MAPP of any of aspects 1-4 and 11, wherein the MAPP comprise an MHC (HLA) aa sequence having at least 90% (e.g., at least 95% or at least 98%) or 100% aa sequence identity to the the α1 and/or α2 domain sequences of DQA1*01:01, DQA1*01:02, DQA1*01:03 or DQA1*01:04, (see FIG. 15), wherein the at least 90% sequence identity may be at least 95%.
47. The MAPP of any of aspects 1-4 and 11, wherein the MAPP comprise an MHC (HLA) aa sequence having at least 90% (e.g., at least 95% or at least 98%) or 100% aa sequence identity to the the α1 and/or α2 domain sequences of DQA1*03:01 or DQA1*03:02.
48. The MAPP of any of aspects 1-4 and 11, wherein the MAPP comprise an MHC (HLA) aa sequence having at least 90% (e.g., at least 95% or at least 98%) or 100% aa sequence identity to the α1 and/or α2 domain sequences of DQA1*04:01 (see FIG. 15), wherein the at least 90% sequence identity may be at least 95%.
49. The MAPP of any of aspects 1-4 and 11, wherein the MAPP comprise an MHC (HLA) aa sequence having at least 90% (e.g., at least 95% or at least 98%) or 100% aa sequence identity to α1 and/or α2 domain sequences of DQA1*05:01 or DQA1*05:05.
50. The MAPP of any of aspects 1-4 and 11, wherein the MAPP comprise an MHC (HLA) aa sequence having at least 90% (e.g., at least 95% or at least 98%) or 100% aa sequence identity to the α1 and/or α2 domain sequences of DQA1*06:01 (see FIG. 15), wherein the at least 90% sequence identity may be at least 95%.
51. The MAPP of any of aspects 1-7, wherein the MAPP comprise an MHC (HLA) aa sequence having at least 90% (e.g., at least 95% or at least 98%) or 100% aa sequence identity to the α1 and/or α2 domain sequences of DRA1*01:01 or DRA1*01:02 (also referred to as DRA*01:01 and DRA*01:02 respectively) (see FIG. 4), wherein the at least 90% sequence identity may be at least 95%.
52. The MAPP of any of aspects 1-4 and 11, wherein: the MAPP comprises an MHC (HLA) aa sequence having at least 90% (e.g., at least 95% or at least 98%) or 100% aa sequence identity to the α1 and/or α2 domain sequences of DQA1*01:01, wherein the at least 90% sequence identity may be at least 95%; and the MAPP comprises and MHC (HLA) aa sequence having at least 90% (e.g., at least 95% or at least 98%) or 100% aa sequence identity to the β1 and/or β2 domains of DQB1*05:01, wherein the at least 90% sequence identity may be at least 95%.
53. The MAPP of any of aspects 1-4 and 11, wherein: the MAPP comprises an MHC (HLA) aa sequence having at least 90% (e.g., at least 95% or at least 98%) or 100% aa sequence identity to the α1 and/or α2 domain sequences of DQA1*01:02, DQA1*01:03, or DQA1*01:04 wherein the at least 90% sequence identity may be at least 95%; and the MAPP comprises and MHC (HLA) aa sequence having at least 90% (e.g., at least 95% or at least 98%) or 100% aa sequence identity to the β1 and/or β2 domains of DQB1*06:02, wherein the at least 90% sequence identity may be at least 95%.
54. The MAPP of any of aspects 1-4 and 11, wherein: the MAPP comprises an MHC (HLA) aa sequence having at least 90% (e.g., at least 95% or at least 98%) or 100% aa sequence identity to the the α1 and/or α2 domain sequences of DQA1*03:02, wherein the at least 90% sequence identity may be at least 95%; and the MAPP comprises and MHC (HLA) aa sequence having at least 90% (e.g., at least 95% or at least 98%) or 100% aa sequence identity to the β1 and/or β2 domains of DQB1*03:01, wherein the at least 90% sequence identity may be at least 95%.
55. The MAPP of any of aspects 1-4 and 11, wherein: the MAPP comprises an MHC (HLA) aa sequence having at least 90% (e.g., at least 95% or at least 98%) or 100% aa sequence identity to the the α1 and/or α2 domain sequences of DQA1*03:01, wherein the at least 90% sequence identity may be at least 95%; and the MAPP comprises and MHC (HLA) aa sequence having at least 90% (e.g., at least 95% or at least 98%) or 100% aa sequence identity to the β1 and/or β2 domains of DQB1*03:03, wherein the at least 90% sequence identity may be at least 95%.
56. The MAPP of any of aspects 1-4 and 11, wherein: the MAPP comprises an MHC (HLA) aa sequence having at least 90% (e.g., at least 95% or at least 98%) or 100% aa sequence identity to the the α1 and/or α2 domain sequences of DRA1*01:01, wherein the at least 90% sequence identity may be at least 95%; and the MAPP comprises and MHC (HLA) aa sequence having at least 90% (e.g., at least 95% or at least 98%) or 100% aa sequence identity to the β1 and/or β2 domains of DRB1*01:01, wherein the at least 90% sequence identity may be at least 95%.
57. The MAPP of any of aspects 1-6, wherein: the MAPP comprises an MHC (HLA) aa sequence having at least 90% (e.g., at least 95% or at least 98%) or 100% aa sequence identity to the α1 and/or α2 domain sequences of DRA1*01:01, wherein the at least 90% sequence identity may be at least 95%; and the MAPP comprises and MHC (HLA) aa sequence having at least 90% (e.g., at least 95% or at least 98%) or 100% aa sequence identity to the β1 and/or β2 domains of DRB1*04:01, wherein the at least 90% sequence identity may be at least 95%.
58. The MAPP of any of aspects 1-6, wherein: the MAPP comprises an MHC (HLA) aa sequence having at least 90% (e.g., at least 95% or at least 98%) or 100% aa sequence identity to the α1 and/or α2 domain sequences of DRA1*01:01, wherein the at least 90% sequence identity may be at least 95%; and the MAPP comprises and MHC (HLA) aa sequence having at least 90% (e.g., at least 95% or at least 98%) or 100% aa sequence identity to the β1 and/or β2 domains of DRB1*05:01, wherein the at least 90% sequence identity may be at least 95%.
59. The MAPP of any of aspects 1-6, wherein: the MAPP comprises an MHC (HLA) aa sequence having at least 90% (e.g., at least 95% or at least 98%) or 100% aa sequence identity to the the α1 and/or α2 domain sequences of DRA1*01:01, wherein the at least 90% sequence identity may be at least 95%; and the MAPP comprises and MHC (HLA) aa sequence having at least 90% (e.g., at least 95% or at least 98%) or 100% aa sequence identity to the β1 and/or β2 domains of DRB1*15:01, wherein the at least 90% sequence identity may be at least 95%.
60. The MAPP of any of aspects 1-6, wherein: the MAPP comprises an MHC (HLA) aa sequence having at least 90% (e.g., at least 95% or at least 98%) or 100% aa sequence identity to the the α1 and/or α2 domain sequences of DRA1*04:01, wherein the at least 90% sequence identity may be at least 95%; and the MAPP comprises and MHC (HLA) aa sequence having at least 90% (e.g., at least 95% or at least 98%) or 100% aa sequence identity to the β1 and/or β2 domains of DRB1*04:02, wherein the at least 90% sequence identity may be at least 95%.
61.MAPP of any of aspects 4-60, wherein the at least 90% sequence identity is at least 95% sequence identity.
62.MAPP of any of aspects 4-60, wherein the at least 90% sequence identity is at least 98% sequence identity.
63. The MAPP of any preceding aspect, wherein the MAPP comprises at least one linker comprising:
   (i) Gly (polyG or polyglycine), Gly and Ala (e.g., GA or AG), Ala and Ser (e.g., AS or SA), Gly and Ser (e.g., GS, GSGGS, GGGS, GGSG, GGSGG, GSGSG, GSGGG, GGGSG, GSSSG, GGGGS), or Ala and Gly (e.g., AAAGG), any of which may be repeated 1, 2, 3, 4, 5, 6, 7, 8, 9, or 10 times; or
   (ii) a cysteine-containing linker sequence selected from CGGGS, GCGGS, GGCGS, GGGCS, and GGGGC, with the remainder of the linker comprised of Gly and Ser residues (e.g., GGGGS units that may be repeated from 1 to 10 times.
64. The MAPP of any preceding aspect, wherein the MAPP comprise at least one rigid peptide linker.
65. The MAPP of any preceding aspect, wherein the MAPP comprise at least one aa sequence independently selected from GCGASGGGGSGGGGS, GCGGSGGGGSGGGGSGGGGS, GCGGSGGGGSGGGGS, and GCGGS(G₄S) where the G₄S unit may be repeated from 1 to 10 times (e.g., repeated 1, 2, 3, 4, 5, 6, 7, 8, 9, or 10 times); wherein the linker cysteine residue optionally forms a disulfide bond (e.g., with another peptide sequence of the MAPP).
66. The MAPP of any preceding aspect, wherein the at least one (e.g., at least two) presenting sequence comprises one or more MOD polypeptide sequences, and wherein the presenting sequence has a structure selected from those FIG. 25 or in FIG. 26.
67. The MAPP of any preceding aspect, wherein when the MAPP comprises a presenting sequence, the presenting sequence comprising, in the N-terminal to C-terminal direction:
   a) the peptide epitope, the β1, α1, α2 and β2 domain polypeptide sequences (see e.g., FIG. 25 structure A);
   b) the peptide epitope, the β1, β2, α1, and α2 domain polypeptide sequences (see e.g., FIG. 25 structure B);
      or
   c) the peptide epitope, the α1, α2, β1, and β2, domain polypeptide sequences (see e.g., FIG. 25 structure C);
   wherein the presenting sequence optionally comprises one or more MOD or variant MOD polypeptide sequences; and
   wherein said presenting sequence optionally comprises one or more independently selected linker sequences (e.g., joining any one or more of the peptide epitope, α1, α2, β1, and/or β2 domains) and/or at the N- or C-terminus.
68. The MAPP of any preceding aspect, wherein the at least one (e.g., at least two) presenting sequence comprises one or more MOD polypeptide sequences.
69. The MAPP of any preceding aspect, wherein the at least one presenting sequence (e.g., at least two presenting sequences) comprises one or more MOD polypeptide sequences and wherein the presenting sequence has a structure selected from those set forth in FIG. 26 structures A to H.
70. The MAPP of any of aspects 1-65, comprising at least one (e.g., at least two) presenting complex, wherein the at least one presenting complex comprises a presenting complex 1^{st} sequence and presenting complex 2^{nd} sequence wherein
   (i) the presenting complex 1^{st} sequence comprises the α1 domain polypeptide sequence, and its associated presenting complex 2^{nd} sequence comprises the peptide epitope sequence and the β1 domain polypeptide sequence (e.g., the epitope is placed N-terminal to the β1) (see e.g., FIG. 27, structures A, B, and D),
   (ii) the presenting complex 1^{st} sequence comprises the α2 domain polypeptide sequence, and its associated presenting complex 2^{nd} sequence comprises the peptide epitope sequence and the β2 domain polypeptide sequence (e.g., the epitope is placed N-terminal to the β2) (see e.g., FIG. 27, structures A, B, and D),
   (iii) the presenting complex 1^{st} sequence comprises the β1 domain polypeptide sequence, and its associated presenting complex 2^{nd} sequence comprises the peptide epitope sequence and the α1 domain polypeptide sequence (e.g., the epitope is placed N-terminal to the α1),
   (iv) the presenting complex 1^{st} sequence comprises the β2 domain polypeptide sequence, and its associated presenting complex 2^{nd} sequence comprises the peptide epitope sequence and the α2 domain polypeptide sequence (e.g., the epitope is placed N-terminal to the α2),
   (v) the presenting complex 1^{st} sequence comprises the α1 domain polypeptide sequence, and its associated presenting complex 2^{nd} sequence comprises the peptide epitope sequence and the β1 and β2 domain polypeptide sequences (e.g., the epitope is placed N-terminal to the β1 and/or β2),
   (vi) the presenting complex 1^{st} sequence comprises the α2 domain polypeptide sequence, and its associated presenting complex 2^{nd} sequence comprises the peptide epitope sequence and the β1 and β2 domain polypeptide sequences (e.g., the epitope is placed N-terminal to the β1 and/or β2),
   (vii) the presenting complex 1^{st} sequence comprises the α1 and/or α2 domain polypeptide sequences, and its associated presenting complex 2^{nd} sequence comprises the peptide epitope sequence and the β1 and β2 domain polypeptide sequences (e.g., the epitope is placed N-terminal to the β1 and/or β2) (see, e.g., FIG. 27 structures A, B, and D, FIG. 28 structures B, D, and F, and FIG. 29 structure B), or
   (viii) the presenting complex 1^{st} sequence comprises the β1 and/or β2 domain polypeptide sequences, and its associated presenting complex 2^{nd} sequence comprises the peptide epitope sequence and the α1 and α2 domain polypeptide sequences (e.g., the epitope is placed N-terminal to the α1 and/or α2) (see e.g., FIG. 29 structures D, E, F, G, and H); and
   wherein the at least one presenting complex optionally comprises one or more, or two more MODs or variant MODs.
71. The MAPP of any of aspects 1-65, comprising at least one (e.g., at least two) presenting complex, wherein the at least one presenting complex comprises a presenting complex 1^{st} sequence and presenting complex 2^{nd} sequence comprise wherein:
   (i) the presenting complex 1^{st} sequence comprises the peptide epitope sequence and the α1 domain polypeptide sequence (e.g., the epitope is placed N-terminal to the α1 sequence), and its associated presenting complex 2^{nd} sequence comprises the β1 domain polypeptide sequence,
   (ii) the presenting complex 1^{st} sequence comprises the peptide epitope sequence and the α2 domain polypeptide sequence (e.g., the epitope is placed N-terminal to the α2), and its associated presenting complex 2^{nd} sequence comprises the β2 domain polypeptide sequence (see e.g., FIG. 27 structure C, FIG. 29 structure C),
   (iii) the presenting complex 1^{st} sequence comprises the peptide epitope sequence and the β1 domain polypeptide sequence (e.g., the epitope is placed N-terminal to the β1), and its associated presenting complex 2^{nd} sequence comprises the α1 domain polypeptide sequence (see e.g., FIG. 27, structure C, FIG. 28 structures A, C, and E, and FIG. 29 structure A),
   (iv) the presenting complex 1^{st} sequence comprises the peptide epitope sequence and the β2 domain polypeptide sequence (e.g., the epitope is placed N-terminal to the β2), and its associated presenting complex 2^{nd} sequence comprises the α2 domain polypeptide sequence (e.g., the epitope is placed N-terminal to the α2) (see e.g., FIG. 27, structure C, FIG. 28 structures A, C, and E, and FIG. 29, structures A, G and C),
   (v) the presenting complex 1^{st} sequence comprises the peptide epitope sequence and the α1 domain polypeptide sequence (e.g., the epitope is placed N-terminal to the α1 sequence), and its associated presenting complex 2^{nd} sequence comprises the β1 and β2 domain polypeptide sequences (e.g., the epitope is placed N-terminal to the β1 and/or β2),
   (vi) the presenting complex 1^{st} sequence comprises the peptide epitope sequence and the α2 domain polypeptide sequence (e.g., the epitope is placed N-terminal to the α2 sequence), and its associated presenting complex 2^{nd} sequence comprises the β1 and β2 domain polypeptide sequences (e.g., the epitope is placed N-terminal to the β1 and/or β2),
   (vii) the presenting complex 1^{st} sequence comprises the peptide epitope sequence and the α1 and/or α2 domain polypeptide sequences (e.g., the epitope is placed N-terminal to the α1 and/or α2), and its associated presenting complex 2^{nd} sequence comprises the β1 and β2 domain polypeptide sequences (e.g., the epitope is placed N-terminal to the β1 and/or β2) (see e.g., FIG. 29 structure C), or
   (viii) the presenting complex 1^{st} sequence comprises the peptide epitope sequence and the β1 and/or β2 domain polypeptide sequences (e.g., the epitope is placed N-terminal to the β1 and/or β2), and its associated presenting complex 2^{nd} sequence comprises the α1 and α2 domain polypeptide sequences (see e.g., FIG. 27 structure C, FIG. 28 structures A, B, C, and E, and FIG. 29 structure A, D, E, and F); and
   wherein the at least one presenting complex optionally comprises one or more, or two more MODs or variant MODs.
72. The MAPP of any of aspects 1-65 or 70-71, comprising at least one presenting complex that comprises one or more, (e.g., two or more) MOD or variant MOD polypeptide sequences.
73. The MAPP of any of aspects 1-65 or 70-71, comprising at least one (e.g., at least two) presenting complex, wherein the at least one presenting complex has a structure selected from those in FIG. 27, 28, or 29.
74. The MAPP of any of aspects 1-65 or 70-71, comprising at least one (e.g., at least two) presenting complex, wherein the at least one presenting complex has a structure selected from those in FIGS. 30 to 32.
75. The MAPP of any preceding aspect, wherein, the at least one presenting sequence, presenting complex 1^{st} sequence, or presenting complex 2^{nd} sequence comprises its peptide epitope sequence within 10 aa, 15 aa, 20 aa, or 25 aa of the its N-terminus.
76. The MAPP of any preceding aspect, comprising:
   (i) a cysteine-containing linker, wherein the cysteine residue in the linker forms a disulfide bond between a between a presenting sequence and another polypeptide of the MAPP, or between presenting complex 1st sequence and another polypeptide of the MAPP (e.g., with a presenting complex 2nd sequence);
   (ii) at least one presenting sequence or a presenting complex comprising a disulfide bond formed between one of MHC α1 or α2 domain polypeptide sequence and one of the β1 or β2 domain polypeptide sequences;
   (iii) at least one presenting sequence or a presenting complex comprising a disulfide bond formed between cysteines positioned at
      α chain position 3 and β chain position 19 or 20,
      α chain position 4 and β chain position 19 or 20,
      α chain position 28 and β chain position 151, 152, or 153,
      α chain position 29 and β chain position 151, 152, or 153,
      α chain position 80, 81, or 82 and β chain position 33,
      α chain position 93 and β chain position 153 of 156,
      α chain position 94 and β chain position 120 or 156, or
      α chain position 95 and β chain position 120 or 156;
   (iv) at least one presenting sequence or a presenting complex comprising a disulfide bond formed between cysteines positioned at
      α chain position 12 and β chain position 7 or 10,
      α chain position 80 and β chain position 5 or 7,
      α chain position 81 and β chain position 5 or 7, or
      α chain position 82 and β chain position 5 or 7; and/or
   (v) at least one presenting sequence or at least one presenting complex that comprises a cysteine-containing polypeptide linker having the structure (aa1-aa2-aa3-aa4-aa5-[remainder of linker if present]) that connects an epitope (e.g., an N-terminal epitope) and a β1 domain polypeptide sequence such that the at least one presenting sequence or at least one presenting complex comprises a substructure of the form {epitope-aa1-aa2-aa3-aa4-aa5-[remainder of linker if present or bond]-β1 domain}, and wherein the presenting sequence or presenting complex comprises a disulfide bond between a cysteine located at any of aa1 to aa5 and an MHC α chain polypeptide sequence (e.g., an α1 or α2 domain polypeptide sequence).
77. The MAPP of any preceding aspect, wherein the dimerization and multimerization sequences are independently selected from non-interspecific sequences or interspecific sequences
78. The MAPP of aspect 77, wherein the interspecific and non-interspecific sequences are selected from the group consisting of: immunoglobulin heavy chain constant regions (Ig Fc e.g., CH2-CH3); collectin polypeptides, coiled-coil domains, leucine-zipper domains; Fos polypeptides; Jun polypeptides; Ig CH1; Ig C_{L} κ; Ig C_{L}λ; knob-in-hole without disulfide (“KiH”); knob-in hole with a stabilizing disulfide bond (“KiHs-s”); HA-TF; ZW-1; 7.8.60; DD-KK; EW-RVT; EW-RVTs-s; and A107 sequences.
79. The MAPP of any preceding aspect, complexed to form a duplex or higher order MAPP comprising at least a first MAPP heterodimer and a second MAPP heterodimer of any of aspects 1-78, wherein:
   (i) the first MAPP comprises a first framework polypeptide having a first multimerization sequence and a first dimerization sequence, and a first dimerization polypeptide having first counterpart dimerization sequence complementary to the first dimerization sequence; and
   (ii) the second MAPP comprises a second framework polypeptide having a second multimerization sequence and a second dimerization sequence, and a second dimerization polypeptide having second counterpart dimerization sequence complementary to the second dimerization sequence; and
   wherein the first and second framework polypeptides are associated by binding interactions between the first and second multimerization sequences optionally including one or more interchain covalent bonds (e.g., one or two disulfide bonds), and the multimerization sequences are not the same (e.g., not the same type and/or not identical to), and do not substantially associate with or bind to, the dimerization sequences or counterpart dimerization sequences. See e.g., the duplexes in FIGS. 19 to 23.
80. The duplex MAPP of aspect 79, wherein the first and second dimerization sequence are identical, and the first and second counterpart dimerization sequences are identical. See e.g., FIGS. 21 and 22.
81. The duplex MAPP of aspect 79, wherein the first and second dimerization sequences do not substantially associate with or bind to each other.
82. The duplex MAPP of aspect 79, wherein the first and second multimerization sequences are interspecific multimerization sequences that form an interspecific pair, the first and second dimerization sequence are identical, and the first and second counterpart dimerization sequences are identical. See e.g., FIG. 19 structure B and FIG. 21, structures B and D.
83. The duplex MAPP of aspect 82, wherein the first and second dimerization sequences do not substantially associate with or bind each other.
84. The duplex MAPP of any of aspects 82 to 83, wherein the first or second framework polypeptide comprises at least one MOD (e.g., two or three MODs) that is/are not present on the other framework polypeptide, optionally wherein the at least one MOD comprises a polypeptide sequence that is a variant of a wild-type MOD sequence.
85. The duplex MAPP of aspect 79, wherein the first and second multimerization sequences are identical, the first dimerization sequence and the first counterpart dimerization sequence are interspecific dimerization sequences forming a first interspecific pair, and the second dimerization sequence and second counterpart dimerization sequence are interspecific dimerization sequences forming a second interspecific pair. See e.g., FIG. 19 structure C.
86. The duplex MAPP of aspect 85, wherein the first and second dimerization sequences are identical and the first and second counterpart dimerization sequences are identical. See e.g., FIG. 21 structures A.
87. The duplex MAPP of aspect 85, wherein the first and second dimerization sequences are not identical do not substantially associate with or bind with each other.
88. The duplex MAPP of aspect 85, wherein the polypeptides of the first interspecific pair are different from (not identical to), and do not bind or interact with the polypeptides of the second interspecific pair.
89. The duplex MAPP of any of aspects 87 to 88, wherein the first or second dimerization polypeptide comprises at least one MOD (e.g., two or three MODs) that is/are not present on the other dimerization polypeptide, optionally wherein the at least one MOD comprises a polypeptide sequence that is a variant of a wild-type MOD sequence.
90. The duplex MAPP of aspect 79, wherein
   the first and second multimerization sequences are interspecific multimerization sequences that form an interspecific multimerization pair,
   the first dimerization sequence and the first counterpart dimerization sequence are interspecific dimerization sequences forming a first interspecific pair, and
   the second dimerization sequence and second counterpart dimerization sequence are interspecific dimerization sequences forming a second interspecific pair. See e.g., FIG. 19 structure D.
91. The duplex MAPP of aspect 90, wherein the first and second dimerization sequences are identical and the first and second counterpart dimerization sequences are identical. See e.g., FIG. 21 structure D.
92. The duplex MAPP of aspect 90, wherein the first and second dimerization sequences do not substantially associate with or bind with each other.
93. The duplex MAPP of aspect 90, wherein the polypeptides of the first interspecific pair polypeptides are different from (not identical to), and do not bind or interact with the polypeptides of the second interspecific pair.
94. The duplex MAPP of any of aspects 92 to 93, wherein the first or second dimerization polypeptide comprises at least one MOD (e.g., two or three MODs or variant MODs or variant MODs) that is/are not present on the other dimerization polypeptide, optionally wherein the at least one MOD comprises a polypeptide sequence that is a variant of a wild-type MOD sequence.
95. The duplex MAPP of any of aspects 90 to 94, wherein the first or second framework polypeptide comprises at least one MOD (e.g., two or three MODs or variant MODs) that is/are not present on the other framework polypeptide, optionally wherein the at least one MOD comprises a polypeptide sequence that is a variant of a wild-type MOD sequence.
96. The duplex MAPP of any of aspects 79 to 95, wherein:
   (i) when the multimerization sequences are not an interspecific multimerization pair, the multimerization sequences are selected from the group consisting of immunoglobulin heavy chain constant regions (e.g., Ig CH2-CH3), collectin family dimerization sequences, coiled-coil domains, and leucine-zipper domains; and
   (ii) when the multimerization sequences are an interspecific multimerization pair, the multimerization sequences are selected from the group consisting of a Fos and Jun polypeptide pair, Ig CH1 and Ig CL κ or λ constant region polypeptide pair, a KiH pair, a KiHs-s pair, a HA-TF polypeptide pair, a ZW-1 polypeptide pair, a 7.8.60 polypeptide pair, a DD-KK polypeptide pair, an EW-RVT polypeptide pair, an EW-RVTs-s polypeptide pair, and an A107 polypeptide pair.
97. The duplex MAPP of aspect 96, wherein the multimerization sequences, the first dimerization sequence and its counterpart first dimerization sequence, and second dimerization sequence and its counterpart dimerization sequence are each selected from the group consisting of: immunoglobulin heavy chain constant regions (e.g., IgFc CH2-CH3), collectin family dimerization sequences, coiled-coil domains, and leucine-zipper domains, and
   wherein the first and second dimerization sequences, which are selected independently and may be the same or different.
98. The duplex MAPP of aspect 96, wherein the multimerization sequences are selected from the group consisting of immunoglobulin heavy chain constant regions (e.g., Ig CH2-CH3), collectin family dimerization sequences, coiled-coil domains, and leucine-zipper domains, and
   wherein a pair comprising the first dimerization sequence and its counterpart dimerization sequence pair, and a pair comprising the second dimerization sequence and it counterpart dimerization sequence, are independently selected from the group consisting of a Fos and Jun polypeptide pair, Ig CH1 and Ig C_{L} κ or λ constant region polypeptide pair, a KiH pair, KiHs-s pair, a HA-TF polypeptide pair, a ZW-1 polypeptide pair, a 7.8.60 polypeptide pair, a DD-KK polypeptide pair, an EW-RVT polypeptide pair, an EW-RVTs-s polypeptide pair, and an A107 polypeptide pair; and
   wherein the pairs may be the same or different.
99. The duplex MAPP of aspect 96, wherein the multimerization sequences are selected from the group consisting of a Fos and Jun polypeptide pair, Ig CH1 and Ig C_{L} κ or λ constant region polypeptide pair, a KiH pair, a KiHs-s pair, a HA-TF polypeptide pair, a ZW-1 polypeptide pair, a 7.8.60 polypeptide pair, a DD-KK polypeptide pair, an EW-RVT polypeptide pair, an EW-RVTs-s polypeptide pair, and an A107 polypeptide pair, and
   wherein the first and second dimerization sequences, which may be the same or different, and their counterpart dimerization sequences are independently selected from the group consisting of immunoglobulin heavy chain constant regions (e.g., Ig CH2-CH3), collectin family dimerization sequences, coiled-coil domains, and leucine-zipper domains.
100. The duplex MAPP of aspect 96, wherein the multimerization sequences, the first dimerization sequence and its counterpart first dimerization sequence, and second dimerization sequence and its counterpart dimerization sequence are each selected as a pair from the group consisting of: Fos and Jun polypeptide pairs, Ig CH1 (see e.g., FIGS. 2A, 2B, and 2E-2I) and Ig C_{L} κ or λ constant region polypeptide pairs, KiH pairs, KiHs-s pairs, HA-TF polypeptide pairs, ZW-1 polypeptide pairs, 7.8.60 polypeptide pairs, a DD-KK polypeptide pairs, EW-RVT polypeptide pairs, EW-RVTs-s polypeptide pairs, and A107 polypeptide pairs, and
   wherein the pairs comprising the first and second dimerization sequences may be the same or different.
101. The duplex MAPP of aspect 96, wherein the multimerization sequences comprise Ig Fc regions and the first and second dimerization sequences comprise independently selected Ig CH1, Ig C_{L} κ or λ, leucine zipper, Fos or Jun domains.
102. The duplex MAPP of aspect 96, wherein the multimerization sequences comprise Ig Fc regions and the first and second dimerization sequences comprise independently selected Ig CH1 or Ig C_{L} κ or λ domains.
103. The duplex MAPP of any of aspects 101 to 102, wherein the Ig CH2-CH3 domains are selected from the group consisting of IgA, IgD, IgE, IgG and IgM Fc regions.
104. The duplex MAPP of any of aspects 101 to 103, wherein the Ig Fc regions are selected from IgG1, IgG2, IgG3, and IgG4 CH2-CH3 domains (e.g., having at least about 70%, at least about 80%, at least about 90%, at least about 95%, at least about 98%, at least about 99%, or 100% aa sequence identity to an aa sequence of the CH2 and/or CH3 domains of an Fc region of SEQ ID NOs: 4-12).
105. The duplex MAPP of any of aspects 101 to 103, wherein the Ig Fc regions are IgG1CH2-CH3 domains.
106. The duplex MAPP of aspect 96, wherein the multimerization sequences are a pair of interspecific immunoglobulin sequences.
107. The duplex MAPP of aspect 106, wherein the pair of interspecific immunoglobulin sequences are selected from the group consisting of KiH pairs, KiHs-s pairs, HA-TF polypeptide pairs, ZW-1 polypeptide pairs, 7.8.60 polypeptide pairs, a DD-KK polypeptide pairs, EW-RVT polypeptide pairs, EW-RVTs-s polypeptide pairs, and A107 polypeptide pairs.
108. The duplex MAPP of aspect 106 or 107, wherein the pair of interspecific immunoglobulin sequences is a KiH) pair.
109. The duplex MAPP of any of aspects 106 to 108, wherein the knob-in hole pair further comprises at least one stabilizing disulfide bond (e.g., a KiHs-s pair).
110. The duplex MAPP of any of aspects 106 to 109, wherein the first and second dimerization sequences comprise independently selected Ig CH1, Ig C_{L} κ or λ, leucine zipper, Fos or Jun domains.
111. The duplex MAPP of any of aspects 106 to 109, wherein the first and second dimerization sequences comprise Ig CH1 or Ig C_{L} κ or λ, domains.
112. The duplex MAPP of any of aspects 106 to 109, wherein the first and second dimerization sequences comprise Ig CH1 domains.
113. The duplex MAPP of any of aspects 108 to 109, wherein the first and/or second dimerization sequences do not comprise Ig CH1 domains.
114. The MAPP or duplex MAPP of any of aspects 1 to 113, wherein:
   the dimerization sequence and its counterpart dimerization sequence of a MAPP of any of aspects 1-78 are covalently linked by at least one (e.g., two) disulfide bond(s); or
   the duplex MAPP of any of aspects 79-113, wherein the dimerization sequence and counterpart dimerization sequence of the first MAPP and/or the dimerization sequence and counterpart dimerization sequence of the second MAPP are covalently linked by at least one (e.g., two) disulfide bond(s).
115. The duplex MAPP of any of aspects 79-114, wherein the multimerization sequences of the first and second framework polypeptides of the first and second MAPP are covalently linked by at least one (e.g., two) disulfide bond(s). See, e.g., FIGS. 21-23.
116. The duplex MAPP of any of aspects 79-115, wherein the first dimerization sequence and its counterpart dimerization sequence and/or the second dimerization sequence and its counterpart dimerization sequence are covalently linked by at least one (e.g., two) disulfide bond(s); and the multimerization sequences of the first and second framework polypeptides of the first and second MAPP are covalently linked by at least one (e.g., two) disulfide bond(s). See e.g., FIGS. 22 and 33.
117. The MAPP or duplex MAPP of any preceding aspect, wherein the MAPP comprises only one presenting sequence or one presenting complex, or the duplex MAPP comprises only two presenting sequences or two presenting complexes. See, e.g., FIGS. 1A and 1B. See, e.g., the 1st or 2nd heterodimer in FIG. 1
118. The MAPP or duplex MAPP of aspect 117, wherein the one presenting sequence or presenting complex of the MAPP, or the two presenting sequences or presenting complexes of the duplex MAPP, is/are provided on the dimerization polypeptide(s), and further, when the MAPP or duplex MAPP comprises a presenting complex, the presenting complex 1^{st} sequence(s) is/are located on the dimerization polypeptide(s) (e.g., located on the N-terminal side of the counterpart dimerization sequence). See, e.g., FIGS. 1A and 1B.
119. The MAPP or duplex MAPP of aspect 117, wherein the one presenting sequence or the presenting complex 1^{st} sequence of the MAPP, or the two presenting sequences or presenting complex 1st sequences of the duplex MAPP, is/are provided on the framework polypeptide(s) (e.g., located on the N-terminal side of the dimerization sequence).
120. The duplex MAPP of any of aspects 79-116, wherein the duplex MAPP comprises only one presenting sequence or one presenting complex.
121. The duplex MAPP of aspect 120, wherein the one presenting sequence or the presenting complex 1^{st} sequence of the one presenting complex is an aa sequence of one dimerization polypeptide (e.g., located on the N-terminal side of the counterpart dimerization sequence of the one dimerization polypeptide).
122. The duplex MAPP of aspect 120, wherein the one presenting sequence or the presenting complex 1^{st} sequence of the one presenting complex is an aa sequence of one framework polypeptide (e.g., located on the N-terminal side of the dimerization sequence).
123. The duplex MAPP of any of aspects 79-116, wherein the duplex MAPP comprises at least two presenting sequences or at least two presenting complexes. See, e.g., the duplex in FIGS. 1A and 1B.
124. The duplex MAPP of aspect 123, wherein one of the at least two presenting sequences or presenting complex 1^{st} sequences of the at least two presenting complexes is part of the first dimerization polypeptide, and the second of the at least two presenting sequences or presenting complex 1^{st} sequences is part of the second dimerization polypeptides (e.g., located on the N-terminal side of their counterpart dimerization sequences). See, e.g., the duplex in FIGS. 1A and 1B.
125. The duplex MAPP of aspect 123, wherein one of the at least two presenting sequences or each of the presenting complex 1^{st} sequences of the at least two presenting complexes is part of the first framework polypeptide, and the second of the at least two presenting sequences or presenting complex 1^{st} sequences is part of the second framework polypeptide (e.g., located on the N-terminal side of their dimerization sequence).
126. The duplex MAPP of any of aspects 79-116, wherein the duplex MAPP comprises at least four presenting sequences or four presenting complexes. See e.g., FIG. 20 structures A-D.
127. The duplex MAPP of aspect 126, wherein each one of the four presenting sequences or each one of the presenting complex 1^{st} sequences of the four presenting complexes are each part of a different one of the first dimerization polypeptide, second dimerization polypeptide, first framework polypeptide and second framework polypeptide (e.g., located on the N-terminal side of their dimerization sequence or counterpart dimerization sequence). See e.g., FIG. 20 structures A-D.
128. The MAPP or duplex MAPP of any preceding aspect, wherein when a framework or dimerization polypeptides of the MAPP or duplex MAPP comprises one or more IgFc regions, and wherein at least one of the one or more IgFc regions comprises one or more substitutions that limit complement activation.
129. The MAPP or duplex MAPP of any preceding aspect, wherein when a framework or dimerization polypeptides of the MAPP or duplex MAPP comprises one or more IgFc regions, and wherein at least one of the one or more IgFc regions comprises one or more substitutions at L234, L235, G236, G237, P238, S239, D270, N297, K322, P329, and/or P331 (respectively, aas L14, L15, G16, G17, P18, 519, N77, D50, K102, P109, and P111 of the wt. IgG1 aa sequence SEQ ID NO: 4 provided in FIG. 2D).
130. The MAPP or the duplex MAPP of aspect 129, wherein when a framework or dimerization polypeptide comprises an IgFc region comprising a substitution at N297 (e.g., N297A).
131. The MAPP or the duplex MAPP of aspect 129, wherein when a framework or dimerization polypeptide comprises an IgFc region comprising a substitution at L234, and/or L235 (e.g., L234A, and/or L235A).
132. The MAPP or the duplex MAPP of aspect 129, wherein when a framework or dimerization polypeptide comprises an IgFc region having a substitution at P331 (e.g., P331A or P331S.
133. The MAPP or the duplex MAPP of aspect 129, wherein when a framework or dimerization polypeptide comprises an Ig Fc region that comprises: (i) one or more substitutions selected from the group consisting of L234, L235, and P331 (e.g., L234F, L235E, and/or P331S substitution(s)); or (ii) any one or more of D270, K322, and P329 (e.g., D270, K322, and/or P329 substitution(s)).
134. The MAPP of any of aspects 1 to 78, complexed to form a duplex MAPP of two heterodimers, triplex MAPP of three heterodimers, a quadraplex MAPP of four heterodimers, a pentaplex MAPP of five heterodimers, or a hexaplex MAPP of six heterodimers.
135. The MAPP or duplex MAPP of any preceding aspect, comprising: at least one MOD (wt or variant), at least one pair of MODs in tandem (both wt, both variant, or one wt and one variant), wherein the at least one MOD or at least one pair of MODs is located at one or more of positions 1, 1′, 2, 2′, 3, 3′, 4, 4′,4″, 4′″, 5, and/or 5′ (see FIGS. 1A and 1B).
136. The MAPP or duplex MAPP of any preceding aspect, wherein:
   (a) at least one MOD (wt or variant), or pair of MODs in tandem (both wt, both variant, or one wt and one variant), is located:
      (i) on the N-terminal side (e.g., at the N-terminus) of at least one framework polypeptide dimerization sequence (see e.g., position 1 and 1′ in any of FIGS. 19 and 21),
      (ii) on the N-terminal side (e.g., at the N-terminus) of at least one framework polypeptide dimerization sequence and any MHC Class II polypeptide sequences that may be part of the framework polypeptide (see e.g., positions 4″ and 4′″ in FIGS. 20 and 22) and/or
      (iii) on the C-terminal side (e.g., at the C-terminus) of at least one framework polypeptide framework multimerization sequence (see e.g., position 3 and 3′ in any of FIGS. 1, 19 to 23); and/or
   (b) at least one MOD (wt or variant), or pair of MODs in tandem (both wt, both variant, or one wt and one variant), located:
      (i) on the N-terminal side (e.g., at the N-terminus) of each framework polypeptide dimerization sequence (see e.g., position 1 and 1′ in any of FIGS. 19 and 21);
      (ii) on the N-terminal side (e.g., at the N-terminus) of each framework polypeptide dimerization sequence and any MHC Class II polypeptide sequences that may be part of the framework polypeptide (see e.g., positions 4″ and 4′″ in FIGS. 20 and 22); and/or
      (iii) on the C-terminal side (e.g., at the C-terminus) of each framework polypeptide framework multimerization sequence (see e.g., position 3 and 3′ in any of FIGS. 1 and 19 to 23).
137. The MAPP or duplex MAPP of aspect 135 or aspect 136 comprising:
   (i) at least one MOD (wt or variant), or pair of MODs in tandem (both wt, both variant, or one wt and one variant) located on the N-terminal side (e.g., at the N-terminus) of at least one (e.g., each) framework polypeptide dimerization sequence (see e.g., position 1 and 1′ in any of FIGS. 19 and 21), or on the N-terminal side (e.g., at the N-terminus) of at least one (e.g., each) framework polypeptide dimerization sequence and any MHC Class II polypeptide sequences that may be part of the framework polypeptide (see e.g., positions 4″ and 4′″ in FIGS. 20 and 22); and/or
   (ii) at least one MOD (wt or variant), or pair of MODs in tandem (both wt, both variant, or one wt and one variant) located on the C-terminal side (e.g., at the C-terminus) of at least one (e.g., each) framework polypeptide framework multimerization sequence (see e.g., position 3 and 3′ in any of FIGS. 1, 19 to 23).
138. The MAPP or duplex MAPP of any preceding aspect, comprising: at least one MOD (wt or variant), or pair of MODs in tandem (both wt, both variant, or one wt and one variant) located:
   (i) on the N-terminal side (e.g., at the N-terminus) of at least one (e.g., two or each) dimerization polypeptide counterpart dimerization sequence (see e.g., positions 4 and 4′ in FIGS. 1, and 20 to 22); and/or
   (ii) on the C-terminal side (e.g., at the C-terminus) of at least one (e.g., two or each) dimerization polypeptide counterpart dimerization sequence (see e.g., position 5 and 5′ in any of FIGS. 1 and 19 to 22).
139. The MAPP or duplex MAPP of any of aspects 135 to 138, wherein when the at least one (e.g., at least two or each) dimerization polypeptide comprises a presenting sequence, the at least one MOD (wt or variant), or pair of MODs in tandem (both wt, both variant, or one wt and one variant) may be located:
   (i) between the counterpart dimerization sequence and the α1, α2, β1, β2 sequences and epitope sequence;
   (ii) between any of the α1, α2, β1, β2 and epitope sequences;
   (iii) between the epitope and either the α1 and α2 or the β1 and β2 sequence; and/or
   (iv) at the N-terminus of the presenting sequence. See FIG. 25.
140. The MAPP or duplex MAPP of any of aspects 135 to 138, wherein when the dimerization polypeptide comprises a presenting complex, the at least one MOD (wt or variant), or pair of MODs in tandem (both wt, both variant, or one wt and one variant) may be located:
   (i) between the counterpart dimerization sequence and any of the α1, α2, β1, and/or β2 or epitope sequences present in the presenting complex 1^{st} sequence;
   (ii) between any of the α1, α2, β1, and/or β2 or epitope sequences present in the presenting complex 1^{st} sequence
   (iii) at the N-terminus of the presenting complex 1^{st} sequence;
   (iv) at the N-terminus of the presenting complex 2^{st} sequence;
   (v) between any of the α1, α2, β1, and/or β2 and epitope sequences present in the presenting complex 2^{st} sequence; and/or
   (vi) at the C-terminus of the presenting complex 2^{st} sequence. See e.g., FIGS. 27A-D, 28C-F, 29A-H, 30C-L, and 31A-F.
141. The duplex MAPP of any of aspects 79 to 140, comprising: at least one MOD (wt or variant), or pair of MODs in tandem (both wt, both variant, or one wt and one variant) at position 1 and/or 1′.
142. The duplex MAPP of any of aspects 79 to 140, comprising: at least one MOD (wt or variant), or pair of MODs in tandem (both wt, both variant, or one wt and one variant) at position 1 and/or 1′, and at least one MOD (wt or variant), or pair of MODs in tandem (both wt, both variant, or one wt and one variant) at position.
143. The duplex MAPP of any of aspects 79 to 140, comprising: at least one MOD (wt or variant), or pair of MODs in tandem (both wt, both variant, or one wt and one variant) at position 1 and/or 1′, and at least one MOD (wt or variant), or pair of MODs in tandem (both wt, both variant, or one wt and one variant) at position 5 and/or 5′.
144. The MAPP or duplex MAPP of any preceding aspect, comprising at least one (e.g., at least two, or at least three) MOD (wt or variant), wherein each MOD is selected independently from the group consisting of: IL-1, IL-2, IL-4, IL-6, IL-7, IL-10, IL-12, IL-15, IL-17, IL-21, IL-23, CD7, CD30L, CD40, CD70, CD80, (B7-1), CD83, CD86 (B7-2), HVEM (CD270), ILT3 (immunoglobulin-like transcript 3), ILT4 (immunoglobulin-like transcript 4), Fas ligand (FasL), ICAM (intercellular adhesion molecule), ICOS-L (inducible costimulatory ligand), JAGI (CD339), lymphotoxin beta receptor, 3/TR6, OX40L (CD252), PD-L1, PD-L2, TGF-β1, TGF-β2, TGF-β3, and 4-1BBL polypeptide sequences.
145. The MAPP or duplex MAPP of any preceding aspect, comprising at least one (e.g., at least two, or at least three) MOD (wt or variant), wherein each MOD is selected independently from the group consisting of: 4-1BBL, PD-L1, IL-2, CD80, CD86, OX40L (CD252), Fas ligand (FasL), ICOS-L, ICAM, CD30L, CD40, CD83, HVEM (CD270), JAGI (CD339), CD70, CD80, CD86, TGF-β1, TGF-β2, and TGF-β3 polypeptide sequences.
146. The MAPP or duplex MAPP of any preceding aspect, comprising at least one (e.g., at least two, or at least three) MOD (wt or variant), wherein each MOD polypeptide sequence is selected independently from the group consisting of IL-2, FasL, PD-L1, TGF-β, CD80, CD86 and 4-1BBL polypeptide sequences. For example, the MAPP or duplex MAPP may comprise at least one IL-2 MOD (wt or variant) polypeptide sequence, and at least one MOD (wt or variant) independently selected from CD80 and CD86 polypeptide sequences.
147. The MAPP or duplex MAPP of any preceding aspect, comprising at least one IL-2 MOD (wt or variant) polypeptide sequence, or at least one pair of IL-2 MOD (wt or variant) polypeptide sequences in tandem (optionally located at postion 1 or 1′).
148. The MAPP or duplex MAPP of aspect 146 or 147, further comprising at least one CD80 and/or CD86 MOD (wt or variant) polypeptide sequence, which is optionally located at postion 1 or 1′.
149. The MAPP or duplex MAPP of aspect 146 or 147, further comprising at least one PD-L1 MOD (wt or variant) or variant MOD polypeptide sequence, which is optionally located at postion 1 or 1′.
150. The MAPP or duplex MAPP of aspect 96, further comprising at least one FasL MOD (wt or variant) polypeptide sequence.
151. The MAPP or duplex MAPP of aspect 96, further comprising at least one 4-1BBL MOD (wt or variant) polypeptide sequence.
152. The MAPP or duplex MAPP of any preceding aspect further, comprising an additional peptide, or a payload covalently attached to one or more framework polypeptides and/or dimerization polypeptides.
153. The MAPP or duplex MAPP of aspect 152, wherein the additional peptide is an epitope tag or an affinity domain.
154. The MAPP of any of aspects 1-78, or a higher order complex thereof, wherein the MAPP comprises:
   a framework polypeptide comprising, from N-terminus to C-terminus, aa sequences of (i) a peptide epitope, (ii) human MHC class II β1 and β2 domains, (iii) human Class II α1 and α2 domains, (iv) a human or humanized IgG CH1, and (v) an Ig Fc region (e.g., CH2 CH3 regions with optional “LALA” substitutions), each of (i) to (v) optionally separated by linker peptide sequences that are selected independently; and
   a dimerization polypeptide comprising from N-terminus to C-terminus aa sequences of (i) a human PD-L1 extracellular domain polypeptide, and (ii) a human or humanized kappa light constant region, with (i) and (ii) optionally separated by a linker peptide sequence. See, e.g., FIG. 24 structure A.
155. The MAPP of any of aspects 1-78, or a higher order complex thereof, wherein the MAPP comprises either:
   (I) a framework polypeptide comprising, from N-terminus to C-terminus, aa sequences of (i) a human PD-L1 extracellular domain polypeptide, (ii) a human or humanized CH3 KIH s-s hole, and (iii) a human IgG Fc sequence (e.g., IgG1 CH2 and CH3 regions with optional “LALA” substitutions), each of (i) to (iii) optionally separated by linker peptide sequences that are selected independently; and
      a dimerization polypeptide comprising from N-terminus to C-terminus aa sequences of (i) a peptide epitope, (ii) human MHC class II β1 and β2 domains, (iii) human Class II α1 and α2 domains, and (iv) a human or humanized IgG1 with CH3 KIHₛ₋ₛ knob, each of (i) to (iv) optionally separated by linker peptide sequences that are selected independently (see, e.g., FIG. 24 structure B); or
   (II) a framework polypeptide comprising, from N-terminus to C-terminus, aa sequences of (i) a human PD-L1 extracellular domain polypeptide, (ii) a human or humanized CH1 (optionally with M13 stabilization substitutions), and (iii) a human Ig Fc sequence (e.g., IgG1 CH2 and CH3 regions with optional “LALA” substitutions), each of (i) to (iii) optionally separated by linker peptide sequences that are selected independently; and
      a dimerization polypeptide comprising from N-terminus to C-terminus aa sequences of (i) a peptide epitope, (ii) human MHC class II β1 and β2 domains, (iii) human Class II α1 and α2 domains, and (iv) a human or humanized kappa light chain sequence (optionally with MD13 stabilization substitutions), each of (i) to (iv) optionally separated by linker peptide sequences that are selected independently. See, e.g., FIG. 24 structure C
156. The MAPP of any of aspects 1-78, or a higher order complex thereof, wherein the MAPP comprises:
   a framework polypeptide comprising, from N-terminus to C-terminus, aa sequences of (i) a peptide epitope, (ii) human MHC Class II β1 and β2 domains, (iii) human Class II α1 and α2 domains, (iv) an IgG Fc region (e.g., CH2 and CH3 regions with optional “LALA” substitutions), (v) a human or humanized IgG CH1, each of (i) to (v) optionally separated by linker peptide sequences that are selected independently; and
   a dimerization polypeptide comprising from N-terminus to C-terminus aa sequences of (i) a human PD-L1 extracellular domain polypeptide, and (ii) a human or humanized kappa light constant region, with (i) and (ii) optionally separated by a linker peptide sequence. See, e.g., FIG. 24 structure D.
157. A duplex of the MAPPs of any of aspects 153 to 156, wherein the duplex is formed by interactions between the IgFc multimerization sequences.
158. The MAPP or duplex MAPP of any of aspects 153 to 157, wherein the immunoglobulin (Ig) Fc sequences are Ig G sequences (e.g., Ig G1 sequences).
159. The MAPP or duplex MAPP of aspects 152-158, wherein the additional peptide is a targeting sequence.
160. The MAPP or duplex MAPP of aspect 159, wherein the targeting sequence is an antibody or an antigen binding fragment thereof, or a single chain T cell receptor.
161. The MAPP or duplex MAPP of any of aspects 159 to 160, wherein the targeting sequence is directed to a protein or non-protein epitope of an infectious agent.
162. The MAPP or duplex MAPP of aspect 161, were the infectious agent is a virus, bacteria, fungi, protozoa, or helminth.
163. The MAPP or duplex MAPP of any of aspects 159 to 160, wherein the targeting sequence is directed to an autoantigen or allergen.
164. The MAPP or duplex MAPP of any of aspects 159 to 160, wherein the targeting sequence is directed to a cancer-associated antigen (“CAA”).
165. The MAPP or duplex MAPP of aspect 164, wherein the CAA is selected from those recited herein (see e.g., Section IV.C.7.a.(i) “Cancer Epitopes”).
166. The MAPP or duplex MAPP of aspect 164, wherein the targeting sequence is selected from an anti-CD51, anti-CD74, anti-CD22, anti-CD20, anti-CD20, anti-CD20, anti-CD38, anti-PD-1 receptor, anti-CTLA-4, anti-TROP-2, anti-mucin, anti-CEA, anti-CEACAM6), anti-colon-specific antigen-p), anti-alpha-fetoprotein, anti-IGF-1R, anti-CD19, anti-PSMA, anti-PSMA dimer, anti-carbonic anhydrase IX, anti-HLA-DR, anti-CD52, anti-EpCAM, anti-VEGF, anti-EGFR, anti-CD33, anti-CD20, anti-EGFR, anti-CD20, anti-HER2, anti-CD79b, anti-BCMA, or anti-mesothelin antibody or antigen binding fragment thereof.
167. The MAPP or duplex MAPP of aspect 164, wherein the targeting sequence is selected from an anti-CD19, anti-HER2, anti-BCMA, or anti-mesothelin antibody or antigen binding fragment thereof.
168. The MAPP or duplex MAPP of aspect 164, wherein the CAA is a peptide presented by an HLA as a peptide/HLA complex.
169. The MAPP or duplex MAPP of aspect 168, wherein the targeting sequence targets a peptide/HLA recited herein (see e.g., Section IV.C.8.c.(i).(b) “Peptide/HLA complexes”).
170. The MAPP or duplex MAPP of any of any preceding aspect, wherein the peptide epitope is from about 4 aas (aa) to about 25 aa (e.g., the epitope can have a length of from 4 aa to 10 aa, from about 6 aa to about 12 aa, from 8 aa to 20 aa, from 10 aa to 15 aa, from 10 aa to 20 aa, from 15 aa to 20 aa, or from 20 aa to 25 aa).
171. The MAPP or duplex MAPP of any preceding aspect, wherein the peptide epitope is from about 8 aa to about 20 aa.
172. The MAPP or duplex MAPP of any preceding aspect, wherein the epitope is an epitope of a cancer associated antigen, an epitope of an infectious agent, an epitope of an autoantigen, or epitope of an allergen.
173. The MAPP or duplex MAPP of any preceding aspect, wherein the epitope is a cancer epitope.
174. The MAPP or duplex MAPP of aspect 173, where the cancer epitope is set forth herein (see, e.g., Section IV.C.7.a.(i) “Cancer Epitopes”).
175. The MAPP or duplex MAPP of aspect 173, where the cancer epitope is an Alpha Feto Protein (AFP) epitope.
176. The MAPP or duplex MAPP of aspect 173, where the cancer epitope is an epitope of Wilms Tumor Antigen (WT-1).
177. The MAPP or duplex MAPP of aspect 173, where the cancer epitope is a Human Papilloma Virus I (HPV) epitope (e.g., E6 or E7 protein peptide epitopes).
178. The MAPP or duplex MAPP of aspect 173, where the cancer epitope is a Hepatitis B Virus (HBV) epitope.
179. The MAPP or duplex MAPP of any of aspects 1 to 172, wherein the epitope is an epitope presented by an infectious agent.
180. The MAPP or duplex MAPP of aspect 179, where the infectious agent is a virus, bacterium, fungi, protozoan, or helminth.
181. The MAPP or duplex MAPP of any of aspects 179 to 180, wherein the infectious agent is a virus.
182. The MAPP or duplex MAPP of aspect 181, wherein the virus is HPV and the epitope is an epitope of an HPV or HBV polypeptide.
183. The MAPP or duplex MAPP of any of aspects 1 to 172, wherein the epitope is an epitope of an autoantigen.
184. The MAPP or duplex MAPP of any of aspects 1 to 172, wherein the epitope is an epitope of an allergen (e.g., an allergic protein).
185. The MAPP or duplex MAPP of aspect 184, where the allergen is selected from protein or non-proteins components of: nuts (e.g., tree and/or peanuts), glutens, pollens, eggs (e.g., chicken, Gallus domesticus), shellfish soy, fish, and insect venoms (e.g., bee and/or wasp venom antigens).
186. A pharmaceutical composition comprising one or more MAPPs, duplex MAPPs, or higher order MAPP complexes of any preceding aspect.
187. A method of treatment or prophylaxis of a patient or subject having a disease (e.g., a cancer or infection) or condition (e.g., an allergy or autoimmunity) comprising:
   (i) administering to a patient/subject (e.g., a patient in need thereof) an effective amount of one or more MAPPs, duplex MAPPs, or higher order MAPP complex's of any of aspects 1 to 185, or a pharmaceutical composition comprising of aspect 186;
   (ii) administering to a patient/subject (e.g., a patient in need thereof) an effective amount of one or more nucleic acids encoding a MAPP, duplex MAPP, or higher order MAPP complex of any of aspects 1 to 185;
   (iii) contacting a cell or tissue, either in vitro or in vivo, with one or more MAPPs duplex MAPPs, or higher order MAPP complexes of any of aspects 1 to 185, and administering the cell, tissue, or progeny thereof to the patient/subject; or
   (iv) contacting a cell or tissue, either in vitro or in vivo, with one or more nucleic acids encoding a MAPP, duplex MAPP, or higher order MAPP complex of any of aspects 1 to 185, and administering the cell, tissue, or progeny thereof to the patient/subject.
188. A method of treatment or prophylaxis of a patient or subject having a disease (e.g., a cancer or infection) or condition (e.g., an allergy or autoimmunity) comprising:
   (i) administering to a patient/subject (e.g., a patient in need thereof) an effective amount of one or more MAPPs or duplex MAPPs of any of aspects 1 to 185, or a pharmaceutical composition comprising of aspect 186.
189. The method of aspect 187 or 188, wherein the MAPP(s), duplex MAPP(s), or higher order MAPP complex(s) further comprises at least one targeting sequence (e.g., a targeting sequence specific for an antigen associated with a cell or tissue).
190. The method of any of aspects 187 to 189, wherein the one or more MAPP(s), duplex MAPP(s), or higher order MAPP complex(s) are administered to a mammalian patient or subject.
191. The method of any of aspects 187 to 190, wherein the subject is human.
192. The method of any of aspects 187 to 190, wherein the subject non-human (e.g., rodent, lagomorph, bovine, canine, feline, rodent, murine, caprine, simian, ovine, equine, lappine, porcine, etc.).
193. The method of any of aspects 187 to 192, wherein the disease or condition is a cancer, and wherein when the one or more MAPPs or duplex MAPPs comprises a targeting sequence it is a CTP.
194. The method of any of aspects 187 to 193, wherein the epitope is a cancer epitope (antigen).
195. The method of aspect 193, wherein the epitope is an epitope of a HPV, HBV, AFP or WT-1 protein.
196. The method of any of aspects 187 to 195, further comprising administering one or more chemotherapeutic agents.
197. The method of aspect 196, wherein the one or more chemotherapeutic agents are selected from: alkylating agents, cytoskeletal disruptors (e.g., taxanes), epothilones, histone deacetylase inhibitors, topoisomerase I inhibitors, topoisomerase II inhibitors, kinase inhibitors, nucleotide analog or precursor analogs, peptide antineoplastic antibiotics (e.g., bleomycin or actinomycin), platinum-based agents, retinoids, or vinca alkaloids and their derivatives.
198. The method of aspect 196, wherein the one or more chemotherapeutic agents are selected from the group consisting of actinomycin all-trans retinoic acid, azacytidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.
199. The method of any of aspects 135 to 140, wherein the MAPP comprises one or more independently selected IL-2, CD80, CD86, PD-L1, FasL, or 4-1BBL MOD (wt. or variant) MOD polypeptide sequences.
200. The method of any of aspects 187 to 192, wherein the disease or condition is an infection.
201. The method of aspect 200, wherein the disease is a viral infection.
202. The method of aspect 201, further comprising administering one or more (e.g., two or more) antiviral agents.
203. The method of aspect 202, wherein the one or more (e.g., two or more) antiviral agents is selected from: abacavir, acyclovir, adefovir, amantadine, amprenavir, arbidol, atazanavir, atripla, balavir, baloxavir marboxil, biktarvy, cidofovir, combivir, darunavir, delavirdine, descovy, didanosine, docosanol, dolutegravir, ecoliever, edoxudine, efavirenz, emtricitabine, enfuvirtide, entecavir, famciclovir, fomivirsen, fosamprenavir, foscarnet, fosfonet, fusion inhibitor, ibacitabine, idoxuridine, imiquimod, imunovir, indinavir, inosine, integrase inhibitor, interferon type i, interferon type ii, interferon type iii, interferon, lamivudine, lopinavir, loviride, maraviroc, methisazone, moroxydine, nelfinavir, nevirapine, nexavir, nitazoxanide, norvir, oseltamivir, peginterferon alfa-2a, penciclovir, peramivir, pleconaril, podophyllotoxin, protease inhibitor, pyramidine, raltegravir, reverse transcriptase inhibitor, ribavirin, rimantadine, rintatolimod, ritonavir, saquinavir, sofosbuvir, stavudine, synergistic enhancer, telaprevir, tenofovir alafenamide, tenofovir disoproxil, tenofovir, tipranavir, trifluridine, trizivir, tromantadine, truvada, valaciclovir, valganciclovir, vicriviroc, vidarabine, viramidine, zalcitabine, zanamivir, and zidovudine.
204. The method of any of aspects 187 to 192, wherein the disease is a bacterial, fungal, protozoan, or helminth infection.
205. The method of aspect 204, further comprising administering one or more antibiotic, chemotherapeutic, anti-fungal, and/or anti-helminth agent.
206. The method of any of aspects 204 to 205, wherein the MAPP comprises one or more independently selected IL-2, CD80, CD86, PD-L1, FasL, or 4-1BBL MOD or variant MOD polypeptide sequences.
207. The method of any of aspects 187 to 192, wherein the disease or condition is an autoimmune disease other than, or in addition to, celiac disease and/or T1D, and the epitope is an epitope of an autoantigen.
208. The method of aspect 207, wherein the autoimmune disease is selected from the group consisting of: Addison's disease, alopecia areata, ankylosing spondylitis, autoimmune encephalomyelitis, autoimmune hemolytic anemia, autoimmune hepatitis, autoimmune-associated infertility, autoimmune thrombocytopenic purpura, bullous pemphigoid, Crohn's disease, Goodpasture's syndrome, glomerulonephritis (e.g., crescentic glomerulonephritis, proliferative glomerulonephritis), Grave's disease, Hashimoto's thyroiditis, mixed connective tissue disease, multiple sclerosis, myasthenia gravis (MG), Pemphigus (e.g., Pemphigus vulgaris), pernicious anemia, polymyositis, psoriasis, psoriatic arthritis, rheumatoid arthritis, scleroderma, Sjögren's syndrome, systemic lupus erythematosus (SLE), vasculitis, and vitiligo.
209. The method of aspect 207, wherein the autoimmune disease is autoimmune gastritis.
210. The method of any of aspects 208 to 209, wherein the MAPP comprises:
   an MHC Class II alpha chain polypeptide having an α1 and α2 domain sequence and/or
   an MHC Class II alpha chain polypeptide having an β1 and β2 domain sequence correlated with an autoimmune disease set forth in FIG. 33.
211. The method of any of aspects 208 to 210, wherein the MAPP comprises an epitope of immunogen associated with the autoimmune disease set forth in FIG. 33.
212. The method of any of aspects 208 to 211, wherein the MAPP comprises one or more PD-L1 or FASL MOD or variant MOD polypeptide sequences.
213. The method of any of aspects 187 to 192, wherein the disease or condition is an allergy and the epitope is an epitope of an allergen.
214. The method of aspect 213, wherein the allergen is selected from: peanuts, tree nuts, plant pollens, latexes, and hymenoptera proteins (e.g., allergens in bee and wasp venoms such as phospholipase A2, melittin, “antigen 5” found in wasp venom, and hyaluronidases)
215. The method of aspect 213, wherein the allergen an allergic reactions to peanut allergens and the epitope is selected from PGQFEDFF, YLQGFSRN, FNAEFNEIRR, QEERGQRR, DITNPINLRE, NNFGKLFEVK, GNLELV, RRYTARLKEG, ELHLLGFGIN, HRIFLAGDKD, IDQIEKQAKD, KDLAFPGSGE, KESHFVSARP, NEGVIVKVSKEHVEELTKHAKSVSK, HASARQQWEL, QWELQGDRRC, DRRCQSQLER, LRPCEQHLMQ, KIQRDEDSYE, YERDPYSPSQ, SQDPYSPSPY, DRLQGRQQEQ, KRELRNLPQQ, QRCDLDVESG, IETWNPNNQEFECAG, GNIFSGFTPEFLAQA, VTVRGGLRILSPDRK, and DEDEYEYDEEDRRRG.
216. The method of any of aspects 214 to 215, further comprising administering an NSAID (e.g., Cox-1 and/or Cox-2 inhibitors such as celecoxib, diclofenac, diflunisal, etodolac, ibuprofen, indomethacin, ketoprofen, and naproxen).
217. The method of any of aspects 214 to 216, further comprising administering a corticosteroid (e.g., cortisone, dexamethasone, hydrocortisone, ethamethasoneb, fludrocortisone, methylprednisolone, prednisone, prednisolone and triamcinolone) before, during (concurrent or combined administration).
218. The method of any of aspects 214 to 217, further comprising administering an agent that block one or more actions of tumor necrosis factor alpha (e.g., an anti-TNF alpha such as golimumab, infliximab, certolizumab, adalimumab or a TNF alpha decoy receptor such as etanercept) (subject to the proviso that the MAPP or duplexed MAPP does not comprise tumor necrosis factor alpha MOD or variant MOD and/or an aa sequence to which the agent binds).
219. The method of any of aspects 214 to 218, further comprising administering one or more agents that bind to the IL-1 receptor competitively with IL-1 (e.g., anakinra) (subject to the proviso that the MAPP or duplexed MAPP does not comprise an IL-1 MOD or variant MOD and/or an aa sequence to which the agent binds).
220. The method of any of aspects 214 to 219, further comprising administering one or more agents that bind to the IL-6 receptor and inhibits IL-6 from signaling through the receptor (e.g., tocilizumab) subject to the proviso that the MAPP or duplexed MAPP does not comprise an IL-6 MOD or variant MOD and/or an aa sequence to which the agent binds).
221. The method of any of aspects 214 to 220, further comprising administering one or more agents that bind to CD80 and/or CD86 receptors and inhibit T cell proliferation and/or B cell immune response (e.g., abatacept) (subject to the proviso that the MAPP or duplexed MAPP does not comprise a CD80 and/or CD86 MOD or variant MOD and/or an aa sequence to which the agent binds).
222. The method of any of aspects 214 to 221, further comprising administering one or more agents that bind to CD20 resulting in B-Cell death (e.g., rituximab) (subject to the proviso that the MAPP or duplexed MAPP does not comprise a CD20 MOD or variant MOD, and/or an aa sequence to which the agent binds).
223. The method of any of aspects 187 to 222, wherein the MAPP or duplex MAPP, or the nucleic acid encoding a MAPP or duplex MAPP is administered in a composition comprising at least one pharmaceutical acceptable excipient.
224. A framework polypeptide of a MAPP or duplex MAPP according to any of aspects 1 to 186, optionally comprising an additional polypeptide.
225. A dimerization polypeptide of a MAPP or duplex MAPP, according to any of aspects 1 to 186, optionally comprising an additional polypeptide.
226. A nucleic acid sequence encoding the framework polypeptide of any of aspects 1 to 186, wherein the framework polypeptide optionally comprises an additional polypeptide.
227. A nucleic acid sequence encoding the dimerization polypeptide of any of aspects 1 to 186, wherein the dimerization polypeptide optionally comprises an additional polypeptide.
228. One or more nucleic acids comprising a nucleic acid sequence encoding a MAPP or duplex MAPP according to any of aspects 1-186.
229. The nucleic acid of any of aspects 226 to 228, wherein the nucleic acid sequence encoding the framework polypeptide and/or the dimerization polypeptide are operably linked to a promoter.
230. One or more nucleic acid molecules comprising sequences encoding a MAPP of any of embodiments 1 to 186.
231. A method of producing cells expressing a MAPP or duplex MAPP, the method comprising introducing one or more nucleic acid molecules according to aspect 230 into the cells in vitro; selecting for cells that produce the MAPP or duplex MAPP; and optionally selecting for cells comprising all or part of the one or more nucleic acids either unintegrated or integrated into at least one cellular chromosome.
232. The method of aspect 231, wherein the cell is a cell of a mammalian cell line is selected from the group consisting of: HeLa cells, CHO cells, 293 cells, Vero cells, NIH 3T3 cells, Huh-7 cells, BHK cells, PC12, COS cells, COS-7 cells, RAT1 cells, mouse L cells, human embryonic kidney (HEK) cells, and HLHepG2 cells.
233. A cell transiently or stably expressing a MAPP or duplex MAPP prepared by the method of aspect 230 or 231.
234. The cell of aspect 175, wherein the cells express from about 25 to about 350 (e.g., 20-50, 50-100, 100-200, 200-300, 300-350) mg/liter or more of the MAPP or duplex MAPP without a substantial reduction (less than a 5%, 10%, or 15% reduction) in cell viability relative to otherwise identical cells not expressing the MAPP or duplex MAPP.
235. A method of selectively delivering one or more MOD polypeptides and/or variant MOD polypeptides to a cell, tissue, patient or subject, the method comprising:
(i) contacting (e.g., administering) a cell, tissue, patient or subject (e.g., a patient in need thereof) an effective amount of one or more MAPPs, duplex MAPPs, or higher order MAPP complexes of any of aspects 1 to 185, or a pharmaceutical composition comprising of aspect 186;
(ii) contacting (e.g., administering) a cell, tissue, patient or subject (e.g., a patient in need thereof) an effective amount of one or more nucleic acids encoding a MAPP, duplex MAPP, or higher order MAPP complex of any of aspects 1 to 185;
(iii) contacting a cell or tissue, either in vitro or in vivo, with one or more MAPPs duplex MAPPs, or higher order MAPP complexes of any of aspects 1 to 185, and administering the cell, tissue, or progeny thereof to the patient/subject; or
(iv) contacting a cell or tissue, either in vitro or in vivo, with one or more nucleic acids encoding a MAPP, duplex MAPP, or higher order MAPP complex of any of aspects 1 to 185, and administering the cell, tissue, or progeny thereof to the patient/subject.
236. The method of aspect 235, wherein the one or more MOD polypeptides and/or variant MOD polypeptide sequences are selected independently from the group consisting of: 4-1BBL, PD-L1, IL-2, CD80, CD86, OX40L (CD252), Fas ligand (FasL), ICOS-L, ICAM, CD30L, CD40, CD83, HVEM (CD270), JAG1 (CD339), CD70, CD80, CD86, TGF-β1, TGF-β2, and TGF-β3 MOD or variant MOD polypeptide sequences.
237. The method of aspect 235, wherein the one or more MOD polypeptides and/or variant MOD polypeptide sequences are selected independently from the group consisting of: 4-1BBL, PD-L1 IL-2, CD80, CD86 and FasL MOD and variant MOD polypeptide sequences of any thereof.
238. The method of aspect 235, wherein the one or more MAPPs, duplex MAPPs, or higher order MAPP complex's comprise at least one IL-2 MOD or variant MOD polypeptide sequence, and at least one CD80, CD86, variant CD80 or variant CD86 polypeptide sequence.
239. The method of aspect 177, wherein the one or more MAPPs, duplex MAPPs, or higher order MAPP complexe comprise at least one IL-2 MOD or IL-2 variant MOD polypeptide sequence, or at least one pair of IL-2 MOD or IL-2 variant MOD polypeptide sequences in tandem.
240. The method of aspect 172, wherein the one or more MAPPs, duplex MAPPs, or higher order MAPP complex's comprise at least one CD80 and/or CD86 MOD or one CD80 and/or CD86 MOD variant MOD polypeptide sequence.
241. The method of aspect 177, wherein the one or more MAPPs, duplex MAPPs, or higher order MAPP complexe comprise at least one PD-L1 MOD or variant MOD polypeptide sequence.
242. The method of aspect 177, wherein the one or more MAPPs, duplex MAPPs, or higher order MAPP complex's comprise at least one FasL MOD or variant FasL MOD polypeptide sequence.

1. A multimeric antigen-presenting polypeptide (MAPP) comprising:
(i) a framework polypeptide comprising a dimerization sequence and a multimerization sequence;
(ii) a dimerization polypeptide comprising a counterpart dimerization sequence complementary to the dimerization sequence of the framework polypeptide, the dimerization sequence and counterpart dimerization sequence dimerizing through covalent and/or non-covalent interactions to form a MAPP heterodimer; and
(iii) at least one presenting sequence and/or presenting complex;
wherein
(a) each presenting sequence comprises (i) a peptide epitope, and (ii) MHC class II α1, α2, β1, and β2 domain polypeptide sequences,
(b) each presenting complex comprises a presenting complex 1^{st} sequence and a presenting complex 2^{nd} sequence, wherein
the presenting complex 1^{st} sequence or presenting complex 2^{nd} sequence comprises a peptide epitope and at least one of the α1, α2, β1, and β2 domain polypeptide sequences, and
the presenting complex 1^{st} sequence and presenting complex 2^{nd} sequence together comprise a T1D peptide epitope and MHC Class II α1, α2, β1, and β2 domain polypeptide sequences,
(c) one or both of the dimerization polypeptide and/or the framework polypeptide comprises a presenting sequence or a presenting complex 1^{st} sequence, and
(d) at least one framework polypeptide, dimerization peptide, presenting sequence, or presenting complex comprises one or more independently selected MOD and/or variant MOD polypeptide sequences; and
wherein the framework polypeptide, dimerization polypeptide, presenting sequence, presenting complex 1^{st} sequence and/or presenting complex 2^{nd} sequence optionally comprise one or more independently selected linker sequences.

2. The MAPP of claim 1 comprising:
a framework polypeptide that comprises from N-terminus to C-terminus a dimerization sequence and a multimerization sequence; and
a dimerization polypeptide that comprises a counterpart dimerization sequence complementary to the dimerization sequence of the framework polypeptide, and dimerizing therewith through covalent and/or non-covalent interactions to form a MAPP heterodimer; and
at least one presenting sequence.

3. The MAPP of claim 1 comprising:
a framework polypeptide that comprises from N-terminus to C-terminus a dimerization sequence and a multimerization sequence;
a dimerization polypeptide that comprises a counterpart dimerization sequence complementary to the dimerization sequence of the framework polypeptide, and dimerizing therewith through covalent and/or non-covalent interactions to form a MAPP heterodimer; and
at least one presenting complex.

4. The MAPP of any of claims 1-3, wherein at least one presenting sequence or presenting complex comprises:
α1 and α2 domain polypeptide sequences each having 85% to 100% sequence identity to at least about 60 contiguous aas of a HLA DR alpha (DRA), DM alpha (DMA), DO alpha (DOA), DP alpha 1 (DPA1), DQ alpha 1 (DQA1), or DQ alpha 2 (DQA2) polypeptide sequence provided in any one of SEQ ID NOs:17, 62, 64, 66, 67, or 89-91 (see FIG. 4, 9, 11, 13, 15, or 16), wherein the α1 and α2 domain polypeptide sequences do not include a transmembrane domain, or a portion thereof, that will anchor the MAPP in a cell membrane; and
β1 and β2 domain polypeptide sequences each having 85% to 100% sequence identity to at least about 60 contiguous aas of a HLA DR beta 1 (DRB1), DR beta 3 (DRB3), DR beta 4 (DRB4), DR beta 5 (DRB5), DM beta (DMB), DO beta (DOB), DP beta 1 (DPB1), DQ beta 1 (DQB1), or DQ beta 2 (DQB2) polypeptide sequence provided in any one of SEQ ID NOs:18-61, 63, 65, 68-79, or 92-105 (see FIG. 5, 6, 7, 8, 10, 12, 14, 17 or 18), wherein the β1 and β2 domain polypeptide sequences do not include a transmembrane domain, or a portion thereof, that will anchor the MAPP in a cell membrane.

5. The MAPP of claim 4, wherein at least one presenting sequence or at least one presenting complex comprises:
α1 and α2 domain polypeptide sequences each having at least 90% or 100% sequence identity to all or at least about 50 contiguous aas of a HLA DR alpha (DRA) α1 and/or α2 domain polypeptide sequence of SEQ ID NO:17 (see FIG. 4); and
β1 and β2 domain polypeptide sequences each having at least 90% (e.g., at least 95% or 98%) or 100% sequence identity to all or at least about 50 contiguous aas of a HLA DR beta 3 (DRB3), DR beta 4 (DRB4), and DR beta 5 (DRB5) β1 and/or β2 domain polypeptide sequences provided in any one of SEQ ID NOs: 55-61 (see FIG. 6, 7, or 8).

6. The MAPP of claim 4, comprising:
(i) a cysteine-containing linker, wherein the cysteine residue in the linker forms a disulfide bond between a presenting sequence and another polypeptide of the MAPP, or between a presenting complex 1st sequence and another polypeptide of the MAPP;
(ii) at least one presenting sequence or a presenting complex comprising a disulfide bond formed between one of the MHC α1 or α2 domain polypeptide sequence and one of the 31 or β2 domain polypeptide sequences;
(iii) at least one presenting sequence or a presenting complex comprising a disulfide bond formed between cysteines positioned at
α chain position 3 and β chain position 19 or 20,
α chain position 4 and β chain position 19 or 20,
α chain position 28 and β chain position 151, 152, or 153,
α chain position 29 and β chain position 151, 152, or 153,
α chain position 80, 81, or 82 and β chain position 33,
α chain position 93 and β chain position 153 of 156,
α chain position 94 and β chain position 120 or 156, or
α chain position 95 and β chain position 120 or 156; and/or
(iv) at least one presenting sequence or a presenting complex comprising a disulfide bond formed between cysteines positioned at
α chain position 12 and β chain position 7 or 10,
α chain position 80 and β chain position 5 or 7,
α chain position 81 and β chain position 5 or 7, or
α chain position 82 and β chain position 5 or 7.

7. The MAPP of claim 4, comprising at least one presenting sequence or at least one presenting complex that comprises a cysteine-containing polypeptide linker having the structure (aa1-aa2-aa3-aa4-aa5-[remainder of linker or a bond]) that connects an epitope and a β1 domain polypeptide sequence such that the at least one presenting sequence or at least one presenting complex comprises a substructure of the form
{epitope-aa1-aa2-aa3-aa4-aa5-[remainder of linker if present]-β1 domain},
and wherein the presenting sequence or presenting complex comprises a disulfide bond between a cysteine located at any of aa1 to aa5 and an aa in an MHC α chain polypeptide sequence.

8. The MAPP of claim 7, wherein the disulfide bond between a cysteine located at any of aa1 to aa5 and an MHC α chain polypeptide sequence is between a cysteine located at any of aa1 to aa5 and MHC α chain sequence comprising a cysteine at position 72 or 75.

9. The MAPP of claim 4, wherein the MAPP comprises a presenting sequence comprising, in the N-terminal to C-terminal direction:
a) the peptide epitope, the β1, α1, α2, and β2 domain polypeptide sequences;
b) the peptide epitope, the β1, β2, α1, and α2 domain polypeptide sequences;
or
c) the peptide epitope, the α1, α2, β1, and β2 domain polypeptide sequences;
wherein the presenting sequence optionally comprises one or more MOD or variant MOD polypeptide sequences; and
wherein said presenting sequence optionally comprises one or more independently selected linker sequences.

10. The MAPP of claim 4, comprising at least one presenting complex, wherein the at least one presenting complex comprises a presenting complex 1^{st} sequence and presenting complex 2^{nd} sequence, and wherein:
(i) the presenting complex 1^{st} sequence comprises the α1 domain polypeptide sequence, and its associated presenting complex 2^{nd} sequence comprises the peptide epitope sequence and the β1 domain polypeptide sequence;
(ii) the presenting complex 1^{st} sequence comprises the α2 domain polypeptide sequence, and its associated presenting complex 2^{nd} sequence comprises the peptide epitope sequence and the β2 domain polypeptide sequence:
(iii) the presenting complex 1^{st} sequence comprises the β1 domain polypeptide sequence, and its associated presenting complex 2^{nd} sequence comprises the peptide epitope sequence and the α1 domain polypeptide sequence;
(iv) the presenting complex 1^{st} sequence comprises the β2 domain polypeptide sequence, and its associated presenting complex 2^{nd} sequence comprises the peptide epitope sequence and the α2 domain polypeptide sequence;
(v) the presenting complex 1^{st} sequence comprises the α1 domain polypeptide sequence, and its associated presenting complex 2^{nd} sequence comprises the peptide epitope sequence and the β1 and β2 domain polypeptide sequences;
(vi) the presenting complex 1^{st} sequence comprises the α2 domain polypeptide sequence, and its associated presenting complex 2^{nd} sequence comprises the peptide epitope sequence and the β1 and β2 domain polypeptide sequences;
(vii) the presenting complex 1^{st} sequence comprises the α1 and/or α2 domain polypeptide sequences, and its associated presenting complex 2^{nd} sequence comprises the peptide epitope sequence and the β1 and β2 domain polypeptide sequences;
(viii) the presenting complex 1^{st} sequence comprises the 31 and/or β2 domain polypeptide sequences, and its associated presenting complex 2^{nd} sequence comprises the peptide epitope sequence and the α1 and α2 domain polypeptide sequences;
(ix) the presenting complex 1^{st} sequence comprises the peptide epitope sequence and the α1 domain polypeptide sequence;
(x) the presenting complex 1^{st} sequence comprises the peptide epitope sequence and the α2 domain polypeptide sequence, and its associated presenting complex 2^{nd} sequence comprises the β2 domain polypeptide sequence;
(xi) the presenting complex 1^{st} sequence comprises the peptide epitope sequence and the β1 domain polypeptide sequence, and its associated presenting complex 2^{nd} sequence comprises the α1 domain polypeptide sequence;
(xii) the presenting complex 1^{st} sequence comprises the peptide epitope sequence and the β2 domain polypeptide sequence, and its associated presenting complex 2^{nd} sequence comprises the α2 domain polypeptide sequence;
(xiii) the presenting complex 1^{st} sequence comprises the peptide epitope sequence and the α1 domain polypeptide sequence;
(xiv) the presenting complex 1^{st} sequence comprises the peptide epitope sequence and the α2 domain polypeptide sequence, and its associated presenting complex 2^{nd} sequence comprises the β1 and β2 domain polypeptide sequences;
(xv) the presenting complex 1^{st} sequence comprises the peptide epitope sequence and the α1 and/or α2 domain polypeptide sequences, and its associated presenting complex 2^{nd} sequence comprises the β1 and β2 domain polypeptide sequences or
(xvi) the presenting complex 1^{st} sequence comprises the peptide epitope sequence and the 31 and/or β2 domain polypeptide sequences, and its associated presenting complex 2^{nd} sequence comprises the α1 and α2 domain polypeptide sequences;
wherein the at least one presenting complex optionally comprises one or more, or two more MODs or variant MODs.

11. The MAPP of claim 4, wherein the dimerization and multimerization sequences are independently selected non-interspecific sequences or interspecific sequences.

12. The MAPP of claim 11, wherein
the non-interspecific sequences are selected from the group consisting of immunoglobulin heavy chain constant regions (Ig Fc), collectin family, coiled-coil domains, and leucine-zipper domains; and
the interspecific sequences are selected from the group consisting of Fos polypeptides that pair with Jun polypeptides, Ig CH1 and Ig C_{L} κ, Ig CH1 and Ig C_{L}λ, knob-in-hole without disulfide (“KiH”), knob-in hole with a stabilizing disulfide bond (“KiHs-s”), HA-TF, ZW-1, 7.8.60, DD-KK, EW-RVT, EW-RVTs-s, and A107 sequences.

13. The MAPP of claim 11, complexed to form a duplex or higher order MAPP comprising at least a first MAPP heterodimer and a second MAPP heterodimer wherein:
(i) the first heterodimer comprises a first framework polypeptide having a first multimerization sequence and a first dimerization sequence, and a first dimerization polypeptide having a first counterpart dimerization sequence complementary to the first dimerization sequence; and
(ii) the second heterodimer comprises a second framework polypeptide having a second multimerization sequence and a second dimerization sequence, and a second dimerization polypeptide having a second counterpart dimerization sequence complementary to the second dimerization sequence; and
wherein the first and second framework polypeptides are associated by binding interactions between the first and second multimerization sequences optionally including one or more interchain covalent bonds, and the multimerization sequences are not the same as, and do not substantially associate with or bind to, the dimerization sequences or counterpart dimerization sequences.

14. The duplex MAPP of claim 13, wherein the multimerization sequences comprise:
(i) an Ig Fc region and the first and second dimerization sequences comprise independently selected Ig CH1, Ig C_{L} κ or λ, leucine zipper, Fos or Jun domains;
(ii) an IgFc region and the first and second dimerization sequences comprise independently selected Ig CH1 or Ig C_{L} κ or λ, domains;
(iii) an Ig Fc region selected from the group consisting of the IgA, IgD, IgE, IgG and IgM Fc regions having at least about 70%, at least about 80%, at least about 90%, at least about 95%, at least about 98%, at least about 99%, or 100% aa sequence identity to an aa sequence of the CH2 and/or CH3 domains of an Fc region of SEQ ID NOs: 1-12 (provided in FIGS. 2A-2H).
(iv) IgG1, IgG2, IgG3, and IgG4 CH2-CH3 domains having at least about 70%, at least about 80%, at least about 90%, at least about 95%, at least about 98%, at least about 99%, or 100% aa sequence identity to an aa sequence of the CH2 and/or CH3 domains of an Fc region of SEQ ID NOs: 4-12;
(v) IgG1 CH2-CH3 domains having at least about 70%, at least about 80%, at least about 90%, at least about 95%, at least about 98%, at least about 99%, or 100% aa sequence identity to an aa sequence of the CH2 and/or CH3 domains of an Fc region of SEQ ID NOs: 4-12;
(vi) interspecific immunoglobulin sequences selected from the group consisting of KiH pairs, KiHs-s pairs, HA-TF polypeptide pairs, ZW-1 polypeptide pairs, 7.8.60 polypeptide pairs, DD-KK polypeptide pairs, EW-RVT polypeptide pairs, EW-RVTs-s polypeptide pairs, and A107 polypeptide pairs;
(vii) a pair of interspecific KiH, or KiHs-s sequences; or
(viii) a pair of interspecific immunoglobulin sequences, and wherein the first and second dimerization sequences comprise independently selected Ig CH1, Ig C_{L} κ or λ, leucine zipper, Fos or Jun domains;
wherein when the multimerization sequences comprise an IgFc region the IgFc regions optionally comprise one or more substitutions that limit complement dependent cytotoxicity (CDC) and/or antibody-dependent cellular cytotoxicity (ADCC).

15. The duplex MAPP of claim 14, wherein the first dimerization sequence and its counterpart dimerization sequence and/or the second dimerization sequence and its counterpart dimerization sequence are covalently linked by at least one disulfide bond; and the multimerization sequences of the first and second framework polypeptides are covalently linked by at least one disulfide bond, and optionally at least two disulfide bonds.

16. The duplex MAPP of claim 15, wherein when a framework or dimerization polypeptide of the MAPP or duplex MAPP comprises one or more IgFc regions, at least one of the one or more IgFc regions comprises one or more substitutions at L234, L235, G236, G237, P238, 5239, D270, N297, K322, P329, and/or P331 (respectively, aas L14, L15, G16, G17, P18, S19, N77, D50, K102, P109, and P111 of the wt. IgG1 aa sequence of SEQ ID NO:4 (provided in FIG. 2D).

17. The duplex MAPP of claim 13, comprising at least one MOD, at least one variant MOD, or at least one pair of MODs and/or one pair of variant MODs in tandem located at one or more of positions 1, 1′, 2, 2′, 3, 3′, 4, 4′,4″, 4′″, 5, and/or 5′.

18. The duplex MAPP of claim 17, comprising:
(i) at least one MOD or variant MOD polypeptide sequence selected independently from the group consisting of IL-1, IL-2, IL-4, IL-6, IL-7, IL-10, IL-12, IL-15, IL-17, IL-21, IL-23, CD7, CD30L, CD40, CD70, CD80, (B7-1), CD83, CD86 (B7-2), HVEM (CD270), ILT3 (immunoglobulin-like transcript 3), ILT4 (immunoglobulin-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, and 4-1BBL polypeptide sequences;
(ii) at least one MOD or variant MOD polypeptide sequence selected independently from the group consisting of 4-1BBL, PD-L1 IL-2, CD80, CD86 and FasL MOD or variant MOD polypeptide sequences;
(iii) at least one IL-2 MOD or variant IL-2 MOD polypeptide sequence, or at least one pair of IL-2 MOD or at least one pair of variant MOD polypeptide sequences in tandem, optionally located at position 1 or 1′ of the duplex MAPP;
(iv) at least one CD80, variant CD80, CD86, or variant CD86 MOD polypeptide sequence;
(v) at least one IL-2 MOD or variant IL2 MOD polypeptide sequence; wherein at least one of said IL-2 MOD or variant IL2 MOD polypeptide sequences is optionally located at position 1 or 1′ of the duplex MAPP;
(vi) at least one CD80, variant CD80, CD86, or variant CD86 MOD polypeptide sequence, which is optionally located at position 1 or 1′ of the duplex MAPP;
(vii) at least one PD-L1 MOD or variant MOD polypeptide sequence, which is optionally located at position 1 or 1′ of the duplex MAPP; or
(viii) at least one FasL MOD or variant MOD polypeptide sequence.

19. The duplex MAPP of claim 18, wherein:
(i) the epitope is a cancer epitope from about 4 to 25 aas in length, and the MAPP optionally comprises a cancer targeting polypeptide as part of at least one framework and/or dimerization polypeptide;
(ii) the epitope is an epitope presented by an infectious agent from about 4 to 25 aas in length;
(iii) the epitope is an epitope of an autoantigen other than, or in addition to, an epitope associated with a T1D or celiac antigen from about 4 to 25 aas in length; or
(iv) the epitope is an epitope of an allergen from about 4 to 25 aas in length.

20. The MAPP or duplex MAPP of claim 19, wherein the cancer epitope is an Alpha Feto Protein (AFP) epitope; a Wilms Tumor Antigen (WT-1) protein epitope; a Human Papilloma Virus I (HPV) epitope; or a Hepatitis B Virus (HBV) epitope.

21. A method of treatment or prophylaxis of a disease or condition comprising:
(i) administering to a patient/subject (e.g., a patient in need thereof) an effective amount of one or more duplex MAPPs of claim 13;
(ii) administering to a patient/subject (e.g., a patient in need thereof) an effective amount of one or more nucleic acids encoding one or more duplex MAPPs of claim 13;
(iii) contacting a cell or tissue in vitro or in vivo with one or more duplex MAPPs of claim 13, and administering the cell, tissue, or progeny thereof to the patient/subject; or
(iv) contacting a cell or tissue in vitro or in vivo with one or more nucleic acids encoding one or more duplex MAPPs of claim 13, and administering the cell, tissue, or progeny thereof to the patient/subject; wherein the patient or subject is selected from a mammalian patient or subject, a human, or a non-human mammal.

22. The method of claim 21, wherein the disease or condition is a cancer, the epitope is a cancer epitope, and wherein when the one or more duplex MAPPs comprises a targeting sequence it is a CTP.

23. The method of claim 22, wherein the epitope is an epitope of a HPV, HBV, AFP or WT-1 protein.

24. The method of claim 21, wherein the disease or condition is: (i) a viral infection, or (ii) a bacterial, fungal, protozoan infection, or (iii) a helminth infection.

25. The method of claim 21, wherein the disease or condition is an autoimmune disease other than, or in addition, to celiac disease and/or TID, and the epitope is an autoantigen, wherein the autoimmune disease is optionally selected from the group consisting of: Addison's disease, alopecia areata, ankylosing spondylitis, autoimmune encephalomyelitis, autoimmune hemolytic anemia, autoimmune hepatitis, autoimmune-associated infertility, autoimmune thrombocytopenic purpura, bullous pemphigoid, Crohn's disease, Goodpasture's syndrome, glomerulonephritis, Grave's disease, Hashimoto's thyroiditis, mixed connective tissue disease, multiple sclerosis, myasthenia gravis (MG), Pemphigus, pernicious anemia, polymyositis, psoriasis, psoriatic arthritis, rheumatoid arthritis, scleroderma, Sjögren's syndrome, systemic lupus erythematosus (SLE), vasculitis, and vitiligo.

26. One or more nucleic acid sequence encoding the framework polypeptide and/or the dimerization polypeptide of a MAPP of any of claims 1-3, wherein the framework polypeptide and/or the dimerization polypeptide optionally comprise an additional polypeptide.

27. A method of producing cells expressing a MAPP or duplex MAPP, the method comprising introducing one or more nucleic acids according to claim 26 into the cells in vitro; selecting for cells that produce the MAPP or duplex MAPP; and optionally selecting for cells comprising all or part of the one or more nucleic acids either unintegrated or integrated into at least one cellular chromosome; wherein the cells are optionally selected from the group consisting of: HeLa cells, CHO cells, 293 cells, Vero cells, NIH 3T3 cells, Huh-7 cells, BHK cells, PC12, COS cells, COS-7 cells, RATI cells, mouse L cells, human embryonic kidney (HEK) cells, and HLHepG2 cells.

28. Cells transiently or stably expressing a MAPP or duplex MAPP prepared by the method of claim 27, wherein optionally the cells express from about 25 to about 350 mg/liter or more of the duplex MAPP without a substantial reduction in cell viability relative to otherwise identical cells not expressing the MAPP or duplex MAPP.

29. A method of selectively delivering one or more MOD polypeptides and/or variant MOD polypeptides to a cell, tissue, patient or subject, the method comprising:
(i) administering to a patient/subject an effective amount of one or more duplex MAPPs of claim 13;
(ii) administering to a patient/subject an effective amount of one or more nucleic acids encoding a duplex MAPP according to claim 13;
(iii) contacting a cell or tissue in vitro or in vivo with one or more duplex MAPPs of claim 13, and optionally administering the cell, tissue, or progeny thereof to the patient/subject; or
(iv) contacting a cell or tissue in vitro or in vivo with one or more nucleic acids encoding a duplex MAPP of claim 13, and optionally administering the cell, tissue, or progeny thereof to the patient/subject;
wherein the duplex MAPP comprises one or more MODs and/or one or more variant MODs.
